(19) United States
(12) Patent Application Publication
(10) Pub. No.: US 2021/0171589 A1
(43) Pub. Date: Jun. 10, 2021

(54) TRUNCATED CARTILAGE-HOMING PEPTIDES AND PEPTIDE COMPLEXES AND METHODS OF USE THEREOF

(71) Applicant: BLAZE BIOSCIENCE, INC., Seattle, WA (US)

(72) Inventors: Natalie Winblade NAIRN, Seattle, WA (US); Scott PRESNELL, Tacoma, WA (US); Claudia JOCHHEIM, Seattle, WA (US); Mark STROUD, Seattle, WA (US)

(21) Appl. No.: 16/980,787

(22) PCT Filed: Mar. 15, 2019
(86) PCT No.: PCT/US19/22630

§ 371 Date: Sep. 14, 2020

Related U.S. Application Data
(60) Provisional application No. 62/644,329, filed on Mar. 16, 2018.
(60) Provisional application No. 62/676,033, filed on May 24, 2018.

(51) Int. Cl. C07K 14/47; A61K 47/64; A61K 47/65; A61K 51/08; A61P 19/04; A61P 19/02; A61P 13/12; A61K 49/00; A61K 9/00

(52) U.S. Cl. CPC C07K 14/47; A61K 47/64; A61K 47/65; A61K 51/088; A61P 19/04; A61K 38/00; A61P 13/12; A61K 49/0056; A61K 9/0019; C07K 2319/30; C07K 2319/50; A61P 19/02

(57) ABSTRACT

Truncated, variant, or mutated peptides that home, target, migrate to, are directed to, are retained by, or accumulate in and/or bind to the cartilage or kidney of a subject are disclosed. Pharmaceutical compositions and uses for truncated or mutated peptides or truncated or mutated peptide-active agent complexes comprising such peptides are also disclosed. Such compositions can be formulated for targeted delivery of an active agent to a target region, tissue, structure or cell in the cartilage. Targeted compositions of the disclosure can deliver truncated or mutated peptide or truncated, variant, or mutated peptides or peptide-active agent complexes to target regions, tissues, structures or cells targeted by the peptide.

### CROSS-REFERENCE

This application claims the benefit of U.S. Provisional Application No. 62/644,329 filed Mar. 16, 2018, and U.S. Provisional Application No. 62/676,033 filed May 24, 2018, which are incorporated herein by reference in their entireties for all purposes.

### SEQUENCE LISTING

The instant application contains a Sequence Listing which has been submitted electronically in ASCII format and is hereby incorporated by reference in its entirety. Said ASCII copy, created on Mar. 13, 2019, is named 45639-716_601_SL.txt and is 98,451 bytes in size.

### BACKGROUND

Cartilage comprises chondrocytes, a specialized cell-type which produces components of the extracellular matrix, mainly including collagen, proteoglycans (e.g., aggrecan), and elastic fibers. The extracellular matrix proteins provide support, cushion, and durability to cartilage-rich portions of the body such as joints, ears, nose and windpipe. Cartilage is one of few tissues in the body which does not contain blood vessels and is considered an avascular tissue. Unlike many cells in the body which rely on a combination of blood flow and diffusion, chondrocytes rely on diffusion. Because it does not have a direct blood supply, compared to other connective tissues, cartilage grows and repairs much more slowly. As a result, cartilage disorders are particularly difficult to treat.

### SUMMARY

In various aspects, the present disclosure provides a peptide comprising: a) any one of SEQ ID NO: 87, SEQ ID NO: 88, SEQ ID NO: 107, SEQ ID NO: 108, SEQ ID NO: 219, SEQ ID NO: 223, SEQ ID NO: 225, or a functional fragment thereof, or b) any one of SEQ ID NO: 89, SEQ ID NO: 106, and SEQ ID NO: 221, or a functional fragment thereof and further comprising at least one amino acid in each of SEQ ID NO: 89, SEQ ID NO: 106, and SEQ ID NO: 221 is selected from the group consisting of: i) for SEQ ID NO: 89, wherein X¹ is selected from N, S, or G, wherein X² is selected from L or Y, wherein X³ is selected from D or E, wherein X⁴ is selected from M or T, wherein X⁵ is selected from N, Q, A, S, T, or L, wherein X⁶ is selected from S, G, or R, wherein X⁷ is selected from H or Y, and wherein X⁸ is selected from T or Y; ii) for SEQ ID NO: 106, wherein X¹ is selected from L or Y, wherein X² is selected from D or E, wherein X³ is selected from M or T, wherein X⁴ is selected from N, Q, A, S, T, or L, wherein X⁵ is selected from S, G, or R, wherein X⁶ is selected from H or Y, and wherein X⁷ is selected from T or Y; and iii) for SEQ ID NO: 221, wherein each X and X₁₋₁₃ are individually any amino acid or no amino acid and at least one of the following residues at the denoted position, more than one of the following residues at the denoted position, or all of the following residues at the denoted position is included in SEQ ID NO: 221: X₁ is K, X₂ is Q, X₃ is Y, X₄ is E, X₅ is K, X₆ is T, X₇ is K, X₈ is M, X₉ is Q, A, S, T, or L, X₁₀ is G, X₁₁ is K, X₁₂ is Y, or X₁₃ is Y; or (iv) a corresponding substitution selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

In some aspects, the peptide comprises any one of SEQ ID NO: 89, SEQ ID NO: 106, and SEQ ID NO: 221, or a functional fragment thereof, and wherein the peptide does not comprise SEQ ID NO: 128 or SEQ ID NO: 149. In some aspects, the peptide comprises a sequence of any one of SEQ ID NO: 89, SEQ ID NO: 106, and SEQ ID NO: 221 and further comprises at least one of KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); or GRCIXXRC (SEQ ID NO: 228) wherein each X can independently be any amino acid or amino acid analogue or null; GRCIX¹X²RC (SEQ ID NO: 229), wherein X=N, Q, A, S, T or L and X²=S, G, or R wherein each X can independently be any amino acid or amino acid analogue or null.

In some aspects, the peptide comprises a sequence of any one of SEQ ID NO: 89, SEQ ID NO: 106, and SEQ ID NO: 221 and the peptide further comprises at least one of RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); or RFGRCI (SEQ ID NO: 259).

In some aspects, the peptide has a sequence selected from the group consisting of any one of SEQ ID NO: 106, and SEQ ID NO: 221.

In other aspects, the peptide comprises SEQ ID NO: 89 and wherein the peptide comprises one or more of the following characteristics: a) X1 is selected from S, or G; b) X2 is selected from Y; c) X3 is selected from E; d) X4 is selected from T; e) X5 is selected from Q, A, S, T, or L; f) X6 is selected from G, or R; g) X7 is selected from Y; or h) X8 is selected from Y.

In some aspects, the peptide comprises two, three, four, five, six, seven or more of the characteristics, or wherein the peptide comprises all of the characteristics. In some aspects, the peptide consists of any one of SEQ ID NO: 89, SEQ ID NO: 106, and SEQ ID NO: 221 and wherein the peptide does not comprise SEQ ID NO: 128 or SEQ ID NO: 149.

In some aspects, the peptide is any of the above peptides, wherein a) the peptide comprises at least 70%, at least at least 75%, at least 80%, at least 85%, at least 90%, at least 95%, at least 97%, at least 98%, at least 99%, or 100% sequence identity with a sequence selected from the group SEQ ID NO: 111-SEQ ID NO: 126 or SEQ ID NO: 233-SEQ ID NO: 240; or b) the peptide comprises at least 70%, at least at least 75%, at least 80%, at least 85%, at least 90%, at least 95%, at least 97%, at least 98%, at least 99%, or 100% sequence identity with a sequence selected from the group SEQ ID NO: 134-SEQ ID NO: 148 and SEQ ID NO: 249-SEQ ID NO: 256.

In some aspects, the peptide is any of the above peptides, wherein a) the peptide comprises a sequence that has at least 70%, at least 75%, at least 80%, at least 85%, at least 90%, at least 92%, at least 95%, at least 97%, or at least 100% sequence identity with any one of SEQ ID NO: 109, SEQ ID NO: 110, SEQ ID NO: 260, or SEQ ID NO: 262 and wherein the peptide further comprises a joining sequence of one or more amino acid residues located immediately adjacent to an N-terminus or a C-terminus of the peptide and wherein i) N−1 is selected from the group consisting of G, A, L, I, M, F, W, P, S, T, C, Y, N, Q, D, E, K, R, and H; ii) N−2 is selected from the group consisting of G, A, V, L, I, M, F, W, P, S, T, C, Y, Q, D, E, K, R, and H; iii) N−3 is selected from the group consisting of G, A, V, L, M, F, W, P, S, T, C, Y, N, Q, D, E, K, R, and H; iv) N−4 is selected from the group consisting of G, A, V, L, I, M, F, W, S, T, C, Y, N, Q, D, E, K, R, and H; v) N−5 is selected from the group consisting of G, A, L, I, M, F, W, P, S, T, C, Y, N, Q, D, E, K, R, and H; or vi) N−6 is selected from the group consisting of A, V, L, I, M, F, W, P, S, T, C, Y, N, Q, D, E, K, R, and H; b) the peptide comprises a sequence that has at least 70%, at least 75%, at least 80%, at least 85%, at least 90%, at least 92%, at least 95%, at least 97%, or at least 100% sequence identity with SEQ ID NO: 129 and wherein the peptide further comprises a joining sequence of one or more amino acid residues located immediately adjacent to an N-terminus or a C-terminus of the peptide and wherein N−1 is selected from the group consisting of A, V, L, I, M, F, W, P, S, T, C, Y, N, Q, D, E, K, R, and H; c) the peptide comprises a sequence that has at least 70%, at least 75%, at least 80%, at least 85%, at least 90%, at least 92%, at least 95%, at least 97%, or at least 100% sequence identity with SEQ ID NO: 130 and wherein the peptide further comprises a joining sequence of one or more amino acid residues located immediately adjacent to an N-terminus or a C-terminus of the peptide and wherein i) N−1 is selected from the group consisting of G, A, L, I, M, F, W, P, S, T, C, Y, N, Q, D, E, K, R, and H; or ii) N−2 is selected from the group consisting of A, V, L, I, M, F, W, P, S, T, C, Y, N, Q, D, E, K, R, and H; d) the peptide comprises a sequence that has at least 70%, at least 75%, at least 80%, at least 85%, at least 90%, at least 92%, at least 95%, at least 97%, or at least 100% sequence identity with SEQ ID NO: 131 and wherein the peptide further comprises a joining sequence of one or more amino acid residues located immediately adjacent to an N-terminus or a C-terminus of the peptide and wherein i) N−1 is selected from the group consisting of G, A, V, L, I, M, F, W, S, T, C, Y, N, Q, D, E, K, R, and H; ii) N−2 is selected from the group consisting of G, A, L, I, M, F, W, P, S, T, C, Y, N, Q, D, E, K, R, and H; or iii) N−3 is selected from the group consisting of A, V, L, I, M, F, W, P, S, T, C, Y, N, Q, D, E, K, R, and H; e) the peptide comprises a sequence that has at least 70%, at least 75%, at least 80%, at least 85%, at least 90%, at least 92%, at least 95%, at least 97%, or at least 100% sequence identity with SEQ ID NO: 132 and wherein the peptide further comprises a joining sequence of one or more amino acid residues located immediately adjacent to an N-terminus or a C-terminus of the peptide and wherein i) N−1 is selected from the group consisting of G, A, V, L, M, F, W, P, S, T, C, Y, N, Q, D, E, K, R, and H; ii) N−2 is selected from the group consisting of G, A, V, L, I, M, F, W, S, T, C, Y, N, Q, D, E, K, R, and H; iii) N−3 is selected from the group consisting of G, A, L, I, M, F, W, P, S, T, C, Y, N, Q, D, E, K, R, and H; or iv) N−4 is selected from the group consisting of A, V, L, I, M, F, W, P, S, T, C, Y, N, Q, D, E, K, R, and H; f) the peptide comprises a sequence that has at least 70%, at least 75%, at least 80%, at least 85%, at least 90%, at least 92%, at least 95%, at least 97%, or at least 100% sequence identity with SEQ ID NO: 133 and wherein the peptide further comprises a joining sequence of one or more amino acid residues located immediately adjacent to an N-terminus or a C-terminus of the peptide and wherein i) N−1 is selected from the group consisting of G, A, V, L, I, M, F, W, P, S, T, C, Y, Q, D, E, K, R, and H; ii) N−2 is selected from the group consisting of G, A, V, L, M, F, W, P, S, T, C, Y, N, Q, D, E, K, R, and H; iii) N−3 is selected from the group consisting of G, A, V, L, I, M, F, W, S, T, C, Y, N, Q, D, E, K, R, and H; iv) N−4 is selected from the group consisting of G, A, L, I, M, F, W, P, S, T, C, Y, N, Q, D, E, K, R, and H; v) N−5 is selected from the group consisting of A, V, L, I, M, F, W, P, S, T, C, Y, N, Q, D, E, K, R, and H; g) the peptide comprises a sequence that has at least 70%, at least 75%, at least 80%, at least 85%, at least 90%, at least 92%, at least 95%, at least 97%, or at least 100% sequence identity with SEQ ID NO: 260 and wherein the peptide further comprises a joining sequence of one or more amino acid residues located immediately adjacent to an N-terminus or a C-terminus of the peptide and wherein i) C+1 is selected from the group consisting of G, A, V, L, I, M, F, W, P, S, C, Y, N, Q, D, E, K, R, and H; or ii) C+2 is selected from the group consisting of G, A, V, L, I, M, F, W, S, T, C, Y, N, Q, D, E, K, R, and H; or h) the peptide comprises a sequence that has at least 70%, at least 75%, at least 80%, at least 85%, at least 90%, at least 92%, at least 95%, at least 97%, or at least 100% sequence identity with SEQ ID NO: 262 and wherein the peptide further comprises a joining sequence of one or more amino acid residues located immediately adjacent to an N-terminus or a C-terminus of the peptide and wherein C+1 is selected from the group consisting of G, A, V, L, I, M, F, W, S, T, C, Y, N, Q, D, E, K, R, and H.

In some aspects, the peptide comprises at least 70%, at least 75%, at least 80%, at least 85%, at least 90%, at least 92%, at least 95%, at least 97%, at least 99%, or at least 100% identity with any one of SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256 and does not comprise SEQ ID NO: 128 or SEQ ID NO: 149. In some aspects, the peptide comprises any one of SEQ ID NO: 109-SEQ ID NO: 110, SEQ ID NO: 129-SEQ ID NO: 133, SEQ ID NO: 260, SEQ ID NO: 262 and does not comprise SEQ ID NO: 128 or SEQ ID NO: 149.

In some aspects, the joining sequence is at the N-terminus or a C-terminus of the peptide, or both the N-terminus and the C-terminus of the peptide. In some aspects, the joining sequence comprises from 1 to 100 amino acid residues. In some aspects, the peptide comprises a sequence that has at least 70%, at least 75%, at least 80%, at least 85%, at least 90%, at least 95%, at least 97%, at least 99% or 100% sequence identity with any one of SEQ ID NO: 109-SEQ ID NO: 110, SEQ ID NO: 260, or SEQ ID NO: 262 and further comprising no more than 5 additional amino acids at the N-terminus.

In some aspects, the peptide consists of a sequence that has at least 70%, at least 75%, at least 80%, at least 85%, at least 90%, at least 95%, at least 97%, at least 99% or 100% sequence identity with any one of the peptides selected from the group consisting of SEQ ID NO: 109-SEQ ID NO: 110, SEQ ID NO: 129-SEQ ID NO: 133, SEQ ID NO: 260, or SEQ ID NO: 262.

In further aspects, the peptide further comprises a joining sequence of one or more amino acid residues located immediately adjacent to an N-terminus or a C-terminus of the peptide and wherein: a) for any one of SEQ ID NO: 109, SEQ ID NO: 110, SEQ ID NO: 260, or SEQ ID NO: 262: i) N−1 is selected from the group consisting of G, A, L, I, M, F, W, P, S, T, C, Y, N, Q, D, E, K, R, and H; ii) N−2 is selected from the group consisting of G, A, V, L, I, M, F, W, P, S, T, C, Y, Q, D, E, K, R, and H; iii) N−3 is selected from the group consisting of G, A, V, L, M, F, W, P, S, T, C, Y, N, Q, D, E, K, R, and H; iv) N−4 is selected from the group consisting of G, A, V, L, I, M, F, W, S, T, C, Y, N, Q, D, E, K, R, and H; v) N−5 is selected from the group consisting of G, A, L, I, M, F, W, P, S, T, C, Y, N, Q, D, E, K, R, and H; or vi) N−6 is selected from the group consisting of A, V, L, I, M, F, W, P, S, T, C, Y, N, Q, D, E, K, R, and H; or b) for SEQ ID NO: 129, N−1 is selected from the group consisting of A, V, L, I, M, F, W, P, S, T, C, Y, N, Q, D, E, K, R, and H; or c) for SEQ ID NO: 130: i) N−1 is selected from the group consisting of G, A, L, I, M, F, W, P, S, T, C, Y, N, Q, D, E, K, R, and H; or ii) N−2 is selected from the group consisting of A, V, L, I, M, F, W, P, S, T, C, Y, N, Q, D, E, K, R, and H; or d) for SEQ ID NO: 131: i) N−1 is selected from the group consisting of G, A, V, L, I, M, F, W, S, T, C, Y, N, Q, D, E, K, R, and H; ii) N−2 is selected from the group consisting of G, A, L, I, M, F, W, P, S, T, C, Y, N, Q, D, E, K, R, and H; or iii) N−3 is selected from the group consisting of A, V, L, I, M, F, W, P, S, T, C, Y, N, Q, D, E, K, R, and H; or e) for SEQ ID NO: 132: i) N−1 is selected from the group consisting of G, A, V, L, M, F, W, P, S, T, C, Y, N, Q, D, E, K, R, and H; ii) N−2 is selected from the group consisting of G, A, V, L, I, M, F, W, S, T, C, Y, N, Q, D, E, K, R, and H; iii) N−3 is selected from the group consisting of G, A, L, I, M, F, W, P, S, T, C, Y, N, Q, D, E, K, R, and H; or iv) N−4 is selected from the group consisting of A, V, L, I, M, F, W, P, S, T, C, Y, N, Q, D, E, K, R, and H; f) for SEQ ID NO: 133: i) N−1 is selected from the group consisting of G, A, V, L, I, M, F, W, P, S, T, C, Y, Q, D, E, K, R, and H; ii) N−2 is selected from the group consisting of G, A, V, L, M, F, W, P, S, T, C, Y, N, Q, D, E, K, R, and H; iii) N−3 is selected from the group consisting of G, A, V, L, I, M, F, W, S, T, C, Y, N, Q, D, E, K, R, and H; iv) N−4 is selected from the group consisting of G, A, L, I, M, F, W, P, S, T, C, Y, N, Q, D, E, K, R, and H; v) N−5 is selected from the group consisting of A, V, L, I, M, F, W, P, S, T, C, Y, N, Q, D, E, K, R, and H; g) for SEQ ID NO: 260: i) C+1 is selected from the group consisting of G, A, V, L, I, M, F, W, P, S, C, Y, N, Q, D, E, K, R, and H; or ii) C+2 is selected from the group consisting of G, A, V, L, I, M, F, W, S, T, C, Y, N, Q, D, E, K, R, and H; or h) for SEQ ID NO: 262, C+1 is selected from the group consisting of G, A, V, L, I, M, F, W, S, T, C, Y, N, Q, D, E, K, R, and H.

In some aspects, the peptide is selected from the group consisting of SEQ ID NO: 109-SEQ ID NO: 110, SEQ ID NO: 129-SEQ ID NO: 133, SEQ ID NO: 260, and SEQ ID NO: 262.

In other aspects, the peptide consists of a sequence that has at least 70%, at least 75%, at least 80%, at least 85%, at least 90%, at least 95%, at least 97%, at least 99% or 100% sequence identity with any one of the peptides selected from the group consisting of SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256.

In further aspects, the peptide is selected from the group consisting of SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256.

In various aspects, the present disclosure provides a peptide, wherein a) the peptide comprises at least 70%, at least at least 75%, at least 80%, at least 85%, at least 90%, at least 95%, at least 97%, at least 98%, at least 99%, or 100% sequence identity with a sequence selected from the group SEQ ID NO: 111-SEQ ID NO: 126 or SEQ ID NO: 233-SEQ ID NO: 240; or b) the peptide comprises at least 70%, at least at least 75%, at least 80%, at least 85%, at least 90%, at least 95%, at least 97%, at least 98%, at least 99%, or 100% sequence identity with a sequence selected from the group SEQ ID NO: 134-SEQ ID NO: 148 and SEQ ID NO: 249-SEQ ID NO: 256.

In various aspects, the present disclosure provides a peptide, wherein a peptide, wherein a) the peptide comprises a sequence that has at least 70%, at least 75%, at least 80%, at least 85%, at least 90%, at least 92%, at least 95%, at least 97%, or at least 100% sequence identity with any one of SEQ ID NO: 109, SEQ ID NO: 110, SEQ ID NO: 260, or SEQ ID NO: 262 and wherein the peptide further comprises a joining sequence of one or more amino acid residues located immediately adjacent to an N-terminus or a C-terminus of the peptide and wherein i) N−1 is selected from the group consisting of G, A, L, I, M, F, W, P, S, T, C, Y, N, Q, D, E, K, R, and H; ii) N−2 is selected from the group consisting of G, A, V, L, I, M, F, W, P, S, T, C, Y, Q, D, E, K, R, and H; iii) N−3 is selected from the group consisting of G, A, V, L, M, F, W, P, S, T, C, Y, N, Q, D, E, K, R, and H; iv) N−4 is selected from the group consisting of G, A, V, L, I, M, F, W, S, T, C, Y, N, Q, D, E, K, R, and H; v) N−5 is selected from the group consisting of G, A, L, I, M, F, W, P, S, T, C, Y, N, Q, D, E, K, R, and H; or vi) N−6 is selected from the group consisting of A, V, L, I, M, F, W, P, S, T, C, Y, N, Q, D, E, K, R, and H; b) the peptide comprises a sequence that has at least 70%, at least 75%, at least 80%, at least 85%, at least 90%, at least 92%, at least 95%, at least 97%, or at least 100% sequence identity with SEQ ID NO: 129 and wherein the peptide further comprises a joining sequence of one or more amino acid residues located immediately adjacent to an N-terminus or a C-terminus of the peptide and wherein N−1 is selected from the group consisting of A, V, L, I, M, F, W, P, S, T, C, Y, N, Q, D, E, K, R, and H; c) the peptide comprises a sequence that has at least 70%, at least 75%, at least 80%, at least 85%, at least 90%, at least 92%, at least 95%, at least 97%, or at least 100% sequence identity with SEQ ID NO: 130 and wherein the peptide further comprises a joining sequence of one or more amino acid residues located immediately adjacent to an N-terminus or a C-terminus of the peptide and wherein i) N−1 is selected from the group consisting of G, A, L, I, M, F, W, P, S, T, C, Y, N, Q, D, E, K, R, and H; or ii) N−2 is selected from the group consisting of A, V, L, I, M, F, W, P, S, T, C, Y, N, Q, D, E, K, R, and H; d) the peptide comprises a sequence that has at least 70%, at least 75%, at least 80%, at least 85%, at least 90%, at least 92%, at least 95%, at least 97%, or at least 100% sequence identity with SEQ ID NO: 131 and wherein the peptide further comprises a joining sequence of one or more amino acid residues located immediately adjacent to an N-terminus or a C-terminus of the peptide and wherein i) N−1 is selected from the group consisting of G, A, V, L, I, M, F, W, S, T, C, Y, N, Q, D, E, K, R, and H; ii) N−2 is selected from the group consisting of G, A, L, I, M, F, W, P, S, T, C, Y, N, Q, D, E, K, R, and H; or iii) N−3 is selected from the group consisting of A, V, L, I, M, F, W, P, S, T, C, Y, N, Q, D, E, K, R, and H; e) the peptide comprises a sequence that has at least 70%, at least 75%, at least 80%, at least 85%, at least 90%, at least 92%, at least 95%, at least 97%, or at least 100% sequence identity with SEQ ID NO: 132 and wherein the peptide further comprises a joining sequence of one or more amino acid residues located immediately adjacent to an N-terminus or a C-terminus of the peptide and wherein i) N−1 is selected from the group consisting of G, A, V, L, M, F, W, P, S, T, C, Y, N, Q, D, E, K, R, and H; ii) N−2 is selected from the group consisting of G, A, V, L, I, M, F, W, S, T, C, Y, N, Q, D, E, K, R, and H; iii) N−3 is selected from the group consisting of G, A, L, I, M, F, W, P, S, T, C, Y, N, Q, D, E, K, R, and H; or iv) N−4 is selected from the group consisting of A, V, L, I, M, F, W, P, S, T, C, Y, N, Q, D, E, K, R, and H; f) the peptide comprises a sequence that has at least 70%, at least 75%, at least 80%, at least 85%, at least 90%, at least 92%, at least 95%, at least 97%, or at least 100% sequence identity with SEQ ID NO: 133 and wherein the peptide further comprises a joining sequence of one or more amino acid residues located immediately adjacent to an N-terminus or a C-terminus of the peptide and wherein i) N−1 is selected from the group consisting of G, A, V, L, I, M, F, W, P, S, T, C, Y, Q, D, E, K, R, and H; ii) N−2 is selected from the group consisting of G, A, V, L, M, F, W, P, S, T, C, Y, N, Q, D, E, K, R, and H; iii) N−3 is selected from the group consisting of G, A, V, L, I, M, F, W, S, T, C, Y, N, Q, D, E, K, R, and H; iv) N−4 is selected from the group consisting of G, A, L, I, M, F, W, P, S, T, C, Y, N, Q, D, E, K, R, and H; v) N−5 is selected from the group consisting of A, V, L, I, M, F, W, P, S, T, C, Y, N, Q, D, E, K, R, and H; g) the peptide comprises a sequence that has at least 70%, at least 75%, at least 80%, at least 85%, at least 90%, at least 92%, at least 95%, at least 97%, or at least 100% sequence identity with SEQ ID NO: 260 and wherein the peptide further comprises a joining sequence of one or more amino acid residues located immediately adjacent to an N-terminus or a C-terminus of the peptide and wherein i) C+1 is selected from the group consisting of G, A, V, L, I, M, F, W, P, S, C, Y, N, Q, D, E, K, R, and H; or ii) C+2 is selected from the group consisting of G, A, V, L, I, M, F, W, S, T, C, Y, N, Q, D, E, K, R, and H; or h) the peptide comprises a sequence that has at least 70%, at least 75%, at least 80%, at least 85%, at least 90%, at least 92%, at least 95%, at least 97%, or at least 100% sequence identity with SEQ ID NO: 262 and wherein the peptide further comprises a joining sequence of one or more amino acid residues located immediately adjacent to an N-terminus or a C-terminus of the peptide and wherein C+1 is selected from the group consisting of G, A, V, L, I, M, F, W, S, T, C, Y, N, Q, D, E, K, R, and H.

In some aspects, the peptide further comprises a joining sequence of one or more amino acid residues located immediately adjacent to an N-terminus or a C-terminus of the peptide. In some aspects, the joining sequence is at an N-terminus or a C-terminus of the peptide, or both the N-terminus and the C-terminus of the peptide. In some aspects, the joining sequence comprises from 1 to 100 amino acid residues. In some aspects, the peptide comprises a sequence that has at least 70%, at least 75%, at least 80%, at least 85%, at least 90%, at least 95%, at least 97%, at least 99% or 100% sequence identity with any one of SEQ ID NO: 109-SEQ ID NO: 110, SEQ ID NO: 260, or SEQ ID NO: 262 and further comprising no more than 5 additional amino acids at the N-terminus.

In some aspects, the peptide consists of a sequence that has at least 70%, at least 75%, at least 80%, at least 85%, at least 90%, at least 95%, at least 97%, at least 99% or 100% sequence identity with any one of the peptides selected from the group consisting of SEQ ID NO: 109-SEQ ID NO: 110, SEQ ID NO: 129-SEQ ID NO: 133, SEQ ID NO: 260, or SEQ ID NO: 262.

In further aspects, the peptide further comprises a joining sequence of one or more amino acid residues located immediately adjacent to an N-terminus or a C-terminus of the peptide and wherein: a) for any one of SEQ ID NO: 109, SEQ ID NO: 110, SEQ ID NO: 260, or SEQ ID NO: 262: i) N−1 is selected from the group consisting of G, A, L, I, M, F, W, P, S, T, C, Y, N, Q, D, E, K, R, and H; ii) N−2 is selected from the group consisting of G, A, V, L, I, M, F, W, P, S, T, C, Y, Q, D, E, K, R, and H; iii) N−3 is selected from the group consisting of G, A, V, L, M, F, W, P, S, T, C, Y, N, Q, D, E, K, R, and H; iv) N−4 is selected from the group consisting of G, A, V, L, I, M, F, W, S, T, C, Y, N, Q, D, E, K, R, and H; v) N−5 is selected from the group consisting of G, A, L, I, M, F, W, P, S, T, C, Y, N, Q, D, E, K, R, and H; or vi) N−6 is selected from the group consisting of A, V, L, I, M, F, W, P, S, T, C, Y, N, Q, D, E, K, R, and H; or b) for SEQ ID NO: 129, N−1 is selected from the group consisting of A, V, L, I, M, F, W, P, S, T, C, Y, N, Q, D, E, K, R, and H; or c) for SEQ ID NO: 130: i) N−1 is selected from the group consisting of G, A, L, I, M, F, W, P, S, T, C, Y, N, Q, D, E, K, R, and H; or ii) N−2 is selected from the group consisting of A, V, L, I, M, F, W, P, S, T, C, Y, N, Q, D, E, K, R, and H; or d) for SEQ ID NO: 131: i) N−1 is selected from the group consisting of G, A, V, L, I, M, F, W, S, T, C, Y, N, Q, D, E, K, R, and H; ii) N−2 is selected from the group consisting of G, A, L, I, M, F, W, P, S, T, C, Y, N, Q, D, E, K, R, and H; or iii) N−3 is selected from the group consisting of A, V, L, I, M, F, W, P, S, T, C, Y, N, Q, D, E, K, R, and H; or e) for SEQ ID NO: 132: i) N−1 is selected from the group consisting of G, A, V, L, M, F, W, P, S, T, C, Y, N, Q, D, E, K, R, and H; ii) N−2 is selected from the group consisting of G, A, V, L, I, M, F, W, S, T, C, Y, N, Q, D, E, K, R, and H; iii) N−3 is selected from the group consisting of G, A, L, I, M, F, W, P, S, T, C, Y, N, Q, D, E, K, R, and H; or iv) N−4 is selected from the group consisting of A, V, L, I, M, F, W, P, S, T, C, Y, N, Q, D, E, K, R, and H; f) for SEQ ID NO: 133: i) N−1 is selected from the group consisting of G, A, V, L, I, M, F, W, P, S, T, C, Y, Q, D, E, K, R, and H; ii) N−2 is selected from the group consisting of G, A, V, L, M, F, W, P, S, T, C, Y, N, Q, D, E, K, R, and H; iii) N−3 is selected from the group consisting of G, A, V, L, I, M, F, W, S, T, C, Y, N, Q, D, E, K, R, and H; iv) N−4 is selected from the group consisting of G, A, L, I, M, F, W, P, S, T, C, Y, N, Q, D, E, K, R, and H; v) N−5 is selected from the group consisting of A, V, L, I, M, F, W, P, S, T, C, Y, N, Q, D, E, K, R, and H; g) for SEQ ID NO: 260: i) C+1 is selected from the group consisting of G, A, V, L, I, M, F, W, P, S, C, Y, N, Q, D, E, K, R, and H; or ii) C+2 is selected from the group consisting of G, A, V, L, I, M, F, W, S, T, C, Y, N, Q, D, E, K, R, and H; or h) for SEQ ID NO: 262, C+1 is selected from the group consisting of G, A, V, L, I, M, F, W, S, T, C, Y, N, Q, D, E, K, R, and H.

In some aspects, the peptide is selected from the group consisting of SEQ ID NO: 109-SEQ ID NO: 110, SEQ ID NO: 129-SEQ ID NO: 133, SEQ ID NO: 260, and SEQ ID NO: 262. In some aspects, the peptide consists of a sequence that has at least 70%, at least 75%, at least 80%, at least 85%, at least 90%, at least 95%, at least 97%, at least 99% or 100% sequence identity with any one of the peptides selected from the group consisting of SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256. In some aspects, the peptide is selected from the group consisting of SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256.

In some aspects, the peptide comprises SEQ ID NO: 28, SEQ ID NO: 45-SEQ ID NO: 51, SEQ ID NO: 109, SEQ ID NO: 150, SEQ ID NO: 199, SEQ ID NO: 110, SEQ ID NO: 127-SEQ ID NO: 133, SEQ ID NO: 149, or SEQ ID NO: 260-SEQ ID NO: 263 and further comprises one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null.

In some aspects, the peptide comprises SEQ ID NO: 27, SEQ ID NO: 29-SEQ ID NO: 44, SEQ ID NO: 52-SEQ ID NO: 66, SEQ ID NO: 109, SEQ ID NO: 150, SEQ ID NO: 199, SEQ ID NO: 111-SEQ ID NO: 126, SEQ ID NO: 134-SEQ ID NO: 148, SEQ ID NO: 233-SEQ ID NO: 256, and SEQ ID NO: 21-SEQ ID NO: 26, SEQ ID NO: 87-SEQ ID NO: 89, SEQ ID NO: 106-SEQ ID NO: 108, SEQ ID NO: 219-SEQ ID NO: 226 and further comprises one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259).

In other aspects, the peptide comprises one or more of the following peptide fragments within its sequence: GKCINKKCKC (SEQ ID NO: 90); KCIN (SEQ ID NO: 91); KKCK (SEQ ID NO: 92); PCKR (SEQ ID NO: 93); KRCSRR (SEQ ID NO: 94); KQC (SEQ ID NO: 95); GRCINRRCRC (SEQ ID NO: 96); RCIN (SEQ ID NO: 97); RRCR (SEQ ID NO: 98); PCRR (SEQ ID NO: 99); RRCSRR (SEQ ID NO: 100); RQC (SEQ ID NO: 101); PCKK (SEQ ID NO: 102), KKCSKK (SEQ ID NO: 103), GKCMNGKC (SEQ ID NO: 104); GRCMNGRC (SEQ ID NO: 105); GRCINSRC (SEQ ID NO: 227), GRCIXXRC (SEQ ID NO: 228) wherein each X can independently be any amino acid or amino acid analogue or null, GRCIX¹X²RC (SEQ ID NO: 229), wherein X¹=N, Q, A, S, T or L and X²=S, G, or R (SEQ ID NO: 229), PCR (SEQ ID NO: 230), CLDPCRRA (SEQ ID NO: 231), CLDPCRR (SEQ ID NO: 232), RCRGSRDC (SEQ ID NO: 257), PCRRAG (SEQ ID NO: 258), and RFGRCI (SEQ ID NO: 259).

In some aspects, the peptide comprises an N-terminal sequence comprising GG, SS, GS, SG, S, or G. In some aspects, the peptide is SEQ ID NO: 219. In other aspects, the peptide is SEQ ID NO: 220. In other aspects, the peptide is SEQ ID NO: 221 and wherein: a) the peptide further comprises one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null; or b) the peptide further comprises one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259); or c) and the peptide further comprises at least one corresponding substitution selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

In other aspects, the peptide is SEQ ID NO: 222. In other aspects, the peptide is SEQ ID NO: 223. In other aspects, the peptide is SEQ ID NO: 224. In other aspects, the peptide is SEQ ID NO: 225. In other aspects, the peptide is SEQ ID NO: 226. In other aspects, the peptide is SEQ ID NO: 87. In other aspects, the peptide is SEQ ID NO: 89 and wherein: a) the peptide further comprises one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null; or b) the peptide further comprises one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259); or c) and the peptide further comprises at least one corresponding substitution selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

In other aspects, the peptide is SEQ ID NO: 106 and wherein: a) the peptide further comprises one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null; or b) the peptide further comprises one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259); or c) and the peptide further comprises at least one corresponding substitution selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

In other aspects, the peptide is SEQ ID NO: 110 and further comprising no more than 5 additional amino acids at the N-terminus. In other aspects, the peptide is SEQ ID NO: 219. In other aspects, the peptide is SEQ ID NO: 221 a) the peptide further comprises one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null; or b) the peptide further comprises one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259); or c) and the peptide further comprises at least one corresponding substitution selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

In various aspects, the present disclosure provides a peptide comprising at least 70%, at least 75%, at least 80%, at least 85%, at least 90%, at least 92%, at least 95%, at least 97%, at least 99%, or 100% sequence identity with any one of SEQ ID NO: 209-SEQ ID NO: 215, wherein the peptide further comprises an active agent and wherein the active agent is complexed with the peptide to form a peptide active agent complex. In some aspects, the peptide further comprises an active agent, wherein the active agent is complexed with the peptide to form a peptide active agent complex. In some aspects, the active agent is selected from TABLE 3, TABLE 4, or TABLE 5.

In some aspects, in any of the above peptides or in any of the above peptide active agent complexes, the peptide comprises at least 70%, at least 80%, at least 90%, at least 95%, at least 97%, or 100% sequence identity to: SEQ ID NO: 27; SEQ ID NO: 28; SEQ ID NO: 29; SEQ ID NO: 30; SEQ ID NO: 31; SEQ ID NO: 32; SEQ ID NO: 33; SEQ ID NO: 34; SEQ ID NO: 35; SEQ ID NO: 36; SEQ ID NO: 37; SEQ ID NO: 38; SEQ ID NO: 39; SEQ ID NO: 40; SEQ ID NO: 41; SEQ ID NO: 42; SEQ ID NO: 43; SEQ ID NO: 44; SEQ ID NO: 45; SEQ ID NO: 234; SEQ ID NO: 47; SEQ ID NO: 48; SEQ ID NO: 49; SEQ ID NO: 50; SEQ ID NO: 51; SEQ ID NO: 109 and wherein the peptide further comprises a joining sequence of one or more amino acid residues located immediately adjacent to an N-terminus or a C-terminus of the peptide and wherein i) N−1 is selected from the group consisting of G, A, L, I, M, F, W, P, S, T, C, Y, N, Q, D, E, K, R, and H; ii) N−2 is selected from the group consisting of G, A, V, L, I, M, F, W, P, S, T, C, Y, Q, D, E, K, R, and H; iii) N−3 is selected from the group consisting of G, A, V, L, M, F, W, P, S, T, C, Y, N, Q, D, E, K, R, and H; iv) N−4 is selected from the group consisting of G, A, V, L, I, M, F, W, S, T, C, Y, N, Q, D, E, K, R, and H; v) N−5 is selected from the group consisting of G, A, L, I, M, F, W, P, S, T, C, Y, N, Q, D, E, K, R, and H; or vi) N−6 is selected from the group consisting of A, V, L, I, M, F, W, P, S, T, C, Y, N, Q, D, E, K, R, and H further comprising no more than 5 additional amino acids at the N-terminus; SEQ ID NO: 110 and wherein the peptide further comprises a joining sequence of one or more amino acid residues located immediately adjacent to an N-terminus or a C-terminus of the peptide and wherein i) N−1 is selected from the group consisting of G, A, L, I, M, F, W, P, S, T, C, Y, N, Q, D, E, K, R, and H; ii) N−2 is selected from the group consisting of G, A, V, L, I, M, F, W, P, S, T, C, Y, Q, D, E, K, R, and H; iii) N−3 is selected from the group consisting of G, A, V, L, M, F, W, P, S, T, C, Y, N, Q, D, E, K, R, and H; iv) N−4 is selected from the group consisting of G, A, V, L, I, M, F, W, S, T, C, Y, N, Q, D, E, K, R, and H; v) N−5 is selected from the group consisting of G, A, L, I, M, F, W, P, S, T, C, Y, N, Q, D, E, K, R, and H; or vi) N−6 is selected from the group consisting of A, V, L, I, M, F, W, P, S, T, C, Y, N, Q, D, E, K, R, and H further comprising no more than 5 additional amino acids at the N-terminus; SEQ ID NO: 111; SEQ ID NO: 112; SEQ ID NO: 113; SEQ ID NO: 114; SEQ ID NO: 115; SEQ ID NO: 116; SEQ ID NO: 117; SEQ ID NO: 118; SEQ ID NO: 119; SEQ ID NO: 120; SEQ ID NO: 121; SEQ ID NO: 122; SEQ ID NO: 123; SEQ ID NO: 124; SEQ ID NO: 125; SEQ ID NO: 126; SEQ ID NO: 87; SEQ ID NO: 129 and wherein the peptide further comprises a joining sequence of one or more amino acid residues located immediately adjacent to an N-terminus or a C-terminus of the peptide and wherein N−1 is selected from the group consisting of A, V, L, I, M, F, W, P, S, T, C, Y, N, Q, D, E, K, R, and H, wherein the peptide further comprises a joining sequence at the N-terminus, and wherein a last residue at a C-terminus of the joining sequence is selected from the group consisting of A, V, L, I, M, F, W, P, S, T, C, Y, N, Q, D, E, K, R, and H; SEQ ID NO: 130 and wherein the peptide further comprises a joining sequence of one or more amino acid residues located immediately adjacent to an N-terminus or a C-terminus of the peptide and wherein i) N−1 is selected from the group consisting of G, A, L, I, M, F, W, P, S, T, C, Y, N, Q, D, E, K, R, and H; or ii) N−2 is selected from the group consisting of A, V, L, I, M, F, W, P, S, T, C, Y, N, Q, D, E, K, R, and H, wherein the peptide further comprises a joining sequence at the N-terminus, and wherein a last residue at a C-terminus of the joining sequence is selected from the group consisting of G, A, L, I, M, F, W, P, S, T, C, Y, N, Q, D, E, K, R, and H; SEQ ID NO: 131 and wherein the peptide further comprises a joining sequence of one or more amino acid residues located immediately adjacent to an N-terminus or a C-terminus of the peptide and wherein i) N−1 is selected from the group consisting of G, A, V, L, I, M, F, W, S, T, C, Y, N, Q, D, E, K, R, and H; ii) N−2 is selected from the group consisting of G, A, L, I, M, F, W, P, S, T, C, Y, N, Q, D, E, K, R, and H; or iii) N−3 is selected from the group consisting of A, V, L, I, M, F, W, P, S, T, C, Y, N, Q, D, E, K, R, and H; wherein the peptide further comprises a joining sequence at the N-terminus, and wherein a last residue at a C-terminus of the joining sequence is selected from the group consisting of G, A, V, L, I, M, F, W, S, T, C, Y, N, Q, D, E, K, R, and H; SEQ ID NO: 132 and wherein the peptide further comprises a joining sequence of one or more amino acid residues located immediately adjacent to an N-terminus or a C-terminus of the peptide and wherein i) N−1 is selected from the group consisting of G, A, V, L, M, F, W, P, S, T, C, Y, N, Q, D, E, K, R, and H; ii) N−2 is selected from the group consisting of G, A, V, L, I, M, F, W, S, T, C, Y, N, Q, D, E, K, R, and H; iii) N−3 is selected from the group consisting of G, A, L, I, M, F, W, P, S, T, C, Y, N, Q, D, E, K, R, and H; or iv) N−4 is selected from the group consisting of A, V, L, I, M, F, W, P, S, T, C, Y, N, Q, D, E, K, R, and H, wherein the peptide further comprises a joining sequence at the N-terminus, and wherein a last residue at a C-terminus of the joining sequence is selected from the group consisting of G, A, V, L, M, F, W, P, S, T, C, Y, N, Q, D, E, K, R, and H; SEQ ID NO: 133 and wherein the peptide further comprises a joining sequence of one or more amino acid residues located immediately adjacent to an N-terminus or a C-terminus of the peptide and wherein i) N−1 is selected from the group consisting of G, A, V, L, I, M, F, W, P, S, T, C, Y, Q, D, E, K, R, and H; ii) N−2 is selected from the group consisting of G, A, V, L, M, F, W, P, S, T, C, Y, N, Q, D, E, K, R, and H; iii) N−3 is selected from the group consisting of G, A, V, L, I, M, F, W, S, T, C, Y, N, Q, D, E, K, R, and H; iv) N−4 is selected from the group consisting of G, A, L, I, M, F, W, P, S, T, C, Y, N, Q, D, E, K, R, and H; or v) N−5 is selected from the group consisting of A, V, L, I, M, F, W, P, S, T, C, Y, N, Q, D, E, K, R, and H, wherein the peptide further comprises a joining sequence at the N-terminus, and wherein a last residue at a C-terminus of the joining sequence is selected from the group consisting of G, A, V, L, I, M, F, W, P, S, T, C, Y, Q, D, E, K, R, and H; SEQ ID NO: 89; SEQ ID NO: 106; SEQ ID NO: 219; or SEQ ID NO: 221; SEQ ID NO: 260 and wherein the peptide further comprises a joining sequence of one or more amino acid residues located immediately adjacent to an N-terminus or a C-terminus of the peptide and wherein i) N−1 is selected from the group consisting of G, A, L, I, M, F, W, P, S, T, C, Y, N, Q, D, E, K, R, and H; ii) N−2 is selected from the group consisting of G, A, V, L, I, M, F, W, P, S, T, C, Y, Q, D, E, K, R, and H; iii) N−3 is selected from the group consisting of G, A, V, L, M, F, W, P, S, T, C, Y, N, Q, D, E, K, R, and H; iv) N−4 is selected from the group consisting of G, A, V, L, I, M, F, W, S, T, C, Y, N, Q, D, E, K, R, and H; v) N−5 is selected from the group consisting of G, A, L, I, M, F, W, P, S, T, C, Y, N, Q, D, E, K, R, and H; vi) N−6 is selected from the group consisting of A, V, L, I, M, F, W, P, S, T, C, Y, N, Q, D, E, K, R, and H; vii) C+1 is selected from the group consisting of G, A, V, L, I, M, F, W, P, S, C, Y, N, Q, D, E, K, R, and H; or viii) C+2 is selected from the group consisting of G, A, V, L, I, M, F, W, S, T, C, Y, N, Q, D, E, K, R, and H; or SEQ ID NO: 262 and wherein the peptide further comprises a joining sequence of one or more amino acid residues located immediately adjacent to an N-terminus or a C-terminus of the peptide and wherein i) N−1 is selected from the group consisting of G, A, L, I, M, F, W, P, S, T, C, Y, N, Q, D, E, K, R, and H; ii) N−2 is selected from the group consisting of G, A, V, L, I, M, F, W, P, S, T, C, Y, Q, D, E, K, R, and H; iii) N−3 is selected from the group consisting of G, A, V, L, M, F, W, P, S, T, C, Y, N, Q, D, E, K, R, and H; iv) N−4 is selected from the group consisting of G, A, V, L, I, M, F, W, S, T, C, Y, N, Q, D, E, K, R, and H; v) N−5 is selected from the group consisting of G, A, L, I, M, F, W, P, S, T, C, Y, N, Q, D, E, K, R, and H; vi) N−6 is selected from the group consisting of A, V, L, I, M, F, W, P, S, T, C, Y, N, Q, D, E, K, R, and H; or vii) C+1 is selected from the group consisting of G, A, V, L, I, M, F, W, S, T, C, Y, N, Q, D, E, K, R, and H.

In some aspects, in the above described peptide active agent complex, the peptide comprises at least 70%, at least 80%, at least 90%, at least 95%, at least 97%, at least 99%, or 100% sequence identity to: SEQ ID NO: 52; SEQ ID NO: 53; SEQ ID NO: 54; SEQ ID NO: 55; SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 134; SEQ ID NO: 135; SEQ ID NO: 136; SEQ ID NO: 137; SEQ ID NO: 138; SEQ ID NO: 139; SEQ ID NO: 140; SEQ ID NO: 141; SEQ ID NO: 142; SEQ ID NO: 143; SEQ ID NO: 144; SEQ ID NO: 145; SEQ ID NO: 146; SEQ ID NO: 147; or SEQ ID NO: 148.

In some aspects, in the peptide or the peptide active agent complex, the peptide homes, targets, migrates to, accumulates in, binds to, is retained by, or is directed to cartilage, to kidney, or to cartilage and kidney. In some aspects, in the peptide or the peptide active agent complex, the peptide homes, targets, migrates to, accumulates in, binds to, is retained by, or is directed to proximal tubules of the kidney. In some aspects, in the peptide or the peptide active agent complex, the peptide is covalently complexed to the active agent. In some aspects, the peptide active agent complex homes, targets, migrates to, accumulates in, binds to, is retained by, or is directed to a cartilage or a kidney of the subject. In some aspects, in the peptide or the peptide active agent complex, the peptide comprises 4 or more cysteine residues.

In some aspects, in the peptide or the peptide active agent complex, the peptide comprises three or more disulfide bridges formed between cysteine residues, wherein one of the disulfide bridges passes through a loop formed by two other disulfide bridges. In some aspects, in the peptide or the peptide active agent complex, the peptide comprises a plurality of disulfide bridges formed between cysteine residues. In some aspects, in the peptide or the peptide active agent complex, the peptide comprises a disulfide through a disulfide knot. In some aspects, at least one amino acid residue of the peptide is in an L configuration or, wherein at least one amino acid residue of the peptide is in a D configuration. In some aspects, the sequence comprises at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 residues.

In some aspects, any one or more K residues are replaced by an R residue or wherein any one or more R residues are replaced by for a K residue. In some aspects, any one or more M residues are replaced by any one of the I, L, or V residues. In some aspects, any one or more L residues are replaced by any one of the V, I, or M residues. In some aspects, any one or more I residues are replaced by any of the M, L, or V residues. In some aspects, any one or more V residues are replaced by any of the M, I, or L residues.

In some aspects, any one or more G residues are replaced by an A residue or wherein any one or more A residues are replaced by a G residue. In some aspects, any one or more S residues are replaced by a T residue or wherein any one or more T residues are replaced by for an S residue. In some aspects, any one or more Q residues are replaced by an N residue or wherein any one or more N residues are replaced by a Q residue. In some aspects, any one or more D residues are replaced by an E residue or wherein any one or more E residues are replaced by a D residue. In some aspects, the peptide has a charge distribution comprising an acidic region and a basic region. In some aspects, the acidic region is a nub. In some aspects, the basic region is a patch.

In some aspects, the peptide comprises 5-12 basic residues. In some aspects, the peptide comprises 0-5 acidic residues. In some aspects, the peptide comprises 6 or more basic residues and 2 or fewer acidic residues. In some aspects, the peptide comprises a 4-19 amino acid residue fragment containing at least 2 cysteine residues, and at least 2 positively charged amino acid residues. In some aspects, the peptide comprises a 20-70 amino acid residue fragment containing at least 2 cysteine residues, no more than 2 basic residues and at least 2 positively charged amino acid residues. In some aspects, the peptide comprises at least 3 positively charged amino acid residues. In some aspects, the positively charged amino acid residues are selected from K, R, or a combination thereof. In some aspects, the peptide has a charge greater than 2 at physiological pH. In some aspects, the peptide has a charge greater than 3.5 at physiological pH. In some aspects, the peptide has a charge greater than 4.5 at physiological pH. In some aspects, the peptide has a charge greater than 5.5 at physiological pH. In some aspects, the peptide has a charge greater than 6.5 at physiological pH. In some aspects, the peptide has a charge greater than 7.5 at physiological pH. In some aspects, the peptide has a charge greater than 8.5 at physiological pH. In some aspects, the peptide has a charge greater than 9.5 at physiological pH. In some aspects, the peptide is selected from a potassium channel agonist, a potassium channel antagonist, a portion of a potassium channel, a sodium channel agonist, a sodium channel antagonist, a calcium channel agonist, a calcium channel antagonist, a hadrucalcin, a theraphotoxin, a huwentoxin, a kaliotoxin, a cobatoxin or a lectin.

In further aspects, the lectin is SHL-Ib2. In some aspects, the peptide is arranged in a multimeric structure with at least one other peptide. In some aspects, at least one residue of the peptide comprises a chemical modification. In further aspects, the chemical modification is blocking the N-terminus of the peptide. In still further aspects, the chemical modification is methylation, acetylation, or acylation. In some aspects, the chemical modification is: methylation of one or more lysine residues or analogue thereof; methylation of the N-terminus; or methylation of one or more lysine residue or analogue thereof and methylation of the N-terminus. In some aspects, the peptide is linked to an acyl adduct. In some aspects, the active agent is fused with the peptide at an N-terminus or a C-terminus of the peptide. In some aspects, the active agent is another peptide. In some aspects, the active agent is an antibody.

In some aspects, the active agent is an Fc domain, Fab domain, scFv, or Fv fragment. In some aspects, the active agent is a glucocorticoid. In some aspects, the active agent is desciclesonide. In some aspects, the peptide fused with the Fc domain comprises a contiguous sequence. In some aspects, 1, 2, 3, 4, 5, 6, 7, 8, 9, or 10 active agents are linked to the peptide. In some aspects, the peptide is linked to the active agent at an N-terminus, at the epsilon amine of a lysine residue, at the carboxylic acid of an aspartic acid or glutamic acid residue, or a C-terminus of the peptide by a linker. In some aspects, the peptide is linked to the active agent via a cleavable linker. In some aspects, the peptide further comprising a non-natural amino acid, wherein the non-natural amino acid is an insertion, appendage, or substitution for another amino acid. In some aspects, the peptide is linked to the active agent at the non-natural amino acid by a linker. In further aspects, linker comprises an amide bond, an ester bond, a carbamate bond, a carbonate bond, a hydrazone bond, an oxime bond, a disulfide bond, a thioester bond, a thioether bond, a triazole, a carbon-carbon bond, or a carbon-nitrogen bond. In some aspects, the linker comprises an ester bond.

In some aspects, the cleavable linker comprises a cleavage site for matrix metalloproteinases, thrombin, cathepsins, or beta-glucuronidase. In some aspects, the linker is a hydrolytically labile linker. In some aspects, the linker is pH sensitive, reducible, glutathione-sensitive, or protease cleavable. In some aspects, the peptide is linked to the active agent via a stable linker. In some aspects, the peptide has an isoelectric point of about 9. In some aspects, the peptide is linked to a detectable agent. In some aspects, the detectable agent is fused with the peptide at an N-terminus or a C-terminus of the peptide. In some aspects, 1, 2, 3, 4, 5, 6, 7, 8, 9, or 10 detectable agents are linked to the peptide. In some aspects, the peptide is linked to the detectable agent via a cleavable linker. In some aspects, the peptide is linked to the detectable agent at an N-terminus, at the epsilon amine of an internal lysine residue, or a C-terminus of the peptide by a linker. In some aspects, the peptide further comprises a non-natural amino acid, wherein the non-natural amino acid is an insertion, appendage, or substitution for another amino acid.

In some aspects, the peptide is linked to the detectable agent at the non-natural amino acid by a linker. In some aspects, the linker comprises an amide bond, an ester bond, a carbamate bond, a hydrazone bond, an oxime bond, or a carbon-nitrogen bond. In some aspects, the cleavable linker comprises a cleavage site for matrix metalloproteinases, thrombin, cathepsins, or beta-glucuronidase. In some aspects, the peptide is linked to the detectable agent via a stable linker. In some aspects, the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator. In some aspects, the detectable agent is a fluorescent dye. In some aspects, the peptide further comprises a joining sequence. In some aspects, the joining sequence is at an N-terminus or a C-terminus of the peptide. In some aspects, the joining sequence comprises from 1 to 100 amino acid residues.

In various aspects, the present disclosure provides a pharmaceutical composition comprising any of the above peptides or a salt thereof, or any of the above peptide active agent complexes or a salt thereof, and a pharmaceutically acceptable carrier. In some aspects, the pharmaceutical composition is formulated for administration to a subject. In some aspects, the pharmaceutical composition is formulated for inhalation, intranasal administration, oral administration, topical administration, parenteral administration, intravenous administration, subcutaneous administration, intra-articular administration, intramuscular administration, intraperitoneal administration, dermal administration, transdermal administration, or a combination thereof.

In various aspects, the present disclosure provides a method of treating a condition in a subject in need thereof, the method comprising administering to the subject any of the above peptides, any of the above peptide active agent complexes, or any of the above pharmaceutical compositions.

In some aspects, the peptide active agent complex, peptide, or pharmaceutical composition is administered by inhalation, intranasally, orally, topically, parenterally, intravenously, subcutaneously, intra-articularly, intramuscularly administration, intraperitoneally, dermally, transdermally, or a combination thereof. In some aspects, the peptide active agent complex or the peptide homes, targets, or migrates to cartilage of the subject following administration. In some aspects, the condition is associated with cartilage. In some aspects, the condition is associated with a joint. In some aspects, the condition is an inflammation, a cancer, a degradation, a growth disturbance, genetic, a tear, an infection, a disease, or an injury. In some aspects, the condition is a chondrodystrophy. In some aspects, the condition is a traumatic rupture or detachment. In some aspects, the condition is a costochondritis. In some aspects, the condition is a herniation. In some aspects, the condition is a polychondritis.

In other aspects, the condition is a chordoma. In some aspects, the condition is a type of arthritis. In some aspects, the type of arthritis is rheumatoid arthritis. In some aspects, the type of arthritis is osteoarthritis. In some aspects, the type of arthritis is lupus arthritis. In some aspects, the condition is Systemic lupus erythematosus. In some aspects, the condition is achondroplasia. In some aspects, the condition is benign chondroma or malignant chondrosarcoma. In some aspects, the condition is bursitis, tendinitis, gout, pseudogout, an arthropathy, psoriatic arthritis, ankylosing spondylitis, or an infection. In some aspects, the peptide active agent complex, peptide, or pharmaceutical composition is administered to treat the injury, to repair a tissue damaged by the injury, or to treat a pain caused by the injury. In some aspects, the peptide active agent complex, peptide, or pharmaceutical composition is administered to treat the tear or to repair a tissue damaged by the tear. In some aspects, the peptide active agent complex, peptide, or pharmaceutical composition homes, targets, or migrates to a kidney of the subject following administration. In some aspects, the condition is associated with a kidney. In some aspects, the condition is lupus nephritis, acute kidney injury (AKI), chronic kidney disease (CKD), hypertensive kidney damage, diabetic nephropathy, lupus nephritis, or renal fibrosis.

In various aspects, the present disclosure provides a method of imaging an organ or body region of a subject, the method comprising: administering to the subject any peptide described above, any peptide active agent complex described above, or any pharmaceutical composition described above; and imaging the subject.

In some aspects, the method further comprises detecting a cancer or diseased region, tissue, structure or cell. In some aspects, the method further comprises performing surgery on the subject. In some aspects, the method further comprises treating the cancer. In some aspects, the surgery further comprises removing the cancer or the diseased region, tissue, structure or cell of the subject. In some aspects, the method further comprises imaging the cancer or diseased region, tissue, structure, or cell of the subject after surgical removal.

In some aspects, any of the above described peptide active agent complexes is expressed as a fusion protein.

In various aspects, the present disclosure provides a method of treating or delivering a peptide or peptide agent complex to a subject in need thereof according to method described above, the method further comprising administering a companion diagnostic, therapeutic, or imaging agent, wherein the companion diagnostic or imaging agent comprises a) any peptide active agent complex described above, b) any peptide described above; or c) a peptide of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, or SEQ ID NO: 233-SEQ ID NO: 256 further comprising a diagnostic, therapeutic, or imaging agent, wherein the diagnostic or imaging agent comprises a chemical agent, a radiolabel agent, radiosensitizing agent, fluorophore, an imaging agent, a diagnostic agent, a protein, a peptide, or a small molecule.

In some aspects, the companion diagnostic, therapeutic, or imaging agent is detected by a device. In some aspects, the device is used to detect the companion diagnostic, therapeutic, or imaging agent or to assess an agent's safety and physiologic effect. In some aspects, the agent's safety and physiologic effect is bioavailability, uptake, distribution and clearance, metabolism, pharmacokinetics, localization, measurement of concentrations in blood and tissues, assessing therapeutic window, range and optimization. In some aspects, the method is combined with or integrated into a surgical microscope, confocal microscope, fluorescence scope, exoscope, endoscope, or a surgical robot comprising a KINEVO 900, QEVO, CONVIVO, OMPI PENTERO 900, OMPI PENTERO 800, INFRARED 800, FLOW 800, OMPI LUMERIA, OMPI Vario, OMPI VARIO 700, OMPI Pico, TREMON 3DHD, a PROVido, ARvido, GLOW 800, Leica M530 OHX, Leica M530 OH6, Leica M720 OHX5, Leica M525 F50, Leica M525 F40, Leica M525 F20, Leica M525 OH4, Leica HD C100, Leica FL560, Leica FL400 Leica FL800, Leica DI C500, Leica ULT500, Leica Rotatable Beam Splitter, Leica M651 MSD, LIGHTENING, Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Haag-Streit 5-1000, Haag-Streit 3-1000, and Intuitive Surgical da Vinci surgical robot. In some aspects, the device incorporates radiology or fluorescence, including the X-ray radiography, magnetic resonance imaging (MRI), ultrasound, endoscopy, elastography, tactile imaging, thermography, flow cytometry, medical photography, nuclear medicine functional imaging techniques, positron emission tomography (PET), single-photon emission computed tomography (SPECT), surgical instrument, operating microscope, confocal microscope, fluorescence scope, exoscope, or a surgical robot.

In some aspects, the peptide comprises at least two of, at least three of, at least four of, at least five of, at least six of, at least seven of, at least eight of, or all of KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); or GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null.

In some aspects, the peptide comprises at least two of, at least three of, at least four of, at least five of, at least six of, at least seven of, at least eight of, at least nine of, at least ten of, or all of RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); or RFGRCI (SEQ ID NO: 259).

In some aspects, the peptide comprises at least two of, at least three of, at least four of, at least five of, at least six of, at least seven of, at least eight of, at least nine of, at least ten of, at least 11 of, at least 12 of, at least 13 of, at least 14 of, at least 15 of, at least 16 of, at least 17 of, at least 18 of, at least 19 of, at least 20 of, at least 21 of, at least 22 of, at least 23 of, at least 24 of, at least 25 of, or all of GKCINKKCKC (SEQ ID NO: 90); KCIN (SEQ ID NO: 91); KKCK (SEQ ID NO: 92); PCKR (SEQ ID NO: 93); KRCSRR (SEQ ID NO: 94); KQC (SEQ ID NO: 95); GRCINRRCRC (SEQ ID NO: 96); RCIN (SEQ ID NO: 97); RRCR (SEQ ID NO: 98); PCRR (SEQ ID NO: 99); RRCSRR (SEQ ID NO: 100); RQC (SEQ ID NO: 101); PCKK (SEQ ID NO: 102), KKCSKK (SEQ ID NO: 103), GKCMNGKC (SEQ ID NO: 104); GRCMNGRC (SEQ ID NO: 105); GRCINSRC (SEQ ID NO: 227), GRCIXXRC (SEQ ID NO: 228) wherein each X can independently be any amino acid or amino acid analogue or null, GRCIX1X2RC (SEQ ID NO: 229), wherein X1=N, Q, A, S, T or L and X2=S, G, or R (SEQ ID NO: 229), PCR (SEQ ID NO: 230), CLDPCRRA (SEQ ID NO: 231), CLDPCRR (SEQ ID NO: 232), RCRGSRDC (SEQ ID NO: 257), PCRRAG (SEQ ID NO: 258), and RFGRCI (SEQ ID NO: 259).

In some aspects, the peptide further comprises at least two of, at least three of, at least four of, at least five of, at least six of, at least seven of, at least eight of, at least nine of, at least ten of, at least 11 of, at least 12 of, at least 13 of, at least 14 of, at least 15 of, at least 16 of, at least 17 of, at least 18 of, at least 19 of, at least 20 of, at least 21 of, at least 22 of, at least 23 of, at least 24 of, at least 25 of, at least 26 of, at least 27 of, at least 28 of, at least 29, of or all of the corresponding substitutions selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

### INCORPORATION BY REFERENCE

All publications, patents, and patent applications mentioned, disclosed or referenced in this specification are herein incorporated by reference in their entirety and to the same extent as if each individual publication, patent, or patent application was specifically and individually indicated to be incorporated by reference.

### BRIEF DESCRIPTION OF THE FIGURES

The novel features of the invention are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the identification of the ¹⁴C signal in the joint and other cartilage of an animal treated with the peptide of SEQ ID NO: 150.

FIG. 2 illustrates an exemplary architecture of constructs expressing sequences of SEQ ID NO: X, where X can be any one of peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

FIG. 3 illustrates a schematic of a method of manufacturing of a peptide of the disclosure.

FIG. 4 illustrates the ¹⁴C signal in the cartilage of an animal with intact kidneys 24 hours after treatment with a peptide of SEQ ID NO: 150.

FIG. 5 shows white light images and corresponding whole body fluorescence images of a mouse administered 10 nmol of a peptide of SEQ ID NO: 149 (also disclosed herein as SEQ ID NO: 46; non-GS version of SEQ ID NO: 149/SEQ ID NO: 46 are shown in SEQ ID NO: 128 and SEQ ID NO: 205) conjugated to a Cy5.5 fluorophore (SEQ ID NO: 149A) at 24 hours post-administration. FIG. 5A illustrates an image of a frozen section of a mouse, 24 hours after administration of 10 nmol of a peptide of SEQ ID NO: 149 conjugated to a Cy5.5 fluorophore (SEQ ID NO: 149A). FIG. 5B illustrates the fluorescence signal in the mouse, corresponding to the section shown in FIG. 5A, 24 hours after administration of 10 nmol of a peptide of SEQ ID NO: 149 conjugated to a Cy5.5 fluorophore (SEQ ID NO: 149A). FIG. 5C illustrates an image of a different frozen section of the mouse, 24 hours after administration of 10 nmol of a peptide of SEQ ID NO: 46 (also disclosed herein as SEQ ID NO: 149; non-GS version of SEQ ID NO: 149/SEQ ID NO: 46 are shown in SEQ ID NO: 128 and SEQ ID NO: 205) conjugated to a Cy5.5 fluorophore (SEQ ID NO: 149A). FIG. 5D illustrates the fluorescence signal in the mouse, corresponding to the section shown in FIG. 5C, 24 hours after administration of 10 nmol of a peptide of SEQ ID NO: 149 conjugated to a Cy5.5 fluorophore (SEQ ID NO: 149A). FIG. 5E illustrates an image of a different frozen section of the mouse, 24 hours after administration of 10 nmol of a peptide of SEQ ID NO: 149 conjugated to a Cy5.5 fluorophore (SEQ ID NO: 149A). FIG. 5F illustrates a fluorescence signal in the mouse, corresponding to the section shown in FIG. 5E, 24 hours after administration of 10 nmol of a peptide of SEQ ID NO: 149 conjugated to a Cy5.5 fluorophore (SEQ ID NO: 149A).

FIG. 6 illustrates a multiple sequence alignment of SEQ ID NO: 198-SEQ ID NO: 215 were used to predict enhanced peptide stability and immunogenicity or other functions. SEQ ID NO: 87 is a consensus sequence, and SEQ ID NO: 21 is the same sequence as SEQ ID NO: 87 but with an N-terminal “GS.” SEQ ID NO: 219-SEQ ID NO: 222 are variant consensus peptide sequences, included within the family of SEQ ID NO: 87 and SEQ ID NO: 21 consensus sequences, which variant consensus sequences include further improved properties of the peptides as described herein, with or without an N-terminal GS.

FIG. 7 illustrates the identification of locations the ¹⁴C signal in the nasal, spinal, tracheal, and other cartilage of an animal treated with the peptide of SEQ ID NO: 150.

FIG. 8 shows IVIS fluorescence imaging of an isolated hind limb from a first mouse and an isolated hind limb from a second mouse after administration of 10 nmol SEQ ID NO: 149 peptide conjugated to a Cy5.5 fluorophore (SEQ ID NO: 46A; also the same as SEQ ID NO: 149A, because SEQ ID NO: 46 and SEQ ID NO: 149 disclose the same sequence; the non-GS version of SEQ ID NO: 149/SEQ ID NO: 46 is shown in SEQ ID NO: 128 and SEQ ID NO: 205). Areas of low signal intensity are shown in a thin solid line, areas of medium signal intensity are shown in a thick sold line, and areas of high signal intensity are shown in a thin dotted line. FIG. 8A shows the right hind limb with skin removed from a first mouse and from a second mouse 3 hours after peptide administration. FIG. 8B shows the right hind limb with muscle removed from a first mouse and from a second mouse 3 hours after peptide administration of 10 nmol SEQ ID NO: 149 peptide conjugated to a Cy5.5 fluorophore (SEQ ID NO: 149A). FIG. 8C shows the right hind limb with skin removed from a first mouse and from a second mouse 24 hours after peptide administration of 10 nmol SEQ ID NO: 149 peptide conjugated to a Cy5.5 fluorophore (SEQ ID NO: 149A). FIG. 8D shows the right hind limb with muscle removed from a first mouse and from a second mouse 24 hours after peptide administration of 10 nmol SEQ ID NO: 149 peptide conjugated to a Cy5.5 fluorophore (SEQ ID NO: 149A). FIG. 8E shows the right hind limb with skin removed from a first mouse and from a second mouse 48 hours after peptide administration of 10 nmol SEQ ID NO: 149 peptide conjugated to a Cy5.5 fluorophore (SEQ ID NO: 149A). FIG. 8F shows the right hind limb with muscle removed from a first mouse and from a second mouse 48 hours after peptide administration of 10 nmol SEQ ID NO: 149 peptide conjugated to a Cy5.5 fluorophore (SEQ ID NO: 149A). FIG. 8G shows the right hind limb with skin removed from a first mouse and from a second mouse 72 hours after peptide administration of 10 nmol SEQ ID NO: 149 peptide conjugated to a Cy5.5 fluorophore (SEQ ID NO: 149A). FIG. 8H shows the right hind limb with muscle removed from a first mouse and from a second mouse 72 hours after administration of 10 nmol SEQ ID NO: 149 peptide conjugated to a Cy5.5 fluorophore (SEQ ID NO: 149A).

FIG. 9 illustrates autoradiography images of frozen sections from a mouse, 3 hours after administration of 100 nmol of a radiolabeled peptide of SEQ ID NO: 149. FIG. 9A illustrates the ¹⁴C signal in a different frozen section of the mouse, 3 hours after administration of 100 nmol of a radiolabeled peptide of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205). FIG. 9B illustrates the ¹⁴C signal in a different frozen section of a mouse, 3 hours after administration of 100 nmol of a radiolabeled peptide of SEQ ID NO: 149.

### DETAILED DESCRIPTION

The present disclosure relates generally to compositions and methods for cartilage therapy. In some embodiments, the compositions and methods herein utilize truncated or mutated peptides that home, target, are directed to, are retained by, accumulate in, migrate to, and/or bind to cartilage following administration to a subject. In some embodiments, the truncated or mutated cartilage homing peptides of the present disclosure exert therapeutic effect in cartilage or tissue or cell thereof. In some embodiments, the truncated or mutated cartilage homing peptides of the present disclosure are used to deliver an active agent to cartilage or tissue or cell thereof. The active agent can exert a therapeutic effect on cartilage or tissue or cell thereof. For example, in certain embodiments, the truncated or mutated peptide itself or the active agent allows for localized delivery of an anti-inflammatory or other agent to cartilage or tissue or cell thereof. As another example, the active agent is a fluorophore that can be used for imaging of cartilage. In certain embodiments, the truncated or mutated peptide itself induces therapeutic responses.

Cartilage disorders are particularly difficult to treat. A direct route for active agent administration can be parenterally (e.g., intravenously, subcutaneously, intramuscularly), intra-articularly, by inhalation, dermally, topically, or orally. However, cartilage can be avascular thus intravenous administration of drugs can fail to reach the cartilage in significant amounts. Drugs for cartilage diseases, such as osteoarthritis, can be injected directly locally into the affected area, for example, directly injected into the joint. Few drugs aimed at treating cartilage disorders have proved therapeutically viable with lack of access to target tissue being a primary reason for failure. The lack of access to the target tissue can also lead to administration of doses that are higher than would be necessary if a drug could home, target, or be directed to, is retained by, and/or binds to a target region, tissue, structure or cell. Thus, treatment of cartilage conditions often requires the use of high concentrations of non-specific drugs. In addition, a number of therapeutics are of interest in treating joint disorders, but are problematic because of the level of side effects caused by systemic administration of the drug (Dancevic and McCulloch, Arthritis Res Ther. 16:429 (2014)).

Specific and potent drugs that are capable of contacting the cartilage can counteract the non-specificity of many treatments by selectively targeting and delivering compounds to specific regions, tissues, cells and structures. Such drugs can also be useful to modulate ion channels, protein-protein interactions, extracellular matrix remodeling (i.e., protease inhibition), and the like. Such targeted therapy can allow for lower dosing, reduced side effects, improved patient compliance, and improvement in therapeutic outcomes, which would be advantageous not only in acute disease of the cartilage, but in chronic conditions as well.

The present disclosure provides truncated or mutated peptides that can comprise or can be derived from cystine-dense peptides. As used herein, the term “cystine-dense peptide” can be interchangeable with the terms “knotted peptide,” “knottin,” and “optide,” and cystine-dense peptides can also be abbreviated as “CDPs.” Hitchins, amongst other disulfide-containing peptides, can also be considered “knotted peptides” or “cystine-dense peptides” for the purposes of this disclosure. Knottins, for example, are a class of cystine-dense peptides comprising from about 11 to about 80 amino acids in length that are often folded into a compact structure. Knottins and other cystine-dense peptides are typically assembled into a complex tertiary structure that is characterized by a number of intramolecular disulfide crosslinks and can contain beta strands, one or more alpha helices, and other secondary structures. The presence of the disulfide bonds can give cystine-dense peptides remarkable environmental stability, allowing them to withstand extremes of temperature and pH, to resist proteolytic enzymes in the blood stream or digestive tract, and can provide specific biodistribution, pharmacokinetic, binding interactions, cellular processing, or other properties of physiologic and therapeutic value. The truncated or mutated peptides disclosed herein can be derived from certain cystine-dense peptides. The present disclosure describes a class of cystine-dense peptides that can effectively contact cartilage and be used either directly or as carriers of active drugs, peptides, or molecules to treat a cartilage condition. For instance, osteoarthritis is a cartilage condition that is associated with the thinning of cartilage covering the ends of bones resulting in bone directly contacting bone within the joint. Over time, the ends of the bones are subjected to increased levels of friction which ultimately causes erosion of the end of the bone. Individuals suffering from osteoarthritis experience reduced motion and increased pain. A therapeutic peptide that could contact the cartilage at the joint and ends of the bone to interact with the chondrocytes and induce increased expression of extracellular matrix proteins could be used in the treatment and prevention of osteoarthritis by increasing expression of collagen through, for example, the rate of production, amount of production, inhibition of proteins which degrade collagen, promote expression of other proteins which maintain the integrity of existing collagen proteins, or other mechanism. A peptide could also affect nearby tissues or cells such as the bone, ligaments, muscle, tendons, bursa, connective tissue, blood vessels, peripheral nerves, osteoclasts, osteoblasts, fibroblasts, synoviocytes, monocytes/macrophages, lymphocytes, plasma cells, adipocytes, endothelial cells, neurons, ligaments, muscle, tendons, and bursa. The truncated or mutated peptides of the disclosure can be used to treat the symptoms of various conditions. The truncated or mutated peptides of the disclosure can bind to, home to, migrate to, accumulate in, be retained by, or be directed to cartilage and its components, including chondrocytes, extracellular matrix, collagen, hyaluranon, aggrecan (also known as cartilage-specific proteoglycan core protein (CSPCP)), or other components of the extracellular matrix and the joint, or to other nearby components such as those described herein in joints and cartilaginous tissues as listed above.

Also described herein are truncated or mutated peptides that selectively home, target, are directed to, migrate to, are retained by, or accumulate in and/or bind to specific regions, tissues, structures or cells of the cartilage that aid in managing, decreasing, ablating or reducing pain (e.g., joint pain) due to chronic disease or cartilage injury or other therapeutic indications as described herein. A truncated or mutated peptide that homes, targets, migrates to, is directed to, is retained by, or accumulates in and/or binds to one or more specific regions, tissues, structures or cells of the cartilage can have fewer off-target and potentially negative effects, for example, side effects that often limit use and efficacy of pain drugs. In addition, such truncated or mutated peptides can reduce dosage and increase the efficacy of existing drugs by directly targeting them to a specific region, tissue, structure or cell of the cartilage and helping the contact the cartilage or increasing the local concentration of agent. The truncated or mutated peptide itself can modulate pain or it can be complexed, conjugated, or fused to an agent that modulates pain. Such pain modulation may operate by various mechanisms such as modulating inflammation, autoimmune responses, direct or indirect action on pain receptors, cell killing, or programmed cell death (whether via an apoptotic and/or non-apoptotic pathway of diseased cells or tissues, and the like (Tait et al., J Cell Sci 127(Pt 10):2135-44 (2014)).

Truncated or mutated peptides of this disclosure that home, target, are directed to, migrate to, are retained by, accumulate in, or bind to specific regions, tissues, structures or cells of the cartilage can do so with different degrees of efficiency. Truncated or mutated Peptides can have a higher concentration in cartilage than in other locations, such as blood or muscle. Peptides can be recorded as having a signal in cartilage as a percentage of signal in blood. For example, a cartilage signal of 200% indicates that the signal in cartilage is twice as high as the signal in blood. In some embodiments, peptides that have cartilage homing properties can have a cartilage signal of >170% by radiographic densitometry measurements. In other embodiments, peptides that are cartilage homers can have a cartilage signal of >200% by radiographic densitometry measurements. In other embodiments, peptides that are more efficient cartilage homers can have a cartilage signal of >300% by radiographic densitometry measurements. In other embodiments, peptides that are more efficient cartilage homers can have a cartilage signal of >400% by radiographic densitometry measurements. In other embodiments, peptides that are strongest cartilage homers of highest interest can have a cartilage signal of >500% by radiographic densitometry measurements. In some embodiments, measurement of the ratio of peptide concentration in blood, muscle, or other tissues relative to the peptide concentration in cartilage can be performed using various methods including measuring the densitometry signal of peptides labeled with radioisotopes (as described above), or by using other assays.

Truncated or mutated peptides that selectively home, target, are directed to, migrate to, are retained by, or accumulate in and/or bind to specific regions, tissues, structures or cells of the cartilage can occur after administration of the peptide to a subject. A subject can be a human or a non-human animal.

The truncated or mutated peptides disclosed herein can be used as active agents, or complexed, conjugated, or fused to detection agents such a fluorophores, iodide-containing X-ray contrast agents, lanthanide chelates (e. g., gadolinium for MRI imaging), perfluorocarbons (for ultrasound), or PET tracers (e. g., 18F or 11C) for imaging and tracing the truncated or mutated peptide, or complexed, conjugated, or fused to agents such as anti-inflammatory active agents or other active agents to the joint to treat inflammation or other disease.

The truncated or mutated peptides disclosed herein can be used to bind cartilage explants ex vivo. Cartilage explants can be from any subject, such as a human or an animal. Assessment of truncated or mutated peptide binding to cartilage explants can be used to screen peptides that may efficiently home to cartilage in vivo

In some embodiments, truncated or mutated peptides of this disclosure home, target, are directed to, migrate to, are retained by, accumulate in, or bind to specific regions, tissues, structures or cells of the kidneys. For example, in some embodiments, truncated or mutated peptides of this disclosure home, target, are directed to, migrate to, are retained by, accumulate in, or bind to the proximal tubules of the kidneys, kidney nephrons, or podocytes. Peptides that selectively home, target, are directed to, migrate to, are retained by, or accumulate in and/or bind to specific regions, tissues, structures or cells of the kidney can occur after administration of the peptide to a subject. A subject can be a human or a non-human animal. The truncated or mutated peptides disclosed herein can be used as active agents, or complexed, conjugated, or fused to detection agents such a fluorophores, iodide-containing X-ray contrast agents, lanthanide chelates (e. g., gadolinium for MRI imaging), perfluorocarbons (for ultrasound), or PET tracers (e. g., 18F or 11C) for imaging and tracing the truncated or mutated peptide, or complexed, conjugated, or fused to agents such as anti-inflammatory agents or other agents to the kidney to treat renal cancer, chronic kidney failure or other kidney disease.

One roadblock in the advancement and wide spread use of peptides as a therapeutic is that peptides can be chemically and physically unstable. During the process of manufacturing of therapeutic peptides essential considerations can include storage conditions, sustained biochemical function, and in vivo delivery. Peptide degradation products can result in the formation of species that alter the safety profile, potency, and immunogenicity of the peptide. These peptide degradation products can form during manufacture and storage, as well as in vivo after delivery to a patient. Furthermore, peptide degradation may limit the shelf-life and increase production cost due to unstable peptides requiring refrigeration or shipment on dry ice. The latter can necessitate continual monitoring and validation of peptides as degradation products could have formed during the manufacturing process. Hence, there is an urgent need for the rationale design and production of therapeutic peptides that have enhanced stability, for example, in the ambient environment, during the process of manufacturing, in storage, and that prevent the likelihood of peptide degradation under a variety of conditions.

In some embodiments, the truncated or mutated peptides and truncated or mutated peptide-drug conjugates of the present disclosure have stability properties that minimize truncated or mutated peptide or truncated or mutated peptide-drug conjugate degradation to enable adequate storage. Long term, accelerated, and intermediate storage conditions for the truncated or mutated peptides and truncated or mutated peptide-drug conjugates of the present disclosure can include long term storage conditions of 25° C. 2° C./60% relative humidity (RH) 5% RH, or 30° C. 2° C./65% RH 5% RH for at least 6 months, at least 12 months, and up to 1 year, up to 2 years, up to 3 years, up to 4 years, or longer than 4 years. In addition, intermediate and short term storage conditions (e.g., during transport, distribution, manufacturing, or handling), or long term storage conditions for certain climates and infrastructures, can include storage conditions of 30° C. 2° C./65% RH 5% RH or 40° C. 2° C./75% RH 5% RH for up to 1 hour, for up to 8 hours, for up to 1 day, for up to 3 days, for up to 1 week, for up to 1 month, for up to 3 months, for up to 6 months or at least 6 months, up to 1 year, up to 2 years, up to 3 years, up to 4 years, or longer than 4 years. Moreover, the truncated or mutated peptides and truncated or mutated peptide-drug conjugates of the present disclosure can be refrigerated, for example between 5° C. 3° C. for at least 6 months, at least 12 months, and up to 1 year, up to 2 years, up to 3 years, up to 4 years, or longer than 4 years. In addition, intermediate and short term refrigeration conditions (e.g., during transport, distribution, manufacturing, or handling) can include 25° C. 2° C./60% RH 5% RH for up to 1 hour, for up to 8 hours, for up to 1 day, for up to 3 days, for up to 1 week, for up to 1 month, for up to 3 months, for up to 6 months or at least 6 months, and potentially longer (at least 12 months and up to 1 year, up to 2 years, up to 3 years, up to 4 years, or longer than 4 years). Such conditions for storage, whether based on ambient or refrigerated conditions can be adjusted based upon the four zones in the world (e.g., the International Council for Harmonisation of Technical Requirements for Pharmaceuticals for Human Use (ICH) stability Zone I, II, III, or IV) that are distinguished by their characteristic prevalent annual climatic conditions. In addition, formulation components can be principally chosen for their ability to preserve the native conformation and chemical structure of the peptides and peptide-drug conjugates of the present disclosure in storage by preventing denaturation due to hydrophobic interactions and aggregation, as well as by preventing chemical degradation, including truncation, oxidation, deamidation, cleavage, hydrolysis, isomerization, disulfide exchange, racemization, and beta elimination (Cleland, et al., Crit Rev Ther Drug Carrier Syst 10(4): 307-377 (1993); Shire et al., J Pharm Sci 93(6): 1390-1402 (2004); Wakankar and Borchardt, J Pharm Sci 95(11): 2321-2336 (2006)).

In some embodiments, the truncated or mutated peptides and truncated or mutated peptide-drug conjugates of the present disclosure have incorporated properties that minimize immunogenicity of the peptides and peptide-drug conjugates. Immunogenicity can be a major concern with the development of therapeutic peptides and proteins, and there is an urgent need for the rationale design and production of therapeutic peptides that have reduced immunogenicity and that increase their safety and efficacy. Immunogenicity can occur against a desired peptide sequence or a peptide degradation product. Immunogenicity can occur when a patient develops an immune response to the therapeutic peptide, protein, conjugate, or other drug, such as by producing antibodies that bind to and/or neutralize the therapeutic peptide, protein, conjugate, or other drug. The likelihood of immunogenicity can increase when drugs are administered more than once or chronically. Immunogenicity can reduce patient exposure to the drug, can reduce effectiveness of the drug, and can also result in safety risks for the patient, such as generating an immune response to self-proteins or other adverse responses related to increased immunogenicity to the therapeutic peptide, protein, conjugate, or other drug. Immunogenic responses can vary from patient to patient and also amongst different groups of HLA alleles, as well as over time. As such, minimizing risk of immunogenicity with a therapeutic peptide or protein can be important for developing a drug that can be effectively and safely used for treatment. Various methods exist for assessment of immunogenic potential, which can include in silico methods, in vitro testing, preclinical in vivo testing, and assessment during clinical dosing. Evaluation early in product design and development of the therapeutic peptides and peptide-drug conjugates of the present disclosure in the in vivo milieu in which they function (e.g., in inflammatory environments or at physiologic pH) can reveal susceptibilities to modifications (e.g., aggregation and deamidation) that can result in loss of efficacy or induction of immune responses. Such information can be used to facilitate product engineering to enhance the stability of the product under such in vivo conditions or reduce immunogenicity. Moreover, the therapeutic truncated or mutated peptides and truncated or mutated peptide-drug conjugates of the present disclosure can be designed to minimize protein aggregation. Strategies to minimize aggregate formation can be used early in drug development, for example, by using an appropriate cell substrate, selecting manufacturing conditions that minimize aggregate formation, employing a robust purification scheme that removes aggregates to the greatest extent possible, and choosing a formulation and container closure system that minimize aggregation during storage.

Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N), glutamine (Q), histidine (H), lysine (K), or arginine (R).

Some embodiments of the disclosure contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxyl terminal direction, in accordance with standard usage and convention.

### Peptides

The cystine-dense peptides herein can bind targets with antibody-like affinity. The cystine-dense peptides can modulate the activity of a plurality of cartilage regions, tissues, structures or cells. For example, in some embodiments, the cystine-dense peptide complexed, conjugated, or fused to a bone-modifying drug homes to the cartilage of a diseased joint and releases the drug, creating a higher local concentration of drug in an area of eroded or damaged bone than would be achieved without the cartilage targeting function of the peptide. The cystine-dense peptide can be complexed, conjugated, or fused to a drug that can affect nearby tissues or cells such as the ligaments, muscle, tendons, bursa, connective tissue, blood vessels, peripheral nerves, osteoclasts, osteoblasts, fibroblasts, synoviocytes, monocytes/macrophages, lymphocytes, plasma cells, adipocytes, endothelial cells, neurons, ligaments, muscle, tendons, and bursa. The cystine-dense peptide complexed, conjugated, or fused to a drug can bind to, home to, migrate to, accumulate in, be retained by, or be directed to cartilage and its components, including chondrocytes, extracellular matrix, collagen of any type, hyaluranon, aggrecan (also known as cartilage-specific proteoglycan core protein (CSPCP)), proteoglycans, glycoasminoglycans, glycoproteins, decorin, biclycan, fibromodulin, or other components of the extracellular matrix and the joint, or to other nearby components such as those described herein in joints and cartilaginous tissues as listed above. Some of the cartilage regions, tissues, and structures that peptides and peptide-drug conjugates can target to treat a cartilage-associated disorder include: (a) elastic cartilage; (b) hyaline cartilage, such as articular cartilage and physeal cartilage; (c) fibrocartilage; and (d) any cells or cell types in (a)-(c) above. Some of the areas where the peptide and peptide-drug conjugates can target to treat a cartilage-associated disorder include: cartilage includes joints such as knees, hips, or digits, nasal cartilage, spinal cartilage, tracheal cartilage, and rib cartilage. In various aspects, cartilage components include aggrecan and type II collagen. Additionally, in some embodiments, cystine-dense peptides can penetrate into cells. In other embodiments, cystine-dense peptides do not enter cells. In other embodiments, cystine-dense peptides exhibit more rapid clearance and cellular uptake compared to other types of molecules.

The peptides of the present disclosure can comprise cysteine amino acid residues. In some cases, the peptide has at least 4 cysteine amino acid residues. In some cases, the peptide has at least 6 cysteine amino acid residues. In other cases, the peptide has at least 8 cysteine amino acid residues, at least 10 cysteine amino acid residues, at least 12 cysteine amino acid residues, at least 14 cysteine amino acid residues or at least 16 cysteine amino acid residues.

A cystine-dense peptide can comprise disulfide bridges. A cystine-dense can be a peptide wherein 5% or more of the residues are cysteines forming intramolecular disulfide bonds as cystines. A disulfide-linked peptide can be a drug scaffold. In some embodiments, the disulfide bridges form an inhibitor knot. A disulfide bridge can be formed between cysteine residues, for example, between cysteines 1 and 4, 2 and 5, or, 3 and 6. In some cases, one disulfide bridge passes through a loop formed by the other two disulfide bridges, for example, to form the inhibitor knot. In other cases, the disulfide bridges can be formed between any two cysteine residues.

The present disclosure further includes peptide scaffolds that, e.g., can be used as a starting point for generating additional peptides that can target and home to cartilage. In some embodiments, these scaffolds can be derived from a variety of cystine-dense peptides. In certain embodiments, cystine-dense peptides are assembled into a complex tertiary structure that is characterized by a number of intramolecular disulfide crosslinks, and optionally contain beta strands and other secondary structures such as an alpha helix. For example, cystine-dense peptides include, in some embodiments, small disulfide-rich proteins characterized by a disulfide through disulfide knot. This knot can be, e.g., obtained when one disulfide bridge crosses the macrocycle formed by two other disulfides and the interconnecting backbone. In some embodiments, the cystine-dense peptides can include growth factor cysteine knots or inhibitor cysteine knots. Other possible peptide structures can include peptide having two parallel helices linked by two disulfide bridges without β-sheets (e.g., hefutoxin).

A cystine-dense peptide can comprise at least one amino acid residue in an L configuration. A cystine-dense peptide can comprise at least one amino acid residue in a D configuration. In some embodiments, a cystine-dense peptide is 15-40 amino acid residues long. In other embodiments, a cystine-dense peptide is 11-57 amino acid residues long. In further embodiments, a cystine-dense peptide is at least 20 amino acid residues long.

In some embodiments, cartilage homing peptides are family members of the sequences GSXVXIXVKCXGSXQCLXPCKXAXGXRXGKCMNGKCXCXPXX (SEQ ID NO: 21) or XVXIXVKCXGSXQCLXPCKXAXGXRXGKCMNGKCXCXPXX (SEQ ID NO: 87), wherein each X can independently be any amino acid or amino acid analogue or null, in which these sequences are based on the most common elements found in the following sequences: -VRIPVSCKHSGQCLKPCKDA-GMRFGKCMNGKCDCTPK- (SEQ ID NO: 198), GVPINVKCRGSRDCLDPCKKA-GMRFGKCINSKCHCTP-- (SEQ ID NO: 199), ---EVIRCSGSKQCYGPCKQQTGCTNSKCMNKVCKCYGCG (SEQ ID NO: 200), GVIINVKCKISRQCLEPCKKA-GMRFGKCMNGKCHCTPK- (SEQ ID NO: 201), GVPTDVKCRGSPQCIQPCKDA-GMRFGKCMNGKCHCTPK- (SEQ ID NO: 202), GVPINVSCTGSPQCIKPCKDA-GMRFGKCMNRKCHCTPK- (SEQ ID NO: 203), -VGINVKCKHSGQCLKPCKDA-GMRFGKCINGKCDCTPK- (SEQ ID NO: 204), GVPINVRCRGSRDCLDPCRRA-GMRFGRCINSRCHCTP-- (SEQ ID NO: 205; also disclosed herein as SEQ ID NO: 128; GS versions of SEQ ID NO: 205 and SEQ ID NO: 128 are shown in SEQ ID NO: 46 and SEQ ID NO: 149), QKILSNRCNNSSECIPHCIRIFGTRAAKCINRKCYCYP-- (SEQ ID NO: 206), -VFINVKCRGSPECLPKCKEAIGKSAGKCMNGKCKCYP-- (SEQ ID NO: 207), -VPTDVKCRGSPQCIQPCKDA-GMRFGKCMNGKCHCTP-- (SEQ ID NO: 208), --AEIIRCSGTRECYAPCQKLTGCLNAKCMNKACKCYGCV (SEQ ID NO: 209), -RPTDIKCSASYQCFPVCKSRFGKTNGRCVNGLCDCF--- (SEQ ID NO: 210), -QFTDVKCTGSKQCWPVCKQMFGKPNGKCMNGKCRCYS-- (SEQ ID NO: 211), -VGINVKCKHSRQCLKPCKDA-GMRFGKCTNGKCHCTPK- (SEQ ID NO: 212), -VVIGQRCYRSPDCYSACKKLVGKATGKCTNGRCDC---- (SEQ ID NO: 213), --NFKVEGACSKPCRKYCIDK-GARNGKCINGRCHCYY-- (SEQ ID NO: 214), and QIDTNVKCSGSSKCVKICIDRYNTRGAKCINGRCTCYP-- (SEQ ID NO: 215). Furthermore, peptides denoted as SEQ ID NO: 128 (also shown as SEQ ID NO: 205), and SEQ ID NO: 149 (also shown as SEQ ID NO: 46; non-GS version of SEQ ID NO: 149/SEQ ID NO: 46 are shown in SEQ ID NO: 128 and SEQ ID NO: 205) are excluded from (or not included in) peptides comprising SEQ ID NO: 21 and SEQ ID NO: 87.

In some embodiments, the cartilage homing peptides are family members of the sequences GSXVXIXVRCXGSXQCLXPCRXAXGXRXGRCMNGRCXCXPXX (SEQ ID NO: 22) or XVXIXVRCXGSXQCLXPCRXAXGXRXGRCMNGRCXCXPXX (SEQ ID NO: 88) wherein each X individually can be any amino acid or amino acid analogue or null, in which these sequences are based on the most common elements found in the following sequences and with K interchanged with R: SEQ ID NO: 198-SEQ ID NO: 215.

In some embodiments, a peptide comprises the sequence GSGVPIX¹VRCRGSRDCX²X³PCRRAGX⁴RFGRCIX⁵X⁶RCX⁷CX⁸P (SEQ ID NO: 23) or GVPIX¹VRCRGSRDCX²X³PCRRAGX⁴RFGRCIX⁵X⁶RCX⁷CX⁸P (SEQ ID NO: 89), where the following residues where X¹ is selected from N, S, or G, wherein X² is selected from L or Y, wherein X³ is selected from D or E, wherein X⁴ is selected from M or T, wherein X⁵ is selected from N, Q, A, S, T, or L, wherein X⁶ is selected from S, G, or R, wherein X⁷ is selected from H or Y, and wherein X⁸ is selected from T or Y. In some embodiments, zero or one or more of the R residues in SEQ ID NO: 23 or SEQ ID NO: 89 can be replaced with K residues. In some embodiments, zero or one or more of the R residues in SEQ ID NO: 23 or SEQ ID NO: 89 can be replaced with A residues in any combination. In other embodiments, zero or one or more R residues in SEQ ID NO: 23 or SEQ ID NO: 89 can each be replaced with either a K or an A residue. In some embodiments, at least one but not all eight of the residues X¹ through X⁸ in SEQ ID NO: 23 or SEQ ID NO: 89 is excluded at the following residue at the denoted position: wherein X¹ is N, wherein X² is L, wherein X³ is D, wherein X⁴ is M, wherein X⁵ is N, wherein X⁶ is S, wherein X⁷ is H and wherein X⁸ is T. Furthermore, the following peptide (also denoted as SEQ ID NO: 128 or SEQ ID NO: 205, and SEQ ID NO: 149 or SEQ ID NO: 46 respectively) is excluded from (or not included in) the peptides of this disclosure: wherein all eight of the residues X¹ through X⁸ in of SEQ ID NO: 23 or SEQ ID NO: 89 include the following residues at the denoted position: X¹ is N, wherein X² is L, wherein X³ is D, wherein X⁴ is M, wherein X⁵ is N, wherein X⁶ is S, wherein X⁷ is H and wherein X⁸ is T. Furthermore, peptides denoted as SEQ ID NO: 128 (also shown as SEQ ID NO: 205), and SEQ ID NO: 149 (also shown as SEQ ID NO: 46; non-GS version of SEQ ID NO: 149/SEQ ID NO: 46 are shown in SEQ ID NO: 128 and SEQ ID NO: 205) are excluded from (or not included in) peptides comprising SEQ ID NO: 23 and SEQ ID NO: 89.

In some embodiments, cartilage homing peptides are family members of the sequences GSRCRGSRDCX¹X²PCRRAGX³RFGRCIX⁴X⁵RCX⁶CX⁷P (SEQ ID NO: 24) or RCRGSRDCX¹X²PCRRAGX³RFGRCIX⁴X⁵RCX⁶CX⁷P (SEQ ID NO: 106), wherein X¹ is selected from L or Y, wherein X² is selected from D or E, wherein X³ is selected from M or T, wherein X⁴ is selected from N, Q, A, S, T, or L, wherein X⁵ is selected from S, G, or R, wherein X⁶ is selected from H or Y, and wherein X⁷ is selected from T or Y. Furthermore, peptides denoted as SEQ ID NO: 128 (also shown as SEQ ID NO: 205), and SEQ ID NO: 149 (also shown as SEQ ID NO: 46) are excluded from (or not included in) peptides comprising SEQ ID NO: 24 and SEQ ID NO: 106. In some embodiments, cartilage homing peptides are family members of the sequences GSKCXGSXQCLXPCKXAXGXRXGKCMNGKCXCXPXX (SEQ ID NO: 25) or KCXGSXQCLXPCKXAXGXRXGKCMNGKCXCXPXX (SEQ ID NO: 107), wherein each X can individually be any amino acid or amino acid analogue or null. In some embodiments, cartilage homing peptides are family members of the sequences GSRCXGSXQCLXPCRXAXGXRXGRCMNGRCXCXPXX (SEQ ID NO: 26) or RCXGSXQCLXPCRXAXGXRXGRCMNGRCXCXPXX (SEQ ID NO: 108), wherein each X can individually be any amino acid or amino acid analogue or null.

In some embodiments, a peptide comprises the sequence XVXIXVX₁CXGSXX₂CX₃X₄PCX₅XAXGX₆RXGX₇CX₈X₉X₁₀X₁₁CX₁₂CX₁₃P (SEQ ID NO: 219), wherein each X and X₁₋₁₃ are individually any amino acid or no amino acid and at least one of the following residues at the denoted position, more than one of the following residues at the denoted position, or all of the following residues at the denoted position is included in SEQ ID NO: 219: X₁ is K, X₂ is Q, X₃ is Y, X₄ is E, X₅ is K, X₆ is T, X₇ is K, X₈ is M, X₉ is Q, A, S, T, or L, X₁₀ is G, X₁₁ is K, X₁₂ is Y, or X₁₃ is Y or GSXVXIXVX₁CXGSXX₂CX₃X₄PCX₅XAXGX₆RXGX₇CX₈X₉X₁₀X₁₁CX₁₂CX₁₃P (SEQ ID NO: 220), wherein each X and X₁₋₁₃ are individually any amino acid or no amino acid and at least one of the following residues at the denoted position, more than one of the following residues at the denoted position, or all of the following residues at the denoted position is included in SEQ ID NO: 220: X₁ is K, X₂ is Q, X₃ is Y, X₄ is E, X₅ is K, X₆ is T, X₇ is K, X₈ is M, X₉ is Q, A, S, T, or L, X₁₀ is G, X₁₁ is K, X₁₂ is Y, or X₁₃ is Y. SEQ ID NO: 219 and SEQ ID NO: 220 are variant consensus peptide sequences, included within the family of SEQ ID NO: 87 and SEQ ID NO: 21 consensus sequences, which variant consensus sequences include variants with further improved properties of the peptides as described herein, with or without an N-terminal GS. In some embodiments, at least two of the following residues at the denoted position, at least three of the following residues at the denoted position, at least four of the following residues at the denoted position, at least five of the following residues at the denoted position, at least six of the following residues at the denoted position, at least seven of the following residues at the denoted position, at least eight of the following residues at the denoted position, at least nine of the following residues at the denoted position, at least 10 of the following residues at the denoted position, at least 11 of the following residues at the denoted position, or at least 12 of the following residues at the denoted position are included in SEQ ID NO: 219 and SEQ ID NO: 220, respectively: X₁ is K, X₂ is Q, X₃ is Y, X₄ is E, X₅ is K, X₆ is T, X₇ is K, X₈ is M, X₉ is Q, A, S, T, or L, X₁₀ is G, X₁₁ is K, X₁₂ is Y, or X₁₃ is Y in SEQ ID NO: 219 and SEQ ID NO: 220.

In some embodiments, a peptide comprises the sequence

(SEQ ID NO: 221)
X₁CXGSXX₂CX₃X₄PCX₅XAXGX₆RXGX₇CX₈X₉X₁₀X₁₁CX₁₂CX₁₃P,

wherein each X and X₁₋₁₃ are individually any amino acid or no amino acid and at least one of the following residues at the denoted position, more than one of the following residues at the denoted position, or all of the following residues at the denoted position is included in SEQ ID NO:221: X₁ is K, X₂ is Q, X₃ is Y, X₄ is E, X₅ is K, X₆ is T, X₇ is K, X₈ is M, X₉ is Q, A, S, T, or L, X₁₀ is G, X₁₁ is K, X₁₂ is Y, or X₁₃ is Y or

(SEQ ID NO: 222)
GSX₁CXGSXX₂CX₃X₄PCX₅XAXGX₆RXGX₇CX₈X₉X₁₀X₁₁CX₁₂CX₁₃P,

wherein each X and X₁₋₁₃ are individually any amino acid or no amino acid and at least one of the following residues at the denoted position, more than one of the following residues at the denoted position, or all of the following residues at the denoted position is included in SEQ ID NO:222: X₁ is K, X₂ is Q, X₃ is Y, X₄ is E, X₅ is K, X₆ is T, X₇ is K, X₈ is M, X₉ is Q, A, S, T, or L, X₁₀ is G, X₁₁ is K, X₁₂ is Y, or X₁₃ is Y. SEQ ID NO: 221 and SEQ ID NO: 222 are variant consensus peptide sequences, included within the family of SEQ ID NO: 87 and SEQ ID NO: 21 consensus sequences, which variant consensus sequences include truncated variants with further improved properties of some of the peptides and fragments of the peptides of the disclosure as described herein, with or without an N-terminal GS. Furthermore, peptides denoted as SEQ ID NO: 128 (also shown as SEQ ID NO: 205), and SEQ ID NO: 149 (also shown as SEQ ID NO: 46; non-GS version of SEQ ID NO: 149/SEQ ID NO: 46 are shown in SEQ ID NO: 128 and SEQ ID NO: 205) are excluded from (or not included in) peptides comprising SEQ ID NO: 221 and SEQ ID NO: 222. In some embodiments, at least two of the following residues at the denoted position, at least three of the following residues at the denoted position, at least four of the following residues at the denoted position, at least five of the following residues at the denoted position, at least six of the following residues at the denoted position, at least seven of the following residues at the denoted position, at least eight of the following residues at the denoted position, at least nine of the following residues at the denoted position, at least 10 of the following residues at the denoted position, at least 11 of the following residues at the denoted position, or at least 12 of the following residues at the denoted position are included in SEQ ID NO: 221 or SEQ ID NO: 222, respectively: X₁ is K, X₂ is Q, X₃ is Y, X₄ is E, X₅ is K, X₆ is T, X₇ is K, X₈ is M, X₉ is Q, A, S, T, or L, X₁₀ is G, X₁₁ is K, X₁₂ is Y, or X₁₃ is Y in SEQ ID NO: 221 and SEQ ID NO: 222.

In some embodiments, a peptide comprises the sequence

(SEQ ID NO: 223)
KCRGSRQCX¹X²PCKRAX⁸GX³RFGKCMX⁴X⁵KCX⁶CX⁷P
or
(SEQ ID NO: 224)
GSKCRGSRQCX¹X²PCKRAX⁸GX³RFGKCMX⁴X⁵KCX⁶CX⁷P,

wherein X¹ is selected from L or Y, wherein X² is selected from D or E, wherein X³ is selected from M or T, wherein X⁴ is selected from N, Q, A, S, T, or L, wherein X⁵ is selected from S, G, or R, wherein X⁶ is selected from H or Y, wherein X⁷ is selected from T or Y, and X⁸ is any amino acid or null. SEQ ID NO: 223 and SEQ ID NO: 224 are variant consensus sequences, and include variants with further improved properties of some of the peptides and fragments of the peptides of the disclosure, with or without an N-terminal GS.

In some embodiments, a peptide comprises the sequence

(SEQ ID NO: 225)
XVXIXVKCRGSRQCX¹X²PCKRAX⁸GX³RFGKCMX⁴X⁵KCX⁶CX⁷P
or
(SEQ ID NO: 226)
GSXVXIXVKCRGSRQCX¹X²PCKRAX⁸GX³RFGKCMX⁴X⁵KCX⁶CX⁷P,

wherein X¹ is selected from L or Y, wherein X² is selected from D or E, wherein X³ is selected from M or T, wherein X⁴ is selected from N, Q, A, S, T, or L, wherein X⁵ is selected from S, G, or R, wherein X⁶ is selected from H or Y, wherein X⁷ is selected from T or Y, and X⁸ is any amino acid or null. SEQ ID NO: 225 and SEQ ID NO: 226 are variant consensus sequences, and include variants with further improved properties of some of the peptides and fragments of the peptides of the disclosure, with or without an N-terminal GS.

In some embodiments, a peptide comprises the sequence KCRGSRDCLDPCKKAGMRFGKCINSKCHCTP (SEQ ID NO: 109), with or without an N-terminal GS. Furthermore, peptides denoted as SEQ ID NO: 150 and SEQ ID NO: 199 (non-GS version of SEQ ID NO: 150) are excluded from (or not included in) peptides comprising SEQ ID NO: 109.

In some embodiments, a peptide is a fragment comprising the sequence GRCINSRC (SEQ ID NO: 227).

In some embodiments, a peptide is a fragment comprising the sequence GRCIXXRC (SEQ ID NO: 228), wherein each X can independently be any amino acid or amino acid analogue or null.

In some embodiments, a peptide is a fragment comprising the sequence GRCIX¹X²RC (SEQ ID NO: 229), wherein X¹=N, Q, A, S, T or L and X²=S, G, or R.

In some embodiments, a peptide is a fragment comprising the sequence PCR (SEQ ID NO: 230).

In some embodiments, a peptide is a fragment comprising the sequence CLDPCRRA (SEQ ID NO: 231).

In some embodiments, a peptide is a fragment comprising the sequence CLDPCRR (SEQ ID NO: 232).

In some embodiments, a peptide is a fragment comprising the sequence RCRGSRDC (SEQ ID NO: 257).

In some embodiments, a peptide is a fragment comprising the sequence PCRRAG (SEQ ID NO: 258).

In some embodiments, a peptide is a fragment comprising the sequence RFGRCI (SEQ ID NO: 259).

The peptide fragments disclosed herein can be used as immunogenic epitopes (i.e., for the creation of antibodies, antibody fragments or CDRs that would bind such fragments) or otherwise used to identify the peptides of the disclosure, or screen additional related variants to the peptides of the disclosure from expression libraries and the like, or to purify the peptides of the disclosure, using variety of methods known in the art including western blot, affinity chromatography, FACS, and the like. Moreover, polynucleotide sequences to such fragments can be used as molecular probes or in PCR screens to isolate and identify polynucleotides encoding the peptides of the present invention or screen additional related variants to the peptides of the disclosure from expression libraries and the like. For example, the fragments of SEQ ID NO: 227-SEQ ID NO: 232, and SEQ ID NO: 257-SEQ ID NO: 259 can be used for this purpose.

The N-terminal GS sequence can be included or excluded between the peptides of the present disclosure.

Moreover certain small fragments identified herein are conserved or functional peptide sequences or peptide domains that can be used to create further variant peptides of the disclosure with enhanced functionality with respect to immunogenicity, stability, manufacturability, cartilage binding, and the like as disclosed herein. For example, the following fragments can be used to enhance the functionality of a peptide comprising SEQ ID NO: 28, SEQ ID NO: 45-SEQ ID NO: 51, SEQ ID NO: 109, SEQ ID NO: 110, SEQ ID NO: 128-SEQ ID NO: 133, SEQ ID NO: 149, or SEQ ID NO: 260-SEQ ID NO: 263 and includes one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null, the peptide having one or more of the foregoing peptide fragments located at the corresponding location in the peptide relative to those fragments (or with reference to those fragments) as located within SEQ ID NO: 128 or SEQ ID NO: 149, SEQ ID NO: 28, SEQ ID NO: 109, SEQ ID NO: 150, SEQ ID NO: 199, SEQ ID NO: 110, SEQ ID NO: 127, SEQ ID NO: 260 or SEQ ID NO: 262, with or without an N-terminal GS (for example, using a sequence alignment or other methodology). Moreover certain fragments identified herein are conserved or functional peptide sequences or peptide domains that can be incorporated into variant peptides of the disclosure to enhanced functionality with respect to cartilage homing, stability, manufacturability, cartilage binding, and the like as disclosed herein. For example, the following fragments can be used to enhance the functionality of a peptide comprising SEQ ID NO: 27, SEQ ID NO: 29-SEQ ID NO: 44, SEQ ID NO: 52-SEQ ID NO: 66, SEQ ID NO: 111-SEQ ID NO: 126, SEQ ID NO: 134-SEQ ID NO: 148, SEQ ID NO: 233-SEQ ID NO: 256, and SEQ ID NO: 21-SEQ ID NO: 26, SEQ ID NO: 87-SEQ ID NO: 89, SEQ ID NO: 106-SEQ ID NO: 108, SEQ ID NO: 219-SEQ ID NO: 226 and includes one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259), the peptide having one or more foregoing fragment sequences located at such corresponding location within the peptide of any one of SEQ ID NO: 87, SEQ ID NO: 89 or SEQ ID NO: 106, SEQ ID NO: 109, SEQ ID NO: 150, SEQ ID NO: 199, SEQ ID NO: 110, SEQ ID NO: 219 or SEQ ID NO: 221, or SEQ ID NO: 224 or SEQ ID NO: 226, with or without an N-terminal GS (for example, using a sequence alignment or other methodology).

In some embodiments, a peptide of the present disclosure comprises one or more of the following peptide fragments within its sequence: GKCINKKCKC (SEQ ID NO: 90); KCIN (SEQ ID NO: 91); KKCK (SEQ ID NO: 92); PCKR (SEQ ID NO: 93); KRCSRR (SEQ ID NO: 94); KQC (SEQ ID NO: 95); GRCINRRCRC (SEQ ID NO: 96); RCIN (SEQ ID NO: 97); RRCR (SEQ ID NO: 98); PCRR (SEQ ID NO: 99); RRCSRR (SEQ ID NO: 100); RQC (SEQ ID NO: 101); PCKK (SEQ ID NO: 102), KKCSKK (SEQ ID NO: 103), GKCMNGKC (SEQ ID NO: 104); GRCMNGRC (SEQ ID NO: 105); GRCINSRC (SEQ ID NO: 227), GRCIXXRC (SEQ ID NO: 228) wherein each X can independently be any amino acid or amino acid analogue or null, GRCIX¹X²RC (SEQ ID NO: 229), wherein X¹=N, Q, A, S, T or L and X²=S, G, or R (SEQ ID NO: 229), PCR (SEQ ID NO: 230), CLDPCRRA (SEQ ID NO: 231), CLDPCRR (SEQ ID NO: 232), RCRGSRDC (SEQ ID NO: 257), PCRRAG (SEQ ID NO: 258), and RFGRCI (SEQ ID NO: 259). In another embodiment, the peptide of the present disclosure comprises a peptide 80%, 85%, 90%, 95%, 96%, 97%, 98%, 99%, or 100% identical to any of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 256 and further comprises one or more of the following peptide fragments within its sequence: GKCINKKCKC (SEQ ID NO: 90); KCIN (SEQ ID NO: 91); KKCK (SEQ ID NO: 92); PCKR (SEQ ID NO: 93); KRCSRR (SEQ ID NO: 94); KQC (SEQ ID NO: 95); GRCINRRCRC (SEQ ID NO: 96); RCIN (SEQ ID NO: 97); RRCR (SEQ ID NO: 98); PCRR (SEQ ID NO: 99); RRCSRR (SEQ ID NO: 100); RQC (SEQ ID NO: 101); PCKK (SEQ ID NO: 102), KKCSKK (SEQ ID NO: 103), GKCMNGKC (SEQ ID NO: 104); GRCMNGRC (SEQ ID NO: 105); GRCINSRC (SEQ ID NO: 227), GRCIXXRC (SEQ ID NO: 228) wherein each X can independently be any amino acid or amino acid analogue or null, GRCIX¹X²RC (SEQ ID NO: 229), wherein X¹=N, Q, A, S, T or L and X²=S, G, or R (SEQ ID NO: 229), PCR (SEQ ID NO: 230), CLDPCRRA (SEQ ID NO: 231), CLDPCRR (SEQ ID NO: 232), RCRGSRDC (SEQ ID NO: 257), PCRRAG (SEQ ID NO: 258), and RFGRCI (SEQ ID NO: 259). In another embodiment, the peptide having one or more of the foregoing peptide fragments within its sequence has the fragment sequences located at the corresponding location in the peptide relative to those fragments (or with reference to those fragments) as located within SEQ ID NO: 128 or SEQ ID NO: 149 (for example, using a sequence alignment or other methodology) (SEQ ID NO: 149 is also disclosed as SEQ ID NO: 46; non-GS version of SEQ ID NO: 149/SEQ ID NO: 46 are shown in SEQ ID NO: 128 and SEQ ID NO: 205). In another embodiment, the fragment sequences disclosed herein are located at such corresponding location within the peptide of any one of SEQ ID NO: 21-SEQ ID NO: 26, SEQ ID NO: 220, SEQ ID NO: 222, SEQ ID NO: 224, SEQ ID NO: 226. In another embodiment, the fragment sequences disclosed herein are located at such corresponding location within the peptide of any one of SEQ ID NO: 87-SEQ ID NO: 89, SEQ ID NO: 106-SEQ ID NO: 108, SEQ ID NO: 219, SEQ ID NO: 221, SEQ ID NO: 223, SEQ ID NO: 225. In another embodiment, the fragment sequences disclosed herein are located at such corresponding location within the peptide of any one of SEQ ID NO: 87, SEQ ID NO: 89 or SEQ ID NO: 106, SEQ ID NO: 110, SEQ ID NO: 219 or SEQ ID NO: 221, or SEQ ID NO: 260 or SEQ ID NO: 262 (for example, using a sequence alignment or other methodology).

In some embodiments, any peptide disclosed herein having an N-terminal GS sequence can have further have one or more of a mutation or corresponding substitution selected from the group consisting of N7S, D18E, M25T, N32Q, N32A, N32S, N32T, N32L, S33G, S33R, L17Y, H36Y, and T38Y.

In some embodiments, any peptide disclosed herein without an N-terminal GS sequence can have further have one or more of a mutation or corresponding substitution selected from the group consisting of N5S, D16E, M23T, N30Q, N30A, N30S, N30T, N30L, S31G, S31R, L15Y, H34Y, and T36Y.

In some embodiments, any peptide disclosed herein without an N-terminal GS sequence can have further have one or more of a mutation or corresponding substitution selected from the group consisting of D10E, M17T, N24Q, N24A, N24S, N24T, N24L, S25G, S25R, L9Y, H28Y, T30Y, R1K, R13K, R14K, R21K and R26K.

In some embodiments, any peptide disclosed herein having an N-terminal GS sequence can have further have one or more of a mutation or corresponding substitution selected from the group consisting of D12E, M19T, N26Q, N26A, N26S, N26T, N26L, S27G, S27R, L11Y, H30Y, T32Y, R3K, R15K, R16K, R23K and R28K.

TABLE 1 lists some exemplary peptides according to the present disclosure.

TABLE 1
Exemplary Amino Acid Sequences

| SEQ ID NO: | Amino Acid Sequence |
|---|---|
| SEQ ID NO: 27 | GSKCRGSRDCLDPCKKAGMRFGKCINSKCHCTP |
| SEQ ID NO: 28 | GSRCRGSRDCLDPCRRAGMRFGRCINSRCHCTP |
| SEQ ID NO: 29 | GSRCRGSRDCLEPCRRAGTRFGRCINGRCHCTP |
| SEQ ID NO: 30 | GSRCRGSRDCLEPCRRAGTRFGRCINRRCHCTP |
| SEQ ID NO: 31 | GSRCRGSRDCLEPCRRAGTRFGRCINSRCHCTP |
| SEQ ID NO: 32 | GSRCRGSRDCLEPCRRAGTRFGRCIQSRCHCTP |
| SEQ ID NO: 33 | GSRCRGSRDCLEPCRRAGTRFGRCIQSRCHCYP |
| SEQ ID NO: 34 | GSRCRGSRDCYEPCRRAGTRFGRCIQSRCHCTP |
| SEQ ID NO: 35 | GSRCRGSRDCLEPCRRAGTRFGRCIQSRCYCTP |
| SEQ ID NO: 36 | GSRCRGSRDCLEPCRRAGTRFGRCIASRCHCYP |
| SEQ ID NO: 37 | GSRCRGSRDCLEPCRRAGTRFGRCISSRCHCYP |
| SEQ ID NO: 38 | GSRCRGSRDCLEPCRRAGTRFGRCITSRCHCYP |
| SEQ ID NO: 39 | GSKCRGSRDCLDPCRRAGMRFGRCINSRCHCTP |
| SEQ ID NO: 40 | GSRCRGSRDCLDPCKRAGMRFGRCINSRCHCTP |
| SEQ ID NO: 41 | GSRCRGSRDCLDPCRKAGMRFGRCINSRCHCTP |
| SEQ ID NO: 42 | GSRCRGSRDCLDPCRRAGMRFGKCINSRCHCTP |
| SEQ ID NO: 43 | GSRCRGSRDCLDPCRRAGMRFGRCINSKCHCTP |
| SEQ ID NO: 44 | GSRCRGSRDCLEPCRRAGTRFGRCINSRCHCYP |
| SEQ ID NO: 45 | GSSGVPINVRCRGSRDCLDPCRRAGMRFGRCINSRCHCTP |
| SEQ ID NO: 46 | GSGVPINVRCRGSRDCLDPCRRAGMRFGRCINSRCHCTP |
| SEQ ID NO: 47 | GSVPINVRCRGSRDCLDPCRRAGMRFGRCINSRCHCTP |
| SEQ ID NO: 48 | GSPINVRCRGSRDCLDPCRRAGMRFGRCINSRCHCTP |
| SEQ ID NO: 49 | GSINVRCRGSRDCLDPCRRAGMRFGRCINSRCHCTP |
| SEQ ID NO: 50 | GSNVRCRGSRDCLDPCRRAGMRFGRCINSRCHCTP |
| SEQ ID NO: 51 | GSVRCRGSRDCLDPCRRAGMRFGRCINSRCHCTP |
| SEQ ID NO: 52 | GSGVPISVRCRGSRDCLEPCRRAGTRFGRCINGRCHCTP |
| SEQ ID NO: 53 | GSGVPISVRCRGSRDCLEPCRRAGTRFGRCIQSRCHCTP |
| SEQ ID NO: 54 | GSGVPISVRCRGSRDCLEPCRRAGTRFGRCINRRCHCTP |
| SEQ ID NO: 55 | GSGVPINVRCRGSRDCLEPCRRAGTRFGRCINSRCHCTP |
| SEQ ID NO: 56 | GSGVPINVRCRGSRDCLEPCRRAGTRFGRCIQSRCHCTP |
| SEQ ID NO: 57 | GSGVPINVRCRGSRDCLEPCRRAGTRFGRCIQSRCHCYP |
| SEQ ID NO: 58 | GSGVPINVRCRGSRDCYEPCRRAGTRFGRCIQSRCHCTP |
| SEQ ID NO: 59 | GSGVPINVRCRGSRDCLEPCRRAGTRFGRCIQSRCYCTP |
| SEQ ID NO: 60 | GSGVPISVRCRGSRDCLEPCRRAGTRFGRCIQSRCHCYP |
| SEQ ID NO: 61 | GSGVPISVRCRGSRDCYEPCRRAGTRFGRCIQSRCHCTP |
| SEQ ID NO: 62 | GSGVPISVRCRGSRDCLEPCRRAGTRFGRCIQSRCYCTP |
| SEQ ID NO: 63 | GSGVPINVRCRGSRDCLEPCRRAGTRFGRCIASRCHCYP |
| SEQ ID NO: 64 | GSGVPINVRCRGSRDCLEPCRRAGTRFGRCISSRCHCYP |
| SEQ ID NO: 65 | GSGVPINVRCRGSRDCLEPCRRAGTRFGRCITSRCHCYP |
| SEQ ID NO: 66 | GSGVPINVRCRGSRDCLEPCRRAGTRFGRCINSRCHCYP |
| SEQ ID NO: 109 | KCRGSRDCLDPCKKAGMRFGKCINSKCHCTP |
| SEQ ID NO: 110 | RCRGSRDCLDPCRRAGMRFGRCINSRCHCTP |
| SEQ ID NO: 111 | RCRGSRDCLEPCRRAGTRFGRCINGRCHCTP |
| SEQ ID NO: 112 | RCRGSRDCLEPCRRAGTRFGRCINRRCHCTP |
| SEQ ID NO: 113 | RCRGSRDCLEPCRRAGTRFGRCINSRCHCTP |
| SEQ ID NO: 114 | RCRGSRDCLEPCRRAGTRFGRCIQSRCHCTP |
| SEQ ID NO: 115 | RCRGSRDCLEPCRRAGTRFGRCIQSRCHCYP |
| SEQ ID NO: 116 | RCRGSRDCYEPCRRAGTRFGRCIQSRCHCTP |
| SEQ ID NO: 117 | RCRGSRDCLEPCRRAGTRFGRCIQSRCYCTP |
| SEQ ID NO: 118 | RCRGSRDCLEPCRRAGTRFGRCIASRCHCYP |
| SEQ ID NO: 119 | RCRGSRDCLEPCRRAGTRFGRCISSRCHCYP |
| SEQ ID NO: 120 | RCRGSRDCLEPCRRAGTRFGRCITSRCHCYP |
| SEQ ID NO: 121 | KCRGSRDCLDPCRRAGMRFGRCINSRCHCTP |
| SEQ ID NO: 122 | RCRGSRDCLDPCKRAGMRFGRCINSRCHCTP |
| SEQ ID NO: 123 | RCRGSRDCLDPCRKAGMRFGRCINSRCHCTP |
| SEQ ID NO: 124 | RCRGSRDCLDPCRRAGMRFGKCINSRCHCTP |
| SEQ ID NO: 125 | RCRGSRDCLDPCRRAGMRFGRCINSKCHCTP |
| SEQ ID NO: 126 | RCRGSRDCLEPCRRAGTRFGRCINSRCHCYP |
| SEQ ID NO: 127 | SGVPINVRCRGSRDCLDPCRRAGMRFGRCINSRCHCTP |
| SEQ ID NO: 128 | GVPINVRCRGSRDCLDPCRRAGMRFGRCINSRCHCTP |
| SEQ ID NO: 129 | VPINVRCRGSRDCLDPCRRAGMRFGRCINSRCHCTP |
| SEQ ID NO: 130 | PINVRCRGSRDCLDPCRRAGMRFGRCINSRCHCTP |
| SEQ ID NO: 131 | INVRCRGSRDCLDPCRRAGMRFGRCINSRCHCTP |
| SEQ ID NO: 132 | NVRCRGSRDCLDPCRRAGMRFGRCINSRCHCTP |
| SEQ ID NO: 133 | VRCRGSRDCLDPCRRAGMRFGRCINSRCHCTP |
| SEQ ID NO: 134 | GVPISVRCRGSRDCLEPCRRAGTRFGRCINGRCHCTP |
| SEQ ID NO: 135 | GVPISVRCRGSRDCLEPCRRAGTRFGRCIQSRCHCTP |
| SEQ ID NO: 136 | GVPISVRCRGSRDCLEPCRRAGTRFGRCINRRCHCTP |
| SEQ ID NO: 137 | GVPINVRCRGSRDCLEPCRRAGTRFGRCINSRCHCTP |
| SEQ ID NO: 138 | GVPINVRCRGSRDCLEPCRRAGTRFGRCIQSRCHCTP |
| SEQ ID NO: 139 | GVPINVRCRGSRDCLEPCRRAGTRFGRCIQSRCHCYP |
| SEQ ID NO: 140 | GVPINVRCRGSRDCYEPCRRAGTRFGRCIQSRCHCTP |
| SEQ ID NO: 141 | GVPINVRCRGSRDCLEPCRRAGTRFGRCIQSRCYCTP |
| SEQ ID NO: 142 | GVPISVRCRGSRDCLEPCRRAGTRFGRCIQSRCHCYP |
| SEQ ID NO: 143 | GVPISVRCRGSRDCYEPCRRAGTRFGRCIQSRCHCTP |
| SEQ ID NO: 144 | GVPISVRCRGSRDCLEPCRRAGTRFGRCIQSRCYCTP |
| SEQ ID NO: 145 | GVPINVRCRGSRDCLEPCRRAGTRFGRCIASRCHCYP |
| SEQ ID NO: 146 | GVPINVRCRGSRDCLEPCRRAGTRFGRCISSRCHCYP |
| SEQ ID NO: 147 | GVPINVRCRGSRDCLEPCRRAGTRFGRCITSRCHCYP |
| SEQ ID NO: 148 | GVPINVRCRGSRDCLEPCRRAGTRFGRCINSRCHCYP |
| SEQ ID NO: 233 | RCRGSRDCLEPCRRAGTRFGRCILSRCHCTP |
| SEQ ID NO: 234 | RCRGSRDCLEPCRRAGTRFGRCILSRCHCYP |
| SEQ ID NO: 235 | RCRGSRDCLEPCRRAGTRFGRCILSRCYCTP |
| SEQ ID NO: 236 | RCRGSRDCLDPCRRAGMRFGRCIQSRCHCTP |
| SEQ ID NO: 237 | RCRGSRDCLDPCRRAGMRFGRCIASRCHCTP |
| SEQ ID NO: 238 | RCRGSRDCLDPCRRAGMRFGRCISSRCHCTP |
| SEQ ID NO: 239 | RCRGSRDCLDPCRRAGMRFGRCITSRCHCTP |
| SEQ ID NO: 240 | RCRGSRDCLDPCRRAGMRFGRCILSRCHCTP |
| SEQ ID NO: 241 | GSGVPINVRCRGSRDCLEPCRRAGTRFGRCILSRCHCTP |
| SEQ ID NO: 242 | GSGVPINVRCRGSRDCLEPCRRAGTRFGRCILSRCHCYP |
| SEQ ID NO: 243 | GSGVPINVRCRGSRDCLEPCRRAGTRFGRCILSRCYCTP |
| SEQ ID NO: 244 | GSGVPINVRCRGSRDCLDPCRRAGMRFGRCIQSRCHCTP |
| SEQ ID NO: 245 | GSGVPINVRCRGSRDCLDPCRRAGMRFGRCIASRCHCTP |
| SEQ ID NO: 246 | GSGVPINVRCRGSRDCLDPCRRAGMRFGRCISSRCHCTP |
| SEQ ID NO: 247 | GSGVPINVRCRGSRDCLDPCRRAGMRFGRCITSRCHCTP |
| SEQ ID NO: 248 | GSGVPINVRCRGSRDCLDPCRRAGMRFGRCILSRCHCTP |
| SEQ ID NO: 249 | GVPINVRCRGSRDCLEPCRRAGTRFGRCILSRCHCTP |
| SEQ ID NO: 250 | GVPINVRCRGSRDCLEPCRRAGTRFGRCILSRCHCYP |
| SEQ ID NO: 251 | GVPINVRCRGSRDCLEPCRRAGTRFGRCILSRCYCTP |
| SEQ ID NO: 252 | GVPINVRCRGSRDCLDPCRRAGMRFGRCIQSRCHCTP |
| SEQ ID NO: 253 | GVPINVRCRGSRDCLDPCRRAGMRFGRCIASRCHCTP |
| SEQ ID NO: 254 | GVPINVRCRGSRDCLDPCRRAGMRFGRCISSRCHCTP |
| SEQ ID NO: 255 | GVPINVRCRGSRDCLDPCRRAGMRFGRCITSRCHCTP |
| SEQ ID NO: 256 | GVPINVRCRGSRDCLDPCRRAGMRFGRCILSRCHCTP |
| SEQ ID NO: 260 | RCRGSRDCLDPCRRAGMRFGRCINSRCHC |
| SEQ ID NO: 261 | GSRCRGSRDCLDPCRRAGMRFGRCINSRCHC |
| SEQ ID NO: 262 | RCRGSRDCLDPCRRAGMRFGRCINSRCHCT |
| SEQ ID NO: 263 | GSRCRGSRDCLDPCRRAGMRFGRCINSRCHCT |

In some embodiments, a peptide of the present disclosure comprises at least 50%0, at least 510, at least 52%, at least 5300, at least 540%, at least 550%, at least 56%, at least 570%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with any one of SEQ ID NO: 89, SEQ ID NO: 106, SEQ ID NO: 221, SEQ ID NO: 23, SEQ ID NO: 24, SEQ ID NO: 87, SEQ ID NO: 88, SEQ ID NO: 107, SEQ ID NO: 108, SEQ ID NO: 219, SEQ ID NO: 223, SEQ ID NO: 225, SEQ ID NO: 21, SEQ ID NO: 22, SEQ ID NO: 25, SEQ ID NO: 26, SEQ ID NO: 220, SEQ ID NO: 222, SEQ ID NO: 224, SEQ ID NO: 226, SEQ ID NO: 109-SEQ ID NO: 110, SEQ ID NO: 129-SEQ ID NO: 133, SEQ ID NO: 260, SEQ ID NO: 262, SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256, SEQ ID NO: 27-SEQ ID NO: 45, SEQ ID NO: 47-SEQ ID NO: 66, SEQ ID NO: 241-SEQ ID NO: 248, SEQ ID NO: 261, and SEQ ID NO: 263, or SEQ ID NO: 209-SEQ ID NO: 215. In some embodiments, a peptide of the present disclosure has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 85.5%, at least 86%, at least 86.5%, at least 87%, at least 87.5%, at least 88%, at least 88.5%, at least 89%, at least 89.5%, at least 90%, at least 90.5%, at least 91%, at least 91.5%, at least 92%, at least 92.5%, at least 93%, at least 93.5%, at least 94%, at least 94.5%, at least 95%, at least 95.5%, at least 96%, at least 96.5%, at least 97%, at least 97.5%, at least 98%, at least 98.5%, at least 99%, at least 99.5%, or 100% sequence identity with a sequence selected from the group consisting of SEQ ID NO: 89, SEQ ID NO: 106, SEQ ID NO: 221, SEQ ID NO: 23, SEQ ID NO: 24, SEQ ID NO: 87, SEQ ID NO: 88, SEQ ID NO: 107, SEQ ID NO: 108, SEQ ID NO: 219, SEQ ID NO: 223, SEQ ID NO: 225, SEQ ID NO: 21, SEQ ID NO: 22, SEQ ID NO: 25, SEQ ID NO: 26, SEQ ID NO: 220, SEQ ID NO: 222, SEQ ID NO: 224, SEQ ID NO: 226, SEQ ID NO: 109-SEQ ID NO: 110, SEQ ID NO: 129-SEQ ID NO: 133, SEQ ID NO: 260, SEQ ID NO: 262, SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256, SEQ ID NO: 27-SEQ ID NO: 45, SEQ ID NO: 47-SEQ ID NO: 66, SEQ ID NO: 241-SEQ ID NO: 248, SEQ ID NO: 261, and SEQ ID NO: 263, and SEQ ID NO: 209-SEQ ID NO: 215. In some embodiments, a peptide of the present disclosure consists of a sequence selected from the group consisting of SEQ ID NO: 89, SEQ ID NO: 106, SEQ ID NO: 221, SEQ ID NO: 23, SEQ ID NO: 24, SEQ ID NO: 87, SEQ ID NO: 88, SEQ ID NO: 107, SEQ ID NO: 108, SEQ ID NO: 219, SEQ ID NO: 223, SEQ ID NO: 225, SEQ ID NO: 21, SEQ ID NO: 22, SEQ ID NO: 25, SEQ ID NO: 26, SEQ ID NO: 220, SEQ ID NO: 222, SEQ ID NO: 224, SEQ ID NO: 226, SEQ ID NO: 109-SEQ ID NO: 110, SEQ ID NO: 129-SEQ ID NO: 133, SEQ ID NO: 260, SEQ ID NO: 262, SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256, SEQ ID NO: 27-SEQ ID NO: 45, SEQ ID NO: 47-SEQ ID NO: 66, SEQ ID NO: 241-SEQ ID NO: 248, SEQ ID NO: 261, and SEQ ID NO: 263, and SEQ ID NO: 209-SEQ ID NO: 215.

In some embodiments, any peptide of the present disclosure further comprises a joining sequence. A joining sequence can be at the N-terminus or the C-terminus. In some embodiments, the joining sequence comprises of one or more amino acid residues and is located immediately adjacent to the N-terminus or the C-terminus of the peptide. A joining sequence can be at least 1 amino acid residue, at least 2 amino acid residues at least 1 amino acid residues, at least 2 amino acid residues, at least 3 amino acid residues, at least 4 amino acid residues, at least 5 amino acid residues, at least 6 amino acid residues, at least 7 amino acid residues, at least 8 amino acid residues, at least 9 amino acid residues, at least 10 amino acid residues, at least 15 amino acid residues, at least 20 amino acid residues, at least 25 amino acid residues, at least 30 amino acid residues, at least 35 amino acid residues, at least 40 amino acid residues, at least 45 amino acid residues, at least 50 amino acid residues, at least 55 amino acid residues, at least 60 amino acid residues, at least 65 amino acid residues, at least 70 amino acid residues, at least 75 amino acid residues, at least 80 amino acid residues, at least 85 amino acid residues, at least 90 amino acid residues, at least 95 amino acid residues, up and including 100 amino acid residues, from 1 to 5 amino acid residues, from 5 to 10 amino acid residues, from 10 to 15 amino acid residues, from 15 to 20 amino acid residues, from 20 to 25 amino acid residues, from 25 to 30 amino acid residues, from 30 to 35 amino acid residues, from 35 to 40 amino acid residues, from 40 to 45 amino acid residues, from 45 to 50 amino acid residues, from 50 to 55 amino acid residues, from 55 to 60 amino acid residues, from 60 to 65 amino acid residues, from 65 to 70 amino acid residues, from 70 to 75 amino acid residues, from 75 to 80 amino acid residues, from 80 to 85 amino acid residues, from 85 to 90 amino acid residues, from 90 to 95 amino acid residues, from 95 to 100 amino acid residues, from 1 to 100 amino acid residues, from 5 to 100 amino acid residues, from 20 to 80 amino acid residues, or from 1 to 10 amino acid residues.

A joining sequence can be immediately adjacent to the N-terminus, the C-terminus, or the N-terminus and the C-terminus of a peptide disclosed herein. Residues located in the joining sequence immediately adjacent to the N-terminus of a peptide are referred to as N−1 (the residue immediately N-terminally of the peptide), N−2 (the residue immediately N-terminally of the N−1 residue), N−3 (the residue immediately N-terminally of the N−2 residue), N−4 (the residue immediately N-terminally of the N−3 residue), N−5 (the residue immediately N-terminally of the N−4 residue), N−6 (the residue immediately N-terminally of the N−5 residue), and so on and so forth. In other words, if a joining sequence is placed at the N-terminus of the peptide, the last residue (reading from left to right, or N-terminus to C-terminus) of said joining sequence is the N−1 residue, the second to last residue of said joining sequence is the N−2 residue, the third to last residue of said joining sequence is the N−3, the fourth to last residue of said joining sequence is the N−4 residue, the fifth to last residue of said joining sequence is the N−5 residue, the sixth to last residue of said joining sequence is the N−6 residue, and so on and so forth.

Residues located in the joining sequence immediately adjacent to the C-terminus of a peptide are referred to as C+1 (the residue immediately C-terminally of the peptide), C+2 (the residue immediately C-terminally of the C+1 residue), C+3 (the residue immediately C-terminally of the C+2 residue), C+4 (the residue immediately C-terminally of the C+3 residue), C+5 (the residue immediately C-terminally of the C+4 residue), C+6 (the residue immediately C-terminally of the C+5 residue), and so on and so forth. In other words, if a joining sequence is placed at the C-terminus of the peptide, the first residue (reading from left to right, or N-terminus to C-terminus) of said joining sequence is the C+1 residue, the second residue of said joining sequence is the C+2 residue, the third residue of said joining sequence is the C+3, the fourth residue of said joining sequence is the C+4 residue, the fifth residue of said joining sequence is the C+5 residue, the sixth residue of said joining sequence is the C+6 residue, and so on and so forth.

In some embodiments, the peptide has at least 70%, at least 75%, at least 80%, at least 85%, at least 90%, at least 95%, at least 97%, at least 99% or 100% sequence identity with any one of: a) SEQ ID NO: 109, SEQ ID NO: 110, SEQ ID NO: 260, or SEQ ID NO: 262, wherein the peptide further comprises a joining sequence at the N-terminus, and wherein a last residue at the C-terminus of the joining sequence is selected from the group consisting of G, A, L, I, M, F, W, P, S, T, C, Y, N, Q, D, E, K, R, and H; b) SEQ ID NO: 129, wherein the peptide further comprises a joining sequence at the N-terminus, and wherein a last residue at a C-terminus of the joining sequence is selected from the group consisting of A, V, L, I, M, F, W, P, S, T, C, Y, N, Q, D, E, K, R, and H; c) SEQ ID NO: 130, wherein the peptide further comprises a joining sequence at the N-terminus, and wherein a last residue at a C-terminus of the joining sequence is selected from the group consisting of G, A, L, I, M, F, W, P, S, T, C, Y, N, Q, D, E, K, R, and H; d) SEQ ID NO: 131, wherein the peptide further comprises a joining sequence at the N-terminus, and wherein a last residue at a C-terminus of the joining sequence is selected from the group consisting of G, A, V, L, I, M, F, W, S, T, C, Y, N, Q, D, E, K, R, and H; e) SEQ ID NO: 132, wherein the peptide further comprises a joining sequence at the N-terminus, and wherein a last residue at a C-terminus of the joining sequence is selected from the group consisting of G, A, V, L, M, F, W, P, S, T, C, Y, N, Q, D, E, K, R, and H; f) SEQ ID NO: 133, wherein the peptide further comprises a joining sequence at the N-terminus, and wherein a last residue at a C-terminus of the joining sequence is selected from the group consisting of G, A, V, L, I, M, F, W, P, S, T, C, Y, Q, D, E, K, R, and H; g) SEQ ID NO: 260, wherein the peptide further comprises a joining sequence at the C-terminus, and wherein a first residue at an N-terminus of the joining sequence is selected from the group consisting of G, A, V, L, I, M, F, W, P, S, C, Y, N, Q, D, E, K, R, and H; or h) SEQ ID NO: 262, wherein the peptide further comprises a joining sequence at the C-terminus, and wherein a first residue at an N-terminus of the joining sequence is selected from the group consisting of G, A, V, L, I, M, F, W, S, T, C, Y, N, Q, D, E, K, R, and H.

In any of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 or fragment thereof, any one or more K residues can be replaced by an R residue or an A residue, any one or more R residues can be replaced by a K residue or an A residue, any one or more A residues can be replaced by a K residue or an R residue, all K residues can be replaced by R residues or A residues, all but one K residue can be replaced by R or A residues, all but two K residues can be replaced by R residues or A residues, or in any combination thereof. In any of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 or any fragment thereof, any one or more M residues can be replaced by any one of I, L, or V residues, any one or more L residues can be replaced by any one of V, I, or M residues, any one or more I residues can be replaced by any one of M, L, or V residues, or any one or more V residues can be replaced by any one of I, L, or M residues. In any embodiment, at least one of the amino acids alone or in combination can be interchanged in the peptides or peptide fragments as follows: K/R, M/I/L/V, G/A, S/T, Q/N, and D/E wherein each letter is each individually any amino acid or amino acid analogue. In some instances, the peptide can contain only one lysine residue, or no lysine residue. In any of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 or fragment thereof, any amino acid can be replaced with citrulline. In any of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 or any fragment thereof, X can independently be any number of any amino acid or no amino acid. In some cases, a peptide can include the first two N-terminal amino acids GS, as with peptides of SEQ ID NO: 21-SEQ ID NO: 45, SEQ ID NO: 47-SEQ ID NO: 66, SEQ ID NO: 220, SEQ ID NO: 222, SEQ ID NO: 224, SEQ ID NO: 226, SEQ ID NO: 241-SEQ ID NO: 248, SEQ ID NO: 261, and SEQ ID NO: 263 or such N-terminal amino acids (GS) can be substituted by any other one or two amino acids. In other cases, a peptide does not include the first two N-terminal amino acids GS, as with peptides of SEQ ID NO: 109-SEQ ID NO: 126, SEQ ID NO: 129-SEQ ID NO: 148, SEQ ID NO: 87-SEQ ID NO: 89, SEQ ID NO: 106-SEQ ID NO: 108, SEQ ID NO: 198, SEQ ID NO: 200-SEQ ID NO: 215, SEQ ID NO: 219, SEQ ID NO: 221, SEQ ID NO: 223, SEQ ID NO: 225, SEQ ID NO: 233-SEQ ID NO: 240, SEQ ID NO: 249-SEQ ID NO: 256, SEQ ID NO: 260, and SEQ ID NO: 262. In some cases, the N-terminus of the peptide is blocked, such as by an acetyl group; in other instances the C-terminus of the peptide is block, such as by an amide group.

In some instances, the peptide is any one of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 or a functional fragment thereof. In other embodiments, the peptide of the disclosure further comprises a peptide with 99%, 95%, 90%, 85%, or 80% homology to any one of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263. In further embodiments, the peptide fragment comprises a contiguous fragment of any one of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 that is at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46 residues long, wherein the peptide fragment is selected from any portion of the peptide. In some embodiments, such peptide fragments contact the cartilage and exhibit properties of those described herein for peptide and peptide-active agent conjugates.

The peptides of the present disclosure can further comprise negative amino acid residues. In some cases, the peptide has 2 or fewer negative amino acid residues. In other cases, the peptide has 4 or fewer negative amino acid residues, 3 or fewer negative amino acid residues, or 1 or fewer negative amino acid residues. The negative amino acid residues can be selected from any negative charged amino acid residues. The negative amino acid residues can selected from either E or D, or a combination of both E and D.

The peptides of the present disclosure can further comprise basic amino acid residues. In some embodiments, basic residues are added to the peptide sequence to increase the charge at physiological pH. The added basic residues can be any basic amino acid. The added basic residues can be selected from K or R, or a combination of K or R.

In some embodiments, the peptide has a charge distribution comprising an acidic region and a basic region. An acidic region can be a nub. A nub is a portion of a peptide extending out of the peptide's three-dimensional structure. A basic region can be a patch. A patch is a portion of a peptide that does not designate any specific topology characteristic of the peptide's three-dimensional structure. In further embodiments, a cystine-dense peptide can be 6 or more basic residues and 2 or fewer acidic residues.

The peptides of the present disclosure can further comprise positively charged amino acid residues. In some cases, the peptide has at least 2 positively charged residues. In other cases, the peptide has at least 3 positively charged residues, at least 4 positively charged residues, at least 5 positively charged residues, at least 6 positively charged residues, at least 7 positively charged residues, at least 8 positively charged residues or at least 9 positively charged residues. The positively charged residues can be selected from any positively charged amino acid residues. The positively charged residues can be selected from either K or R, or a combination of K and R.

In addition, the peptides herein can comprise a 4-19 amino acid residue fragment of any of the above sequences containing at least 2 cysteine residues, and at least 2 or 3 positively charged amino acid residues (for example, arginine, lysine or histidine, or any combination of arginine, lysine or histidine). In other embodiments, the peptides herein is a 20-70 amino acid residue fragment of any of the above sequences containing at least 2 cysteine residues, no more than 2 basic residues, and at least 2 or 3 positively charged amino acid residues (for example, arginine, lysine or histidine, or any combination of arginine, lysine or histidine). In some embodiments, such peptide fragments contact the cartilage and exhibit properties of those described herein for peptide and peptide-active agent conjugates.

Furthermore, peptides denoted as SEQ ID NO: 128 (also shown as SEQ ID NO: 205), and SEQ ID NO: 149 (also shown as SEQ ID NO: 46; non-GS version of SEQ ID NO: 149/SEQ ID NO: 46 are shown in SEQ ID NO: 128 and SEQ ID NO: 205) are excluded from (or not included in) peptides comprising any one of the peptides comprising SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263. Peptides denoted as SEQ ID NO: 128 (also shown as SEQ ID NO: 205), and SEQ ID NO: 149 (also shown as SEQ ID NO: 46) are specifically excluded from (or not included in) peptides comprising any one of the peptides comprising SEQ ID NO: 110, SEQ ID NO: 260, SEQ ID NO: 262.

In some embodiments, the peptide contains one or more disulfide bonds and has a positive net charge at neutral pH. At physiological pH, peptides can have a net charge, for example, of −5, −4, −3, −2, −1, 0, +1, +2, +3, +4, or +5. When the net charge is zero, the peptide can be uncharged or zwitterionic. In some instances, the peptide can have a positive charge at physiological pH. In some instances, the peptide can have a charge ≥+2 at physiological pH, ≥+3.5 at physiological pH, ≥+4.5 at physiological pH. In some embodiments, the peptide contains one or more disulfide bonds and has a positive net charge at neutral pH where the net charge can be +0.5 or less than +0.5, +1 or less than +1, +1.5 or less than +1.5, +2 or less than +2, +2.5 or less than +2.5, +3 or less than +3, +3.5 or less than +3.5, +4 or less than +4, +4.5 or less than +4.5, +5 or less than +5, +5.5 or less than +5.5, +6 or less than +6, +6.5 or less than +6.5, +7 or less than +7, +7.5 or less than +7.5, +8 or less than +8, +8.5 or less than +8.5, +9 or less than +9.5, +10 or less than +10. In some embodiments, the peptide has a negative net charge at physiological pH where the net charge can be −0.5 or less than −0.5, −1 or less than −1, −1.5 or less than −1.5, −2 or less than −2, −2.5 or less than −2.5, −3 or less than −3, −3.5 or less than −3.5, −4 or less than −4, −4.5 or less than −4.5, −5 or less than −5, −5.5 or less than −5.5, −6 or less than −6, −6.5 or less than −6.5, −7 or less than −7, −7.5 or less than −7.5, −8 or less than −8, −8.5 or less than −8.5, −9 or less than −9.5, −10 or less than −10. In some cases, the engineering of one or more mutations or corresponding substitutions within a peptide yields a peptide with an altered isoelectric point, charge, surface charge, or rheology at physiological pH. Such engineering of a mutation or corresponding substitutions to a peptide derived from a scorpion or spider can change the net charge of the complex, for example, by decreasing the net charge by 1, 2, 3, 4, or 5, or by increasing the net charge by 1, 2, 3, 4, or 5. In such cases, the engineered mutation or corresponding substitutions may facilitate the ability of the peptide to contact the cartilage. Suitable amino acid modifications for improving the rheology and potency of a peptide can include conservative or non-conservative mutations or corresponding substitutions. A peptide can comprises at most 1 amino acid mutation or corresponding substitution, at most 2 amino acid mutations or corresponding substitutions, at most 3 amino acid mutations or corresponding substitutions, at most 4 amino acid mutations or corresponding substitutions, at most 5 amino acid mutations or corresponding substitutions, at most 6 amino acid mutations or corresponding substitutions, at most 7 amino acid mutations or corresponding substitutions, at most 8 amino acid mutations or corresponding substitutions, at most 9 amino acid mutations or corresponding substitutions, at most 10 amino acid mutations or corresponding substitutions, or another suitable number as compared to the sequence of the venom or toxin that the peptide is derived from. In other cases, a peptide, or a functional fragment thereof, comprises at least 1 amino acid mutation or corresponding substitution, at least 2 amino acid mutations or corresponding substitutions, at least 3 amino acid mutations or corresponding substitutions, at least 4 amino acid mutations or corresponding substitutions, at least 5 amino acid mutations or corresponding substitutions, at least 6 amino acid mutations or corresponding substitutions, at least 7 amino acid mutations or corresponding substitutions, at least 8 amino acid mutations or corresponding substitutions, at least 9 amino acid mutations or corresponding substitutions, at least 10 amino acid mutations or corresponding substitutions, or another suitable number as compared to the sequence of the venom or toxin that the peptide is derived from. In some embodiments, mutations or corresponding substitutions can be engineered within a peptide to provide a peptide that has a desired charge or stability at physiological pH.

Peptides can be mutated to add function or remove function. For example, peptides and peptide-conjugates of the present disclosure can be mutated to retain, remove, or add the ability to bind to ion channels, or to promote agonizing or antagonizing ion channels, such as potassium channel binding that may occur with the peptide or peptide-conjugates (e.g., the potassium channel hERG). In some instances, it can be advantageous to remove potassium channel binding from a peptide used for delivery of an active agent. Mutations and or corresponding substitutions can include one or more N to S, D to E, M to T, N to Q, N to A, N to S, N to T, N to L, S to G, and S to R amino acid substitutions, or one or more L to Y, H to Y, and T to Y amino acid substitutions, or any combination of thereof, depending on whether the variant is designed to retain function or to remove function of binding to the ion channel. In some embodiments the peptides and peptide-drug conjugates of the present disclosure are mutated to minimize ion channel binding in order to minimize side effects or enhance the safety either in the target tissue or systemically.

In some embodiments, charge can play a role in cartilage homing. The interaction of a peptide of this disclosure in solution and in vivo can be influenced by the isoelectric point (pI) of the peptide and/or the pH of the solution or the local environment it is in. The charge of a peptide in solution can impact the solubility of the protein as well as parameters such as biodistribution, bioavailability, and overall pharmacokinetics. Additionally, positively charged molecules can interact with negatively charged molecules. Positively charged molecules such as the peptides disclosed herein can interact and bind with negatively charged molecules such as the negatively charged extracellular matrix molecules in the cartilage including hyaluranon and aggrecan. Positively charged residues can also interact with specific regions of other proteins and molecules, such as negatively charged residues of receptors or electronegative regions of an ion channel pore on cell surfaces. As such, the pI of a peptide can influence whether a peptide of this disclosure can efficiently home to cartilage. Identifying a correlation between pI and cartilage homing can be an important strategy in identifying lead peptide candidates of the present disclosure. The pI of a peptide can be calculated using a number of different methods including the Expasy pI calculator and the Sillero method. The Expasy pI can be determined by calculating pKa values of amino acids as described in Bjellqvist et al., which were defined by examining polypeptide migration between pH 4.5 to pH 7.3 in an immobilized pH gradient gel environment with 9.2M and 9.8M urea at 15° C. or 25° C. (Bjellqvist et al. Electrophoresis. 14(10):1023-31 (1993)). The Sillero method of calculating pI can involve the solution of a polynomial equation and the individual pKas of each amino acid. This method does not use denaturing conditions (urea) (Sillero et al. 179(2): 319-35 (1989)) Using these pI calculation methods and quantifying the cartilage to blood ratio of peptide signal after administration to a subject can be a strategy for identifying a trend or correlation in charge and cartilage homing. In some embodiments, a peptide with a pI above biological pH (˜pH 7.4) can exhibit efficient homing to cartilage. In some embodiments, a peptide with a pI of at least 8, at least 9, at least 10, or at least 11 can efficiently home to cartilage. In other embodiments, a peptide with a pI of 11-12 can home most efficiently to cartilage. In certain embodiments, a peptide can have a pI of about 9. In other embodiments, a peptide can have a pI of 8-10. In some embodiments, more basic peptides can home more efficiently to cartilage. In other embodiments, a high pI alone may not be sufficient to cause cartilage homing of a peptide.

In some embodiments, the tertiary structure and electrostatics of a peptide of the disclosure can impact cartilage homing. Structural analysis or analysis of charge distribution can be a strategy to predict residues important in biological function, such as cartilage homing. For example, several peptides of this disclosure that home to cartilage can be grouped into a structural class defined herein as “hitchins,” and can share the properties of disulfide linkages between C1-C4, C2-C5, and C3-C6. The folding topologies of peptides linked through three disulfide linkages (C1-C4, C2-C5, and C3-C6), can be broken down into structural families based on the three-dimensional arrangement of the disulfides. Some cystine-dense peptides have the C3-C6 disulfide linkage passing through the macrocycle formed by the C1-C4 and C2-C5 disulfide linkages, hitchins have the C2-C5 disulfide linkage passing through the macrocycle formed by the C1-C4 and C3-C6 disulfide linkages, and yet other structural families have the C1-C4 disulfide linkage passing through the macrocycle formed by the C2-C5 and C3-C6 disulfide linkages. Variants of “hitchin” class peptides with preserved disulfide linkages at these cysteine residues, primary sequence identity, and/or structural homology can be a method of identifying or predicting other potential peptide candidates that can home to cartilage. Additionally, members and related members of the calcin family of peptides can also home to cartilage, despite having a distinct tertiary structure from the “hitchin” class of peptides. Calcin peptides are structurally a subset of the cystine-dense peptides, with cystine-dense disulfide connectivity and topology, but are further classified on the basis of functioning to bind and activate ryanodine receptors (RyRs). These receptors are calcium channels that act to regulate the influx and efflux of calcium in muscle (Schwartz et al. Br J Pharmacol 157(3):392-403. (2009)). Variants of the calcin family of peptides with preserved key residues can be one way to predict promising candidates that can home to cartilage. In some embodiments, structural analysis of a peptide of this disclosure can be determined by evaluating peptides for resistance to degradation in buffers with various proteases or reducing agents. Structural analysis of the distribution of charge density on the surface of a peptide can also be a strategy for predicting promising candidates that can home to cartilage. Peptides with large patches of positive surface charge (when at pH 7.5) can home to cartilage.

The NMR solution structures, x-ray crystallography, or crystal structures of related structural homologs can be used to inform mutational strategies that can improve the folding, stability, and manufacturability, while maintaining the ability of a peptide to home to cartilage. They can be used to predict the 3D pharmacophore of a group of structurally homologous scaffolds, as well as to predict possible graft regions of related proteins to create chimeras with improved properties. For example, this strategy can be used to identify critical amino acid positions and loops that can be used to design drugs with improved properties or to correct deleterious mutations that complicate folding and manufacturability for the peptides. These key amino acid positions and loops can be retained while other residues in the peptide sequences can be mutated to improve, change, remove, or otherwise modify function, homing, and activity of the peptide.

Additionally, the comparison of the primary sequences and the tertiary sequences of two or more peptides can be used to reveal sequence and 3D folding patterns that can be leveraged to improve the peptides and parse out the biological activity of these peptides. For example, comparing two different peptide scaffolds that home to cartilage can lead to the identification of conserved pharmacophores that can guide engineering strategies, such as designing variants with improved folding properties. Important pharmacophore, for example, can comprise aromatic residues or basic residues, which can be important for binding.

Improved peptides can also be engineered based upon immunogenicity information, such as immunogenicity information predicted by TEPITOPE and TEPITOPEpan. TEPITOPE is a computational approach which uses position specific scoring matrix to provide prediction rules for whether a peptide will bind to 51 different HLA-DR alleles, and TEPITOPEpan is method that uses TEPITOPE to extrapolate from HLA-DR molecules with known binding specificities to HLA-DR molecules with unknown binding specificities based on pocket similarity. For example, TEPITOPE and TEPITOPEpan can be used to determine immunogenicity of peptides that home to cartilage. Immunogenicity information can also be predicted using the program NetMHCII version 2.3, which can determine the likelihood that a sequence might be presented as an immunogenic peptide via the major histocompatibility complex (MHC) presentation system of antigen presenting cells (APCs). (Nielson, M et al. BMC Bioinformatics, 8: 238 (2007); Nielsen, M. et al. BMC Bioinformatics, 10: 296 (2009)). This program can create an immunogenicity score by predicting the binding of a peptide to MIIC alleles. Strong binding alleles and weak binding alleles in each major MIIC allele group (DR, DQ, and DP) can be tallied separately. The number of peptides of a specific length within the sequence (e.g., a ‘core’ peptide that can be nine residues long) that are immunogenic can also be tallied. Comparison of peptides or ‘core’ peptides with high immunogenicity to peptides or ‘core’ peptides with low immunogenicity can guide engineering strategies for designing variants with decreased immunogenicity. Stronger binding peptides can be more likely to generate an immune response in patient carrying that given MIIC alleles. Mutating stronger binding amino acids or peptides out of a peptide sequence can reduce the immunogenicity of the entire peptide. Another aspect of immunogenicity, in addition to whether a peptide binds to a patient's MIIC allele, can be whether the patient's immune cells, such as a professional antigen presenting cells such as a macrophage, a B cell, or a dendritic cell, can process the peptide. A dendritic cell can take up a protein or peptide, and then can process a peptide, such as by cleaving to form a nine residue long peptide, which then can bind to the MHC and can be presented on the surface of the dendritic cell to the immune system's various T cells, including helper T cells and cytotoxic T cells, and thus can stimulate an immune response. The processing can involve peptide bond cleavage by enzymes and disulfide bond reduction, and thus a peptide or protein that is resistant to enzymatic cleavage and/or reduction can be resistant to processing and subsequent MHC presentation to the immune system. Therefore, having a peptide or protein that is resistant to enzymatic cleavage and/or reduction can reduce its immunogenic potential.

Furthermore, multiple sequence alignment can also be used to inform mutational strategies using previously identified sequences, and thus providing a guide to making changes that would eliminate labile residues and immunogenic regions of a peptide sequence. Peptides can be evaluated for residues of potential biochemical instability and regions of potential immunogenicity. Then, a residue that can allow for greater peptide stability at a certain location in a peptide can be identified from a multiple sequence alignment. For example, a specific residue can be identified from a multiple sequence alignment as providing greater stability for a peptide at position previously identified as a possible risk for a significant rate of deamidation, cleavage, degradation, oxidation, hydrolysis, isomerization, disulfide exchange, racemization, beta elimination, or aggregation. This information can then be used to create peptides with greater stability or reduced immunogenicity.

In addition to utilizing co-crystal x-ray structures, NMR solution structures, and mutagenesis studies, a multiple alignment of peptide sequences can be used to identify specific amino acids or regions of high conservation that indicate an important interaction with a target or receptor (e.g., binding to a potassium channel protein) or are important for folding and structure or other properties. Once the conserved amino acid or region is identified, then amino acids replacements can be determined that maintain the important properties of the peptide, such as maintenance of the structure, reduction in immunogenicity, reduction in binding to an ion channel protein, increased stability, or any combination of thereof.

The multiple sequence alignment can also identify possible locations to add a tyrosine or tryptophan residue for spectrophotometric reporting. Incorporation of aromatic amino acids such as Tyrosine or Tryptophan into a peptide such as SEQ ID NO: 149 (also disclosed herein as SEQ ID NO: 46; non-GS version of SEQ ID NO: 149/SEQ ID NO: 46 are shown in SEQ ID NO: 128 and SEQ ID NO: 205), which otherwise contains only amino acids of low UV absorbance at 280 nm, can be analytically advantageous. Tyrosine and Tryptophan amino acids contain aromatic ring structures. These residues have distinct absorption and emission wavelengths and good quantum yields, as shown in TABLE 2, not present in other amino acids. Both Tyrosine and Tryptophan can provide a good ‘handle’ for analytical detection of a peptide in solution since UV absorbance in the 250-300 nm range and peptide fluorescence is specific for these aromatic molecules. While chromatographic detection of a peptide such as SEQ ID NO: 149 relies on the absorbance of the peptide bond at 220 nm, where many other materials including minor impurities in solvents also often contribute to signal, the absorbance and fluorescence properties of Tryptophan and Tyrosine containing peptides can provide for a significantly more selective and sensitive detection. Thus incorporating an aromatic amino acid can create peptides better suited for concentration and purity measurements, which can be useful during analytics, process development, manufacturing, and other drug development and drug manufacturing activities. Incorporation can be achieved either through substitutions of one or more amino acids in the peptide to Tyr and/or Trp, insertion of Tyr and/or Trp into the peptide, or via addition of Tyr and/or Trp to the N-terminus or C-terminus of the peptide.

TABLE 2
Absorbance and Fluorescence Characteristics of Tryptophan and Tyrosine.

| | Absorbance | | Fluorescence | |
|---|---|---|---|---|
| Amino | Wavelength | Absorbtivity | Wavelength | Quantum |
| Acid | (nm) | (M*cm)⁻¹ | (nm) | Yield |
| Tryptophan | 280 | 5,600 | 348 | 0.20 |
| Tyrosine | 274 | 1,400 | 303 | 0.14 |

A peptide of this disclosure can bind to chloride, potassium, or sodium channels. The peptide can also bind to calcium channels. The peptide can block potassium channels and/or sodium channels. The peptide can block calcium channels. In some embodiments, the peptide can activate any one or more of such channels. In some embodiments, the peptide can block any one or more of such channels. In some embodiments, the peptide cannot interact with any of such channels or can be mutated to reduce or remove binding to any such channels. In still other embodiments, the peptide can be a potassium channel agonist, a potassium channel antagonist, a portion of a potassium channel, a sodium channel agonist, a sodium channel antagonist, a chloride channel agonist, a chloride channel antagonist, a calcium channel agonist, a calcium channel antagonist, a hadrucalcin, a theraphotoxin, a huwentoxin, a kaliotoxin, a cobatoxin or a lectin. In some embodiments, the lectin can be SHL-Ib2. In some embodiments, the peptide can interact with, binds, inhibits, inactivates, or alters expression of ion channels or chloride channels. In some embodiments, the peptide can interact with an Nav. 7 ion channel. In some embodiments, the peptide can interact with a Kv 1.3 ion channel. In still other embodiments, the peptide interacts with proteases, matrix metalloproteinase, inhibits cancer cell migration or metastases, has antimicrobial activity, or has antitumor activity. In addition to acting on matrix metalloproteinases, the peptide can interact with other possible proteases (e.g., elastases). In some embodiments, a peptide of this disclosure can bind to multidrug resistance transporters. Peptide and peptide drug conjugate binding to and blocking multidrug resistance transporters can be used to treat bacterial infections or cancers of the joint and/or bone.

In some embodiments, the peptide has other therapeutic effects on the cartilage or structures thereof or nearby. Beta defensin expression in articular cartilage can be correlated with immunomodulatory functions as we well as osteoarthritis, autoimmune rheumatic disorders such as systemic lupus erythematosus and rheumatoid arthritis (Vordenbaumen and Schneider 2011, Varoga 2004 and Varoga 2005). In some embodiments, the peptides or their mutants inhibit beta defensins, supplement beta defensins, are competitive inhibitors of beta defensins, active or block activation of beta defensin targets, and are used as immune modulators, or to treat autoimmune, arthritis, infections, and other articular disorders.

The present disclosure can also encompass multimers of the various peptides described herein. Examples of multimers include dimers, trimers, tetramers, pentamers, hexamers, heptamers, and so on. A multimer can be a homomer formed from a plurality of identical subunits or a heteromer formed from a plurality of different subunits. In some embodiments, a peptide of the present disclosure is arranged in a multimeric structure with at least one other peptide, or two, three, four, five, six, seven, eight, nine, ten, or more other peptides. In certain embodiments, the peptides of a multimeric structure each have the same sequence. In alternative embodiments, some or all of the peptides of a multimeric structure have different sequences.

The present disclosure further includes peptide scaffolds that, e.g., can be used as a starting point for generating additional peptides. In some embodiments, these scaffolds can be derived from a variety of cystine-dense peptides. Some suitable peptides for scaffolds can include, but are not limited to, chlorotoxin, brazzein, circulin, stecrisp, hanatoxin, midkine, hefutoxin, potato carboxypeptidase inhibitor, bubble protein, attractin, α-GI, α-GID, p-PIIIA, ω-MVIIA, ω-CVID, χ-MrIA, ρ-TIA, conantokin G, contulakin G, GsMTx4, margatoxin, shK, toxin K, chymotrypsin inhibitor (CTI), and EGF epiregulin core.

In some embodiments, the peptide sequences of the disclosure are flanked by additional amino acids. One or more additional amino acids can, for example, confer a desired in vivo charge, isoelectric point, chemical conjugation site, stability, or physiologic property to a peptide.

Identifying sequence homology can be important for determining key residues that preserve cartilage homing function. For example, in some embodiments identification of conserved positively charged residues can be important in preserving cartilage homing in any homologous variants that are made. In other embodiments, identification of basic or aromatic dyads, can be important in preserving interaction and activity with Kv ion channels in homologous variants.

Two or more peptides can share a degree of homology and share similar properties in vivo. For instance, a peptide can share a degree of homology with a peptide of the present disclosure. In some cases, a peptide of the disclosure can have up to about 20% pairwise homology, up to about 25% pairwise homology, up to about 30% pairwise homology, up to about 35% pairwise homology, up to about 40% pairwise homology, up to about 45% pairwise homology, up to about 50% pairwise homology, up to about 55% pairwise homology, up to about 60% pairwise homology, up to about 65% pairwise homology, up to about 70% pairwise homology, up to about 75% pairwise homology, up to about 80% pairwise homology, up to about 85% pairwise homology, up to about 90% pairwise homology, up to about 95% pairwise homology, up to about 96% pairwise homology, up to about 97% pairwise homology, up to about 98% pairwise homology, up to about 99% pairwise homology, up to about 99.5% pairwise homology, or up to about 99.9% pairwise homology with a second peptide. In some cases, a peptide of the disclosure can have at least about 20% pairwise homology, at least about 25% pairwise homology, at least about 30% pairwise homology, at least about 35% pairwise homology, at least about 40% pairwise homology, at least about 45% pairwise homology, at least about 50% pairwise homology, at least about 55% pairwise homology, at least about 60% pairwise homology, at least about 65% pairwise homology, at least about 70% pairwise homology, at least about 75% pairwise homology, at least about 80% pairwise homology, at least about 85% pairwise homology, at least about 90% pairwise homology, at least about 95% pairwise homology, at least about 96% pairwise homology, at least about 97% pairwise homology, at least about 98% pairwise homology, at least about 99% pairwise homology, at least about 99.5% pairwise homology, at least about 99.9% pairwise homology with a second peptide. Various methods and software programs can be used to determine the homology between two or more peptides, such as NCBI BLAST, Clustal W, MAFFT, Clustal Omega, AlignMe, Praline, or another suitable method or algorithm.

In still other instances, the variant nucleic acid molecules of a peptide of any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 can be identified by either a determination of the sequence identity or homology of the encoded peptide amino acid sequence with the amino acid sequence of any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263, or by a nucleic acid hybridization assay. Such peptide variants can include nucleic acid molecules (1) that remain hybridized with a nucleic acid molecule having the nucleotide sequence of any one of S SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 (or any complement of the previous sequences) under stringent washing conditions, in which the wash stringency is equivalent to 0.5×-2×SSC with 0.1% SDS at 55-65° C., and (2) that encode a peptide having at least 70%, at least 80%, at least 90%, at least 95% or greater than 95% sequence identity or homology to the amino acid sequence of any one SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Alternatively, peptide variants of any one SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 can be characterized as nucleic acid molecules (1) that remain hybridized with a nucleic acid molecule having the nucleotide sequence of any one SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 (or any complement of the previous sequences) under highly stringent washing conditions, in which the wash stringency is equivalent to 0.1×-0.2×SSC with 0.1% SDS at 50-65° C., and (2) that encode a peptide having at least 70%, at least 80%, at least 90%, at least 95% or greater than 95% sequence identity or homology to the polynucleotide encoding the amino acid sequence of any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

Percent sequence identity or homology can be determined by conventional methods. See, for example, Altschul et al., Bull. Math. Bio. 48:603 (1986), and Henikoff and Henikoff, Proc. Natl. Acad. Sci. USA 89:10915 (1992). Briefly, two amino acid sequences are aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Additionally, there are many established algorithms available to align two amino acid sequences. For example, the “FASTA” similarity search algorithm of Pearson and Lipman is a suitable protein alignment method for examining the level of sequence identity or homology shared by an amino acid sequence of a peptide disclosed herein and the amino acid sequence of a peptide variant. The FASTA algorithm is described by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., a reference sequence such as SEQ ID NO: 128, SEQ ID NO: 110 or SEQ ID NO: 260) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity or homology of nucleic acid molecules using a ratio as disclosed above. For nucleotide sequence comparisons, the ktup value can range between one to six, preferably from three to six, most preferably three, with other parameters set as described above.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that may be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e. g., 1, 2 or 3), while more preferred conservative amino acid substitutions are characterized by a BLOSUM62 value of at least 2 (e. g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., molecular modelling software such as PyMol, Chimera, Rosetta, Modeller, the Insight II, Discover or CHARMm and the like, homology modeling tools, or other appropriate program), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments, determination of structure can typically be accompanied by evaluating activity of modified molecules.

Pairwise sequence alignment is used to identify regions of similarity that may indicate functional, structural and/or evolutionary relationships between two biological sequences (protein or nucleic acid). By contrast, multiple sequence alignment (MSA) is the alignment of three or more biological sequences. From the output of MSA applications, homology can be inferred and the evolutionary relationship between the sequences assessed. One of skill in the art would recognize as used herein, “sequence homology” and “sequence identity” and “percent (%) sequence identity” and “percent (%) sequence homology” have been used interchangeably to mean the sequence relatedness or variation, as appropriate, to a reference polynucleotide or amino acid sequence.

### Chemical Modifications

A peptide can be chemically modified one or more of a variety of ways. In some embodiments, the peptide can be mutated to add function, delete function, or modify the in vivo behavior. One or more loops between the disulfide linkages can be modified or replaced to include active elements from other peptides (such as described in Moore and Cochran, Methods in Enzymology, 503, p. 223-251, 2012). Amino acids can also be mutated, such as to increase half-life or bioavailability, modify, add or delete binding behavior in vivo, add new targeting function, modify surface charge and hydrophobicity, or allow conjugation sites. N-methylation is one example of methylation that can occur in a peptide of the disclosure. In some embodiments, the peptide can be modified by methylation on free amines. For example, full methylation can be accomplished through the use of reductive methylation with formaldehyde and sodium cyanoborohydride.

A chemical modification can, for instance, extend the terminal half-life, the absorption half-life, the distribution half-life of a peptide, change the biodistribution or pharmacokinetic profile, or the modification itself can be useful to provide viscosupplementation to a joint. A chemical modification can comprise a polymer, a polyether, polyethylene glycol, a biopolymer, a polyamino acid, a fatty acid, a dendrimer, an Fc region, a simple saturated carbon chain such as palmitate or myristolate, sugars, hyaluronic acid, or albumin. The chemical modification of a peptide with an Fc region can be a fusion Fc-peptide. A polyamino acid can include, for example, a polyamino acid sequence with repeated single amino acids (e. g., polyglycine), and a polyamino acid sequence with mixed polyamino acid sequences (e. g., gly-ala-gly-ala (SEQ ID NO: 264)) that can or cannot follow a pattern, or any combination of the foregoing.

In some embodiments, the peptides of the present disclosure may be modified such that the modification increases the stability and/or the half-life of the peptides. In some embodiments, the attachment of a hydrophobic moiety, such as to the N-terminus, the C-terminus, or an internal amino acid, can be used to extend half-life of a peptide of the present disclosure. In other embodiments, the peptide of the present disclosure can include post-translational modifications (e. g., methylation and/or amidation), which can affect, e.g., serum half-life. In some embodiments, simple carbon chains (e. g., by myristoylation and/or palmitylation) can be complexed, conjugated, or fused to the peptides. In some embodiments, for example, the simple carbon chains may render complexed, conjugated, or fused peptides easily separable from uncomplexed, unconjugated, or non-fused material. For example, methods that may be used to separate the desired peptides of the invention from uncomplexed, unconjugated, or non-fused material include, but are not limited to, solvent extraction and reverse phase chromatography. In some embodiments, lipophilic moieties can be complexed, conjugated, or fused to the peptide and can extend half-life through reversible binding to serum albumin. Moreover, the complexed, conjugated, or fused moieties can be lipophilic moieties that extend half-life of the peptides through reversible binding to serum albumin. In some embodiments, the lipophilic moiety can be cholesterol or a cholesterol derivative including cholestenes, cholestanes, cholestadienes and oxysterols. In some embodiments, the peptides can be complexed, conjugated, or fused to myristic acid (tetradecanoic acid) or a derivative thereof. In other embodiments, the peptides of the present disclosure are coupled (e. g., complexed, conjugated, or fused) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, antibodies, or a molecule that binds to albumin.

In some embodiments, the first two N-terminal amino acids (GS) of SEQ ID NO: 21-SEQ ID NO: 45, SEQ ID NO: 47-SEQ ID NO: 66, SEQ ID NO: 220, SEQ ID NO: 222, SEQ ID NO: 224, SEQ ID NO: 226, SEQ ID NO: 241-SEQ ID NO: 248, SEQ ID NO: 261, and SEQ ID NO: 263 can serve as a spacer or linker in order to facilitate conjugation or fusion to another molecule, as well as to facilitate cleavage of the peptide from such complexed, conjugated, or fused molecules. In some embodiments, the peptides of the present disclosure can be complexed, conjugated, or fused to other moieties that can modify or effect changes to the properties of the peptides.

### Active Agent Conjugates

As used herein, peptides according to the present disclosure can be complexed to an active agent. Such “complexes” can interchangeably be used herein with the terms “conjugates”, “linked”, “attached”, “joined”, “fused” (e.g., as a result of peptide fusion, expression via one or more vectors, viral methods, recombinant methodologies, or otherwise), or any combination of the foregoing can be used, to describe complexing of any peptide of the present disclosure to one or more active agent disclosed herein. Complexes can be formed by creating chemical bonds linking molecules such as linking the peptide and the active agent, including by chemical synthesis or conjugation or by recombinant expression. Complexes can also be formed non-covalently, such as by inclusion in particles, nanoparticles, liposomes, cells, cell fragments, membranes, or other methods of physical or chemical association. It is understood that the peptide-active agent complexes of the present disclosure are not limited by the methodology used to complex, conjugate, fuse, or link the active agent to the peptide. Peptides according to the present disclosure can be complexed, conjugated, or fused to a peptide biological agent or other agent comprising amino acids (e.g., an antibody or antibody fragment, receptor or receptor fragment, ligand or ligand fragment, hormone or hormone fragment, growth factors and growth factor fragments, biological toxins and fragments thereof, or other active portion of a peptide), a protein, a peptide, or to a small molecule, RNA, DNA, or other active agent molecular structure for use in the treatment of cartilage diseases, disorders, or injuries. A small molecule active agent can include a corticosteroid or glucocorticoid. A peptide active agent conjugate can be a peptide complexed, conjugated, or fused to an active agent by any mechanism described herein. For example, a peptide can be covalently complexed, conjugated, or fused to an active agent to form a peptide active agent conjugate. A peptide can be chemically complexed, conjugated, or fused to an active agent to form a peptide active agent conjugate. A peptide and active agent can be expressed as a fusion protein to form a peptide active agent conjugate. For example, an antibody or fragment thereof and a peptide can be expressed as a fusion protein to form a peptide active agent conjugate. For example, in certain embodiments, a peptide as described herein can be fused to another molecule, such as an active agent that provides a functional capability. A peptide can be complexed, conjugated, or fused with an active agent through expression of a vector containing the sequence of the peptide with the sequence of the active agent. In various embodiments, the sequence of the peptide and the sequence of the active agent are expressed from the same Open Reading Frame (ORF). In various embodiments, the sequence of the peptide and the sequence of the active agent can comprise a contiguous sequence. Various vectors and recombinant systems known in the art can be employed to make such fusion peptides. The peptide and the active agent can each retain similar functional capabilities in the fusion peptide compared with their functional capabilities when expressed separately.

Furthermore, for example, in certain embodiments, the peptides described herein are attached to another molecule, such as an active agent that provides a functional capability. In some embodiments, 1, 2, 3, 4, 5, 6, 7, 8, 9, or 10 active agents can be linked to a peptide. Multiple active agents can be attached by methods such as conjugating to multiple lysine residues and/or the N-terminus, or by linking the multiple active agents to a scaffold, such as a polymer or dendrimer and then attaching that agent-scaffold to the peptide (such as described in Yurkovetskiy, A. V., Cancer Res 75(16): 3365-72 (2015).

Described herein are active agents that can be complexed, conjugated, or fused to the peptides of the present invention for use in either cartilage disorders or kidney disorders, or both. In some embodiments, certain compounds or drugs are appropriate for use in either cartilage or kidney disorders, certain drug classes may be preferred for specific treatment depending on the indication or disorder. As described herein, it is understood that certain active agents are described in a non-limiting exemplary manner for use in treatments of cartilage and/or kidney indications. One or more of such active agents can be complexed, conjugated, or fused to a peptide of the present invention alone or in combination with one or more detectable agents described herein. In some embodiments, active agents that can be complexed, conjugated, or fused to any peptide of this disclosure can be classified by mechanism. For example, active agents can belong to the class of anti-inflammatory drugs, immunosuppressive (immune suppression) drugs, analgesics/pain relief drugs, disease modifying osteoarthritic drugs (DMOADs), cell depleting agents/apoptosis modifiers, bone resorptive agents and viscosupplementing agents, and tissue normalization (disease modifying) drugs.

Anti-inflammatory active agents can include, but are not limited to, corticosteroids, glucocorticoids, nonsteroidal anti-inflammatory drugs (NSAIDs), biologics, and other small molecules. Examples of corticosteroid active agents that can be complexed, conjugated, or fused to any peptide of this disclosure for delivery to the joints and kidneys include triamcinolone dexamethasone, budesonide, desciclesonide, and triamcinolone acetonide. Examples of NSAID active agents that can be complexed, conjugated, or fused to any peptide of this disclosure for delivery to the joints and kidneys include naproxen and ibuprofen. Other active agents include acetylsalicylic acid and acetaminophen. NSAID active agents can be further classified into COX2 inhibitors. An example of a COX2 inhibitor active agent directed to a prostaglandin pathway that can be complexed, conjugated, or fused to any peptide of this disclosure for delivery to the joint includes celecoxib. An example of a COX2 inhibitor active agent with anti-leukotriene receptor antagonist that can be complexed, conjugated, or fused to any peptide of this disclosure for delivery to the joint includes montelukast. An example of a COX2 inhibitor active agent that can be complexed, conjugated, or fused to any peptide of this disclosure for delivery to the kidneys includes iguratimod. Biologic active agents can be further classified into active agents that are IL-1 family inhibitors, IL-17 or IL-23 pathway inhibitors, IL-6 family inhibitors, interferon receptor inhibitors, tumor necrosis factor (TNF) inhibitors, RANK pathway inhibitors, B cell inhibitors, anti-IgE active agents, and co-stimulation inhibitors. An example of an IL-1 family inhibitor active agent that can be complexed, conjugated, or fused to any peptide of this disclosure for delivery to the joints includes anakinra. An example of an IL-17/IL-23 pathway inhibitor active agent that can be complexed, conjugated, or fused to any peptide of this disclosure for delivery to the joints includes secukinumab. An example of an IL-6 family inhibitor active agent that can be complexed, conjugated, or fused to any peptide of this disclosure for delivery to the kidneys includes sirukumab. An example of an interferon receptor inhibitor active agent that can be complexed, conjugated, or fused to any peptide of this disclosure for delivery to the kidneys includes anifrolumab. An example of a TNF inhibitor active agent that can be complexed, conjugated, or fused to any peptide of this disclosure for delivery to the joints includes infliximab or etanercept. An example of a RANK pathway inhibitor active agent that can be complexed, conjugated, or fused to any peptide of this disclosure for delivery to the joints includes denosumab. An example of a B cell inhibitor active agent that can be complexed, conjugated, or fused to any peptide of this disclosure for delivery to the joints and kidneys includes rituximab. An example of an anti-IgE active agent that can be complexed, conjugated, or fused to any peptide of this disclosure for delivery to the kidneys includes omalizumab. An example of a co-stimulation inhibitor active agent that can be complexed, conjugated, or fused to any peptide of this disclosure for delivery to the joints includes abatacept.

Pain relief active agents can include, but are not limited to analgesics, counter-irritants, and pain receptor blocking drugs. Analgesics can be further classified into non-narcotic agents and narcotic agents. An example of a non-narcotic active agent that can be complexed, conjugated, or fused to any peptide of this disclosure for delivery to the joints includes acetaminophen. An example of a narcotic active agent that can be complexed, conjugated, or fused to any peptide of this disclosure for delivery to joints includes oxycodone. Counter-irritant active agents can be further classified as natural products. An example of a natural product that can be complexed, conjugated, or fused to any peptide of this disclosure for delivery to the joints includes capsaicin. Pain receptor blocking active agents can be further classified as TRPV4 inhibitors. An example of a TRPV4 inhibitor active agent that can be complexed, conjugated, or fused to any peptide of this disclosure for delivery to the joints includes GSK2193874.

Apoptosis modifier active agents can include, but are not limited to, biologics and small molecules. Biologic apoptosis modifier active agents can be further classified as Fas/FasL inhibitors, TNF/TNFR inhibitors, TRAIL/TRAILR inhibitors, TWEAK/Fn14 inhibitors, IL-1 inhibitors, IL-1 receptor antagonists, growth factors, and sclerostin inhibitors. An example of a TNF/TNFR inhibitor active agent that can be complexed, conjugated, or fused to any peptide of this disclosure for delivery to the joints includes infliximab. An example of a TRAIL/TRAILR inhibitor active agent that can be complexed, conjugated, or fused to any peptide of this disclosure for delivery to the joints includes osteoprotegrin. An example of a TWEAK/Fn14 inhibitor active agent that can be complexed, conjugated, or fused to any peptide of this disclosure for delivery to the kidneys includes BIIB023. An example of an IL-1 receptor antagonist that can be complexed, conjugated, or fused to any peptide of this disclosure for delivery to the joints includes anakinra. An example of a growth factor active agent that can be complexed, conjugated, or fused to any peptide of this disclosure for delivery to the joints includes IGF-1. An example of a growth factor active agent that can be complexed, conjugated, or fused to any peptide of this disclosure for delivery to the kidneys includes EGF. An example of a sclerostin inhibitor active agent that can be complexed, conjugated, or fused to any peptide of this disclosure for delivery to the joints includes romosozumab. Small molecule apoptosis modifier active agents can be further classified as caspase inhibitors, iNOS inhibitors, surfactants, and bisphosphonates. An example of a caspase inhibitor active agent that can be complexed, conjugated, or fused to any peptide of this disclosure for delivery to the joints includes ZVAD-fmk. An example of an iNOS inhibitor active agent that can be complexed, conjugated, or fused to any peptide of this disclosure for delivery to the joints include S-methylisothiourea. An example of a surfactant active agent that can be complexed, conjugated, or fused to any peptide of this disclosure for delivery to the joints include P188. An example of a bisphosphonate active agent that can be complexed, conjugated, or fused to any peptide of this disclosure for delivery to the joints includes alendronate. Moreover, the known class of drugs called senotherapeutics, also referred to as senolytics or senolytic drugs or senolytic compounds, refers to small molecules that can selectively induce death of senescent cells and for example by directly or indirectly inducing apoptosis in senescent cells. In addition, senolytics may also act via non-apoptotic mechanisms of cell death including by necroptis, autophagic cell death, pyroptis and caspase-independent cell death (Journal of Cell Science 127; 2135-2144 (2014)). Such drugs can attenuate age-related deterioration of tissues or organs. Examples of drugs that can be complexed, conjugated, or fused to any peptide of this disclosure to induce apoptosis or induce cell death via non-apoptotic mechanisms include quercetin, dasatinib, bortezomib, carfilzomib, and navitoclax amongst other compounds disclosed herein. Additional active agents are described in the following references: Zhu, Y et al., Aging Cell 14(4):644-58 (2015); Kirkland, J L, Exp Gerontol. 48(1): 1-5 (2013); Kirkland J L and Tchkonia T, Exp Gereontol. 68: 19-25 (2015) Tchkonia, T et al., J Clin Invest., 123(3): 966-72 (2013); WO2016118859; Sugumar, D et al., Pharmagenomics Pers Med. 8: 23-33 (2015); Jiafa, R et al., Sci Rep. 6: 23968 (2016); Swanson, C D et al., Nat Rev Rheumatol., 5(6): 317-324 (2009); Oh, C J et al., PLoS One, 7(10):e45870 (2012); and Adebajo, A and Boehncke, W, Psoriatic Arthritis and Psoriasis: Pathology and Clinical Aspects, Springer (2016).

Tissue normalization (disease modifying) active agents can include, but are not limited to, biologics and small molecules. Biologic active agents can be further classified as chemokines (e.g., for stem cell recruitment) and growth factors. An example of a tissue normalization chemokine active agent that can be complexed, conjugated, or fused to any peptide of this disclosure for delivery to the joints includes MIP-3α. An example of a tissue normalization growth factor active agent that can be complexed, conjugated, or fused to any peptide of this disclosure for delivery to the joints includes BMP-2. Small molecule active agents can be further classified as flavonoids, ACE inhibitors, and anti-proliferative active agents. An example of a tissue normalization flavonoid active agent that can be complexed, conjugated, or fused to any peptide of this disclosure for delivery to the joints includes icariin. An example of a tissue normalization ACE inhibitor active agent that can be complexed, conjugated, or fused to any peptide of this disclosure for delivery to the kidneys includes captopril. An example of a tissue normalization anti-proliferative active agent that can be complexed, conjugated, or fused to any peptide of this disclosure for delivery to the joints includes methotrexate. As used herein, corticosteroid can refer to both a glucocorticoid and a mineralocorticoid compound (e.g., as, for example, mimics of hormones produced by the adrenal cortex), but corticosteroid is also used herein in a non-limiting manner as a synonym for glucocorticoid.

TABLE 3 describes active agents for treatment of a cartilage disorder that can be complexed, conjugated, or fused to any peptide of the present disclosure to form peptide-drug conjugates.

TABLE 3
Exemplary Active Agents for Cartilage Disorders

| Active Agent Class | Active Agent |
|---|---|
| Gold compound | Gold |
| Gold compound | Auranofin |
| Gold compound | Gold Sodium Thiomalate |
| Gold compound | Gold Thioglucose |
| Gold compound | Thiomalic Acid |
| Gold compound | Gold Thiosulphate |
| Analgesics | Tramadol (e.g., Ultram, Ultracet) |
| | and derivatives |
| Analgesics | Oxycodone (e.g., Percocet, |
| | Oxycontin) and derivatives |
| Analgesics | Hydrocodone (e.g., Norco, |
| | Vicoprofen) |
| Analgesics | Morphine |
| Analgesics | Fentanyl |
| Analgesics | Oxymorphone |
| Analgesics | Hydromorphone |
| Analgesics | Meperidine |
| Analgesics | Buprenorphine |
| Analgesics | Methadone |
| Bisphosphonate | Alendronate |
| Bisphosphonate | Ibandronate |
| Bisphosphonate | Risedronate |
| Bisphosphonate | Pamidronate |
| Bisphosphonate | Zoledronate |
| Non-Nitrogen Containing First | Clodronate |
| Generation Bisphosphonate | |
| Non-Nitrogen Containing First | Etidronate |
| Generation Bisphosphonate | |
| Non-Nitrogen Containing First | Tiludronate |
| Generation Bisphosphonate | |
| Bone resorption Inhibitors | Osteoprotegerin (OPG) |
| Sclerostin Antagonist Apoptosis | AMG785 (Romosozumab) |
| Inhibitors | |
| Caspase-1 ICE Inhibitors | VX-740 (Pralnacasan) |
| Counter-irritants | Menthol |
| Counter-irritants | Capsaicin |
| RANKL Targeting Agents | Denosumab |
| Cathepsin K Targeting Agents | Odanacatib |
| TNF-α Antagonists | CDP571 |
| TNF-α Antagonists | ISIS 104838 |
| Anti-Pain Drugs | Duloxetine |
| Polymers | Low Molecular Weight Chitosan |
| Matrix Drugs | Chondroitin sulfate glucosamine |
| Cytokines/Growth Factors | TGF-beta |
| Matrix | Laminin |
| Matrix | Fibronectin |
| Matrix | Lubricin |
| Matrix | Hyaluronic acid injections |
| Matrix | Glucosamine |
| Immunosuppressants | Rapamycin |
| HIF-1α Modulators | |
| HIF-2α Modulators | |
| Corticosteroid | Tixocortol pivalate |
| Glucocorticoid/Corticosteroid | Hydrocortisone Acetate |
| Glucocorticoid/Corticosteroid | Hydrocortisone t-Butyl Acetate |
| Glucocorticoid/Corticosteroid | Prednisolone Acetate |
| Glucocorticoid/Corticosteroid | Prednisolone t-Butyl Acetate |
| Corticosteroid | Dexamethasone Acetate |
| Corticosteroid | Dexamethasone t-Butyl Acetate |
| Glucocorticoid/Corticosteroid | Triamcinolone Diacetate |

TABLE 4 describes active agents for treatment of a kidney disorder that can be complexed, conjugated, or fused to any peptide of the present disclosure to form peptide-drug conjugates

TABLE 4
Exemplary Active Agents for Kidney Disorders

| Active Agent Class | Active Agent |
|---|---|
| ACE Inhibitors | Captopril |
| Angiotensin receptor blockers | Angiotensin receptor blocker |
| | losartan (Cozaar) |
| Hormones | Adrenocorticotropic hormone |
| Hormones | corticotropin-releasing hormone |
| Anti-fungal | amphotericin B |
| Cardiac glycoside | digitalis (and related glycosides) |
| Diuretics | potassium-depleting diuretics |
| Anti-coagulant | Coumadin |
| NLRP3 Inflammosome Targeted | MCC950 |
| Drugs | |
| NLRP3 Inflammosome Targeted | BHB |
| Drugs | |
| NLRP3 Inflammosome Targeted | Type I interferon/interferon alpha |
| Drugs | |
| NLRP3 Inflammosome Targeted | IFN-beta |
| Drugs | |
| NLRP3 Inflammosome Targeted | Resveratrol |
| Drugs | |
| NLRP3 Inflammosome Targeted | Arglabin |
| Drugs | |
| NLRP3 Inflammosome Targeted | CB2R agonist |
| Drugs | |
| NLRP3 Inflammosome Targeted | MicroRNA-223 |
| Drugs | |
| Biguanide | metformin |
| Immunosuppressive | mTOR modulators |
| Immunosuppressive and | FOXO4 peptide |
| antiproliferative | |
| Anti-inflammatory, | Triptolide |
| immunosuppressive | |
| Antioxidant | Alpha-lipoic acid |
| Checkpoint inhibitors | Nivolumab |
| Checkpoint inhibitors | Pembrolizumab |
| Checkpoint inhibitors | Pidilizumab |
| Checkpoint inhibitors | Bmx-936559 |
| Checkpoint inhibitors | Atezolizumab |
| Checkpoint inhibitors | Avelumab |
| Antibiotics | Penicillins |
| Penicillins | Amoxicillin |
| Antibiotics | Cephalosporins |
| Cephalosporins | Cephalexin |
| Antibiotics | Macrolides |
| Macrolides | Azithromycin |
| Antibiotics | Fluoroquinolones |
| Fluoroquinolones | Ciprofloxacin |
| Antibiotics | Sulfonamides |
| Sulfonamides | Co-trimoxazole |
| Antibiotics | Tetracyclines |
| Tetracyclines | Doxycycline |
| Antibiotics | Aminoglycosides |
| Diuretics | Loop Diuretics |
| Diuretics | Potassium Sparing Diuretics |
| Diuretics | Chlorothiazide |
| Diuretics | Chlorthalidone |
| Diuretics | Metolazone |
| Diuretics | Indapamide |
| Mineralocorticoid | |
| Renin Inhibitors | |
| Renin Inhibitors | aliskiren |
| Renin Inhibitors | pepstatin |
| Renin Inhibitors | statine |
| Renin Inhibitors | cgp2928 |
| Renin Inhibitors | remikiren |
| Renin Inhibitors | enalkiren |
| Renin Inhibitors | zankiren |
| SGLT modulator | Dapagliflozin |
| SGLT modulator | Canagliflozin |
| SGLT modulator | Empagliflozin |
| | Acetylsalicylic acid |
| Steroid | Beclomethasone monopropionate |
| IL-17 inhibitor | |
| | Caspaicin |
| | Deferasirox |
| | Olmesartan |
| | L-glutamic acid polymer |
| | Tirilazad |
| Dietary flavonols | |
| siRNA | |
| Rapamycin analogs | RAD001 |
| Counter-irritants | Piperine |
| Counter-irritants | Mustard Oil |
| Counter-irritants | Eugenol |
| Counter-irritants | Curcumin |
| Counter-irritant capsaicin-like | Resiniferatoxin (RTX) |
| molecule | |

TABLE 5 describes active agents for treatment of a cartilage disorder and a kidney disorder that can be complexed, conjugated, or fused to any peptide of the present disclosure to form peptide-drug conjugates.

TABLE 5
Exemplary Active Agents for Cartilage Disorders and Kidney Disorders

| Active Agent Class | Active Agent |
|---|---|
| IL-6 Receptor Modulators | Tocilizumab |
| IL-6 Receptor Modulators | Sarilumab |
| IL-6 Receptor Modulators | ALX-0061 |
| IL-6 Receptor Modulators | Sirukumab |
| IL-6 Receptor Modulators | Clazakizumab |
| IL-6 Receptor Modulators | Olokizumab |
| IL-6 Receptor Modulators | MEDI5117 |
| IL-17 Antagonists | Secukinumab |
| IL-17 Antagonists | Brodalumab |
| IL-17 Antagonists | Ixekizumab |
| Antagonists of p40 Subunit of IL-12/IL-23 | Ustekinumab |
| Antagonists of p40 Subunit of IL-12/IL-23 | Briakinumab |
| Antagonists of p19 Subunit of IL-23 | Tildrakizumab |
| Antagonists of p19 Subunit of IL-23 | Guselkumab |
| IL-23 Antagonists | Soluble IL-23 (or cytokine-binding |
| | homology region of soluble IL-23) |
| IL-1 Antagonists | Canakinumab |
| IL-1 Antagonists | Rilonacept |
| IL-1 Antagonists | Gevokizumab |
| IL-1 Antagonists | LY2189102 |
| IL-1 Antagonists | Lentiviral-mediated RNAi |
| IL-12 Antagonists | |
| IL-1 Receptor Antagonists | Anakinra/Kineret |
| IL-1 Receptor Antagonists | MEDI-8968 |
| IL-1 Antagonists | AMG-108 |
| Interleukins/Pro-Inflammatory | Pro-inflammatory IL-1α or IL-1β |
| Cytokines | |
| Interleukins | IL-8 |
| Interleukins | IL-15 |
| Interleukins | IL-18 |
| Interleukins | IL-4 |
| Interleukins | IL-10 |
| Interleukins | IL-13 |
| Interleukins | IL-22 |
| Interleukins | IL-17 |
| Interleukins | IL-6, IL-12, IL-23 |
| p38 Inhibitors | VX-745 |
| p38 Inhibitors | BIRB 796 |
| p38 Inhibitors | SCIO-469 |
| p38 Inhibitors | VX-702 |
| p38 Inhibitors | Pamapimod |
| p38 Inhibitors | ARRY-797 |
| Corticosteroids | beclomethasone 17-monopropionate |
| Corticosteroids | Desciclesonide (also known as des- |
| | isobutyrylciclesonide, des-ciclesonide, |
| | C21-desisobutyryl-ciclesonide, or RM1) |
| Corticosteroids | Flunisolide |
| Corticosteroids | Mometasone furoate |
| Corticosteroids | 22-hydroxy intermediate budesonide |
| | derivative |
| Corticosteroids | 6β-hydroxy budesonide derivative |
| Corticosteroids | Δ6-budesonide derivative |
| Corticosteroids | 23-hydroxy budesonide derivative |
| Corticosteroids | 16α-butryloxyprednisolone budesonide |
| | derivative |
| Corticosteroids | 16α-hydroxyprednisolone budesonide |
| | derivative |
| Corticosteroid (Beclomethasone) | QVAR inhalation |
| Corticosteroid (Budesonide) | pulmicort respules |
| Corticosteroid | Flovent HFA 44 |
| Corticosteroid (Mometasone) | Asmanex HFA |
| Corticosteroid (Mometasone) | Budesonide symbicort |
| Corticosteroid | Dexamethasone sodium phosphate |
| Corticosteroid | Tixocortol pivalate |
| Corticosteroid | Ciclesonide |
| Glucocorticoids | 21-nortriamcincolone acetonide |
| Glucocorticoids | Δ6-triamcinolone |
| Glucocorticoids | 6b-hydroxy triamcinolone acetonide |
| Glucocorticoids | 21-carboxy triamcinolone acetonide |
| Glucocorticoids | 6b-OH, 21-COOH triamcinolone acetonide |
| Glucocorticoids | 6α fluorocortisol |
| Glucocorticoids | 9α fluorocortisol |
| Glucocorticoids | Δl-dehydro configuration in prednisolone |
| Glucocorticoids | 16-methylene dexamethasone derivative |
| Glucocorticoids | 16α-methyl dexamethasone derivative |
| Glucocorticoids | 16β-methyl betamethasone derivative |
| Glucocorticoids | Cyclophosphamide |
| Immunosuppresive | Mycophenolate |
| Glucocorticoids/Mineralocorticoids | Cortisol |
| Glucocorticoids/Mineralocorticoids | Hydrocortisone |
| Glucocorticoids/Mineralocorticoids | Prednisolone |
| Glucocorticoids/Mineralocorticoids | Betamethasone |
| Glucocorticoid | Fluticasone |
| Glucocorticoid | Fluticasone propionate |
| Steroid (flunisolide) | Aerobid |
| Steroid (flunisolide) | Aerobid-M |
| Steroid (flunisolide) | Aerospan |
| Steroid (Flunisolide) | Fluticasone Furoate |
| Steroid (Fluticasone) | Flovent HFA 110 |
| Steroid (Fluticasone) | Flovent HFA 220 |
| Steroid (Fluticasone) | Flovent Diskus 50 |
| Steroid (Fluticasone) | Asmanex |
| Steroid | Betamethasone acetate |
| Steroid | Betamethasone sodium phosphate |
| Steroid | Betamethasone valerate |
| Steroid | Beclomethasone dipropionate |
| Local Anesthetic | procaine hydrochloride |
| Local Anesthetic | Novacain |
| Anesthetic | bupivacaine hydrochloride |
| Anesthetic | lidocaine hydrochloride |
| Local Anesthetic | ropivacaine hydrochloride |
| Analgesics | Morphine |
| Analgesics | Fentanyl |
| Quinazolines | Feitinib/Iressa |
| Quinazolines | Sorafenib/Nexavar |
| Quinazolines | Lapatinib ditosylate/Tykerb/Tyverb |
| Quinazolines | Sunitinib/Sutent |
| Quinazolines | Bortezomib/Velcade/Cytomib |
| Quinazolines | Everolimus/Temsirolimus |
| Quinazolines | Inhibitors of IAPS |
| Quinazolines | Activators of caspase pathway |
| Quinazolines | Activators of AKT pathway |
| Quinazolines | Propylpeptidase inhibitors |
| Quinazolines | Activators of p53 |
| Quinazolines | Inhibitors of anti-apoptotic protein |
| | inhibitors |
| Prolyl Hydroxylase (PHD) Inhibitors | Desferrioxamine |
| Prolyl Hydroxylase (PHD) Inhibitors | Dimethyloxalylglycine (DMOG) |
| Prolyl Hydroxylase (PHD) Inhibitors | L-mimosine (L-mim) |
| Aptamers | Peptide aptamers |
| Aptamers | RNA aptamer A-p50 |
| Aptamers | Peptide A aptamer TrxLeflD |
| Aptamers | Aptamer E07 |
| Aptamers | Aptamer gemcitabine polymers |
| Aptamers | RAGE |
| Aptamers | Pegaptanib |
| Proteosome Inhibitors | Bortezomib |
| Proteosome Inhibitors | Carfilzomib |
| Second Generation Proteosome | Ixazomib |
| Inhibitors | |
| Second Generation Proteosome | Delanzomib |
| Inhibitors | |
| Second Generation Proteosome | Oprozomib |
| Inhibitors | |
| Second Generation Proteosome | Marizomib |
| Inhibitors | |
| Apoptosis Inhibitors | FLIP agonist |
| Apoptosis Inhibitors | nitric oxide synthase inhibitors |
| Apoptosis Inhibitors | caspase-3 inhibitors (Z-DEVD- |
| | fmk (SEQ ID NO: 265)) |
| Apoptosis Inhibitors | caspase-9 inhibitors (Z-LEHD- |
| | fmk (SEQ ID NO: 266)) |
| Apoptosis Inhibitors | Sclerostin antagonists |
| Apoptosis Inhibitors/Growth Factor | IGF-1 |
| BCL-2 Agonist Apoptosis Inhibitors | Oblimersen |
| BCL-2 Agonist Apoptosis Inhibitors | Obatoclax |
| BCL-2 Agonist Apoptosis Inhibitors | Navitoclax |
| BCL-2 Agonist Apoptosis Inhibitors | Venetoclax (ABT-199) |
| BCL-2 Agonist Apoptosis Inhibitors | Navotoclax (ABT-263) |
| BCL-2 Agonist Apoptosis Inhibitors | GX01 series of compounds |
| BCL-2 Agonist Apoptosis Inhibitors | BCL-2 small molecule antagonists |
| BCL-2 Agonist Apoptosis Inhibitors | Tetraocarcin-A derivatives |
| BCL-2 Agonist Apoptosis Inhibitors | Chelerythrine |
| BCL-2 Agonist Apoptosis Inhibitors | Antimycin A derivatives |
| BCL-2 Agonist Apoptosis Inhibitors | HA14-1 |
| BCL-2 Agonist Apoptosis Inhibitors | Synthetic compound antagonist of BH3 |
| BCL-2 Agonist Apoptosis Inhibitors | Genasense |
| BCL-2 Agonist Apoptosis Inhibitors | ISIS 22783 |
| BCL-2/BCL-XL Agonist Apoptosis | Bispecific Antisense |
| Inhibitors | |
| Proapoptotic BCL-2 Targeting Drugs | Bax, Bak, Bid, Bad-derived BH3 Peptides |
| Proapoptotic BCL-2 Targeting Drugs | SAHbs |
| Proapoptotic BCL-2 Targeting Drugs | BH3Is |
| BCL-2/BCL-XL Agonist Apoptosis | ABT-737 |
| Inhibitors | |
| BCL-X Inhibitors | |
| Apoptosis Modifiers | Caspase-1 Inhibitors |
| Apoptosis Modifiers | Caspase-8 Inhibitors |
| Pan-caspase Caspase Inhibitor | IDN-6556 |
| Pan-caspase Caspase Inhibitor | IDN-6734 |
| Pan-caspase Caspase Inhibitor | VX-799 |
| Pan-caspase Inhibitor | MX1013 |
| Pan-caspase Caspase Inhibitor | M-920 |
| Pan-caspase Caspase Activator | MX-2060 derivatives |
| Pan-caspase Caspase Activators | Small-molecule compounds |
| Pan-caspase Caspase Activators | RGD peptides |
| Pan-caspase inhibitors | ZVAD-fmk |
| Caspase-1 ICE Inhibitors | IDN-11104 |
| Caspase-1 ICE Inhibitors | VX-756 |
| Caspase-3 Inhibitors | M-826 |
| Caspase-3 Inhibitors | M-791 |
| Caspase-3 Inhibitors | Immunocasp-3 |
| Caspase-3 Inhibitors | Ad-G/iCasp3 |
| Caspase-3 Inhibitors | PEF-F8-CP3 |
| Caspase-6 Inhibitors | Immunocasp-6 |
| Caspase-9 Inhibitors | FKBP12/caspase-9 fusion protein |
| IAP Antagonists | BIR3 antagonists |
| XIAP Antagonists | Capped tripeptide XIAP Antagonists |
| XIAP Antagonists | Smac-mimetic compounds |
| XIAP Antagonists | AEG35156/GEM ® 640 |
| XIAP Inhibitors | Embelin |
| XIAP Inhibitors | XIAP antisense and RNA constructs |
| XIAP/cIAP-1/cIAP-2 Inhibitors | Small molecule SMAC mimetics |
| IAP/Caspase Inhibitors | HIV-Tat/polyarginine-complexed |
| | SMAC peptides |
| BIR2/Caspase-3 Inhibitors | TWX024 |
| BIR2 Inhibitors | Polyphenylurea derivatives |
| Survivin Targeting Drugs | LY2181308 |
| Survivin Targeting Drugs | Ad-Survivin T34A |
| Anti-TWEAK Apoptosis Modifiers | BIIB023 |
| Xanthine Oxidase Inhibitors | Allopurinol |
| Xanthine Oxidase Inhibitors | Febuxostat |
| Xanthine Oxidase Inhibitors | Zyloprin |
| Growth Factor | bFGF |
| Growth Factor | IGF |
| Growth Factor | TFG-beta |
| Growth Factor | BMP-2 |
| Growth Factor | BMP-9 |
| Growth Factor | BMP-13 |
| Growth Factor | BMP-7 |
| Growth Factor | BMP-3 inhibitors |
| Growth Factor | TFG-β1 |
| Growth Factor | OP-1 |
| Growth Factor | PDGF |
| Growth Factor | PTH |
| Growth Factor | PTHrP |
| Growth Factor | MIP-3α |
| Growth Factor | EPO |
| Growth Factor | FGF |
| Growth Factor | FGF-2 |
| Growth Factor | FGF-18 |
| Growth Factor | TGF-β3 |
| Growth Factor | VEGF |
| Growth Factor | Wnt proteins |
| Growth Factor | EGF |
| Growth Factor | GM-CSF |
| Flavonoid | Icariin |
| Flavonoid | Quercetin |
| Tyrosine Kinase Inhibitor | Dasatinib |
| (Lck/Btk Inhibitor) | |
| TRPV4 Activators | GSK1016790A |
| TRPV4 Activators | 4alpha-PDD |
| TRPV4 Inhibitors | HC-067047 |
| TRPV4 Inhibitors | GSK2193874 |
| NSAID | Ampion |
| NSAID | Phenylbutazone |
| NSAID | Naproxen lysozyme conjugate |
| NSAID | Acetal salicylic acid |
| DMARDs | Sulfasalazine |
| DMARDs | Leflunomide |
| DMARDs | Hydroxychloroquine (Plaquenil) |
| Disease-Modifying Osteoarthritis Drugs | FGF-18 |
| (DMOADs) | |
| Uricosurics | Sulfinpyrazone |
| MSC Matrix | Collagen |
| MSC Matrix | Fibrin |
| MSC Matrix | Polylactatous |
| Surfactant | P188 and other surfactants |
| Molecules for Bone Marrow Niches | Angiopoetin |
| Molecules for Bone Marrow Niches | Bone morphogenitic proteins |
| Catecholamines | Epinephrine |
| Molecules for Bone Marrow Niches | Norepinephrine |
| Molecules for Bone Marrow Niches | GDF 5 |
| Molecules for Bone Marrow Niches | ICAN1 |
| Molecules for Bone Marrow Niches | Jagged1 |
| Molecules for Bone Marrow Niches | Osteopontin |
| Molecules for Bone Marrow Niches | parathyoid hormone |
| Molecules for Bone Marrow Niches | Calcitonin |
| Molecules for Bone Marrow Niches | steel factor |
| Molecules for Bone Marrow Niches | Thrombopoetin |
| Molecules for Bone Marrow Niches | vascular cell adhesion molecule 1 |
| Chemokine Molecules for Bone | CXCL12 |
| Marrow Niches | |
| B Cell Targeting Agents | Rituximab |
| B Cell Targeting Agents | BLys |
| B Cell Targeting Agents | TACI |
| T Cell Co-stimulation Antagonists | Abatacept |
| JAK Targeting Agents | Tofacitinib |
| Calcineurin Inhibitors | Tacrolimus |
| Calcineurin Inhibitors | Cyclosporin |
| Calcineurin Inhibitors | Voclosporin |
| COX-2 Inhibitors | Iguratimod |
| Leukotriene inhibitor | Montelukast |
| COX-2 Inhibitors | Rofecoxib |
| COX-2 Inhibitors | Valdecoxib |
| Interferon Receptor Inhibitors | Anifrolumab |
| IFN-α Inhibitors | Sifalimumab |
| Anti-IgE Agents | Omalizumab |
| iNOS Inhibitors | S-methylisothiourea |
| CD20 Antagonists/B Cell Inhibitors | Ocrelizumab |
| BAFF Antagonists/B Cell Inhibitors | Belimumab |
| TNF Superfamily BAFF and APRIL | Atacicept |
| Antagonists/B cell Inhibitors | |
| TNF-α Antagonists | Thalidomide |
| TNF-α Antagonists | Lenalidomide |
| TNF-α Antagonists | Pomalidomide |
| TNF-α Antagonists | Pentocifylline |
| TNF-α Antagonists | Bupropion |
| TNF Antagonists | Lentiviral-mediated RNAi |
| TNF Agonists | Recombinant TNF-α |
| TRAIL Receptor Agonists | HGS-ETR1 |
| TRAIL Receptor Agonists | HGS-ETR2 |
| TRAIL Receptor Agonists | HGS-TR2J |
| TRAIL Receptor Agonists | PRO1762 |
| TRAIL Receptor Agonists | TRA-8 |
| CD95/Fas Agonists | CD95-Fc |
| Marine Bioactive Compounds | TRAIL-Resistance Overcoming |
| | Marine Bioactive Compounds |
| Marine Bioactive Compounds | mazamine A |
| Marine Bioactive Compounds | marine-derived chroomycins |
| Marine Bioactive Compounds | Carotenoids |
| Marine Bioactive Compounds | Aplysin |
| Marine Bioactive Compounds | Aplidin |
| Marine Bioactive Compounds | Siphonaxanthin |
| Marine Bioactive Compounds | pectinotoxin-2 |
| Anti-Complement Drugs | Eculizumab |
| PAR-2 Modulators | Pepducin P2pal-18 |
| miR-2013 Blockers | Anti-sense oligonucleotides |
| Nrf2 Activator | Dimethyl fumarate |
| p53 Targeting Drugs | INGN201 |
| p53 Targeting Drugs | SCH58500 |
| p53 Targeting Drugs | ONYX-015 |
| p53 Targeting Drugs | C-terminal p53 peptides |
| p53 Targeting Drugs | CDB3 |
| p53 Targeting Drugs | CP31398 |
| p53 Targeting Drugs | Prima-1 |
| p53 Targeting Drugs | HPV E6-binding peptide aptamers |
| p53 Targeting Drugs | Nutlins |
| p53 Targeting Drugs | Chalcones |
| p53 Targeting Drugs | Small peptides |
| p53 Targeting Drugs | Pifithrin-α |
| p53 Targeting Drugs/Apoptosis | QP1-1002 |
| Modifiers (T cells) | |
| Apaf-1 Targeting Drugs/Apoptosis | QM56 |
| Modifiers (T cells) | |
| Apaf-1 Targeting Drugs/Apoptosis | SVT016426 |
| Modifiers (T cells) | |
| Ferrostatin | 16/86 |
| BASP1 Targeting Drugs/Apoptosis | BASP siRNA |
| Modifiers (T cells) | |
| Anti-Inflammatory Drugs | CCX140 |
| Anti-Inflammatory Drugs | CXA-10 |
| Anti-Inflammatory Drugs/ | Alkaline phosphatase |
| Anti-Fibrotic Drugs | |
| Anti-Fibrotic Drugs | Dnmt1 inhibitors |
| Anti-Inflammatory Drugs/Apoptosis | |
| Modifiers (T cells) | THR-184 |
| Immunomodulation | Lithium |
| β2-Adrenergic Agonists | Formoterol |
| Anti-Inflammatory Drugs | CRMD-001 |
| Endothelin-1 Targeting Drugs | Astrasentan |
| Vasopressin Receptor Antagonists | Tolvaptan |
| Vasopressin Receptor Antagonists | RWJ-676070 |
| Immunosuppressants | Azathioprine |
| Immunosuppressants | Mycophenolic acid |
| Immunosuppressants | Cyclosporine |
| Immune Modulators | Laquinimod |
| Slow-acting antirheumatic | |
| drugs (SAARDs) | |
| | Colcrys |
| Hormones | parathyroid hormone |
| Hormones | growth hormone |
| | 11-beta hydroxysteroid dehydrogenases |
| | Mineralocorticoid |
| | Proopiomelanocortin |
| | fludrocortisonesoxycorticosterone acetate |
| | vaccines from live attenuated viruses |
| | Aspirin |
| | Insulin |
| | Isonizaid |
| | Oral hypoglycemic agents |
| | Antacids |
| | Carbamazepine |
| | Cholestyramine |
| | Colestipol |
| | Ephedrine |
| | Erythromycin |
| | Mitotane |
| | oral contraceptives |
| | Phenobarbital |
| | Phenytoin |
| | Rifampin |
| | Troleandomycin |
| | Non-selective caspase inhibitor |
| | okadaic acid |
| | Camptothetic |
| | Staurosporine |
| | HFA |
| | Alvesco inhalation |
| | Breo Ellipta |
| | Advair |
| | Mometasone |
| | Dulera |
| | Umeclidinium |
| | Anoro |
| Reactive Oxygen Species | |
| Targeting Drugs | |
| Cytokines/Growth Factors | TGF-beta |
| NOD-like receptor protein 3-dependent | |
| caspase 1 Targeting Drugs | |
| NSAID | Etoricoxib |
| Apoptosis Modifiers | MCL1 inhibitors |
| | Teriparatide |
| | BH3 mimetics |
| | AZD 4320 |
| Carrier Proteins | Low molecular weight human |
| | serum albumin |
| Ceramide Targeting Drugs | |
| DMARDs | Penicillamine |
| Chondrogenic factors | |
| Anti-oxidative factors | |
| A(1)AR agonist | |
| S1P(2)R antagonist | |
| Antimalarials | |
| BAX/BAK activating drugs | |
| Selective GR Activators (SEGRAs) | |
| Rap1 Targeted Drugs | |
| Senolytic | Ephrin Ligand (EFN) B1 blockers |
| Senolytic | Cyclin-dependent kinase inhibitor 1A |
| | (p21) phosphatidylinositol-4,5-bishophate |
| | 3-kinase delta catlyatic subunit |
| | (PI3KCD) blockers |
| Senolytic | Plasminogen-activated inhibitor-2 |
| | (PAI-2) blockers |
| Senesce-associated secretory | |
| phenotype (SASP) inhibitors | |
| Hormone | Tetracosactide |
| Peptide | Oligopeptide |
| Peptide | Polypeptide |
| Peptide | Peptidomimetic |
| Nucleic Acid | Polynucleotide |
| Nucleic Acid | Polyribonucleotide |
| Nucleic Acid | Oligonucleotide |
| Nucleic Acid | DNA |
| Nucleic Acid | cDNA |
| Nucleic Acid | ssDNA |
| Nucleic Acid | RNA |
| Nucleic Acid | dsRNA |
| Nucleic Acid | micro RNA |
| Nucleic Acid | Interfering RNA |
| Nucleic Acid | Aptamer |
| Antibody | single chain variable Fragment (scFv) |
| Antibody | Antibody Fragment |
| Peptide | Aptamer |
| Antibody | Fc domains |
| Antibody | Fc regions |
| Antibody | Fc active fragments or |
| | modifications thereof |
| Cytokine | |
| Cytokine antagonists | Mavrilimumab |
| Cytokine antagonists | Ixekizumab |
| Cytokine antagonists | Tocilizumab |
| Cytokine antagonists | Anakinra |
| Cytokine antagonists | Ustekinumab |
| Cytokine antagonists | Secukinumab |
| Interferon | |
| Hormone | |
| Enzymes | |
| Growth Factor | |
| Checkpoint Inhibitor | |
| CD Antigen | |
| Chemokines | |
| Neurotransmitters | |
| Ion Channel Inhibitors | |
| G-protein coupled receptor inhibitors | |
| G-protein coupled receptor activators | |
| Tumor necrosis factor inhibitors | |
| Chemical Agents | |
| Radiosensitizers | |
| Radioprotectants | |
| Radionuclide | |
| Therapeutic Small Molecules | |
| Steroids | |
| Corticosteroids | |
| Anti-inflammatory Agents | |
| Immune Modulators | Abatacept |
| Immune Modulators | Rituximab |
| Complement Fixing Peptides or Proteins | |
| Tumor Necrosis Factor Family Inhibitors | Tumor Necrosis Factor (TNF) soluble |
| | receptor or antibody |
| Tumor Necrosis Factor Family Activators | |
| Tumor Necrosis Factor (TNF) soluble | |
| receptor or antibody | |
| Caspase protease inhibitors or activators | |
| NF-kB, RIPK1 and/or RIPK3 Inhibitors | |
| NF-kB, RIPK1 and/or RIPK3 Activators | |
| Death-receptor ligand activator or inhibitor | |
| Tumor Necrosis Factor Family Agonists | TNFR1 |
| Tumor Necrosis Factor Family Agonists | TNFR2 |
| Tumor Necrosis Factor Family Agonists | CD27/TNFRSF7 |
| Tumor Necrosis Factor Family Agonists | CD30/TNFRSF8 |
| Tumor Necrosis Factor Family Agonists | OX40/TNFRSF4 |
| Tumor Necrosis Factor Family Agonists | CD40/TNFRSF5 |
| Tumor Necrosis Factor Family Agonists | 4-1BB/TNFRSF9 |
| Tumor Necrosis Factor Family Agonists | RANK (receptor activator of |
| | NF-kappa B/TNFRSF11A) |
| Tumor Necrosis Factor Family Agonists | TWEAK receptor/TNFRSF12A |
| Tumor Necrosis Factor Family Agonists | TAC1/TNFRSF13B |
| Tumor Necrosis Factor Family Agonists | BAFF-R (BAFF receptor/TNFRSF13C) |
| Tumor Necrosis Factor Family Agonists | HVEM (herpes virus entry |
| | mediator/TNFRSF14) |
| Tumor Necrosis Factor Family Agonists | RELT/TNFRSF19L |
| Tumor Necrosis Factor Family Agonists | ectodysplasin A2 isoform |
| | receptor/TNFRS27 |
| Tumor Necrosis Factor Family Agonists | ectodysplasin Al |
| TNF Family Member | Anhidrotic Receptor |
| Tumor Necrosis Factor Family Antagonists | Decoy Receptor 3/TNFRSF6B |
| Tumor Necrosis Factor Family Antagonists | Decoy Receptor 1/TNFRSF10C |
| Tumor Necrosis Factor Family Antagonists | Decoy Receptor 2/TNFRSF10D |
| Tumor Necrosis Factor Family Antagonists | DR3 (death receptor 3/TNFRSF25) |
| Tumor Necrosis Factor Family Antagonists | DR4 (death receptor 4/TNFRSF10A) |
| Tumor Necrosis Factor Family Antagonists | DR5 (death receptor 5/TNFRSF10B) |
| Tumor Necrosis Factor Family Antagonists | DR6 (death receptor 6/TNFRSF21) |
| Tumor Necrosis Factor Family Antagonists | Fas/TNFRSF6 |
| Tumor Necrosis Factor Family Antagonists | Lymphotoxin b receptor/TNFRS3 |
| Tumor Necrosis Factor Family Antagonists | OPG (osteoprotegerin/TNFRSF11B) |
| Tumor Necrosis Factor Family Antagonists | Nerve Growth Factor Receptor/TNFRSF16 |
| Tumor Necrosis Factor Family Antagonists | BCMA (B Cell Maturation |
| | Antigen/TNFRSF17) |
| Tumor Necrosis Factor Family Antagonists | GITR (Glucocorticoid-Induced TNF |
| | Receptor/TNFRSF18) |
| Tumor Necrosis Factor Family Antagonists | TAJ (Toxicity and JNK |
| | Inducer/TNFRSF19) |
| Tumor Necrosis Factor Family Antagonists | TNFRSF22 |
| Tumor Necrosis Factor Family Antagonists | TNFRSF23 |
| TNF Receptor Superfamily Ligands | TNF alpha |
| TNF Receptor Superfamily Ligands | Lymphotoxin-a |
| TNF Receptor Superfamily Ligands | Tumor Necrosis Factor Membrane Form |
| TNF Receptor Superfamily Ligands | Tumor Necrosis Factor Shed Form |
| TNF Receptor Superfamily Ligands | LIGHT |
| TNF Receptor Superfamily Ligands | Lymphotoxin b2a1 heterotrimer |
| TNF Receptor Superfamily Ligands | OX-40 Ligand |
| TNF Receptor Superfamily Ligands | Compound 1 [PMID: 24930776] |
| TNF Receptor Superfamily Ligands | CD40 Ligand |
| TNF Receptor Superfamily Ligands | Fas Ligand |
| TNF Receptor Superfamily Ligands | TL1A |
| TNF Receptor Superfamily Ligands | CD70 |
| TNF Receptor Superfamily Ligands | CD30 Ligand |
| TNF Receptor Superfamily Ligands | TRAF1 |
| TNF Receptor Superfamily Ligands | TRAF2 |
| TNF Receptor Superfamily Ligands | TRAF3 |
| TNF Receptor Superfamily Ligands | TRAIL |
| TNF Receptor Superfamily Ligands | RANK Ligand |
| TNF Receptor Superfamily Ligands | APRIL |
| TNF Receptor Superfamily Ligands | BAFF |
| TNF Receptor Superfamily Ligands | B and T lymphocyte Attenuators |
| TRK Receptor Superfamily Ligands | NGF |
| TRK Receptor Superfamily Ligands | BDNF |
| TRK Receptor Superfamily Ligands | Neurotrophin-3 |
| TRK Receptor Superfamily Ligands | Neurotrophin-4 |
| TNF Receptor Superfamily Ligands | TL6 |
| TNF Receptor Superfamily Ligands | Ectodysplasin A2 |
| TNF Receptor Superfamily Ligands | Ectodysplasin A1 |
| TNF blockers | Remicade (infliximab) |
| TNF blockers | Enbrel (etanercept) |
| TNF blockers | Humira (adalimumab) |
| TNF blockers | Cimzia (certolizumab pegol) |
| TNF blockers | Simponi (golimumab) |
| Tumor Necrosis Factor Receptor | |
| Family Agonists | |
| Toll Like Receptors Agonist | |
| TIMP-3 Inhibitors | |
| BCL-2 Family Inhibitors | |
| IAP Disruptors | |
| Protease Inhibitors | |
| Amino Sugars | |
| Chemotherapeutic | |
| Cytotoxic chemical | |
| Toxins | |
| Tyrosine Kinase inhibitors | Imatinib Mesylate |
| Protons | |
| Antivascular Agents | Bevacizumab |
| EGFR Inhibitors | Erlotinib |
| Anti-Infective Agents | |
| Antibiotics | |
| Anti-Viral Agents | |
| Anti-Fungal Agents | |
| Aminoglycoside | |
| Statins | |
| Nanoparticles | |
| Liposomes | |
| Polymers | Biopolymers |
| Polysaccharide | |
| Proteoglycan | |
| Glycosaminoglycans | |
| Polyethylene glycol | |
| Lipids | |
| Dendrimers | |
| Fatty Acids | |
| Glucocorticoid | |
| Corticosteroid | |
| Collagenase Inhibitor | |
| Matrix Metalloprotease Inhibitors | MMP-13 inhibitor |
| Vitamins | Vitamin D |
| Antibiotics | |
| Antiviral | |
| Antifungal | |
| Statins | |
| Immune Modulators | |
| Radioisotopes | |
| Toxins | |
| Enzymes | |
| Sensitizing drugs | |
| Anti-Angiogenic Agents | Cisplatin |
| Anti-Angiogenic Agents | Anti-Metabolites |
| Anti-Angiogenic Agents | Mitotic Inhibitors |
| Anti-Angiogenic Agents | Growth Factor Inhibitors |
| Chemotherapeutic Agent | Paclitaxel |
| Chemotherapeutic Agent | Temozolomide |
| Chemotherapeutic Agent | Topotecan |
| Chemotherapeutic Agent | Fluorouracil |
| Chemotherapeutic Agent | Vincristine |
| Chemotherapeutic Agent | Vinblastine |
| Chemotherapeutic Agent | Procarbazine |
| Chemotherapeutic Agent | Decarbazine |
| Chemotherapeutic Agent | Altretamine |
| Chemotherapeutic Agent | Methotrexate |
| Chemotherapeutic Agent | Mercaptopurine |
| Chemotherapeutic Agent | Thioguanine |
| Chemotherapeutic Agent | Fludarabine Phosphate |
| Chemotherapeutic Agent | Cladribine |
| Chemotherapeutic Agent | Pentostatin |
| Chemotherapeutic Agent | Cytarabine |
| Chemotherapeutic Agent | Azacitidine |
| Chemotherapeutic Agent | Etoposide |
| Chemotherapeutic Agent | Teniposide |
| Chemotherapeutic Agent | Irinotecan |
| Chemotherapeutic Agent | Docetaxel |
| Chemotherapeutic Agent | Doxorubicin |
| Chemotherapeutic Agent | Daunorubicin |
| Chemotherapeutic Agent | Dactinomycin |
| Chemotherapeutic Agent | Idarubicin |
| Chemotherapeutic Agent | Plicamycin |
| Chemotherapeutic Agent | Mitomycin |
| Chemotherapeutic Agent | Bleomycin |
| Chemotherapeutic Agent | Tamoxifen |
| Chemotherapeutic Agent | Flutamide |
| Chemotherapeutic Agent | Leuprolide |
| Chemotherapeutic Agent | Goserelin |
| Chemotherapeutic Agent | Aminogluthimide |
| Chemotherapeutic Agent | Anastrozole |
| Chemotherapeutic Agent | Amsacrine |
| Chemotherapeutic Agent | Asparaginase |
| Chemotherapeutic Agent | Mitoxantrone |
| Chemotherapeutic Agent | Mitotane |
| Chemotherapeutic Agent | Amifostine |
| Apoptotic Agents | |
| Cell Death or Cell Killing Agents | Caspases |
| Apoptosis Activators | |
| Apoptosis Inhibitors | XBP- 1 |
| Apoptosis Inhibitors | Bcl-2 |
| Apoptosis Inhibitors | Bcl-X1 |
| Apoptosis Inhibitors | Bcl-w |
| Nonsteroidal Anti-Inflammatory | COX-2 Inhibitors |
| Drugs (NSAID) | |
| Nonsteroidal Anti-Inflammatory | Ketorolac |
| Drugs (NSAID) | |
| Nonsteroidal Anti-Inflammatory | Indomethacin |
| Drugs (NSAID) | |
| Nonsteroidal Anti-Inflammatory | Etodolac |
| Drugs (NSAID) | |
| Nonsteroidal Anti-Inflammatory | Tolemetin |
| Drugs (NSAID) | |
| Nonsteroidal Anti-Inflammatory | Naproxen |
| Drugs (NSAID) | |
| Nonsteroidal Anti-Inflammatory | Enolic Acid Derivatives |
| Drugs (NSAID) | |
| Nonsteroidal Anti-Inflammatory | Anthranilic Acid Derivatives |
| Drugs (NSAID) | |
| Nonsteroidal Anti-Inflammatory | Celecoxib |
| Drugs (NSAID) | |
| Nonsteroidal Anti-Inflammatory | Sulfonanilides |
| Drugs (NSAID) | |
| Nonsteroidal Anti-Inflammatory | Salicylates |
| Drugs (NSAID) | |
| Nonsteroidal Anti-Inflammatory | Aceclofenac |
| Drugs (NSAID) | |
| Nonsteroidal Anti-Inflammatory | Nabumetone |
| Drugs (NSAID) | |
| Nonsteroidal Anti-Inflammatory | Sulindac |
| Drugs (NSAID) | |
| Nonsteroidal Anti-Inflammatory | Diclofenac |
| Drugs (NSAID) | |
| Nonsteroidal Anti-Inflammatory | Ibuprofen |
| Drugs (NSAID) | |
| Steroids | Dexamethasone |
| Steroids | Budesonide |
| Steroids | Triamcinolone |
| Steroids | Triamcinolone acetonide |
| Steroids | Cortisone |
| Steroids | Prednisone |
| Steroids | Prednisolone |
| Steroids | Triamcinolone Hexacetonide |
| Steroids | Methylprednisolone |
| Pain Reliever | Acetaminophen |
| Opioids | |
| Local Anesthetics | |
| Anti-Depressants | |
| Glutamate Receptor Antagonists | |
| | Adenosine |
| Neuropeptides | |
| Uricase | |
| Elastase | |

Further examples of active agents include but are not limited to: a peptide, an oligopeptide, a polypeptide, a peptidomimetic, a polynucleotide, a polyribonucleotide, a DNA, a cDNA, a ssDNA, a RNA, a dsRNA, a micro RNA, an RNAi, an oligonucleotide, an antibody, a single chain variable fragment (scFv), an antibody fragment, an aptamer, a cytokine, an interferon, a hormone, an enzyme, a growth factor, a checkpoint inhibitor, a PD-1 inhibitor, a PD-L1 inhibitor, a CTLA4 inhibitor, a CD antigen, aa chemokine, a neurotransmitter, an ion channel inhibitor, a G-protein coupled receptor inhibitor, a G-protein coupled receptor activator, a chemical agent, a radiosensitizer, a radioprotectant, a radionuclide, a therapeutic small molecule, a steroid, a corticosteroid, an anti-inflammatory agent, an immune modulator, a complement fixing peptide or protein, a tumor necrosis factor inhibitor, a tumor necrosis factor activator, a tumor necrosis factor receptor family agonist, a tumor necrosis receptor antagonist, a tumor necrosis factor (TNF) soluble receptor or antibody, caspase protease activator or inhibitor, an NF-κB a RIPK1 and/or RIPK3 inhibitor or activator (e.g., through Toll-like receptors (TLRs) TLR-3 and/or TLR-4, or T-cell receptor (TCR) and the like), a death-receptor ligand (e.g., Fas ligand) activator or inhibitor, TNF receptor family (e.g., TNFR1, TNFR2, lymphotoxin R receptor/TNFRS3, OX40/TNFRSF4, CD40/TNFRSF5, Fas/TNFRSF6, decoy receptor 3/TNFRSF6B, CD27/TNFRSF7, CD30/TNFRSF8, 4-1BB/TNFRSF9, DR4 (death receptor 4/TNFRS10A), DR5 (death receptor 5/TNFRSF10B), decoy receptor 1/TNFRSF10C, decoy receptor 2/TNFRSF10D, RANK (receptor activator of NF-kappa B/TNFRSF11A), OPG (osteoprotegerin/TNFRSF11B), DR3 (death receptor 3/TNFRSF25), TWEAK receptor/TNFRSF12A, TAC1/TNFRSF13B, BAFF-R (BAFF receptor/TNFRSF13C), HVEM (herpes virus entry mediator/TNFRSF14), nerve growth factor receptor/TNFRSF16, BCMA (B cell maturation antigen/TNFRSF17), GITR (glucocorticoid-induced TNF receptor/TNFRSF18), TAJ (toxicity and JNK inducer/TNFRSF19), RELT/TNFRSF19L, DR6 (death receptor 6/TNFRSF21), TNFRSF22, TNFRSF23, ectodysplasin A2 isoform receptor/TNFRS27, ectodysplasin 1, and anhidrotic receptor, a TNF receptor superfamily ligand including—TNF alpha, lymphotoxin-α, tumor necrosis factor membrane form, tumor necrosis factor shed form, LIGHT, lymphotoxin β₂α₁ heterotrimer, OX-40 ligand, compound 1 [PMID: 24930776], CD40 ligand, Fas ligand, TL1A, CD70, CD30 ligand, TRAF1, TRAF2, TRAF3, TRAIL, RANK ligand, APRIL, BAFF, B and T lymphocyte attenuator, NGF, BDNF, neurotrophin-3, neurotrophin-4, TL6, ectodysplasin A2, ectodysplasin A1—a TIMP-3 inhibitor, a BCL-2 family inhibitor, navitoclax (Aging Cell. 15(3): 428-435. (2016)) an IAP disruptor, a protease inhibitor, an amino sugar, a chemotherapeutic (whether acting through an apoptotic or non-apoptotic pathway) (Ricci et al. Oncologist 11(4):342-57 (2006)), a cytotoxic chemical, a toxin, a tyrosine kinase inhibitor (e.g., imatinib mesylate), protons, bevacuzimab (antivascular agent), erlotinib (EGFR inhibitor), an anti-infective agent, an antibiotic, an anti-viral agent, an anti-fungal agent, an aminoglycoside, a nonsteroidal anti-inflammatory drug (NSAID), a statin, a nanoparticle, a liposome, a polymer, a biopolymer, a polysaccharide, a proteoglycan, a glycosaminoglycan, polyethylene glycol, a lipid, a dendrimer, a fatty acid, or an Fc domain or an Fc region, or an active fragment or a modification thereof. Any combination of the above active agents can be co-delivered with peptides or peptide conjugates of this disclosure. Additionally, in some embodiments, other co-therapies such as proton therapy or ablative radiotherapy can be administered to a subject in need thereof along with peptides or peptide conjugates of this disclosure. In some embodiments, the peptide is covalently or non-covalently linked to an active agent, e.g., directly or via a linker. TNF blockers suppress the immune system by blocking the activity of TNF, a substance in the body that can cause inflammation and lead to immune-system diseases, such as Crohn's disease, ulcerative colitis, rheumatoid arthritis, ankylosing spondylitis, psoriatic arthritis and plaque psoriasis. The drugs in this class include Remicade (infliximab), Enbrel (etanercept), Humira (adalimumab), Cimzia (certolizumab pegol) and Simponi (golimumab). The peptide disclosed herein can be used to home, distribute to, target, directed to, is retained by, accumulate in, migrate to, and/or bind to cartilage, and thus also be used for localizing the attached or fused active agent. Furthermore, cystine-dense chlorotoxin peptide can be internalized in cells (Wiranowska, M., Cancer Cell Int., 11: 27 (2011)). Therefore, cellular internalization, subcellular localization, and intracellular trafficking after internalization of the peptide itself, or an active agent peptide conjugate or fusion peptide can be important factors in the efficacy of an active agent conjugate or fusion. (Ducry, L., Antibody Drug Conjugates (2013); and Singh, S. K., Pharm Res., 32(11): 3541-3571 (2015)). Exemplary linkers suitable for use with the embodiments herein are discussed in further detail below.

The peptides or peptide-active agent fusions of the present disclosure can also be complexed, conjugated, or fused to other moieties that can serve other roles, such as providing an affinity handle (e.g., biotin) for retrieval of the peptides from tissues or fluids. For example, peptides or peptide-active agent fusions of the present disclosure can also be complexed, conjugated, or fused to biotin. In addition to extension of half-life, biotin could also act as an affinity handle for retrieval of peptides or peptide-active agent fusions from tissues or other locations. In some embodiments, fluorescent biotin conjugates that can act both as a detectable label and an affinity handle can be used. Non limiting examples of commercially available fluorescent biotin conjugates include Atto 425-Biotin, Atto 488-Biotin, Atto 520-Biotin, Atto-550 Biotin, Atto 565-Biotin, Atto 590-Biotin, Atto 610-Biotin, Atto 620-Biotin, Atto 655-Biotin, Atto 680-Biotin, Atto 700-Biotin, Atto 725-Biotin, Atto 740-Biotin, fluorescein biotin, biotin-4-fluorescein, biotin-(5-fluorescein) conjugate, and biotin-B-phycoerythrin, Alexa fluor 488 biocytin, Alexa flour 546, Alexa Fluor 549, lucifer yellow cadaverine biotin-X, Lucifer yellow biocytin, Oregon green 488 biocytin, biotin-rhodamine and tetramethylrhodamine biocytin. In some other examples, the conjugates could include chemiluminescent compounds, colloidal metals, luminescent compounds, enzymes, radioisotopes, and paramagnetic labels. In some embodiments, the peptide-active agent fusions described herein can be attached to another molecule. For example, the peptide sequence also can be attached to another active agent (e.g., small molecule, peptide, polypeptide, polynucleotide, antibody, aptamer, cytokine, growth factor, neurotransmitter, an active fragment or modification of any of the preceding, fluorophore, radioisotope, radionuclide chelator, acyl adduct, chemical linker, or sugar, etc.). In some embodiments, the peptide can be fused with, or covalently or non-covalently linked to an active agent.

Additionally, more than one peptide sequence can be present on or fused with a particular peptide. A peptide can be incorporated into a biomolecule by various techniques, for example by a chemical transformation, such as the formation of a covalent bond, such as an amide bond, or by solid phase or solution phase peptide synthesis, or by preparing a nucleic acid sequence encoding the biomolecule, wherein the nucleic acid sequence includes a subsequence that encodes the peptide. The subsequence can be in addition to the sequence that encodes the biomolecule, or can substitute for a subsequence of the sequence that encodes the biomolecule.

### Detectable Agent Conjugates

Described herein are agents that can be complexed, conjugated, or fused to the peptides of the present invention for use in detection and tracing either cartilage disorders or kidney disorders, or both. As described herein, it is understood that certain active agents are described in a non-limiting exemplary manner for use in diagnostics, aiding surgery and treatment, prognosis and tracking of progress or remission of cartilage and/or kidney disorders, diseases or injury. One or more of such detectable agents can be complexed, conjugated, or fused to a peptide of the present invention alone or in combination with one or more active agents described herein. Moreover some detectable agents (e.g., radionuclides, radioisotopes, radiosensitizers and photosensitizers amongst others) may also exert therapeutic activity as well. A peptide can be complexed, conjugated, or fused to an agent used in imaging, research, therapeutics, theranostics, pharmaceuticals, chemotherapy, chelation therapy, targeted drug delivery, and radiotherapy. The agent can be a detectable agent. In some embodiments, a peptide of the present invention is complexed, conjugated, or fused to detectable agents, such as a metal, a radioisotope, a dye, fluorophore, or another suitable material that can be used in imaging. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the fluorophore is a fluorescent agent emitting electromagnetic radiation at a wavelength between 650 nm and 4000 nm, such emissions being used to detect such agent. In some embodiments the fluorophore is a fluorescent agent is selected from the group consisting of non-limiting examples of fluorescent dyes that could be used as a conjugating molecule (or as applied to each class of molecules) in the present disclosure include DyLight-680, DyLight-750, VivoTag-750, DyLight-800, IRDye-800, VivoTag-680, Cy5.5, or indocyanine green (ICG class of dyes). In some embodiments, near infrared dyes include cyanine dyes. Additional non-limiting examples of fluorescent dyes for use as a conjugating molecule in the present disclosure include acradine orange or yellow, Alexa Fluors and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other Suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), indocyanine green dyes, and the like. For each of the above listed fluorescent dyes various activated forms can be used for conjugation. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Other embodiments of the present disclosure provide peptides complexed, conjugated, or fused to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of diseased cells (e.g., cancer cells) using both a therapeutic agent (e.g., drug) and electromagnetic energy (e.g., radiation or light) concurrently. In some embodiments, the peptide is covalently or non-covalently linked to the agent, e.g., directly or via a linker. Exemplary linkers suitable for use with the embodiments herein are discussed in further detail below.

The peptides or peptide-agent complexes of the present disclosure can be administered alone or in combination with a companion diagnostic, therapeutic agent, or imaging agent (said diagnostic or imaging agent can be linked to the peptides or peptide-agent complexes or, alternatively, can be used as a separate companion diagnostic, therapeutic agent, or imaging agent linked to the peptide for use in conjunction with the peptides or peptide-agent complex), such as chemical agents, radiolabel agents, radiosensitizing agents, fluorophores, imaging agents, diagnostic agents, proteins, peptides, or small molecules, wherein said agents are intended to have or have diagnostic or imaging effects. Agents used for companion diagnostic agents and companion imaging agents can include the diagnostic, therapeutic agent, and imaging agents described herein, or other diagnostic, therapeutic agent, and imaging agents consistent with the present disclosure. Diagnostic tests can be used to enhance the use of therapeutic products, such as those disclosed herein. The development of therapeutic products with a corresponding diagnostic test, such as a test that uses diagnostic imaging (whether in vivo or in vitro) can aid in diagnosis, treatment, identification of patient populations for treatment, and enhancement of the therapeutic effect of the corresponding therapy. Detection of therapeutic agents, such as those peptide and peptide agent complexes disclosed can also aid in the application of a therapy and to measure it to assess the agent's safety and physiologic effect, e.g. to measure bioavailability, uptake, distribution and clearance, metabolism, pharmacokinetics, localization, measurement of concentrations in blood and tissues, assessing therapeutic window, range and optimization, and the like of the therapeutic agent. Thus systems and methods can be employed in the context of therapeutic, imaging and diagnostic applications of such agents including peptides or peptide-agent complexes disclosed herein. Tests also aid therapeutic product development to obtain the data FDA uses to make regulatory determinations. For example, such a test can identify appropriate subpopulations for treatment or identify populations who should not receive a particular treatment because of an increased risk of a serious side effect, making it possible to individualize, or personalize, medical therapy by identifying patients who are most likely to respond, or who are at varying degrees of risk for a particular side effect. Thus, the present disclosure, in some embodiments, includes the joint development of therapeutic products and diagnostic devices (used to detect the peptide or peptide agent complexes themselves, or used to detect the companion diagnostic, therapeutic, or imaging agent, whether said diagnostic, therapeutic, or imaging agent is linked to the peptides or peptide-agent complex or used as a separate companion diagnostic, therapeutic, or imaging agent linked to the peptide for use in conjunction with the peptides or peptide-agent complex) that are used in conjunction with safe and effective use of the peptides or peptide-agent complexes as therapeutic products. Non-limiting examples of companion devices include a surgical instrument, such as an operating microscope, confocal microscope, fluorescence scope, exoscope, endoscope, or a surgical robot and devices used in biological diagnosis or imaging or that incorporate radiology, including the imaging technologies of X-ray radiography, magnetic resonance imaging (MRI), medical ultrasonography or ultrasound, endoscopy, elastography, tactile imaging, thermography, medical photography, and nuclear medicine functional imaging techniques, such as positron emission tomography (PET) and single-photon emission computed tomography (SPECT). Companion diagnostics, therapeutics, imaging agents, and devices can comprise tests that are conducted ex vivo, including detection of signal from tissues or cells that are removed following administration of the companion diagnostic to the subject, or application of the companion diagnostic, therapeutics, or companion imaging agent directly to tissues or cells following their removal from the subject and then detecting the signal. Examples of devices used for ex vivo detection include fluorescence microscopes, flow cytometers, and the like. Moreover, the systems and devices for such use in companion diagnostics include a surgical microscope, confocal microscope, fluorescence scope, exoscope, endoscope, or a surgical robot, including a KINEVO system (e.g., KINEVO 900), QEVO system, CONVIVO system, OMPI PENTERO system (e.g., PENTERO 900, PENTERO 800), INFRARED 800 system, FLOW 800 system, YELLOW 560 system, BLUE 400 system, OMPI LUMERIA systems OMPI Vario system (e.g., OMPI Vario and OMPI VARIO 700), OMPI Pico system, TREMON 3DHD system (and any additional exemplary surgical microscope, confocal microscope, fluorescence scope, exoscope, endoscope, and surgical robot systems from Carl Zeiss A/G); a PROVido system, ARvido system, GLOW 800 system, Leica M530 system (e.g., Leica M530 OHX, Leica M530 OH6), Leica M720 system (e.g., Leica M720 OHX5), Leica M525 System (e.g., Leica M525 F50, Leica M525 F40, Leica M525 F20, Leica M525 OH4), Leica HD C100 system, Leica FL system (e.g., Leica FL560, Leica FL400, Leica FL800), Leica DI C500, Leica ULT500, Leica Rotatable Beam Splitter, Leica M651 MSD, LIGHTENING, Leica TCS and SP8 systems (e.g., Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8 (and any additional exemplary surgical microscope, confocal microscope, fluorescence scope, exoscope, endoscope, and surgical robot systems from Leica Microsystems or Leica Biosystems); Haag-Streit 5-1000 and Haag-Streit 3-1000 systems (and any additional exemplary surgical microscope, confocal microscope, fluorescence scope, exoscope, endoscope, and surgical robot systems from Haag-Streit A/G); Intuitive Surgical da Vinci surgical robot systems (and any additional exemplary surgical microscope, confocal microscope, fluorescence scope, exoscope, endoscope, and surgical robot systems from Intuitive Surgical, Inc.).

### Linkers

Peptides according to the present disclosure that home, target, migrate to, are retained by, accumulate in, and/or bind to, or are directed to the cartilage can be attached to another moiety (e.g., an active agent), such as a small molecule, a second peptide, a protein, an antibody, an antibody fragment, an aptamer, polypeptide, polynucleotide, a fluorophore, a radioisotope, a radionuclide chelator, a polymer, a biopolymer, a fatty acid, an acyl adduct, a chemical linker, or sugar or other active agent described herein through a linker, or directly in the absence of a linker.

A peptide can be directly attached to another molecule by a covalent attachment. For example, the peptide is attached to a terminus of the amino acid sequence of a larger polypeptide or peptide molecule, or is attached to a side chain, such as the side chain of a lysine, serine, threonine, cysteine, tyrosine, aspartic acid, a non-natural amino acid residue, or glutamic acid residue. The attachment can be via an amide bond, an ester bond, an ether bond, a carbamate bond, a carbon-nitrogen bond, a triazole, a macrocycle, an oxime bond, a hydrazone bond, a carbon-carbon single double or triple bond, a disulfide bond, or a thioether bond. In some embodiments, similar regions of the disclosed peptide(s) itself (such as a terminus of the amino acid sequence, an amino acid side chain, such as the side chain of a lysine, serine, threonine, cysteine, tyrosine, aspartic acid, a non-natural amino acid residue, or glutamic acid residue, via an amide bond, an ester bond, an ether bond, a carbamate bond, a carbon-nitrogen bond, a triazole, a macrocycle, an oxime bond, a hydrazone bond, a carbon-carbon single double or triple bond, a disulfide bond, or a thioether bond, or linker as described herein) can be used to link other molecules.

Attachment via a linker can involve incorporation of a linker moiety between the other molecule and the peptide. The peptide and the other molecule can both be covalently attached to the linker. The linker can be cleavable, labile, non-cleavable, stable, stable self-immolating, hydrophilic, or hydrophobic. As used herein, the term “non-cleavable” or “stable” (such as used in association with an amide, cyclic, or carbamate linker or as otherwise as described herein) is often used by a skilled artisan to distinguish a relatively stable structure from one that is more labile or “cleavable” (e.g., as used in association with cleavable linkers that may be dissociated or cleaved structurally by enzymes, proteases, self-immolation, pH, reduction, hydrolysis, certain physiologic conditions, or as otherwise described herein). It is understood that “non-cleavable” or “stable” linkers offer stability against cleavage or other dissociation as compared to “cleavable” linkers, and the term is not intended to be considered an absolute non-cleavable or non-dissociative structure under any conditions. Consequently, as used herein, a “non-cleavable” linker is also referred to as a “stable” linker. The linker can have at least two functional groups with one bonded to the peptide, the other bonded to the other molecule, and a linking portion between the two functional groups.

Non-limiting examples of the functional groups for attachment can include functional groups capable of forming an amide bond, an ester bond, an ether bond, a carbonate bond, a carbamate bond, or a thioether bond. Non-limiting examples of functional groups capable of forming such bonds can include amino groups; carboxyl groups; hydroxyl groups; aldehyde groups; azide groups; alkyne and alkene groups; ketones; hydrazides; acid halides such as acid fluorides, chlorides, bromides, and iodides; acid anhydrides, including symmetrical, mixed, and cyclic anhydrides; carbonates; carbonyl functionalities bonded to leaving groups such as cyano, succinimidyl, and N-hydroxysuccinimidyl; hydroxyl groups; sulfhydryl groups; and molecules possessing, for example, alkyl, alkenyl, alkynyl, allylic, or benzylic leaving groups, such as halides, mesylates, tosylates, triflates, epoxides, phosphate esters, sulfate esters, and besylates.

Non-limiting examples of the linking portion can include alkylene, alkenylene, alkynylene, polyether, such as polyethylene glycol (PEG), hydroxy carboxylic acids, polyester, polyamide, polyamino acids, polypeptides, cleavable peptides, valine-citrulline, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, and ester groups.

A peptide and drug complexed, conjugated, or fused via a linker is described with the formula Peptide-A-B-C-Drug, wherein the linker is A-B-C. A can be a stable amide link, is an amine on the peptide and the linker and can be achieved via a tetrafluorophenyl (TFP) ester or an NHS ester. B can be (—CH2-)ₓ- or a short PEG (—CH₂CH₂O—)ₓ (x is 1-10), and C can be the ester bond to the hydroxyl or carboxylic acid on the drug. In some embodiments, C can refer to the “cleavable” or “stable” part of the linker. In other embodiments, A can also be the “cleavable” part. In some embodiments, A can be amide, carbamate, thioether via maleimide or bromoacetamide, triazole, oxime, or oxacarboline. The cleaved active agent or drug can retain the chemical structure of the active agent before cleavage, or can be modified as a result of cleavage. Moreover, depending on the desired therapeutic properties of the peptide-drug conjugate, such active agent can be active while linked to the peptide, remain active after cleavage or become inactivated, be inactive while linked to the peptide, or it can be activated upon cleavage.

In some embodiments, peptide conjugates have stable linkers. A peptide of the disclosure can be expressed recombinantly or chemically synthesized. The peptide can be complexed, conjugated, or fused to a detectable agent or an active agent via a stable linker, such as an amide linkage or a carbamate linkage. The peptide can be complexed, conjugated, or fused to a detectable agent or an active agent via a stable linker, such as an amide bond using standard 1-ethyl-3-(3-dimethylaminopropyl)carbodiimide (EDC) or dicyclohexylcarbodiimide (DCC) based chemistry or thionyl chloride or phosphorous chloride-based bioconjugation chemistries. A stable linker may or may not be cleaved in buffer over extended periods of time (e.g., hours, days, or weeks). A stable linker may or may not be cleaved in body fluids such as plasma or synovial fluid over extended periods of time (e.g., hours, days, or weeks). A stable linker, may or may not be cleaved after exposure to enzymes, reactive oxygen species, other chemicals or enzymes that can be present in cells (e.g., macrophages), cellular compartments (e.g., endosomes and lysosomes), inflamed areas of the body (e.g., inflamed joints), tissues or body compartments. A stable linker may be cleaved by unknown mechanisms. A stable linker may or may not be cleaved in vivo but remains an active agent after peptide conjugation.

A peptide and drug complexed, conjugated, or fused via a linker can be described with the formula Peptide-A-B-C-Drug, wherein the linker is A-B-C. A can be a stable amide link such as that formed by reacting an amine on the peptide with a linker containing a tetrafluorophenyl (TFP) ester or an NHS ester. A can also be a stable carbamate linker such as that formed by reacting an amine on the peptide with an imidazole carbamate active intermediate formed by reaction of CDI with a hydroxyl on the linker. A can also be a stable secondary amine linkage such as that formed by reductive alkylation of the amine on the peptide with an aldehyde or ketone group on the linker. A can also be a stable thioether linker formed using a maleimide or bromoacetamide in the linker with a thiol in the peptide, a triazole linker, a stable oxime linker, or a oxacarboline linker. B can be (—CH2-)ₓ- or a short PEG (—CH₂CH₂O—)ₓ (x is 0-20) or other spacers or no spacer. C can be an amide bond formed with an amine or a carboxylic acid on the drug, a thioether formed between a maleimide on the linker and a sulfhydroyl on the drug, a secondary or tertiary amine, a carbamate, or other stable bonds. Any linker chemistry described in “Current ADC Linker Chemistry,” Jain et al., Pharm Res, 2015 DOI 10.1007/s11095-015-1657-7 can be used.

The resulting peptide conjugates can be administered to a human or animal subcutaneously, intravenously, orally, or injected directly into a joint to treat disease. The peptide is not specifically cleaved from the detectable agent or active agent via a targeted mechanism. The peptide can be degraded by mechanisms such as catabolism, releasing a drug that is modified or not modified form its native form (Antibody-Drug Conjugates: Design, Formulation, and Physicochemical Stability, Singh, Luisi, and Pak. Pharm Res (2015) 32:3541-3571). The peptide drug conjugate exerts its pharmacological activity while still intact, or while partially or fully degraded, metabolized, or catabolized.

In some embodiments, peptide conjugates can have cleavable linkers. In some embodiments, a peptide and drug can be complexed, conjugated, or fused via a linker and can be described with the formula Peptide-A-B-C-Drug, wherein the linker is A-B-C. In some embodiments, A can be a stable amide link such as that formed by reacting an amine on the peptide with a linker containing a tetrafluorophenyl (TFP) ester or an NHS ester. In certain embodiments, A can also be a stable carbamate linker that is formed by an amine reaction on the peptide with an imidazole carbamate active intermediate formed by reaction of CDI with a hydroxyl on the linker. In other embodiments, A can also be a stable secondary amine linkage such as that formed by reductive alkylation of the amine on the peptide with an aldehyde or ketone group on the linker. In some embodiments, A can also be a stable thioether linker formed using a maleimide or bromoacetamide in the linker with a thiol in the peptide, a triazole linker, a stable oxime linker, or an oxacarboline linker. B can be (—CH2-)ₓ- or a short PEG (—CH₂CH₂O—)ₓ (x is 0-20) or other spacers or no spacer. C can be an ester bond to the hydroxyl or carboxylic acid on the drug, or a carbonate, hydrazone, or acylhydrazone, designed for hydrolytic cleavage. The hydrolytic rate of cleavage can be varied by varying the local environment around the bond, including carbon length (—CH2-)x, steric hindrance (including adjacent side groups such as methyl, ethyl, cyclic), hydrophilicity or hydrophobicity. In some embodiments, peptide conjugates can have a linear or cyclic ester linkage, which can include or do not include side chains such as methyl or ethyl groups. A linear ester linkage can be more susceptible to cleavage (such as by hydrolysis, an enzyme such as esterase, or other chemical reaction) than a cyclic ester due to steric hindrance or hydrophobicity/hydrophilicity effects. Likewise, side chains such as methyl or ethyl groups on the linear ester linkage can optionally make the linkage less susceptible to cleavage than without the side chains. In some embodiments, hydrolysis rate can be affected by local pH, such as lower pH in certain compartments of the body or of the cell such as endosomes and lysosomes or diseased tissues. In some embodiments, C can also be a pH sensitive group such as a hydrazone or oxime linkage. In other embodiments, C can be a disulfide bond designed to be released by reduction, such as by glutathione. In other embodiments, (or A-B-C) can be a peptidic linkage design for cleavable by enzymes. Optionally, a self-immolating group such as pABC can be included to cause release of a free unmodified drug upon cleavage (Antibody-Drug Conjugates: Design, Formulation, and Physicochemical Stability, Singh, Luisi, and Pak. Pharm Res (2015) 32:3541-3571). The linker can be cleaved by enzymes such as esterases, matrix metalloproteinases, cathepsins such as cathepsin B, glucuronidases, a protease, or thrombin. Alternatively, the bond designed for cleavage can be at A, rather than C, and C can be a stable bond or a cleavable bond. An alternative design can be to have stable linkers (such as amide or carbamate) at A and C and have a cleavable linker in B, such as a disulfide bond. The rate of reduction can be modulated by local effects such as steric hindrance from methyl or ethyl groups or modulating hydrophobicity/hydrophilicity. In some embodiments, peptide conjugates can have an ester carbonyl linkage, a long hydrocarbon linker, or carbamate linker, each of which can include hydrophilic groups, such as alcohols, acids, or ethers, or include a hydrocarbon side chain or other moiety that tunes the rate of cleavage. For example, the rate of hydrolysis can be faster with hydrophilic groups, such as alcohols, acids, or ethers, near an ester carbonyl. In another example, hydrophobic groups present as side chains or as a longer hydrocarbon linker can slow the cleavage rate of the ester. Likewise, cleavage of a carbamate group can also be tuned by hindrance, hydrophobicity, and the like. In another example, using a less labile linking group, such as a carbamate rather than an ester, can slow the cleavage rate of the linker.

Non-limiting examples of linkers include:

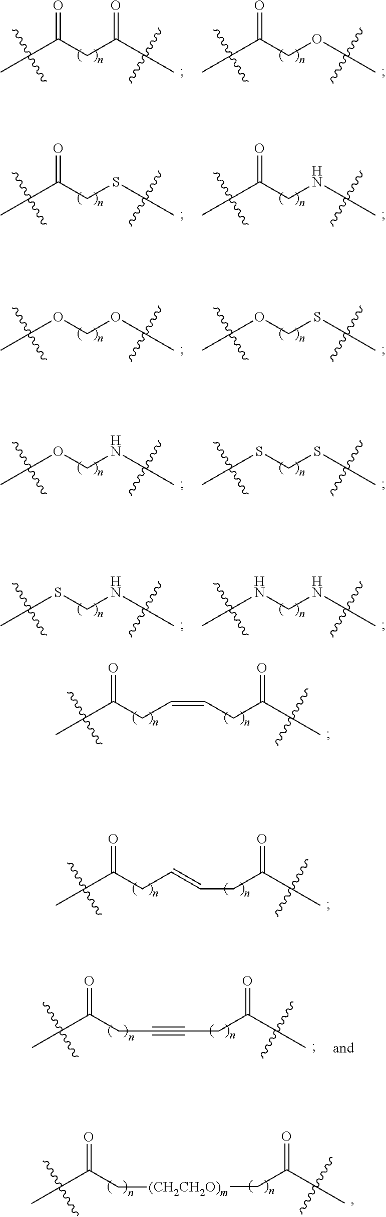

wherein each n is independently 0 to about 1,000; 1 to about 1,000; 0 to about 500; 1 to about 500; 0 to about 250; 1 to about 250; 0 to about 200; 1 to about 200; 0 to about 150; 1 to about 150; 0 to about 100; 1 to about 100; 0 to about 50; 1 to about 50; 0 to about 40; 1 to about 40; 0 to about 30; 1 to about 30; 0 to about 25; 1 to about 25; 0 to about 20; 1 to about 20; 0 to about 15; 1 to about 15; 0 to about 10; 1 to about 10; 0 to about 5; or 1 to about 5. In some embodiments, each n is independently 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50. In some embodiments, m is 1 to about 1,000; 1 to about 500; 1 to about 250; 1 to about 200; 1 to about 150; 1 to about 100; 1 to about 50; 1 to about 40; 1 to about 30; 1 to about 25; 1 to about 20; 1 to about 15; 1 to about 10; or 1 to about 5. In some embodiments, m is 0, about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50.

In some cases a linker can be a succinic linker, and a drug can be attached to a peptide via an ester bond or an amide bond with two methylene carbons in between. In other cases, a linker can be any linker with both a hydroxyl group and a carboxylic acid, such as hydroxy hexanoic acid or lactic acid.

The linker can be a cleavable or a stable linker. The use of a cleavable linker permits release of the complexed, conjugated, or fused moiety (e.g., a therapeutic agent) from the peptide, e.g., after targeting to the cartilage. In some cases the linker is enzyme cleavable, e.g., a valine-citrulline linker. In some embodiments, the linker contains a self-immolating portion. In other embodiments, the linker includes one or more cleavage sites for a specific protease, such as a cleavage site for matrix metalloproteases (MMPs), thrombin, or a cathepsin. Alternatively or in combination, the linker is cleavable by other mechanisms, such as via pH, reduction, or hydrolysis. A hydrolytically labile linker, (amongst other cleavable linkers described herein) can be advantageous in terms of releasing active agents from the peptide. For example, an active agent in a conjugate form with the peptide may not be active, but upon release from the conjugate after targeting to the cartilage, the active agent is active.

The rate of hydrolysis of the linker can be tuned. For example, the rate of hydrolysis of linkers with unhindered esters is faster compared to the hydrolysis of linkers with bulky groups next an ester carbonyl. A bulky group can be a methyl group, an ethyl group, a phenyl group, a ring, or an isopropyl group, or any group that provides steric bulk. In some cases, the steric bulk can be provided by the drug itself, such as by ketorolac when complexed, conjugated, or fused via its carboxylic acid. The rate of hydrolysis of the linker can be tuned according to the residency time of the conjugate in the cartilage. For example, when a peptide is cleared from the cartilage relatively quickly, the linker can be tuned to rapidly hydrolyze. In contrast, for example, when a peptide has a longer residence time in the cartilage, a slower hydrolysis rate can allow for extended delivery of an active agent. This can be important when the peptide is used to deliver a drug to the cartilage. “Programmed hydrolysis in designing paclitaxel prodrug for nanocarrier assembly” Sci Rep 2015, 5, 12023 Fu et al., provides an example of modified hydrolysis rates.

### Peptide Stability

A peptide of the present disclosure can be stable in various biological conditions, as well as during manufacturing, handling, storage, and other conditions in either a liquid or a dried state. Additionally, a peptide of the present disclosure can be resistant to enzymatic cleavage needed for peptide processing by the immune system. For example, any peptide of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 can exhibit resistance to reducing agents, proteases, oxidative conditions, or acidic conditions.

In some cases, biologic molecules (such as peptides and proteins) can provide therapeutic functions, but such therapeutic functions are decreased or impeded by instability caused by the in vivo environment. (Moroz et al., Adv Drug Deliv Rev 101:108-21 (2016), Mitragotri et al., Nat Rev Drug Discov 13(9):655-72 (2014), Bruno et al., Ther Deliv (11):1443-67 (2013), Sinha et al., Crit Rev Ther Drug Carrier Syst. 24(1):63-92 (2007), Hamman et al., BioDrugs 19(3):165-77 (2005)). Peptide degradation can be a result of a number of processes involving hydrolytic pathways, peptide oxidation such as oxidation of methionine (Met) residues, deamidation of asparagine (Asn) and glutamine (Gln) residues, and isomerization and hydrolysis of an adjacent asparagine (Asp) residue. (Manning et al., Pharmaceutical Research, Vol. 27 No. 4 (2010)). The amino acid immediately following the Asn or Gln residue can also affect the rate of deamidation, whereas: Asn-Gly, Asn-Ser, Asn-His, and Gln-Gly can be more likely to undergo deamidation. Additionally, the peptide bond adjacent to amino acids such as Asp can undergo hydrolysis with amino acid pairings such as Asp-Gly, Asp-Ser, Asp-Tyr, and Asp-Pro, which can be more likely to undergo hydrolysis. Oxidation of amino acid residues such as Met can form a sulfoxide species. The specific degradation reactions rates can vary for any given peptide or protein sequence.

Furthermore, the microenvironment within the molecular structure of the peptide, solvent accessibility, and conformational stability of each residue can impact the likelihood of peptide degradation. Therefore, by modifying a peptide sequence to reduce occurrence of such degradation events, a the modified peptide or peptide-conjugate can have increased beneficial properties over unmodified peptides or peptide-drug conjugates, such as improved therapeutic efficacy, an increased safety profile, and can be less expensive to manufacture and develop. Key formulaic considerations that can prevent peptide decay can include the use of excipients, formulation at a desired pH, and storage under specific conditions (e.g., temperature, oxygen, light exposure, solid or liquid state, and container excipient materials). To circumvent degradation, peptide residues can be substituted with amino acids that increase stability, which can result in more efficacious and durable therapeutic peptides.

With respect to in vivo stability, the GI tract can contain a region of low pH (e.g., pH ˜1), a reducing environment, or a protease-rich environment that can degrade peptides and proteins. Proteolytic activity in other areas of the body, such as the mouth, eye, lung, intranasal cavity, joint, skin, vaginal tract, mucous membranes, and serum, can also be an obstacle to the delivery of functionally active peptides and polypeptides. Additionally, the half-life of peptides in serum can be very short, in part due to proteases, such that the peptide can be degraded too quickly to have a lasting therapeutic effect when administering a therapeutic and safe dosing regimen. Likewise, proteolytic activity in cellular compartments, such as lysosomes, and reduction activity in lysosomes and the cytosol can degrade peptides and proteins such that they may be unable to provide a therapeutic function on intracellular targets. Therefore, peptides that are resistant to reducing agents, proteases, and low pH may be able to provide enhanced therapeutic effects or enhance the therapeutic efficacy of co-formulated or complexed, conjugated, or fused active agents in vivo.

Additionally, oral delivery of drugs can be desirable in order to target certain areas of the body (e.g., disease in the GI tract such as colon cancer, irritable bowel disorder, infections, metabolic disorders, and constipation) despite the obstacles to the delivery of functionally active peptides and polypeptides presented by this method of administration. For example, oral delivery of drugs can increase compliance by providing a dosage form that is more convenient for patients to take as compared to parenteral delivery. Oral delivery can be useful in treatment regimens that have a large therapeutic window. Therefore, peptides that are resistant to reducing agents, proteases, and low pH can allow for oral delivery of peptides without nullifying their therapeutic function.

Peptide Resistance to Reducing Agents.

Peptides of this disclosure can contain one or more cysteines, which can participate in disulfide bridges that can be integral to preserving the folded state of the peptide. Exposure of peptides to biological environments with reducing agents can result in unfolding of the peptide and loss of functionality and bioactivity. For example, glutathione (GSH) is a reducing agent that can be present in many areas of the body and in cells, and can reduce disulfide bonds. As another example, a peptide can become reduced upon cellular internalization during trafficking of a peptide across the gastrointestinal epithelium after oral administration a peptide can become reduced upon exposure to various parts of the GI tract. The GI tract can be a reducing environment, which can inhibit the ability of therapeutic molecules with disulfide bonds to have optimal therapeutic efficacy, due to reduction of the disulfide bonds. A peptide can also be reduced upon entry into a cell, such as after internalization by endosomes or lysosomes or into the cytosol, or other cellular compartments. Reduction of the disulfide bonds and unfolding of the peptide can lead to loss of functionality or affect key pharmacokinetic parameters such as bioavailability, peak plasma concentration, bioactivity, and half-life. Reduction of the disulfide bonds can also lead to increased susceptibility of the peptide to subsequent degradation by proteases, resulting in rapid loss of intact peptide after administration. In some embodiments, a peptide that is resistant to reduction can remain intact and can impart a functional activity for a longer period of time in various compartments of the body and in cells, as compared to a peptide that is more readily reduced.

In certain embodiments, the peptides of this disclosure can be analyzed for the characteristic of resistance to reducing agents to identify stable peptides. In some embodiments, the peptides of this disclosure can remain intact after being exposed to different molarities of reducing agents such as 0.00001M-0.0001M, 0.0001M-0.001M, 0.001M-0.01M, 0.01 M-0.05 M, 0.05 M-0.1 M, for greater 15 minutes or more. In some embodiments, the reducing agent used to determine peptide stability can be dithiothreitol (DTT), Tris (2-carboxyethyl) phosphine HCl (TCEP), 2-Mercaptoethanol, (reduced) glutathione (GSH), or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a reducing agent.

Peptide Resistance to Proteases.

The stability of peptides of this disclosure can be determined by resistance to degradation by proteases. Proteases, also referred to as peptidases or proteinases, can be enzymes that can degrade peptides and proteins by breaking bonds between adjacent amino acids. Families of proteases with specificity for targeting specific amino acids can include serine proteases, cysteine proteases, threonine proteases, aspartic proteases, glutamic proteases, esterases, serum proteases, and asparagine proteases. Additionally, metalloproteases, matrix metalloproteases, elastase, carboxypeptidases, Cytochrome P450 enzymes, and cathepsins can also digest peptides and proteins. Proteases can be present at high concentration in blood, in mucous membranes, lungs, skin, the GI tract, the mouth, nose, eye, and in compartments of the cell. Misregulation of proteases can also be present in various diseases such as rheumatoid arthritis and other immune disorders. Degradation by proteases can reduce bioavailability, biodistribution, half-life, and bioactivity of therapeutic molecules such that they are unable to perform their therapeutic function. In some embodiments, peptides that are resistant to proteases can better provide therapeutic activity at reasonably tolerated concentrations in vivo.

In some embodiments, peptides of this disclosure can resist degradation by any class of protease. In certain embodiments, peptides of this disclosure resist degradation by pepsin (which can be found in the stomach), trypsin (which can be found in the duodenum), serum proteases, or any combination thereof. In certain embodiments, peptides of this disclosure can resist degradation by lung proteases (e. g., serine, cysteinyl, and aspartyl proteases, metalloproteases, neutrophil elastase, alpha-1 antitrypsin, secretory leucoprotease inhibitor, elafin), or any combination thereof. In some embodiments, the proteases used to determine peptide stability can be pepsin, trypsin, chymotrypsin, or any combination thereof. In some embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a protease. Peptides of, SEQ ID NO: 150, and SEQ ID NO: 149 (also disclosed as SEQ ID NO: 46; non-GS version of SEQ ID NO: 149/SEQ ID NO: 46 are shown in SEQ ID NO: 128 and SEQ ID NO: 205) can have particular structural qualities, which make them more resistant to protease degradation. For example, peptide of SEQ ID NO: 150 and SEQ ID NO: 149 exhibit the “hitchin” topology as described previously, which can be associated with resistance to protease and chemical degradation.

Peptide Stability in Acidic Conditions.

Peptides of this disclosure can be administered in biological environments that are acidic. For example, after oral administration, peptides can experience acidic environmental conditions in the gastric fluids of the stomach and gastrointestinal (GI) tract. The pH of the stomach can range from ˜1-4 and the pH of the GI tract ranges from acidic to normal physiological pH descending from the upper GI tract to the colon. In addition, the vagina, late endosomes, and lysosomes can also have acidic pH values, such as less than pH 7. These acidic conditions can lead to denaturation of peptides and proteins into unfolded states. Unfolding of peptides and proteins can lead to increased susceptibility to subsequent digestion by other enzymes as well as loss of biological activity of the peptide.

In certain embodiments, the peptides of this disclosure can resist denaturation and degradation in acidic conditions and in buffers, which simulate acidic conditions. In certain embodiments, peptides of this disclosure can resist denaturation or degradation in buffer with a pH less than 1, a pH less than 2, a pH less than 3, a pH less than 4, a pH less than 5, a pH less than 6, a pH less than 7, or a pH less than 8. In some embodiments, peptides of this disclosure remain intact at a pH of 1-3. In certain embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH less than 1, a pH less than 2, a pH less than 3, a pH less than 4, a pH less than 5, a pH less than 6, a pH less than 7, or a pH less than 8. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to a buffer with a pH of 1-3. In other embodiments, the peptides of this disclosure can be resistant to denaturation or degradation in simulated gastric fluid (pH 1-2). In some embodiments, at least 5-10%, at least 10%-20%, at least 20%-30%, at least 30%-40%, at least 40%-50%, at least 50%-60%, at least 60%-70%, at least 70%-80%, at least 80%-90%, or at least 90-100% of the peptide remains intact after exposure to simulated gastric fluid. In some embodiments, low pH solutions such as simulated gastric fluid or citrate buffers can be used to determine peptide stability.

Peptide Stability at High Temperatures.

Peptides of this disclosure can be administered in biological environments with high temperatures. For example, after oral administration, peptides can experience high temperatures in the body. Body temperature can range from 36° C. to 40° C. High temperatures can lead to denaturation of peptides and proteins into unfolded states. Unfolding of peptides and proteins can lead to increased susceptibility to subsequent digestion by other enzymes as well as loss of biological activity of the peptide. In some embodiments, a peptide of this disclosure can remain intact at temperatures from 25° C. to 100° C. High temperatures can lead to faster degradation of peptides. Stability at a higher temperature can allow for storage of the peptide in tropical environments or areas where access to refrigeration is limited. In certain embodiments, 5%-100% of the peptide can remain intact after exposure to 25° C. for 6 months to 5 years. 5%-100% of a peptide can remain intact after exposure to 70° C. for 15 minutes to 1 hour. 5%-100% of a peptide can remain intact after exposure to 100° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 25° C. for 6 months to 5 years. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 70° C. for 15 minutes to 1 hour. In other embodiments, at least 5%-10%, at least 10%-20%, at least 20%-30%, at least 30%-40%, at least 40%-50%, at least 50%-60%, at least 60%-70%, at least 70%-80%, at least 80%-90%, or at least 90%-100% of the peptide remains intact after exposure to 100° C. for 15 minutes to 1 hour.

### Pharmacokinetics of Peptides

The pharmacokinetics of any of the peptides of this disclosure can be determined after administration of the peptide via different routes of administration. For example, the pharmacokinetic parameters of a peptide of this disclosure can be quantified after intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, intra-articular, peritoneal, buccal, synovial, or topical administration. Peptides of the present disclosure can be analyzed by using tracking agents such as radiolabels or fluorophores. For example, a radiolabeled peptide of this disclosure can be administered via various routes of administration. Peptide concentration or dose recovery in various biological samples such as plasma, urine, feces, any organ, skin, muscle, and other tissues can be determined using a range of methods including HPLC, fluorescence detection techniques (TECAN quantification, flow cytometry, iVIS), or liquid scintillation counting.

The methods and compositions described herein can relate to pharmacokinetics of peptide administration via any route to a subject. Pharmacokinetics can be described using methods and models, for example, compartmental models or noncompartmental methods. Compartmental models include but are not limited to monocompartmental model, the two compartmental model, the multicompartmental model or the like. Models can be divided into different compartments and can be described by the corresponding scheme. For example, one scheme is the absorption, distribution, metabolism and excretion (ADME) scheme. For another example, another scheme is the liberation, absorption, distribution, metabolism and excretion (LADME) scheme. In some aspects, metabolism and excretion can be grouped into one compartment referred to as the elimination compartment. For example, liberation can include liberation of the active portion of the composition from the delivery system, absorption includes absorption of the active portion of the composition by the subject, distribution includes distribution of the composition through the blood plasma and to different tissues, metabolism, which includes metabolism or inactivation of the composition and finally excretion, which includes excretion or elimination of the composition or the products of metabolism of the composition. Compositions administered intravenously to a subject can be subject to multiphasic pharmacokinetic profiles, which can include but are not limited to aspects of tissue distribution and metabolism/excretion. As such, the decrease in plasma or serum concentration of the composition is often biphasic, including, for example an alpha phase and a beta phase, occasionally a gamma, delta or other phase is observed

Pharmacokinetics includes determining at least one parameter associated with administration of a peptide to a subject. In some aspects, parameters include at least the dose (D), dosing interval (τ), area under curve (AUC), maximum concentration (Cₘₐₓ), minimum concentration reached before a subsequent dose is administered (Cₘᵢₙ), minimum time (Tₘᵢₙ), maximum time to reach C max (Tₘₐₓ), volume of distribution (V_{d}), steady-state volume of distribution (Vₛₛ), back-extrapolated concentration at time 0 (C₀), steady state concentration (Cₛₛ), elimination rate constant (kₑ), infusion rate (kᵢₙ), clearance (CL), bioavailability (f), fluctuation (% PTF) and elimination half-life (t_{1/2}).

In certain embodiments, the peptides of any of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 exhibit optimal pharmacokinetic parameters after oral administration. In other embodiments, the peptides of any of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 exhibit optimal pharmacokinetic parameters after any route of administration, such as oral administration, inhalation, intranasal administration, topical administration, parenteral administration, intravenous administration, subcutaneous administration, intra-articular administration, intramuscular administration, intraperitoneal administration, transdermal administration, dermal administration, or any combination thereof.

In some embodiments any peptide of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 exhibits an average Tₘₐₓ of 0.5-12 hours, or 1-48 hours at which the Cₘₐₓ is reached, an average bioavailability in serum of 0.1%-10% in the subject after administering the peptide to the subject by an oral route, an average bioavailability in serum of less than 0.1% after oral administration to a subject for delivery to the GI tract, an average bioavailability in serum of 10-100% after parenteral administration, an average t of 0.1 hours-168 hours, or 0.25 hours-48 hours in a subject after administering the peptide to the subject, an average clearance (CL) of 0.5-100 L/hour or 0.5-50 L/hour of the peptide after administering the peptide to a subject, an average volume of distribution (V_{d}) of 200-20,000 mL in the subject after systemically administering the peptide to the subject, or optionally no systemic uptake, any combination thereof.

### Methods of Manufacture

Various expression vector/host systems can be utilized for the production of the recombinant expression of peptides described herein. Non-limiting examples of such systems include microorganisms such as bacteria transformed with recombinant bacteriophage DNA, plasmid DNA or cosmid DNA expression vectors containing a nucleic acid sequence encoding peptides or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV) or transformed with recombinant plasmid expression vectors (e.g., Ti plasmid) containing the aforementioned nucleic acid sequence, or animal cell systems infected with recombinant virus expression vectors (e.g., adenovirus, vaccinia virus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines). Disulfide bond formation and folding of the peptide could occur during expression or after expression or both.

A host cell can be adapted to express one or more peptides described herein. The host cells can be prokaryotic, eukaryotic, or insect cells. In some cases, host cells are capable of modulating the expression of the inserted sequences, or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide products can be important for the function of the peptide. Host cells can have characteristic and specific mechanisms for the post-translational processing and modification of a peptide. In some cases, the host cells used to express the peptides secretes minimal amounts of proteolytic enzymes.

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, Peptides can also be synthesized in a cell-free system using a variety of known techniques employed in protein and peptide synthesis.

In some cases, a host cell produces a peptide that has an attachment point for a drug. An attachment point could comprise a lysine residue, an N-terminus, a cysteine residue, a cysteine disulfide bond, or a non-natural amino acid or a unique peptide sequence such as targeted by an enzyme. The peptide could also be produced synthetically, such as by solid-phase peptide synthesis, or solution-phase peptide synthesis. The peptide could be folded (formation of disulfide bonds) during synthesis or after synthesis or both. Peptide fragments could be produced synthetically or recombinantly and then joined together synthetically, recombinantly, or via an enzyme.

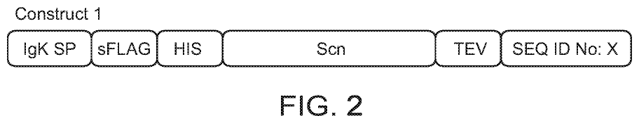

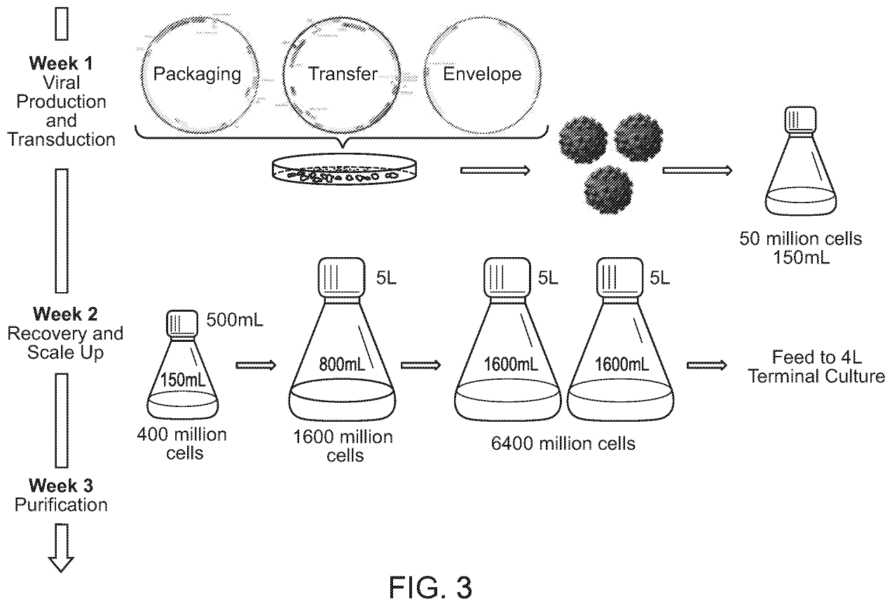

FIG. 3 illustrates a schematic of a method of manufacturing a construct that expresses a peptide of the disclosure, such as the constructs illustrated in FIG. 2 and as described throughout the disclosure and in SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 provided herein.

In other aspects, the peptides of the present disclosure can be prepared by conventional solid phase chemical synthesis techniques, for example according to the Fmoc solid phase peptide synthesis method (“Fmoc solid phase peptide synthesis, a practical approach,” edited by W. C. Chan and P. D. White, Oxford University Press, 2000), Boc solid phase peptide synthesis, or solution phase peptide synthesis. The disulfide bonds can be formed after cleavage from the resin, such as by air oxidation or a buffer system with a set pH range such as from 7-10 and can contain a redox system such as glutathione/oxidized glutathione or cysteine/cystine. The disulfide bonds can also be formed by selective protection and deprotection of specific cysteine residues followed by oxidation, or on the resin. The peptide can be purified, such as by reversed-phase chromatography at any one or more steps during the production process. The peptide can be isolated by lyophilization and can be in various salt forms, such as TFA salt or ammonium and acetate salt.

### Pharmaceutical Compositions of Peptides

A pharmaceutical composition of the disclosure can be a combination of any peptide described herein with other chemical components, such as carriers, stabilizers, diluents, dispersing agents, suspending agents, thickening agents, antioxidants, solubilizers, buffers, osmolytes, salts, surfactants, amino acids, encapsulating agents, bulking agents, cryoprotectants, and/or excipients. The pharmaceutical composition facilitates administration of a peptide described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, intra-articular, and topical administration. A pharmaceutical composition can be administered in a local or systemic manner, for example, via injection of the peptide described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water soluble form. Suspensions of peptides described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduce the aggregation of such peptides described herein to allow for the preparation of highly concentrated solutions. Alternatively, the peptides described herein can be lyophilized or in powder form for re-constitution with a suitable vehicle, e.g., sterile pyrogen-free water, before use. In some embodiments, a purified peptide is administered intravenously.

A peptide of the disclosure can be applied directly to an organ, or an organ tissue or cells, such as brain or brain tissue or cancer cells, during a surgical procedure. The recombinant peptides described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptide described herein described herein can be administered in pharmaceutical compositions to a subject suffering from a condition that affects the immune system. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of peptides described herein comprising the compounds described herein include formulating the peptide described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa. Mack Publishing Company, 1995); Hoover, John E., Remington's Pharmaceutical Sciences, Mack Publishing Co., Easton, Pa. 1975; Liberman, H. A. and Lachman, L., Eds; Pharmaceutical Dosage Forms, Marcel Decker, New York, N. Y., 1980; and Pharmaceutical Dosage Forms and Drug Delivery Systems, Seventh Ed. (Lippincott Williams & Wilkins 1999), each of which is incorporated by reference in its entirety.

### Administration of Pharmaceutical Compositions

A pharmaceutical composition of the disclosure can be a combination of any peptide described herein with other chemical components, such as carriers, stabilizers, diluents, dispersing agents, suspending agents, thickening agents, and/or excipients. The pharmaceutical composition facilitates administration of a peptide described herein to an organism. Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, inhalation, dermal, intra-articular, intrathecal, intranasal, and topical administration. A pharmaceutical composition can be administered in a local or systemic manner, for example, via injection of the peptide described herein directly into an organ, optionally in a depot.

Parenteral injections can be formulated for bolus injection or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water-soluble form. Suspensions of peptides described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduce the aggregation of such peptides described herein to allow for the preparation of highly concentrated solutions. Alternatively, the peptides described herein can be lyophilized or in powder form for re-constitution with a suitable vehicle, e.g., sterile pyrogen-free water, before use. In some embodiments, a purified peptide is administered intravenously. A peptide described herein can be administered to a subject, home, target, migrates to, is retained by, and/or binds to, or be directed to an organ, e.g., the cartilage.

A peptide of the disclosure can be applied directly to an organ, or an organ tissue or cells, such as cartilage or cartilage tissue or cells, during a surgical procedure. The recombinant peptides described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

In practicing the methods of treatment or use provided herein, therapeutically-effective amounts of the peptide described herein described herein are administered in pharmaceutical compositions to a subject suffering from a condition. In some instances the pharmaceutical composition will affect the physiology of the animal, such as the immune system, inflammatory response, or other physiologic affect. In some embodiments, the subject is a mammal such as a human. A therapeutically-effective amount can vary widely depending on the severity of the disease, the age and relative health of the subject, the potency of the compounds used, and other factors.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically-acceptable salt form.

Methods for the preparation of peptides described herein comprising the compounds described herein include formulating the peptide described herein with one or more inert, pharmaceutically-acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically-acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds; Pharmaceutical Dosage Forms, Marcel Decker, New York, N. Y., 1980; and Pharmaceutical Dosage Forms and Drug Delivery Systems, Seventh Ed. (Lippincott Williams & Wilkins 1999), each of which is incorporated by reference in its entirety.

### Use of Peptide in Imaging and Surgical Methods

The present disclosure generally relates to peptides that home, target, migrate to, are retained by, accumulate in, and/or bind to, or are directed to specific regions, tissues, structures, or cells within the body and methods of using such peptides. These peptides have the ability to contact the cartilage, which makes them useful for a variety of applications. In particular, the peptides can have applications in site-specific modulation of biomolecules to which the peptides are directed to. End uses of such peptides can include, for example, imaging, research, therapeutics, theranostics, pharmaceuticals, chemotherapy, chelation therapy, targeted drug delivery, and radiotherapy. Some uses can include targeted drug delivery and imaging.

In some embodiments, the present disclosure provides a method for detecting a cancer, cancerous tissue, or tumor tissue, the method comprising the steps of contacting a tissue of interest with a peptide of the present disclosure, wherein the peptide is complexed, conjugated, or fused to a detectable agent and measuring the level of binding of the peptide, wherein an elevated level of binding, relative to normal tissue, is indicative that the tissue is a cancer, cancerous tissue or tumor tissue.

In some embodiments, the disclosure provides a method of imaging an organ or body region or region, tissue or structure of a subject, the method comprising administrating to the subject the peptide or a pharmaceutical composition disclosed herein and imaging the subject. In some embodiments such imaging is used to detect a condition associated with cartilage, or a function of the cartilage. In some cases the condition is an inflammation, a cancer, a degradation, a growth disturbance, genetic, a tear or an injury, or another suitable condition. In some cases the condition is a chondrodystrophy, a traumatic rupture or detachment, pain following surgery in regions of the body containing cartilage, costochondritis, herniation, polychondritis, arthritis, osteoarthritis, rheumatoid arthritis, ankylosing spondylitis (AS), Systemic Lupus Erythematosus (SLE or “Lupus”), Psoriatic Arthritis (PsA), gout, achondroplasia, or another suitable condition. In some case the condition is associated with a cancer or tumor of the cartilage. In some cases the condition is a type of chondroma or chondrosarcoma, whether metastatic or not, or another suitable condition. In some embodiments, such as those associated with cancers, the imaging may be associated with surgical removal of the diseased region, tissue, structure or cell of a subject.

Furthermore, the present disclosure provides methods for intraoperative imaging and resection of a diseased or inflamed tissue, cancer, cancerous tissue, or tumor tissue using a peptide of the present disclosure complexed, conjugated, or fused with a detectable agent. In some embodiments, the diseased or inflamed tissue, cancer, cancerous tissue, or tumor tissue is detectable by fluorescence imaging that allows for intraoperative visualization of the cancer, cancerous tissue, or tumor tissue using a peptide of the present disclosure. In some embodiments, the peptide of the present disclosure is complexed, conjugated, or fused to one or more detectable agents. In a further embodiment, the detectable agent comprises a fluorescent moiety coupled to the peptide. In another embodiment, the detectable agent comprises a radionuclide. In some embodiments, imaging is achieved during open surgery. In further embodiments, imaging is accomplished using endoscopy or other non-invasive surgical techniques.

### Treatment of Cartilage Disorders

The term “effective amount,” as used herein, can refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case can be determined using techniques, such as a dose escalation study.

The methods, compositions, and kits of this disclosure can comprise a method to prevent, treat, arrest, reverse, or ameliorate the symptoms of a condition. The treatment can comprise treating a subject (e.g., an individual, a domestic animal, a wild animal or a lab animal afflicted with a disease or condition) with a peptide of the disclosure. In treating a disease, the peptide can contact the cartilage of a subject. The subject can be a human. A subject can be a human; a non-human primate such as a chimpanzee, or other ape or monkey species; a farm animal such as a cattle, horse, sheep, goat, swine; a domestic animal such as a rabbit, dog, and cat; a laboratory animal including a rodent, such as a rat, mouse and guinea pig, or the like. A subject can be of any age. A subject can be, for example, an elderly adult, adult, adolescent, pre-adolescent, child, toddler, infant, or fetus in utero.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment can also include treating a human in a clinical trial. A treatment can comprise administering to a subject a pharmaceutical composition, such as one or more of the pharmaceutical compositions described throughout the disclosure. A treatment can comprise a once daily dosing. A treatment can comprise delivering a peptide of the disclosure to a subject, either parenterally, intravenously, subcutaneously, intramuscularly, by inhalation, dermally, intra-articular injection, orally, intrathecally, transdermally, intranasally, via a peritoneal route, or directly onto or into a joint, e.g., via topical, intra-articular injection route or injection route of application. A treatment can comprise administering a peptide-active agent complex to a subject, either parenterally, intravenously, subcutaneously, intramuscularly, by inhalation, dermally, intra-articular injection, orally, intrathecally, transdermally, intranasally, via a peritoneal route, or directly onto or into a joint or directly onto, near or into the cartilage, e.g., via topical, intra-articular injection route or injection route of application.

Types of cartilage diseases or conditions that can be treated with a peptide of the disclosure can include inflammation, pain management, anti-infective, pain relief, anti-cytokine, cancer, injury, degradation, genetic basis, remodeling, hyperplasia, surgical injury/trauma, or the like. Diseases or conditions of bone adjacent to cartilage can also be treated with a peptide of the disclosure. Examples of cartilage diseases or conditions that can be treated with a peptide of the disclosure include Costochondritis, Spinal disc herniation, Relapsing polychondritis, Injury to the articular cartilage, any manner of rheumatic disease (e.g., Rheumatoid Arthritis (RA), ankylosing spondylitis (AS), Systemic Lupus Erythematosus (SLE or “Lupus”), lupus arthritis, Psoriatic Arthritis (PsA), Osteoarthritis, Gout, and the like), Herniation, Achondroplasia, Benign or non-cancerous chondroma, Malignant or cancerous chondrosarcoma, Chondriodystrophies, Chondromalacia patella, Costochondritis, Halus rigidus, Hip labral tear, Osteochondritis dssecans, Osteochondrodysplasias, Torn meniscus, Pectus carinatum, Pectus excavatum, Chondropathy, Chondromalacia, Polychondritis, Relapsing Polychondritis, Slipped epiphysis, Osteochondritis Dissecans, Chondrodysplasia, Costochondritis, Perichondritis, Osteochondroma, Knee osteoarthritis, Finger osteoarthritis, Wrist osteoarthritis, Hip osteoarthritis, Spine osteoarthritis, Chondromalacia, Osteoarthritis Susceptibility, Ankle Osteoarthritis, Spondylosis, Secondary chondrosarcoma, Small and unstable nodules as seen in osteoarthritis, Osteochondroses, Primary chondrosarcoma, Cartilage disorders, scleroderma, collagen disorders, Chondrodysplasia, Tietze syndrome, Dermochondrocorneal dystrophy of Francois, Epiphyseal dysplasia multiple 1, Epiphyseal dysplasia multiple 2, Epiphyseal dysplasia multiple 3, Epiphyseal dysplasia multiple 4, Epiphyseal dysplasia multiple 5, Ossified Ear cartilages with Mental deficiency, Muscle Wasting and Bony Changes, Periosteal chondrosarcoma, Carpotarsal osteochondromatosis, Achondroplasia, Genochondromatosis II, Genochondromatosis, Chondrodysplasia—disorder of sex development, Chondroma, Chordoma, Atelosteogenesis, type 1, Atelosteogenesis Type III, Atelosteogenesis, type 2, Pyknoachondrogenesis, Osteoarthropathy of fingers familial, Dyschondrosteosis—nephritis, Coloboma of Alar-nasal cartilages with telecanthus, Alar cartilages hypoplasia—coloboma—telecanthus, Pierre Robin syndrome—fetal chondrodysplasia, Dysspondyloenchondromatosis, Achondroplasia regional—dysplasia abdominal muscle, Osteochondritis Dissecans, Familial Articular Chondrocalcinosis, Tracheobronchomalacia, Chondritis, Dyschondrosteosis, Jequier-Kozlowski-skeletal dysplasia, Chondrodystrophy, Cranio osteoarthropathy, Tietze's syndrome, Hip dysplasia—ecchondromata, Bessel-Hagen disease, Chondromatosis (benign), Enchondromatosis (benign), Chondrocalcinosis due to apatite crystal deposition, Meyenburg-Altherr-Uehlinger syndrome, Enchondromatosis-dwarfism-deafness, premature growth plate closure (e.g., due to dwarfism, injury, therapy such as retinoid therapy for adolescent acne, or ACL repair), Astley-Kendall syndrome, Synovial osteochondromatosis, Severe achondroplasia with developmental delay and acanthosis nigricans, Chondrocalcinosis, Stanescu syndrome, Familial osteochondritis dissecans, Achondrogenesis type 1A, Achondrogenesis type 2, Achondrogenesis, Langer-Saldino Type, Achondrogenesis type 1B, Achondrogenesis type 1A and 1B, Type II Achondrogenesis-Hypochondrogenesis, Achondrogenesis, Achondrogenesis type 3, Achondrogenesis type 4, Chondrocalcinosis 1, Chondrocalcinosis 2, Chondrocalcinosis familial articular, Diastrophic dysplasia, Fibrochondrogenesis, Hypochondroplasia, Keutel syndrome, Maffucci Syndrome, Osteoarthritis Susceptibility 6, Osteoarthritis Susceptibility 5, Osteoarthritis Susceptibility 4, Osteoarthritis Susceptibility 3, Osteoarthritis Susceptibility 2, Osteoarthritis Susceptibility 1, Pseudoachondroplasia, Cauliflower ear, Costochondritis, Growth plate fractures, Pectus excavatum, septic arthritis, gout, pseudogout (calcium pyrophosphate deposition disease or CPPD), gouty arthritis, bacterial, viral, or fungal infections in or near the joint, bursitis, tendinitis, arthropathies, or a joint disease condition. Examples of bone diseases or conditions that can be treated with a peptide of the disclosure include osteopenia, post-menopausal bone loss, bone maintenance, bone fracture, arthroplasty recovery, osteoporosis, bone loss due to metastatic cancer, fractures due to bone loss (e.g., hip fractures in patients with osteoporosis), pathological fracture, or atypical fracture.

In some embodiments, a peptide or peptide conjugate of this disclosure can be administered to a subject in order to target, an arthritic joint. In other embodiments, a peptide or peptide conjugate of this disclosure can be administered to a subject in order to treat an arthritic joint.

In some embodiments, the present disclosure provides a method for treating a cancer, the method comprising administering to a subject in need thereof an effective amount of a peptide of the present disclosure.

In some embodiments, the present disclosure provides a method for treating a cancer, the method comprising administering to a patient in need thereof an effective amount of a pharmaceutical composition comprising a peptide of the present disclosure and a pharmaceutically acceptable carrier.

In some embodiments, the peptides of the present disclosure can be used to treat chondrosarcoma. Chondrosarcoma is a cancer of cartilage producing cells and is often found in bones and joints. It falls within the family of bone and soft-tissue sarcomas. In certain embodiments, administration of a peptide or peptide conjugate of the present disclosure can be used to image and diagnose or target and treat a subject with chondrosarcoma. The administration of a peptide or peptide conjugate of the present disclosure can be used in combination with ablative radiotherapy or proton therapy to treat chondrosarcoma. The subject can be a human or an animal.

In some embodiments, a peptide or peptide conjugate of this disclosure can be used to treat Chordoma. In certain embodiments, administration of a peptide or peptide conjugate of the present disclosure can be used to image and diagnose or target and treat a subject with chordoma. The administration of a peptide or peptide conjugate of the present disclosure can be used in combination with a tyrosine kinase inhibitor, such as imatinib mesylate, and ablative radiotherapy or proton therapy to treat chordoma. The administration of a peptide or peptide conjugate of the present disclosure can be used in combination with an antivascular agent such as bevacizumab and an epidermal growth factor receptor inhibitor such as erlotinib to treat chordoma. The subject can be a human or an animal.

In some embodiments, the present disclosure provides a method for inhibiting invasive activity of cells, the method comprising administering an effective amount of a peptide of the present disclosure to a subject.

In some embodiments, the peptides of the present disclosure are complexed, conjugated, or fused to one or more therapeutic agents. In further embodiments, the therapeutic agent is a chemotherapeutic, anti-cancer drug, or anti-cancer agent selected from, but are not limited to: anti-inflammatories, such as for example a glucocorticoid, a corticosteroid, a protease inhibitor, such as for example collagenase inhibitor or a matrix metalloprotease inhibitor (i.e., MMP-13 inhibitor), an amino sugar, vitamin (e.g., Vitamin D), and antibiotics, antiviral, or antifungal, a statin, an immune modulator, radioisotopes, toxins, enzymes, sensitizing drugs, nucleic acids, including interfering RNAs, antibodies, anti-angiogenic agents, cisplatin, anti-metabolites, mitotic inhibitors, growth factor inhibitors, paclitaxel, temozolomide, topotecan, fluorouracil, vincristine, vinblastine, procarbazine, decarbazine, altretamine, methotrexate, mercaptopurine, thioguanine, fludarabine phosphate, cladribine, pentostatin, cytarabine, azacitidine, etoposide, teniposide, irinotecan, docetaxel, doxorubicin, daunorubicin, dactinomycin, idarubicin, plicamycin, mitomycin, bleomycin, tamoxifen, flutamide, leuprolide, goserelin, aminogluthimide, anastrozole, amsacrine, asparaginase, mitoxantrone, mitotane and amifostine, and their equivalents, as well as photo-ablation. Some of these active agents induce programmed cell death such as apoptosis in target cells and thereby improve symptoms or ameliorate disease. Apoptosis can be induced by many active agents, including, for example, chemotherapeutics, anti-inflammatories, corticosteroids, NSAIDS, tumor necrosis factor alpha (TNF-α) modulators, tumor necrosis factor receptor (TNFR) family modulators. In some embodiments, peptides of this disclosure can be used to target active agents to pathways of cell death or cell killing, such as caspases, apoptosis activators and inhibitors, XBP-1, Bcl-2, Bcl-Xl, Bcl-w, and other disclosed herein. In other embodiments, the therapeutic agent is any nonsteroidal anti-inflammatory drug (NSAID). The NSAID can be any heterocyclic acetic acid derivatives such as ketorolac, indomethacin, etodolac, or tolemetin, any propionic acid derivatives such as naproxen, any enolic acid derivatives, any anthranilic acid derivatives, any selective COX-2 inhibitors such as celecoxib, any sulfonanilides, any salicylates, aceclofenac, nabumetone, sulindac, diclofenac, or ibuprofen. In other embodiments, the therapeutic agent is any steroid, such as dexamethasone, budesonide, beclomethasone monopropionate, desciclesonide, triamcinolone, cortisone, prednisone, prednisolone, triamcinolone hexacetonide, or methylprednisolone. In other embodiments, the therapeutic agent is a pain reliever, such as acetaminophen, opioids, local anesthetics, anti-depressants, glutamate receptor antagonists, adenosine, or neuropeptides. In some embodiments, a treatment consists of administering a combination of any of the above therapeutic agents and a peptide conjugate, such as a treatment in which both a dexamethasone-peptide conjugate and an NSAID are administered to a patient. Peptides of the current disclosure that target the cartilage can be used to treat the diseases conditions as described herein, for example, any diseases or conditions including tears, injuries (i.e., sports injuries), genetic factors, degradation, thinning, inflammation, cancer or any other disease or condition of the cartilage or to target therapeutically-active substances to treat these diseases amongst others. In other cases, a peptide of the disclosure can be used to treat traumatic rupture, detachment, chostochondritis, spinal disc herniation, relapsing and non-relapsing polychondritis, injury to the articular cartilage, osteoarthritis, arthritis or achondroplasia. In some cases, the peptide or peptide-active agent can be used to target cancer in the cartilage, for example benign chondroma or malignant chondrosarcoma, by contacting the cartilage by diffusion into chondrocytes and then having antitumor function, targeted toxicity, inhibiting metastases, etc. As well, such peptide or peptide-active agent can be used to label, detect, or image such cartilage lesions, including tumors and metastases amongst other lesions, which may be removed through various surgical techniques or by targeting with peptide-active agents that induce programmed cell death or kill cells.

Venom or toxin derived peptide(s), peptides, modified peptides, labeled peptides, peptide-active agent conjugates and pharmaceutical compositions described herein can be administered for prophylactic and/or therapeutic treatments. In therapeutic applications, the composition can be administered to a subject already suffering from a disease or condition, in an amount sufficient to cure or at least partially arrest the symptoms of the disease or condition, or to cure, heal, improve, or ameliorate the condition. Such peptides described herein can also be administered to prevent (either in whole or in part), lessen a likelihood of developing, contracting, or worsening a condition. Amounts effective for this use can vary based on the severity and course of the disease or condition, previous therapy, the subject's health status, weight, response to the drugs, and the judgment of the treating physician. Venom or toxin derived peptide(s), peptides, modified peptides, labeled peptides, peptide-active agent conjugates and pharmaceutical compositions described herein can allow for targeted homing of the peptide and local delivery of any conjugate. For example, a peptide complexed, conjugated, or fused to a steroid allows for local delivery of the steroid, which is significantly more effective and less toxic than traditional systemic steroids. A peptide complexed, conjugated, or fused to an NSAID is another example. In this case, the peptide complexed, conjugated, or fused to an NSAID allows for local delivery of the NSAID, which allows for administration of a lower NSAID dose and is subsequently less toxic. By delivering an active agent to the joint, pain relief can be more rapid, may be more long lasting, and can be obtained with a lower systemic dose and off-site undesired effects than with systemic dosing without targeting.

Peptides of the current disclosure that target the cartilage can be used to treat or manage pain associated with a cartilage injury or disorder, or any other cartilage or joint condition as described herein. The peptides can be used either directly or as carriers of active drugs, peptides, or molecules. For example, since ion channels can be associated with pain and can be activated in disease states such as arthritis, peptides that interact with ion channels can be used directly to reduce pain. In another embodiment, the peptide is complexed, conjugated, or fused to an active agent with anti-inflammatory activity, in which the peptide acts as a carrier for the local delivery of the active agent to reduce pain. Peptides or peptide-active agent complexes may exert their effects via a variety of activities including anti-inflammatory, stopping cartilage destruction, stimulating cartilage regrowth, restoring cartilage, amongst other effects described herein.

In some embodiments, the peptides described herein provide a method of treating a cartilage condition of a subject, the method comprising administering to the subject a therapeutically-effective amount of a peptide comprising the sequence SEQ ID NO: 110, SEQ ID NO: 115, SEQ ID NO: 234, SEQ ID NO: 242, SEQ ID NO: 139, SEQ ID NO: 242, SEQ ID NO: 260, or fragment thereof. In some embodiments, the peptides described herein provide a method of treating a cartilage condition of a subject, the method comprising administering to the subject a therapeutically-effective amount of a peptide comprising the sequence SEQ ID NO: 52-SEQ ID NO: 66, SEQ ID NO: 241-SEQ ID NO: 248, SEQ ID NO: 134-SEQ ID NO: 148, SEQ ID NO: 249-SEQ ID NO: 256, SEQ ID NO: 111-SEQ ID NO: 126, SEQ ID NO: 233-SEQ ID NO: 240, or fragment thereof. In some embodiments, the peptides described herein provide a method of treating a cartilage condition of a subject, the method comprising administering to the subject a peptide of any one of SEQ ID NO: 21-SEQ ID NO: 45, SEQ ID NO: 47-SEQ ID NO: 66, SEQ ID NO: 87-SEQ ID NO: 89, SEQ ID NO: 106-SEQ ID NO: 126, SEQ ID NO: 129-SEQ ID NO: 148, SEQ ID NO: 198, SEQ ID NO: 200-SEQ ID NO: 215, SEQ ID NO: 219-SEQ ID NO: 263 or fragment thereof. Optionally, the peptide or fragment thereof may be administered itself, or may be administered as a peptide-active agent complex.

### Treatment of Kidney Disorders

In some embodiments, peptides of this disclosure that home, target, are directed to, migrate to, are retained by, accumulate in, or bind to specific regions, tissues, structures or cells of the kidneys can be used to treat a kidney disorder. In other embodiments, peptides are used in peptide conjugates of the present disclosure to deliver an active agent for treatment of a kidney disorder.

In some embodiments, the peptides and peptide-conjugates of the present disclosure are used to treat a condition of the kidney, or a region, tissue, structure, or cell thereof. In certain embodiments, the condition is associated with kidney, or a function of a subject's kidneys. The present disclosure encompasses various acute and chronic renal diseases, including glomerular, tubule-interstitial, and microvascular diseases. Examples of conditions applicable to the present disclosure include but are not limited to: hypertensive kidney damage, acute kidney diseases and disorders (AKD), acute kidney injury (AKI) due to ischemia-reperfusion injury, drug treatment such as chemotherapy, cardiovascular surgery, surgery, medical interventions or treatment, radiocontrast nephropathy, or induced by cisplatin or carboplatin, which can be treated prophylactically, established AKI including ischemic renal injury, endotoxemia-induced AKI, endotoxemia/sepsis syndrome, or established nephrotoxic AKI (e.g., rhabdomyolysis, radiocontrast nephropathy, cisplatin/carboplatin AKI, aminoglycoside nephrotoxicity), end stage renal disease, acute and rapidly progressive glomerulonephritis, acute presentations of nephrotic syndrome, acute pyelonephritis, acute renal failure, chronic glomerulonephritis, chronic heart failure, chronic interstitial nephritis, graft versus host disease after renal transplant, chronic kidney disease (CKD) such as diabetic nephropathy, hypertensive nephrosclerosis, idiopathic chronic glomerulonephritis (e.g., focal glomerular sclerosis, membranous nephropathy, membranoproliferative glomerulonephritis, minimal change disease transition to chronic disease, anti-GBM disease, rapidly progressive cresentic glomerulonephritis, IgA nephropathy), secondary chronic glomerulonephritis (e.g., systemic lupus, polyarteritis nodosa, scleroderma, amyloidosis, endocarditis), hereditary nephropathy (e.g., polycystic kidney disease, Alport's syndrome), interstitial nephritis induced by drugs (e.g., Chinese herbs, NSAIDs), multiple myeloma or sarcoid, or renal transplantation such as donor kidney prophylaxis (treatment of donor kidney prior to transplantation), treatment post transplantation to treat delayed graft function, acute rejection, or chronic rejection, chronic liver disease, chronic pyelonephritis, diabetes, diabetic kidney disease, fibrosis, focal segmental glomerulosclerosis, Goodpasture's disease, hypertensive nephrosclerosis, IgG4-related renal disease, interstitial inflammation, lupus nephritis, nephritic syndrome, partial obstruction of the urinary tract, polycystic kidney disease, progressive renal disease, renal cell carcinoma, renal fibrosis, and vasculitis. For example, in certain embodiments, the peptides and peptide-conjugates of the present disclosure are used to reduce acute kidney injury in order to prevent it from progressing to chronic kidney disease.

Alternatively or in combination, in some embodiments, the peptide and peptide-conjugates of the present disclosure are used to elicit a protective response such as ischemic preconditioning and/or acquired cytoresistance in a kidney of the subject. In some embodiments, ischemic preconditioning and/or acquired cytoresistance is induced by administering an agent (e.g., a peptide or peptide-conjugate of the present disclosure) that upregulates the expression of protective stress proteins, such as antioxidants, anti-inflammatory proteins, or protease inhibitors. In certain embodiments, the induced response protects the kidney by preserving kidney function in whole or in part and/or by reducing injury to renal tissues and cells, e.g., relative to the situation where no protective response is induced. The peptides and peptide-conjugates of the present disclosure can provide certain benefits compared to other agents for inducing ischemic preconditioning and/or acquired cytoresistance, such as a well-defined chemical structure and avoidance of low pH precipitation.

In some embodiments, the protective response is induced in order to protect the kidney or tissues or cells thereof from an injury or insult that is predicted to occur (e.g., associated with a planned event such as a medical procedure, is likely to occur due to a condition in the subject) or has already occurred. In certain embodiments, the induced response prevents or reduces the extent of damage to the kidney or tissues or cells thereof caused by the injury or insult. For instance, in certain embodiments, the peptides and peptide-conjugates induce acquired cytoresistance by activating protective pathways and/or upregulating expression of protective stress proteins. Optionally, the peptides and peptide-conjugates are capable of inducing such protective responses while causing minimal or no injury to the kidney.

In various embodiments, the injury or insult is associated with one or more of: surgery, radiocontrast imaging, cardiopulmonary bypass, balloon angioplasty, induced cardiac or cerebral ischemic-reperfusion injury, organ transplantation, sepsis, shock, low blood pressure, high blood pressure, kidney hypoperfusion, chemotherapy, drug administration, nephrotoxic drug administration, blunt force trauma, puncture, poison, or smoking. For instance, in certain embodiments, the injury or insult is associated with a medical procedure that has been or will be performed on the subject, such as one or more of: surgery, radiocontrast imaging, cardiopulmonary bypass, balloon angioplasty, induced cardiac or cerebral ischemic-reperfusion injury, organ transplantation, chemotherapy, drug administration, or nephrotoxic drug administration.

In some embodiments, the peptide itself exhibits a renal therapeutic effect. For example, in certain embodiments, the cystine-dense peptide interacts with a renal ion channel, inhibits a protease, has antimicrobial activity, has anticancer activity, has anti-inflammatory activity, induces ischemic preconditioning or acquired cytoresistance, or produces a protective or therapeutic effect on a kidney of the subject, or a combination thereof. Optionally, the renal therapeutic effect exhibited by the peptide is a renal protective effect or renal prophylactic effect (e.g., ischemic preconditioning or acquired cytoresistance) that protects the kidney or a tissue or cell thereof from an upcoming injury or insult. Peptides or peptide-active agent complexes may exert their effects via a variety of activities including anti-inflammatory, stopping kidney destruction, stimulating kidney regrowth, restoring kidney function, amongst other effects described herein.

For example, in certain embodiments, a peptide of the present disclosure activates protective pathways and/or upregulates expression of protective stress proteins in the kidney or tissues or cells thereof. As another example, in certain embodiments, a peptide of the present disclosure accesses and suppresses intracellular injury pathways. In yet another example, in certain embodiments, a peptide of the present disclosure inhibits interstitial inflammation and prevents renal fibrosis. As a further example, in certain embodiments, a peptide of the present disclosure is administered prior to or currently with the administration of a nephrotoxic agent (e.g., aminoglycoside antibiotics such as gentamicin and minocycline, chemotherapeutics such as cisplatin, immunoglobulins or fragments thereof, mannitol, NSAIDs such as ketorolac or ibuprofen, cyclosporin, cyclophosphamide, radiocontrast dyes) in order to minimize its damaging effects, e.g., by blocking megalin-cubulin binding sites so that the nephrotoxic agent passes through the kidneys.

In some embodiments, the present disclosure provides that any peptide of the disclosure including SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 can as a peptide conjugate with an active agent for treatment of a kidney disorder can be complexed, conjugated, or fused to an active agent and administered to a subject in need thereof to treat a kidney disorder.

In some embodiments, homing of a peptide of this disclosure to cartilage or the kidneys can be assessed in an animal model such as those described in Alves et al. (Clin Rev Allergy Immunol. 2016 August; 51(1):27-47. doi: 10.1007/s12016-015-8522-7), Kuyinu et al. (J Orthop Surg Res. 2016 Feb. 2; 11:19. doi: 10.1186/s13018-016-0346-5), Li et al. (Exp Biol Med (Maywood). 2015 August; 240(8):1029-38. doi: 10.1177/1535370215594583), and Mullins et al. (Dis Model Mech. 2016 Dec. 1; 9(12):1419-1433), all of which are incorporated herein by reference.

Multiple peptides described herein can be administered in any order or simultaneously. In some cases, multiple functional fragments of peptides derived from toxins or venom can be administered in any order or simultaneously. If simultaneously, the multiple peptides described herein can be provided in a single, unified form, such as an intravenous injection, or in multiple forms, such as subsequent intravenous dosages.

Peptides can be packaged as a kit. In some embodiments, a kit includes written instructions on the use or administration of the peptides.

### EXAMPLES

The following examples are included to further describe some embodiments of the present disclosure, and should not be used to limit the scope of the disclosure.

### Example 1

### Manufacture of Peptides

The peptide sequence was reverse-translated into DNA, synthesized, and cloned in-frame with siderocalin using standard molecular biology techniques. (M. R. Green, Joseph Sambrook. Molecular Cloning. 2012 Cold Spring Harbor Press.). The resulting construct was packaged into a lentivirus, transfected into HEK293 cells, expanded, isolated by immobilized metal affinity chromatography (IMAC), cleaved with tobacco etch virus protease, and purified to homogeneity by reverse-phase chromatography. Following purification, each peptide was lyophilized and stored frozen.

### Example 2

### Radiolabeling of Peptide

This example describes radiolabeling of peptides with standard techniques. See J Biol Chem. 254(11):4359-65 (1979). The sequences were engineered to have the amino acids, “G” and “S” at the N terminus. See Methods in Enzymology V91:1983 p. 570 and Journal of Biological Chemistry 254(11):1979 p. 4359. An excess of formaldehyde was used to ensure complete methylation (dimethylation of every free amine). The labeled peptides were isolated via solid-phase extraction on Strata-X columns (Phenomenex 8B-S100-AAK), rinsed with water with 5% methanol, and recovered in methanol with 2% formic acid. Solvent was subsequently removed in a blowdown evaporator with gentle heat and a stream of nitrogen gas.

### Example 3

### Peptide Detectable Agent Complexes

This example describes the dye labeling of peptides. A peptide of the disclosure is expressed recombinantly or chemically synthesized, and then the N-terminus of the peptide is complexed, conjugated, or fused to a detectable agent via an NHS ester using DCC or EDC to produce a peptide-detectable agent conjugate. The detectable agent is the fluorophore dye is a cyanine dye, such as Cy5.5 or an Alexa fluorophore, such as Alexa647.

The peptide detectable agent conjugates are administered to a subject. The subject can be a human or a non-human animal. After administration, the peptide detectable agent conjugates home to cartilage. The subject, or a biopsy from the subject, can be imaged to visualize localization of the peptide detectable agent conjugates to cartilage. In some aspects, visualization of the peptide detectable agent conjugates in cartilage after administration results in diagnosis of arthritis, cartilage damage, or any cartilage disorder.

### Example 4

### Dosing of Peptide with Kidney Ligation

This example describes a dosing scheme for administering peptides to mice in conjunction with kidney ligation. Different dosages of the peptides of SEQ ID NO:149 and SEQ ID NO:150 were administered to Female Harlan athymic nude mice, weighing 20 g-25 g, via tail vein injection (n=2 mice per peptide). The experiment was done in duplicates. The kidneys were ligated to prevent renal filtration of the peptides. Each peptide was radiolabeled by methylating lysines and the N-terminus, so the actual binding agent may contain methyl or dimethyl lysine(s) and a methylated or dimethylated amino terminus.

A target dosage of 50-100 nmol of each peptide carrying 10-25 uCi of ¹⁴C was administered to Female Harlan athymic nude mice while anesthetized. Each peptide was allowed to freely circulate within the animal before the animals were euthanized and sectioned.

This method is applied to any of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

### Example 5

### Peptide Homing with Kidney Ligation

This example illustrates peptide homing to cartilage of mice with kidneys that were ligated prior to peptide administration. At the end of the dosing period in EXAMPLE 4, mice were frozen in a hexane/dry ice bath and then frozen in a block of carboxymethylcellulose. Whole animal sagittal slices were prepared that resulted in thin frozen sections being available for imaging. Thin, frozen sections of animal including imaging of tissues such as brain, tumor, liver, kidney, lung, heart, spleen, pancreas, muscle, adipose, gall bladder, upper gastrointestinal tract, lower gastrointestinal tract, bone, bone marrow, reproductive track, eye, cartilage, stomach, skin, spinal cord, bladder, salivary gland, and other types of tissues were obtained with a microtome, allowed to desiccate in a freezer, and exposed to phosphoimager plates for about ten days.

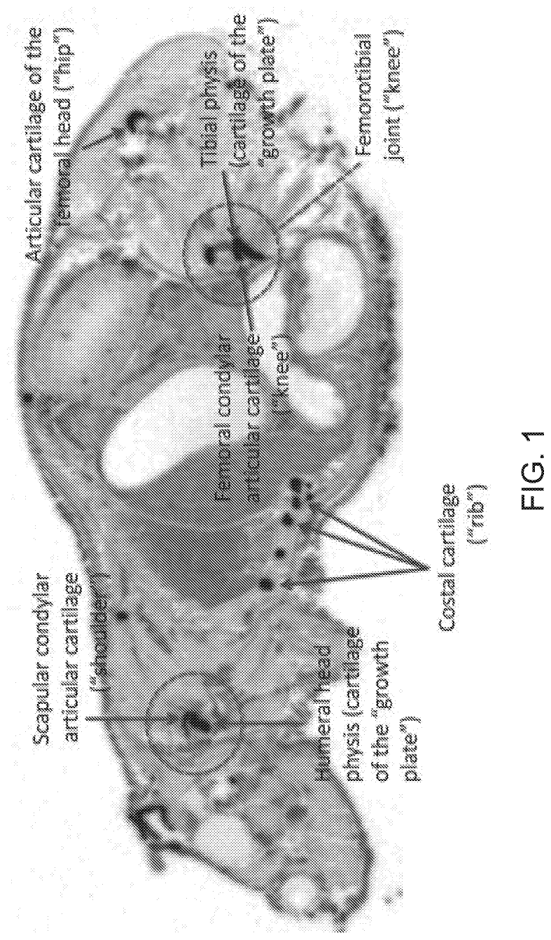

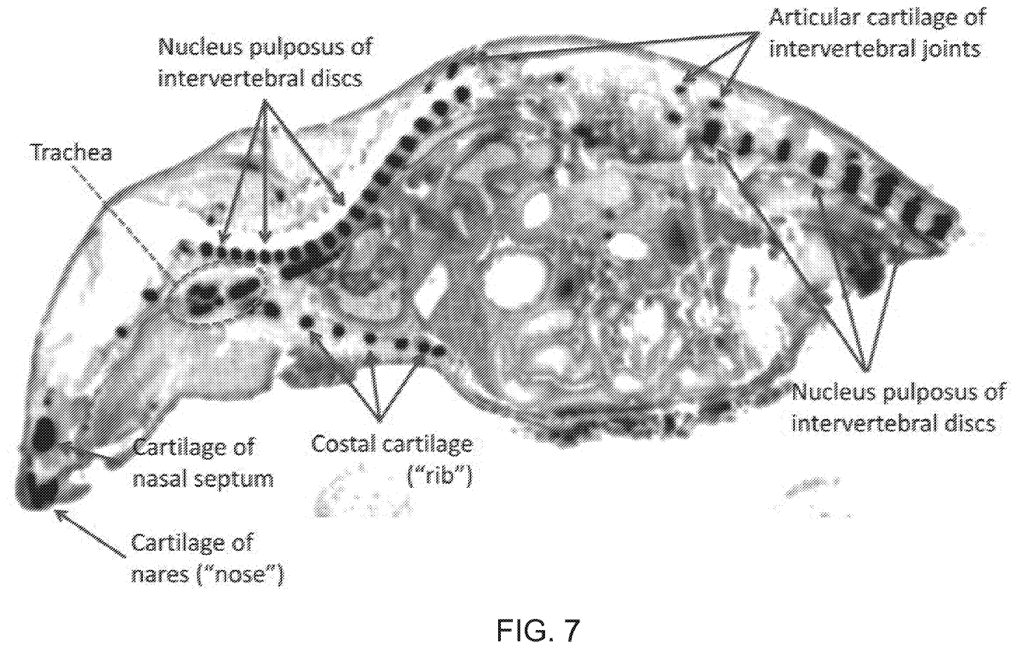

These plates were developed, and the signal (densitometry) from each organ was normalized to the signal found in the heart blood of each animal. A signal in tissue darker than the signal expected from blood in that tissue indicates peptide accumulation in a region, tissue, structure or cell. For instance, the cartilage is avascular and contains minute amounts of blood. A ratio of at least 170% signal in the cartilage versus heart ventricle was chosen as a reference level for significant targeting to cartilage, which also correlated with clear accumulation in cartilaginous tissues in the images of the slices. FIG. 1 identifies the locations of the SEQ ID NO: 150 peptide distribution in joint and other cartilage. FIG. 7 identifies the locations of the SEQ ID NO: 27 peptide distribution in nasal, spinal, tracheal, and other cartilage, including to hyaline cartilage such as articular cartilage and physeal cartilage, as well as fibrocartilage.

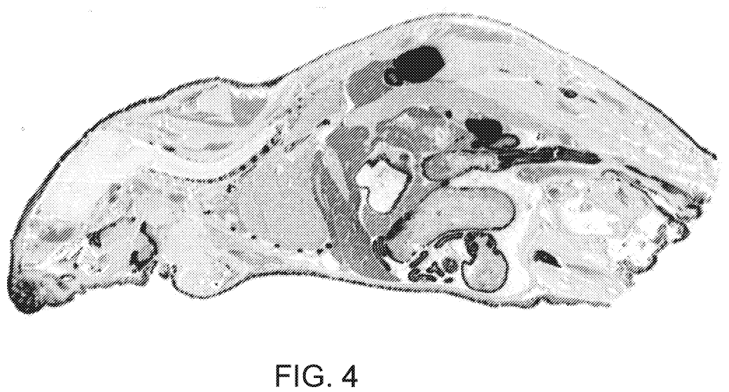

Additionally, the peptide can be retained in cartilage for hours after treatment. The SEQ ID NO: 150 peptide was radiolabeled as in EXAMPLE 4 and 100 nmol of peptide was injected into a mouse with intact kidneys. FIG. 4 illustrates the retention of and the tissue distribution in the cartilage of a peptide of SEQ ID NO: 150, 24 hours after administration.

This method is applied to any of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

### Example 6

### Dosing of Peptide without Kidney Ligation

This example describes a dosing scheme for administering peptides to mice without kidney ligation. The peptide administered had the sequence of SEQ ID NO: 150. The peptide was radiolabeled by methylating lysines and the N-terminus, so the actual binding agent may contain methyl or dimethyl lysine(s) and a methylated or dimethylated amino terminus.

A target dosage of 100 nmol of each peptide carrying 10-25 μCi of ¹⁴C was administered to Female Harlan athymic nude mice by a tail vein injection. Each peptide was allowed to freely circulate within the animal for either 4 hours or 24 hours before the animals were euthanized and sectioned.

This method is applied to any of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

### Example 7

### Peptide Homing with Intact Kidneys

This example illustrates peptide homing to cartilage in animals with intact kidneys. At the end of the 4 hour or 24 hour dosing periods in EXAMPLE 6, mice were frozen in a hexane/dry ice bath and then frozen in a block of carboxymethylcellulose. Whole animal sagittal slices were prepared that resulted in thin frozen sections being available for imaging. Thin, frozen sections of animal including imaging of tissues such as brain, tumor, liver, kidney, lung, heart, spleen, pancreas, muscle, adipose, gall bladder, upper gastrointestinal track, lower gastrointestinal track, bone, bone marrow, reproductive track, eye, cartilage, stomach, skin, spinal cord, bladder, salivary gland, and other types of tissues were obtained with a microtome, allowed to desiccate in a freezer, and exposed to phosphoimager plates for about ten days.

These plates were developed. A signal in tissue darker than the signal expected from blood in that tissue indicates peptide accumulation in a region, tissue, structure or cell. For instance, the cartilage is avascular and contains minute amounts of blood. High signal in the kidneys indicates presence and accumulation of the peptide in the kidneys. FIG. 1 identifies the locations of the SEQ ID NO: 150 peptide distribution in joint and other cartilage as well as kidneys.

This method is applied to any of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

### Example 8

### Peptide Homing with Therapeutic Agents

This example describes certain exemplary therapeutic agents that are complexed, conjugated, or fused to a peptide. A peptide of the disclosure is expressed recombinantly or chemically synthesized and then is complexed, conjugated, or fused to an exemplary drug, such as paclitaxel or triamcinolone acetonide or budesonide using techniques known in the art, such as those described in Bioconjugate Techniques by Greg Hermanson (Elsevier Inc., 3rd edition, 2013). One or more drugs are complexed, conjugated, or fused per peptide, or an average of less than one drug is complexed, conjugated, or fused per peptide.

Coupling of these drugs to a peptide of any of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 targets the drug to the cartilage of the subject. One or more drug-peptide conjugates are administered to a human or animal.

### Example 9

### Peptide Homing to an Arthritic Joint

This example illustrates peptide homing to cartilage in humans or animals with arthritis. A peptide of the present disclosure is expressed recombinantly or chemically synthesized and is used directly, after radiolabeling, or after conjugation to a fluorophore or therapeutic compound. A peptide is selected from any one of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. The peptide or peptide conjugate is administered to a human or animal subcutaneously, intravenously, or orally, or is injected directly into a joint intraarticularly. The peptide or peptide conjugate homes to cartilage.

### Example 10

### Peptide Homing to Cartilage in Non-Human Animals

This example illustrates a peptide or peptide conjugate of this disclosure homing to cartilage in non-human animals. Non-human animals include but are not limited to guinea pigs, rabbits, dog, cats, horses, rats, mice, cows, pigs, non-human primates, and other non-human animals. A peptide of the present disclosure is recombinantly expressed or chemically synthesized and are used directly, after radiolabeling, or after conjugation to a fluorophore or therapeutic compound. The peptide is selected from any one of the peptides SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. The resulting peptide or peptide conjugate is administered to a non-human animal subcutaneously, intravenously, or orally, or is injected directly into a joint intra-articularly. Biodistribution is assessed by LC/MS, autoradiography, positron emission tomography (PET), or fluorescence imaging. A peptide or peptide conjugate is homed to cartilage in non-human animals.

### Example 11

### Whole Body Fluorescence and Isolated Limb Fluorescence of Homing Peptides

This example illustrates whole body fluorescence and isolated limb fluorescence of peptide homers of this disclosure. Any peptide of the present disclosure is chemically complexed, conjugated, or fused to one molecule of a near infrared fluorophore, at the N-terminus of the peptide via an active NHS ester on the dye. A dose of 10 nmol of each peptide complexed, conjugated, or fused to a fluorophore is administered to Female Harlan athymic nude mice, weighing 20-25 g, and is administered via tail vein injection. Each experiment is done at least in duplicate (n=2 mice per group). The peptide fluorophore conjugate is allowed to freely circulate for the described time period before the mice were euthanized at various time points. Mice are evaluated for peptide distribution of the peptide fluorescence in whole body imaging and in isolated hind limb imaging.

For Whole body fluorescence (WBF), at the end of the dosing period, mice are frozen in a hexane/dry ice bath and then embedded in a frozen block of carboxymethylcellulose. Whole animal sagittal slices are prepared that resulted in thin frozen sections for imaging. Thin frozen sections are obtained using a microtome and allowed visualization of tissues. Sections are allowed to dessicate in a freezer prior to imaging. WBF is performed on fluorescent sections, which are scanned on a Li-Cor Odyssey scanner at a setting of 169 μm resolution, medium quality, 700 channel, L-2.0 intensity.

For isolated hind limb fluorescence studies, mice are euthanized by CO₂ asphyxiation at the end of the dosing period. The right hind limb is removed at the hip joint and imaged on a Sepctrum IVIS imager (ex/em: 675 nm. 720 nm) with a 1 second exposure length and a focal height of 0.5 cm. Limbs are imaged with skin removed and with muscle removed.

### Example 12

### Whole Body Autoradiography of Homing Peptides

This example illustrates whole body autoradiography of peptide homers of this disclosure. Peptides are radiolabeled by methylating lysines at the N-terminus as described in EXAMPLE 2. As such, the peptide may contain methyl or dimethyl lysines and a methylated or dimethlyated amino terminus. A dose of 100 nmol radiolabeled peptide is administered via tail vein injection in Female Harlan athymic nude mice, weighing 20-25 g. The experiment is done in at least duplicate (n=2 animals per group). In some animals, kidneys are ligated to prevent renal filtration of the radiolabled peptides and extend plasma half-life. Each radiolabeled peptide is allowed to freely circulate within the animal for the described time period before the animals were euthanized and sectioned.

Whole body autoradiography (WBA) sagittal sectioning is performed as follows. At the end of the dosing period, mice are frozen in a hexane/dry ice bath and then embedded in a frozen block of carboxymethylcellulose. Whole animal sagittal slices are prepared that resulted in thin frozen sections for imaging. Thin frozen sections are obtained using a microtome and allowed visualization of tissues such as brain, tumor, liver, kidney, lung, heart, spleen, pancreas, muscle, adipose, gall bladder, upper gastrointestinal tract, lower gastrointestinal tract, bone, bone marrow, reproductive tract, eye, cartilage, stomach, skin, spinal cord, bladder, salivary gland, and more. Sections are allowed to dessicate in a freezer prior to imaging.

For the autoradiography imaging, tape mounted thin sections are freeze dried and radioactive samples were exposed to phophoimager plates for 7 days. These plates are developed and the signal (densitometry) from each organ was normalized to the signal found in the cardiac blood of each animal. A signal in tissue darker than the signal expected from blood in that tissue indicates accumulation in a region, tissue, structure, or cell.

### Example 13

### Peptide Localization in Chondrocytes

This example illustrates binding of peptides of this disclosure to chondrocytes within cartilage in animals with intact kidneys. In one embodiment, animals are dosed and are processed as described in EXAMPLE 11 and EXAMPLE 12. At the end of the dosing period, animals are euthanized and cartilage is optionally removed for use in staining and imaging procedures. Whole animal sagittal slices are prepared that result in thin frozen sections being available for staining and imaging. One or more of the following cartilage components are identified in thin frozen sections or live cartilage explants using standard staining techniques: collagen fibrils, glycosaminoglycans, or chondrocytes. A peptide of this disclosure is found to localize to chondrocytes in cartilage, localized intracellularly or extracellularly bound or both. Localization is visualized and confirmed by microscopy.

In another embodiment, peptides or peptide-drug conjugates of this disclosure are administered in humans and are localized on or in chondrocytes in cartilage.

### Example 14

### Peptide Localization in Cartilage Extracellular Matrix

This example illustrates localization of peptides of this disclosure in cartilage extracellular matrix. In one embodiment, animals are dosed and are processed as described in EXAMPLE 11 and EXAMPLE 12 in animals with intact kidneys. At the end of the dosing period, animals are euthanized and cartilage is optionally removed for use in staining and imaging procedures. Whole animal sagittal slices are prepared that result in thin frozen sections being available for staining and imaging. Thin frozen sections or live cartilage explants are acquired, stained, and visualized as described in EXAMPLE 13. A peptide of the present disclosure is found to localize to the extracellular matrix in cartilage. The peptide may be bound to one or more components of the extracellular matrix, such as proteoglycans, glycosaminoglycans, aggrecan, decorin, or collagen. Localization is visualized and confirmed by microscopy.

In another embodiment, peptides or peptide-drug conjugates of this disclosure are administered in humans and are localized in cartilage extracellular matrix.

### Example 15

### Peptide Binding to Cartilage Explants

This example illustrates a peptide or peptide conjugation of this disclosure homing, targeting, being directed to, migrating to, being retained by, accumulating in, or binding to human and animal cartilage explants in culture. A peptide is selected from any one of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Peptides are recombinantly expressed or chemically synthesized and are used directly, after radiolabeling, or after conjugation to a fluorophore or therapeutic compound. A peptide of peptide conjugate of this disclosure is incubated with cartilage explants derived from humans or animals. Peptides of peptide conjugate are found to bind to cartilage explants. The interaction with cartilage is confirmed using various methods that include but are not limited to liquid scintillation counting, confocal microscopy, immunohistochemistry, HPLC, or LC/MS. The peptide shows a higher level of signal than a control peptide that is administered that is not a cartilage binding peptide.

### Example 16

### Effects of Peptide on Ion Channels

This example describes the interaction between peptides of the present disclosure and ion channels. Ion channels can be associated with pain and can be activated in disease states such as arthritis. A peptide of the disclosure is expressed and administered in a pharmaceutical composition to a patient to treat a joint condition or disease associated with an ion channel and treatable by binding, blocking, or interacting with the ion channel. Ion channels, such as Nav 1.7, are inhibited by peptides of the present disclosure. A given peptide is expressed recombinantly or chemically synthesized, wherein the peptide selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Following expression or synthesis, the peptide is used directly or complexed, conjugated, or fused to a therapeutic compound, such as those described herein. A peptide of the present disclosure selectively interacts with ion channels, or is mutated in order to interact with ion channels. For example, a peptide of this disclosure is bound to Nav 1.7 or Nav 1.7 is blocked by a peptide of this disclosure. When the peptide is administered to a human subject, Nav 1.7 signaling is reduced in the tissues in proximity to the joints, and pain relief is thereby provided.

### Example 17

### Peptide-Fc Protein Fusions

This example illustrates making and using peptide-Fc protein fusions. A peptide of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205) was recombinantly expressed with the sequence for the human IgG1 Fc protein in HEK293 cells to yield a sequence of SEQ ID NO:

SEQ ID NO: 216
(METDTLLLWVLLLWVPGSTGGSGVPINVRCRGSRDCLDPCRRAGMRFG
RCINSRCHCTPGGSGGSDKTHTCPPCPAPELLGGPSVFLFPPKPKDTLM
ISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGK)

The sequence of any peptide of this disclosure is expressed as a fusion protein with either murine or human Fc by adding a secretion signal sequence to the N-terminus and an Fc sequence to the C-terminus. This creates a bivalent molecule with improved secretion properties. The larger peptide-Fc fusion is expressed in different mammalian or insect cell lines and is useful as a research reagent and a therapeutic.

Fc fusion to a peptide of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205) to yield a sequence of SEQ ID NO: 216 extends half-life and improves biodistribution of the peptide to cartilage. Any peptide of this disclosure is co-expressed with Fc protein to yield Fc-fusion peptides with longer half-life and improved homing to cartilage. In SEQ ID NO: 216, the secretion signal sequence METDTLLLWVLLLWVPGSTG (SEQ ID NO: 217) is followed by the peptide of SEQ ID NO: 149, and is followed by the sequence for Fc protein. Cleaving can be imprecise, resulting in cleavage at position 20 or position 21 of SEQ ID NO: 216.

### Example 18

### Peptide Conjugate Hydrolysis

This example describes preparation of peptide conjugates having tunable hydrolysis rates. The peptide-drug conjugates described below are synthesized with the modification that instead of using succinic anhydride, other molecules are used to provide steric hindrance to hydrolysis or an altered local environment at the carbon adjacent to the final hydrolyzable ester. In one exemplary conjugate, the peptide-drug conjugate is synthesized with tetramethyl succinic anhydride to generate hindered esters, which causes a decreased rate of hydrolysis. In another exemplary conjugate, one methyl group is present at the adjacent carbon. In another exemplary conjugate, two methyl groups are present at the adjacent carbon. In another exemplary conjugate, one ethyl group is present at the adjacent carbon. In another exemplary conjugate, two ethyl groups are present at the adjacent carbon. In another exemplary conjugate, the carbon linker length is increased such as by using glutaric anhydride instead of succinic anhydride, increasing the local hydrophobicity and lowering the hydrolysis rate. In another exemplary conjugate, a hydroxyl group is located on the adjacent carbon, increasing the local hydrophilicity and increasing the hydrolysis rate. The rate of hydrolysis in these exemplary conjugates is therefore adjusted, preventing premature cleavage and ensuring that the majority of peptide-dexamethasone conjugates accumulate in cartilage prior to release of the drug by hydrolysis but that the dexamethasone is also released in the cartilage in a timely manner.

The resulting peptide conjugates are administered to a human or animal subcutaneously, intravenously, orally, or injected directly into a joint to treat disease.

### Example 19

### Peptide Complexes with Stable Linkers

This example describes preparation of peptide conjugates with stable linkers. A peptide of the disclosure is expressed recombinantly or is chemically synthesized. The peptide is complexed, conjugated, or fused to a detectable agent or an active agent via a stable linker, such as an amide linkage or a carbamate linkage. The peptide is complexed, conjugated, or fused to a detectable agent or an active agent via a stable linker, such as an amide bond using standard 1-ethyl-3-(3-dimethylaminopropyl)carbodiimide (EDC) or dicyclohexylcarbodiimide (DCC) based chemistry or thionyl chloride or phosphorous chloride-based bioconjugation chemistries.

A peptide and drug complexed, conjugated, or fused via a linker are described with the formula Peptide-A-B-C-Drug, wherein the linker is A-B-C. A can be a stable amide link that is formed by reacting with an amine on the peptide with a linker containing a tetrafluorophenyl (TFP) ester or an NHS ester. A can also be a stable carbamate linker that is formed by reacting with an amine on the peptide imidazole carbamate active intermediate formed by the reaction of CDI with a hydroxyl on the linker. A can also be a stable secondary amine linkage that is formed by reductive alkylation of the amine on the peptide with an aldehyde or ketone group on the linker. A can also be a stable thioether linker formed using a maleimide or bromoacetamide in the linker with a thiol in the peptide, a triazole linker, a stable oxime linker, or an oxacarboline linker. B is (—CH2-)ₓ-, a short PEG (—CH₂CH₂O-)x (x is 0-20). Alternatively, spacers within the linker is optional and can be included or not at all. C is an amide bond formed with an amine or a carboxylic acid on the drug, a thioether formed between a maleimide on the linker and a sulfhydroyl on the drug, a secondary or tertiary amine, a carbamate, or other stable bonds. Any linker chemistry described in “Current ADC Linker Chemistry,” Jain et al., Pharm Res, 2015 DOI 10.1007/s11095-015-1657-7 can be used.

The resulting peptide conjugates are administered to a human or animal subcutaneously, intravenously, orally, or injected directly into a joint to treat disease. The peptide is not specifically cleaved from the detectable agent or active agent via a targeted mechanism. The peptide can be degraded by mechanisms such as catabolism, releasing a drug that is modified or not modified form its native form (Singh, Luisi, and Pak, Pharm Res 32:3541-3571 (2015)). The peptide drug conjugate exerts its pharmacological activity while still intact, or while partially or fully degraded, metabolized, or catabolized.

### Example 20

### Peptide Complexes with Cleavable Linkers

This example describes preparation of peptide conjugates having cleavable linkers. A peptide of the disclosure is expressed recombinantly or chemically synthesized. A peptide and drug are complexed, conjugated, or fused via a linker and is described with the formula Peptide-A-B-C-Drug, wherein the linker is A-B-C. A is a stable amide link such as that formed by reacting an amine on the peptide with a linker containing a tetrafluorophenyl (TFP) ester or an NHS ester. A can also be a stable carbamate linker such as that formed by reacting an amine on the peptide with an imidazole carbamate active intermediate formed by reaction of CDI with a hydroxyl on the linker. A can also be a stable secondary amine linkage such as that formed by reductive alkylation of the amine on the peptide with an aldehyde or ketone group on the linker. A can also be a stable thioether linker formed using a maleimide or bromoacetamide in the linker with a thiol in the peptide, a triazole linker, a stable oxime linker, or a oxacarboline linker. B is (—CH2-)ₓ- or a short PEG (—CH₂CH₂O—)ₓ (x is 0-20) or other spacers or no spacer. C is an ester bond to the hydroxyl or carboxylic acid on the drug, or a carbonate, hydrazone, or acylhydrazone, designed for hydrolytic cleavage. The hydrolytic rate of cleavage is varied by varying the local environment around the ester, including carbon length (—CH2-)x, steric hindrance (including adjacent side groups such as methyl, ethyl, cyclic), hydrophilicity or hydrophobicity. Hydrolysis rate is affected by local pH, such as lower pH in certain compartments of the body or of the cell such as endosomes and lysosomes or diseased tissues. C is a pH sensitive group such as a hydrazone or oxime linkage. Alternatively C is a disulfide bond designed to be released by reduction, such as by glutathione. Alternatively C (or A-B-C) is a peptidic linkage design for cleavable by enzymes. Optionally, a self-immolating group such as pABC is included to cause release of a free unmodified drug upon cleavage (Antibody-Drug Conjugates: Design, Formulation, and Physicochemical Stability, Singh, Luisi, and Pak. Pharm Res (2015) 32:3541-3571). The linker is cleaved by enzymes such as esterases, matrix metalloproteinases, cathepsins such as cathepsin B, glucuronidases, a protease, or thrombin. Alternatively, the bond designed for cleavage is at A, rather than C, and C could be a stable bond or a cleavable bond. An alternative design is to have stable linkers (such as amide or carbamate) at A and C and have a cleavable linker in B, such as a disulfide bond. The rate of reduction is modulated by local effects such as steric hindrance from methyl or ethyl groups or modulating hydrophobicity/hydrophilicity.

The resulting peptide conjugates are administered to a human or animal subcutaneously, intravenously, orally, or injected directly into a joint to treat disease.

### Example 21

### Acetylsalicylic Acid Peptide Complex

This example describes the conjugation of acetylsalicylic acid to a peptide using a lactic acid linker. A conjugate is produced from a mixture of (R,S)-acetylsalicylic acid, lactic acid, and a peptide:

The acetylsalicylic acid-lactic acid linker conjugate depicted above is then reacted with a lysine or the N-terminus of a cystine-dense peptide to create an acetylsalicylic acid-lactic acid-peptide conjugate. The cystine-dense peptide is selected from the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

Acetylsalicylic acid is currently dosed as an enantiomeric mixture, in which enantiomers with a single racemic stereocenter are very difficult to separate. As in the reaction scheme (I), a diastereomer with two chiral centers is created by the addition of a chiral linker such as L-lactic acid. Since diastereomers are easily separated, the active enantiomer of acetylsalicylic acid complexed, conjugated, or fused to the lactic acid linker can be purified prior to conjugation to a cystine-dense peptide. The chemical synthesis can use any conjugation techniques known in the art, such as described in Bioconjugate Techniques by Greg Hermanson (Elsevier Inc., 3rd edition, 2013) and in “Ketorolac-dextran conjugates: synthesis, in vitro, and in vivo evaluation:” Acta Pharm. 57 (2007) 441-450, Vyas, Trivedi, and Chaturvedi. The conjugate can display anti-inflammatory activity, or free acetylsalicylic acid is released from the conjugate to provide anti-inflammatory activity. The free acetylsalicylic acid can result from hydrolysis that occurs after administration, such as hydrolysis at the ester bond. By dosing the conjugate containing the cartilage homing peptide, a higher AUC of acetylsalicylic acid delivery to the joint may be achieved than would be achieved by systemic dosing of acetylsalicylic acid alone.

Such peptide-drug conjugates can be made using either a cleavable or stable linker as described herein (e. g., EXAMPLES 19 and 20).

### Example 22

### Ibuprofen Peptide Complex

This example describes the conjugation of ibuprofen to a peptide using a PEG linker. A conjugate is produced using ibuprofen and a PEG linker, which forms an ester bond that can hydrolyze as described in “In vitro and in vivo study of poly(ethylene glycol) complexed, conjugated, or fused ibuprofen to extend the duration of action,” Scientia Pharmaceutica, 2011, 79:359-373, Nayak and Jain. Fischer esterification is used to conjugate ibuprofen with a short PEG, e.g., with triethylene glycol, to yield ibuprofen-ester-PEG-OH.

Following preparation of the PEG-ibuprofen conjugate as shown above, the hydroxyl moiety of PEG is activated with N,N′-disuccinimidyl carbonate (DSC) to form ibuprofen-ester-PEG-succinimidyl carbonate, which is then reacted with a lysine or the N-terminus of a cystine-dense peptide to form an ibuprofen-ester-PEG-peptide conjugate. The cystine-dense peptide is selected from any one of the peptides of sequence SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. The conjugate can display anti-inflammatory activity, or free ibuprofen is released from the conjugate to provide anti-inflammatory activity. The free ibuprofen can result from hydrolysis that occurs after administration, such as hydrolysis at the ester bond.

Ibuprofen-peptide conjugates are administered to a subject in need thereof. The subject can be a human or a non-human animal.

Such peptide-drug conjugates can be made using either a cleavable or stable linker as described herein (e. g., EXAMPLES 19 and 20). Similar peptide-drug conjugates can be made using acetylsalicylic acid.

### Example 23

### Dexamethasone Peptide Complex

This example describes different methods of complexing dexamethasone with a peptide of this disclosure. A peptide of any one of SEQ ID NO: 109-SEQ ID NO: 126 or SEQ ID NO: 129-SEQ ID NO: 133 was recombinantly expressed. Dexamethasone was readily chemically conjugated to a peptide of this disclosure using a dicarboxylic acid linker. The peptide-dexamethasone conjugate was made by first converting dexamethasone to a hemisuccinate by reacting it with succinic anhydride. The hemisuccinate was then converted to a succinate carboxylic acid containing an active ester, using dicyclohexyl carbodiimide (DCC) or 1-ethyl-3-(3-dimethylaminopropyl)carbodiimide (EDC) in the presence of N-hydroxy succinimide (NHS). This active ester was then reacted with a lysine or the N-terminus of a cystine-dense peptide to create a dexamethasone-carboxylic acid-peptide conjugate. Methods such as those described in “Functionalized derivatives of hyaluronic acid oligosaccharides: drug carriers and novel biomaterials” Bioconjugate Chemistry 1994, 5, 339-347, Pouyani and Prestwich, and Bioconjugate Techniques by Greg Hermanson (Elsevier Inc., 3rd edition, 2013) can be used.

Peptide-dexamethasone conjugates were prepared by coupling dexamethasone to the peptides of this disclosure using standard coupling-reagent chemistry. For example, dexamethasone conjugates were made by reacting dexamethasone hemigluterate with 1.05 molar equivalents of 1,1′-carbonyldiimidazole in anhydrous DMSO in an inert atmosphere. After 30 minutes, excess dexamethasone in anhydrous DMSO was added along with two molar equivalents of anhydrous trimethylamine. The N-hydroxysuccinimide ester of the peptide-dexamethasone conjugate was generated to form a shelf-stable intermediate for later reaction with an amine-containing carrier. The N-terminal dexamethasone-peptide conjugate (SEQ ID NO: 108B) was verified by electrospray mass spectrometry (ES-MS) within a 10 ppm error.

The peptide can be a peptide of SEQ ID NO: 115. The peptide can be a peptide of SEQ ID NO: 234. A peptide of any of the sequences of this disclosure including SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263, are conjugated to dexamethasone using the methods described above.

Such peptide-drug conjugates can be made using either a cleavable or stable linker as described herein (e. g., EXAMPLES 19 and 20).

Any peptide of any sequence of this disclosure can be complexed, conjugated, or fused to dexamethasone.

### Example 24

### Beclomethasone Monopropionate Peptide Complexes

This example describes conjugation of a peptide any one of SEQ ID NO: 109-SEQ ID NO: 126 or SEQ ID NO: 129-SEQ ID NO: 133 to beclomethasone monopropionate. Beclomethasone monopropionate is readily complexed, conjugated, or fused to any peptide disclosed herein via a dicarboxylic acid linker. The dicarboxylic acid linker is a linear dicarboxylic acid, such as succinic acid, or a related cyclic anhydride, such as succinic anhydride. Reactions with anhydrides can proceed under simple conditions. For example, the reaction of beclomethasone monopropionate with five molar equivalents of glutaric anhydride is carried out in anhydrous pyridine at room temperature. Reactions with dicarboxylic acids can occur using standard carbodiimide coupling methods. For example, beclomethasone monopropionate is reacted with one molar equivalent dimethylsuccinic acid, one molar equivalent 1-ethyl-3-(3-dimethylaminopropyl)carbodiimide (or another carbodiimide), and 0.2 molar equivalents of 40-dimethylamino pyridine.

The same methods as described in EXAMPLE 18 are used to adjust the rate of hydrolysis of peptide-beclomethasone monopropionate conjugates, preventing premature cleavage and ensuring that the beclomethasone monopropionate of peptide-beclomethasone monopropionate conjugates accumulate in cartilage.

Peptide-beclomethasone monopropionate conjugates are prepared by coupling beclomethasone monopropionate to the peptides of this disclosure using standard coupling-reagent chemistry. The peptide-beclomethasone monopropionate conjugate was made by first converting beclomethasone monopropionate to a hemisuccinate by reacting it with succinic anhydride. The hemisuccinate was then converted to a succinate carboxylic acid containing an active ester, using dicyclohexyl carbodiimide (DCC) or 1-ethyl-3-(3-dimethylaminopropyl)carbodiimide (EDC) in the presence of N-hydroxy succinimide (NHS). This active ester was then reacted with a lysine or the N-terminus of a peptide to create a beclomethasone monopropionate-carboxylic acid-peptide conjugate. Methods such as those described in “Functionalized derivatives of hyaluronic acid oligosaccharides: drug carriers and novel biomaterials” Bioconjugate Chemistry 1994, 5, 339-347, Pouyani and Prestwich, and Bioconjugate Techniques by Greg Hermanson (Elsevier Inc., 3rd edition, 2013) can be used.

Peptide-beclomethasone monopropionate conjugates were prepared by coupling beclomethasone monopropionate to the peptides of this disclosure using standard coupling-reagent chemistry. For example, beclomethasone monopropionate conjugates were made by reacting beclomethasone monopropionate hemigluterate with 1.05 molar equivalents of 1,1′-carbonyldiimidazole in anhydrous DMSO in an inert atmosphere. After 30 minutes, excess beclomethasone monopropionate in anhydrous DMSO was added along with two molar equivalents of anhydrous trimethylamine. The N-hydroxysuccinimide ester of the peptide-beclomethasone monopropionate conjugate was generated to form a shelf-stable intermediate for later reaction with an amine-containing carrier.

Beclomethasone monopropionate is also readily complexed, conjugated, or fused to any peptide disclosed herein via a dicarboxylic acid linker. The dicarboxylic acid linker is a linear dicarboxylic acid, such as succinic acid, or a related cyclic anhydride, such as succinic anhydride. Reactions with anhydrides can proceed under simple conditions. For example, the reaction of beclomethasone monopropionate with five molar equivalents of glutaric anhydride is carried out in anhydrous pyridine at room temperature. Reactions with dicarboxylic acids can occur using standard carbodiimide coupling methods. For example, beclomethasone monopropionate is reacted with one molar equivalent dimethylsuccinic acid, one molar equivalent 1-ethyl-3-(3-dimethylaminopropyl)carbodiimide (or another carbodiimide), and 0.2 molar equivalents of 40-dimethylamino pyridine. The peptide-beclomethasone monopropionate conjugates are administered to a subject in need thereof and home, target, are directed to, are retained by, accumulate in, migrate to, and/or bind to cartilage and/or kidneys. The subject is a human or animal and has inflammation in the cartilage or kidney tissues. Upon administration of the peptide-beclomethasone monopropionate conjugates, the cartilage and/or kidney inflammation is alleviated.

The peptide can be a peptide of SEQ ID NO: 115. The peptide can be a peptide of SEQ ID NO: 234. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

Such peptide-drug conjugates are made using either a cleavable or stable linker as described herein (e. g., EXAMPLES 19 and 20).

### Example 25

### Desciclesonide Peptide Complexes

This example describes conjugation of a peptide of any one of SEQ ID NO: 109-SEQ ID NO: 126 or SEQ ID NO: 129-SEQ ID NO: 133 to desciclesonide. Ciclesonide is a prodrug that is metabolized in vivo to the active metabolite desciclesonide. By conjugating desciclesonide to a peptide via an ester linker, upon hydrolysis the released drug would be desciclesonide, just as after systemic administration of ciclesonide the active metabolite desciclesonide is present and active. Desciclesonide is readily complexed, conjugated, or fused to any peptide disclosed herein via a dicarboxylic acid linker. The dicarboxylic acid linker is a linear dicarboxylic acid, such as succinic acid, or a related cyclic anhydride, such as succinic anhydride. Reactions with anhydrides can proceed under simple conditions. For example, the reaction of desciclesonide with five molar equivalents of glutaric anhydride is carried out in anhydrous pyridine at room temperature. Reactions with dicarboxylic acids can occur using standard carbodiimide coupling methods. For example, desciclesonide is reacted with one molar equivalent dimethylsuccinic acid, one molar equivalent 1-ethyl-3-(3-dimethylaminopropyl)carbodiimide (or another carbodiimide), and 0.2 molar equivalents of 40-dimethylamino pyridine.

The same methods as described in EXAMPLE 18 are used to adjust the rate of hydrolysis of peptide-desciclesonide conjugates, preventing premature cleavage and ensuring that the desciclesonide of peptide-desciclesonide conjugates accumulate in cartilage.

Desciclesonide is also readily complexed, conjugated, or fused to any peptide disclosed herein via a dicarboxylic acid linker. The dicarboxylic acid linker is a linear dicarboxylic acid, such as succinic acid, or a related cyclic anhydride, such as succinic anhydride. Reactions with anhydrides can proceed under simple conditions. For example, the reaction of desciclesonide with five molar equivalents of glutaric anhydride is carried out in anhydrous pyridine at room temperature. Reactions with dicarboxylic acids can occur using standard carbodiimide coupling methods. For example, desciclesonide is reacted with one molar equivalent dimethylsuccinic acid, one molar equivalent 1-ethyl-3-(3-dimethylaminopropyl)carbodiimide (or another carbodiimide), and 0.2 molar equivalents of 40-dimethylamino pyridine. The peptide-desciclesonide conjugates are administered to a subject in need thereof and home, target, are directed to, are retained by, accumulate in, migrate to, and/or bind to cartilage and/or kidneys. The subject is a human or animal and has inflammation in the cartilage or kidney tissues. Optionally, the subject has osteoarthritis, rheumatoid arthritis, ankylosing spondylitis, lupus arthritis, systemic lupus erythematosus, or lupus nephritis. Upon administration of the peptide-desciclesonide conjugates, the cartilage and/or kidney inflammation is alleviated.

The peptide can be a peptide of SEQ ID NO: 115. The peptide can be a peptide of SEQ ID NO: 234. The peptide can be a peptide of SEQ ID NO: 114, SEQ ID NO: 126, or SEQ ID NO: 109. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

Such peptide-drug conjugates are made using either a cleavable or stable linker as described herein (e. g., EXAMPLES 19 and 20).

### Example 26

### Desciclesonide Peptide Complexes

This example describes conjugation of a peptide of any one of SEQ ID NO: 109-SEQ ID NO: 126 or SEQ ID NO: 129-SEQ ID NO: 133 to desciclesonide. Ciclesonide is a prodrug that is metabolized in vivo to the active metabolite desciclesonide. By conjugating desciclesonide to a peptide via a linker such as an ester, a carbonate, or a carbamate, upon hydrolysis the released drug can be desciclesonide, just as after systemic administration of ciclesonide the active metabolite desciclesonide is present and active. Desciclesonide is readily complexed, conjugated, or fused to any peptide disclosed herein via a stable or cleavable linker.

The peptide-desciclesonide conjugates are administered to a subject in need thereof and home, target, are directed to, are retained by, accumulate in, migrate to, and/or bind to cartilage and/or kidneys. The subject is a human or animal and has inflammation in the cartilage or kidney tissues. Upon administration of the peptide desciclesonide conjugates, the cartilage and/or kidney inflammation is alleviated.

The peptide can be a peptide of SEQ ID NO: 115. The peptide can be a peptide of SEQ ID NO: 234. The peptide can be a peptide of SEQ ID NO: 114, SEQ ID NO: 126, or SEQ ID NO: 109. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

Such peptide-drug conjugates are made using either a cleavable or stable linker as described herein (e. g., EXAMPLES 19 and 20).

### Example 27

### Peptide-Ustekinumab Complexes

This example describes conjugation of a peptide of any one of SEQ ID NO: 109-SEQ ID NO: 126 or SEQ ID NO: 129-SEQ ID NO: 133 to ustekinumab. Ustekinimab is readily complexed, conjugated, or fused to any peptide disclosed herein via standard chemistries such as those described in, but not limited to, Bioconjugate Techniques by Greg Hermanson (Elsevier Inc., 3rd edition, 2013). Alternatively the peptide-active agent of this Example can be expressed as a fusion protein. From one to eight peptides are linked to ustekinumab.

The peptide-ustekinumab conjugates are administered to a subject in need thereof and home, target, are directed to, are retained by, accumulate in, migrate to, and/or bind to cartilage. The subject is a human or animal and has psoriatic arthritis. Upon administration of the peptide-ustekinumab conjugates, the psoriatic arthritis condition is alleviated.

The peptide can be a peptide of SEQ ID NO: 115. The peptide can be a peptide of SEQ ID NO: 234. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

### Example 28

### Peptide-Xeljanz Complexes

This example describes conjugation of a peptide of any one of SEQ ID NO: 109-SEQ ID NO: 126 or SEQ ID NO: 129-SEQ ID NO: 133 to xeljanz. Xeljanz is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to xeljanz.

The peptide-xeljanz conjugates are administered to a subject in need thereof and home, target, are directed to, are retained by, accumulate in, migrate to, and/or bind to cartilage. The subject is a human or animal and has rheumatoid arthritis. Upon administration and homing of peptide-xeljanz conjugates, the rheumatoid arthritis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 29

### Peptide-IL-17 Inhibitor Complexes

This example describes conjugation of a peptide of any one of SEQ ID NO: 109-SEQ ID NO: 126 or SEQ ID NO: 129-SEQ ID NO: 133 to an IL-17 inhibitor. An IL-17 inhibitor is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-IL-17 inhibitor conjugates are administered to a subject in need thereof and home, target, are directed to, are retained by, accumulate in, migrate to, and/or bind to cartilage. The subject is a human or animal and has ankylosing spondylitis. Upon administration and homing of peptide-IL-17 inhibitor conjugates, the ankylosing spondylitis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 30

### Peptide-Iguratimod Complexes

This example describes conjugation of a peptide of any one of SEQ ID NO: 109-SEQ ID NO: 126 or SEQ ID NO: 129-SEQ ID NO: 133 to iguratimod. Iguratimod is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-iguratimod conjugates are administered to a subject in need thereof and home, target, are directed to, are retained by, accumulate in, migrate to, and/or bind to kidneys. The subject is a human or animal and has rheumatoid arthritis. Upon administration and homing of peptide-iguratimod conjugates, the rheumatoid arthritis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 31

### Peptide Mycophenolic Acid Complexes

This example describes conjugation of a peptide of any one of SEQ ID NO: 109-SEQ ID NO: 126 or SEQ ID NO: 129-SEQ ID NO: 133 to mycophenolic acid. Mycophenolic acid is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-mycophenolic acid conjugates are administered to a subject in need thereof and home, target, are directed to, are retained by, accumulate in, migrate to, and/or bind to kidneys. The subject is a human or animal and has organ transplantation, infection, cancer, or other kidney disorders. Upon administration and homing of peptide-mycophenolic acid conjugates, the organ transplantation, infection, cancer, other kidney disorders condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 32

### Peptide-Tacrolimus Complexes

This example describes conjugation of a peptide of any one of SEQ ID NO: 109-SEQ ID NO: 126 or SEQ ID NO: 129-SEQ ID NO: 133 to tacrolimus. Tacrolimus is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-tacrolimus conjugates are administered to a subject in need thereof and home, target, are directed to, are retained by, accumulate in, migrate to, and/or bind to kidneys. The subject is a human or animal and has organ transplantation, any other kidney disease. Upon administration and homing of peptide-tacrolimus conjugates, the organ transplantation, any other kidney disease condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 33

### Peptide-Secukinumab Complexes

This example describes conjugation of a peptide of any one of SEQ ID NO: 109-SEQ ID NO: 126 or SEQ ID NO: 129-SEQ ID NO: 133 to secukinumab. Secukinumab is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to secukinumab. Alternatively the peptide-active agent of this Example can be expressed as a fusion protein.

The peptide-secukinumab conjugates are administered to a subject in need thereof and home, target, are directed to, are retained by, accumulate in, migrate to, and/or bind to cartilage. The subject is a human or animal and has ankylosing spondylitis. Upon administration and homing of peptide-secukinumab acid conjugates, the ankylosing spondylitis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 34

### Peptide-Sirukumab Complexes

This example describes conjugation of a peptide of any one of SEQ ID NO: 109-SEQ ID NO: 126 or SEQ ID NO: 129-SEQ ID NO: 133 to sirukumab. Sirukumab is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to sirukumab. Alternatively the peptide-active agent of this Example can be expressed as a fusion protein.

The peptide-sirukumab conjugates are administered to a subject in need thereof and home, target, are directed to, are retained by, accumulate in, migrate to, and/or bind to kidneys. The subject is a human or animal and has rheumatoid arthritis, immune diseases of the kidneys. Upon administration and homing of peptide-sirukumab conjugates, the rheumatoid arthritis, immune diseases of the kidneys condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 35

### Peptide-Anifrolumab Complexes

This example describes conjugation of a peptide of any one of SEQ ID NO: 109-SEQ ID NO: 126 or SEQ ID NO: 129-SEQ ID NO: 133 to anifrolumab. Anifrolumab is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to anifrolumab. Alternatively the peptide-active agent of this Example can be expressed as a fusion protein.

The peptide-anifrolumab conjugates are administered to a subject in need thereof and home, target, are directed to, are retained by, accumulate in, migrate to, and/or bind to kidneys. The subject is a human or animal and has lupus nephritis. Upon administration and homing of peptide-anifrolumab conjugates, the lupus nephritis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 36

### Peptide-Denosumab Complexes

This example describes conjugation of a peptide of any one of SEQ ID NO: 109-SEQ ID NO: 126 or SEQ ID NO: 129-SEQ ID NO: 133 to denosumab. Denosumab is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to denosumab. Alternatively the peptide-active agent of this Example can be expressed as a fusion protein.

The peptide-denosumab conjugates are administered to a subject in need thereof and home, target, are directed to, are retained by, accumulate in, migrate to, and/or bind to cartilage. The subject is a human or animal and has osteoporosis. Upon administration and homing of peptide-denosumab conjugates, the osteoporosis condition is alleviated.

The peptide can be a peptide of SEQ ID NO: 115. The peptide can be a peptide of SEQ ID NO: 234. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 37

### Peptide-Rituximab Complexes

This example describes conjugation of a peptide of any one of SEQ ID NO: 109-SEQ ID NO: 126 or SEQ ID NO: 129-SEQ ID NO: 133 to rituximab. Rituximab is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to rituximab. Alternatively the peptide-active agent of this Example can be expressed as a fusion protein.

The peptide-rituximab conjugates are administered to a subject in need thereof and home, target, are directed to, are retained by, accumulate in, migrate to, and/or bind to cartilage or kidneys. The subject is a human or animal and has rheumatoid arthritis, kidney transplant. Upon administration and homing of peptide-rituximab conjugates, the rheumatoid arthritis, kidney transplant condition is alleviated.

The peptide can be a peptide of SEQ ID NO: 115. The peptide can be a peptide of SEQ ID NO: 234. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 38

### Peptide-Omalizumab Complexes

This example describes conjugation of a peptide of any one of SEQ ID NO: 109-SEQ ID NO: 126 or SEQ ID NO: 129-SEQ ID NO: 133 to omalizumab. Omalizumab is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to omalizumab. Alternatively the peptide-active agent of this Example can be expressed as a fusion protein.

The peptide-omalizumab conjugates are administered to a subject in need thereof and home, target, are directed to, are retained by, accumulate in, migrate to, and/or bind to kidneys. The subject is a human or animal and has kidney inflammation. Upon administration and homing of peptide-omalizumab conjugates, the kidney inflammation condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 39

### Peptide-Abatacept Complexes

This example describes conjugation of a peptide of any one of SEQ ID NO: 109-SEQ ID NO: 126 or SEQ ID NO: 129-SEQ ID NO: 133 to abatacept. Abatacept is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to abatacept. Alternatively the peptide-active agent of this Example can be expressed as a fusion protein.

The peptide-abatacept conjugates are administered to a subject in need thereof and home, target, are directed to, are retained by, accumulate in, migrate to, and/or bind to kidneys. The subject is a human or animal and has rheumatoid arthritis, lupus nephritis, organ transplant, focal segmental glomerulosclerosis. Upon administration and homing of peptide-abatacept conjugates, the rheumatoid arthritis, lupus nephritis, organ transplant, focal segmental glomerulosclerosis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 40

### Peptide-Oxycodone Complexes

This example describes conjugation of a peptide of any one of SEQ ID NO: 109-SEQ ID NO: 126 or SEQ ID NO: 129-SEQ ID NO: 133 to oxycodone. Oxycodone is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-oxycodone conjugates are administered to a subject in need thereof and home, target, are directed to, are retained by, accumulate in, migrate to, and/or bind to cartilage. The subject is a human or animal and has cartilage or kidney-related pain. Upon administration and homing of peptide-oxycodone conjugates, the cartilage-related pain condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 41

### Peptide Capsaicin Complexes

This example describes conjugation of a peptide of any one of SEQ ID NO: 109-SEQ ID NO: 126 or SEQ ID NO: 129-SEQ ID NO: 133 to capsaicin. Capsaicin is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-capsaicin conjugates are administered to a subject in need thereof and home, target, are directed to, are retained by, accumulate in, migrate to, and/or bind to cartilage. The subject is a human or animal and has cartilage-related pain. Upon administration and homing of peptide-capsaicin conjugates, the cartilage or kidney-related pain condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 42

### Peptide-GSK2193874 Complexes

This example describes conjugation of a peptide of any one of SEQ ID NO: 109-SEQ ID NO: 126 or SEQ ID NO: 129-SEQ ID NO: 133 to GSK2193874. GSK2193874 is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-GSK2193874 conjugates are administered to a subject in need thereof and home, target, are directed to, are retained by, accumulate in, migrate to, and/or bind to GSK2193874. The subject is a human or animal and has cartilage-related pain. Upon administration and homing of peptide-GSK2193874 conjugates, the cartilage-related pain condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 43

### Peptide BIB023 Complexes

This example describes conjugation of a peptide of any one of SEQ ID NO: 109-SEQ ID NO: 126 or SEQ ID NO: 129-SEQ ID NO: 133 to BIIB023. BIIB023 is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to BIIB023. Alternatively the peptide-active agent of this Example can be expressed as a fusion protein.

The peptide-BIIB023 conjugates are administered to a subject in need thereof and home, target, are directed to, are retained by, accumulate in, migrate to, and/or bind to cartilage. The subject is a human or animal and has lupus nephritis or rheumatoid arthritis. Upon administration and homing of peptide-BIIB023 conjugates, the lupus nephritis or rheumatoid arthritis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 44

### Peptide-Anakinra Complexes

This example describes conjugation or fusion of a peptide any one of SEQ ID NO: 109-SEQ ID NO: 126 or SEQ ID NO: 129-SEQ ID NO: 133 to anakinra. A linker is optionally used to conjugate the peptide to anakinra. Anakinra is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to anakinra. Alternatively the peptide-active agent of this Example can be expressed as a fusion protein.

The peptide-anakinra conjugates or fusions are administered to a subject in need thereof and home, target, are directed to, are retained by, accumulate in, migrate to, and/or bind to cartilage. The subject is a human or animal and has lupus nephritis or rheumatoid arthritis. Upon administration and homing of peptide-anakinra conjugates or fusions, the lupus nephritis or rheumatoid arthritis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 45

### Peptide-IGF-1 Complexes

This example describes conjugation of a peptide of any one of SEQ ID NO: 109-SEQ ID NO: 126 or SEQ ID NO: 129-SEQ ID NO: 133 to IGF-1. IGF-1 is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to IGF-1. Alternatively the peptide-active agent of this Example can be expressed as a fusion protein.

The peptide-IGF-1 conjugates are administered to a subject in need thereof and home, target, are directed to, are retained by, accumulate in, migrate to, and/or bind to cartilage. The subject is a human or animal and has renal cancer or arthritis. Upon administration and homing of peptide-IGF-1 conjugates, the renal cancer or arthritis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 46

### Peptide-Romosozumab Complexes

This example describes conjugation of a peptide of any one of SEQ ID NO: 109-SEQ ID NO: 126 or SEQ ID NO: 129-SEQ ID NO: 133 to Romosozumab. Romosozumab is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to romosozumab. Alternatively the peptide-active agent of this Example can be expressed as a fusion protein.

The peptide-romosozumab conjugates are administered to a subject in need thereof and home, target, are directed to, are retained by, accumulate in, migrate to, and/or bind to cartilage. The subject is a human or animal and has osteoporosis. Upon administration and homing of peptide-romosozumab conjugates, the osteoporosis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 47

### Peptide-ZVAD-fmk Complexes

This example describes conjugation of a peptide of any one of SEQ ID NO: 109-SEQ ID NO: 126 or SEQ ID NO: 129-SEQ ID NO: 133 to ZVAD-fmk. ZVAD-fmk is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to ZVAD-fmk.

The peptide-ZVAD-fmk conjugates are administered to a subject in need thereof and home, target, are directed to, are retained by, accumulate in, migrate to, and/or bind to cartilage. The subject is a human or animal and has cartilage grafting, arthritis, surgical intervention, surgery for cartilage repair. Upon administration and homing of peptide-ZVAD-fmk conjugates, the cartilage grafting, arthritis, surgical intervention, surgery for cartilage repair condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 48

### Peptide-S-methylisothiourea Complexes

This example describes conjugation of a peptide of any one of SEQ ID NO: 109-SEQ ID NO: 126 or SEQ ID NO: 129-SEQ ID NO: 133 to S-methylisothiourea. S-methylisothiourea is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-S-methylisothiourea conjugates are administered to a subject in need thereof and home, target, are directed to, are retained by, accumulate in, migrate to, and/or bind to cartilage. The subject is a human or animal and has arthritis surgery, kidney iron overload, renal ischemia reperfusion injury, or acute kidney injury. Upon administration and homing of peptide-S-methylisothiourea conjugates, the arthritis surgery, kidney iron overload, renal ischemia reperfusion injury, or acute kidney injury condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 49

### Peptide-P188 Complexes

This example describes conjugation of a peptide of any one of SEQ ID NO: 109-SEQ ID NO: 126 or SEQ ID NO: 129-SEQ ID NO: 133 to P188. P188 is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-P188 conjugates are administered to a subject in need thereof and home, target, are directed to, are retained by, accumulate in, migrate to, and/or bind to cartilage. The subject is a human or animal and has arthritis surgery. Upon administration and homing of peptide-P188 conjugates, the arthritis surgery condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 50

### Peptide-Alendronate Complexes

This example describes conjugation of a peptide of any one of SEQ ID NO: 109-SEQ ID NO: 126 or SEQ ID NO: 129-SEQ ID NO: 133 to alendronate. Alendronate is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-alendronate conjugates are administered to a subject in need thereof and home, target, are directed to, are retained by, accumulate in, migrate to, and/or bind to cartilage. The subject is a human or animal and has bone erosion. Upon administration and homing of peptide-alendronate conjugates, the bone erosion condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 51

### Peptide-MIP-3α Complexes

This example describes conjugation of a peptide of any one of SEQ ID NO: 109-SEQ ID NO: 126 or SEQ ID NO: 129-SEQ ID NO: 133 to MIP-3α. MIP-3α is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to MIP-3α. Alternatively the peptide-active agent of this Example can be expressed as a fusion protein.

The peptide-MIP-3α conjugates are administered to a subject in need thereof and home, target, are directed to, are retained by, accumulate in, migrate to, and/or bind to cartilage. The subject is a human or animal and has Joint injury, repair and regeneration of cartilage and bone. Upon administration and homing of peptide-MIP-3α conjugates, the Joint injury, repair and regeneration of cartilage and bone condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 52

### Peptide-BMP-2 Complexes

This example describes conjugation of a peptide of any one of SEQ ID NO: 109-SEQ ID NO: 126 or SEQ ID NO: 129-SEQ ID NO: 133 to BMP-2. BMP-2 is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to BMP-2. Alternatively the peptide-active agent of this Example can be expressed as a fusion protein.

The peptide-BMP-2 conjugates are administered to a subject in need thereof and home, target, are directed to, are retained by, accumulate in, migrate to, and/or bind to cartilage. The subject is a human or animal and has Joint repair. Upon administration and homing of peptide-BMP-2 conjugates, the Joint repair condition is alleviated.

The peptide can also be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 53

### Peptide-Icariin Complexes

This example describes conjugation of a peptide of any one of SEQ ID NO: 109-SEQ ID NO: 126 or SEQ ID NO: 129-SEQ ID NO: 133 to icariin. Icariin is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-icariin conjugates are administered to a subject in need thereof and home, target, are directed to, are retained by, accumulate in, migrate to, and/or bind to cartilage. The subject is a human or animal and has Joint repair. Upon administration and homing of peptide-icariin conjugates, the Joint repair condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or a stable linker as described herein (e. g., EXAMPLES 19 and 20).

### Example 54

### Peptide-Captopril Complexes

This example describes conjugation of a peptide of any one of SEQ ID NO: 109-SEQ ID NO: 126 or SEQ ID NO: 129-SEQ ID NO: 133 to captopril. Captopril is readily complexed, conjugated, or fused to any peptide disclosed herein via standard chemistries such as those described in, but not limited to, Bioconjugate Techniques by Greg Hermanson (Elsevier Inc., 3rd edition, 2013).

The peptide-captopril conjugates are administered to a subject in need thereof and home, target, are directed to, are retained by, accumulate in, migrate to, and/or bind to kidneys. The subject is a human or animal and has diabetic nephropathy. Upon administration and homing of peptide-captopril conjugates, the diabetic nephropathy condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 55

### Peptide-Tofacitinib Complexes

This example describes conjugation of a peptide of any one of SEQ ID NO: 109-SEQ ID NO: 126 or SEQ ID NO: 129-SEQ ID NO: 133 to tofacitinib. Tofacitinib is readily complexed, conjugated, or fused to any peptide disclosed herein via standard chemistries such as those described in, but not limited to, Bioconjugate Techniques by Greg Hermanson (Elsevier Inc., 3rd edition, 2013). From one to eight peptides are linked to tofacitinib.

The peptide-tofacitinib conjugates are administered to a subject in need thereof and home, target, are directed to, are retained by, accumulate in, migrate to, and/or bind to cartilage. The subject is a human or animal and has rheumatoid arthritis and kidney transplant, ankyloses spondylitis. Upon administration and homing of peptide-tofacitinib conjugates, the rheumatoid arthritis and kidney transplant, ankyloses spondylitis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 56

### Peptide-Dimethyl Fumarate Complexes

This example describes conjugation of a peptide of any one of SEQ ID NO: 109-SEQ ID NO: 126 or SEQ ID NO: 129-SEQ ID NO: 133 to dimethyl fumarate. Dimethyl fumarate is readily complexed, conjugated, or fused to any peptide disclosed herein via standard chemistries such as those described in, but not limited to, Bioconjugate Techniques by Greg Hermanson (Elsevier Inc., 3rd edition, 2013). Alternatively, peptide-dimethyl fumarate conjugates can be synthesized by Michael addition of a thiol (on the peptide of linker) to dimethyl fumarate as described by Schmidt et al. (Bioorg Med Chem. 2007 Jan. 1; 15(1):333-42. Epub 2006 Sep. 29.).

The peptide-dimethyl fumarate conjugates are administered to a subject in need thereof and home, target, are directed to, are retained by, accumulate in, migrate to, and/or bind to kidneys. The subject is a human or animal and has Kidney fibrosis, psoriatic arthritis, rheumatoid arthritis. Upon administration and homing of peptide-dimethyl fumarate conjugates, the Kidney fibrosis, psoriatic arthritis, rheumatoid arthritis condition is alleviated.

The peptide can be a peptide of SEQ ID NO: 115. The peptide can be a peptide of SEQ ID NO: 234. The peptide can be any peptide with the sequence selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 57

### Intra-Articular Administration of Peptides and Peptide Complexes

This example illustrates intra-articular administration of peptides or peptide conjugates of this disclosure. A peptide of this disclosure is expressed recombinantly or chemically synthesized. In some cases, the peptide is subsequently complexed, conjugated, or fused to a detectable agent or an active agent. The peptide or peptide conjugate is administered to a subject in need thereof via intra-articular administration. The cartilage is penetrated by the peptide or peptide conjugate due to the small size of the peptide or peptide conjugate, and due to binding of cartilage components by the peptide or peptide conjugate. The peptide or peptide conjugate is bound to cartilage and the residence time in the cartilage is longer due to this binding. Optionally, the injected material is aggregated, is crystallized, or complexes are formed, further extending the depot effect and contributing to longer residence time.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 58

### Treatment of Osteoarthritis

This example describes a method for treating osteoarthritis using peptides of the present disclosure. This method is used as a treatment for acute and/or chronic symptoms associated with osteoarthritis. A peptide of the present disclosure is expressed recombinantly or chemically synthesized and then is used directly or complexed, conjugated, or fused to an anti-inflammatory compound, such as aspirin, desciclesonide, or secukinumab. The resulting peptide or peptide-drug conjugate is administered in a pharmaceutical composition subcutaneously, intravenously, or orally, or is injected directly into a joint of a patient and targeted to cartilage. The formulation can be modified physically or chemically to increase the time of exposure in the cartilage. One or more anti-inflammatory peptide conjugates are administered to a human or animal.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 59

### Treatment of Cartilage Degradation

This example describes a method for treating and/or preventing cartilage degradation using a peptide of the present disclosure. This method is used as a treatment for acute and/or chronic symptoms associated with cartilage degradation. Progressive degradation or thinning of the cartilage is difficult to treat in part because molecules such as small molecule drugs and antibodies typically do not reach the avascular cartilage. A peptide of the present disclosure is used for its homing and/or native activity, or is mutated to generate activity such as MMP protease inhibition. It is expressed recombinantly or chemically synthesized and then is used directly or complexed, conjugated, or fused to an extracellular matrix targeting active agent, such as an inhibitor of MMP activity or an anti-apoptosis agent (e. g., osteoprotegrin, romosozumab, P188, ZVAD-fmk, quercetin, dasatinib, dimethyl fumarate, bortezomib, carilzomib, or navitoclax). The resulting peptide or peptide-drug conjugate is administered in a pharmaceutical composition subcutaneously, intravenously, or orally, or is injected directly into a joint of a patient and targeted to extracellular matrix. One or more extracellular matrix targeting conjugates are administered to a human or animal.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 60

### Treatment of a Cartilage Injury

This example describes a method for treating a cartilage injury using a peptide of the present disclosure. A peptide of the present disclosure is expressed recombinantly or chemically synthesized and then is used directly or complexed, conjugated, or fused to a therapeutic compound, such as those described herein, including, but not limited to BMP-2, BMP-7, BMP-9, BMP-13, PDGF, PTH, PTHrP, IL-8, MIP-3α. The resulting peptide or peptide-drug conjugate is administered in a pharmaceutical composition to a patient and targeted to cartilage. One or more therapeutic compound-peptide conjugates are administered to a human or animal.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 61

### Treatment of Rheumatoid Arthritis

This example describes a method for treating rheumatoid arthritis. This method is used as a treatment for acute and/or chronic symptoms associated with rheumatoid arthritis. A peptide of the present disclosure is expressed recombinantly or chemically synthesized and then is used directly, or is complexed, conjugated, or fused to an anti-inflammatory compound, such as adalimumab, certolizumab, golimumab. thalidomide, lenalidomide, pomalidomide, pentocifylline, bupropion, or desciclesonide. Optionally, when the peptide is used directly, the peptide can, for example, bind or inhibit ion channels such as Kv 1.3. The resulting peptide or peptide-drug conjugate is administered in a pharmaceutical composition to a patient and is targeted to cartilage. One or more anti-inflammatory compound-peptide conjugates are administered to a human or animal subcutaneously, intravenously, or orally, or is injected directly into a joint.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 62

### Treatment of Gout

This example describes a method for treating gout using peptides of the present disclosure. This method is used as a treatment for acute and/or chronic symptoms associated with gout. A peptide of the present disclosure is expressed and administered in a pharmaceutical composition to a patient as a therapeutic for gout. A peptide of the disclosure is recombinantly or chemically synthesized and then is used directly or complexed, conjugated, or fused to pegloticase to treat a cartilage disorder. A peptide of the disclosure is recombinantly or chemically synthesized and then is used directly or complexed, conjugated, or fused to probenecid to treat a kidney disorder. The peptide is administered in a pharmaceutical composition to a patient and the peptide is targeted to the cartilage or kidney affected by gout. One or more peptides are administered to a human or animal subcutaneously, intravenously, or orally, or is injected directly into a joint.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 63

### Treatment or Management of Pain

This example describes a method for treating or managing pain associated with a cartilage injury or disorder. This method is used as a treatment for acute and/or chronic symptoms associated with a cartilage injury or disorder. A peptide of the disclosure is expressed and administered in a pharmaceutical composition to a patient as a therapeutic for pain as a result of injury or other cartilage or joint condition as described herein. The peptide of the present disclosure inhibits ion channels, such as Nav 1.7. The peptide is expressed recombinantly or chemically synthesized, wherein the peptide selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Alternatively, the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 are mutated to maintain the cartilage homing function, but to add or increase ion channel inhibition, such as to Nav 1.7. Following expression or synthesis, the peptide is used directly or complexed, conjugated, or fused to a narcotic (e.g., oxycodone), a non-narcotic analgesic, a natural counter-irritant (capsaicin), or a pain receptor channel inhibitor (such as the TRPV4 inhibitor GSK2193874). Following administration of the peptide, the peptide targets to the cartilage affected by pain. One or more peptides are administered to a human or animal subcutaneously, intravenously, or orally, or is injected directly into a joint.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 64

### Treatment or Management of Pain with Peptides Only

This example describes a method for treating or managing pain associated with a cartilage injury or disorder. This method is used as a treatment for acute and/or chronic symptoms associated with a cartilage injury or disorder. A peptide of the disclosure is expressed and administered in a pharmaceutical composition to a patient as a therapeutic for pain as a result of injury or other cartilage or joint condition as described herein. The peptide of the present disclosure inhibits ion channels, such as Nav 1.7. The peptide is expressed recombinantly or chemically synthesized, wherein the peptide selected from SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Alternatively, the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 are mutated to maintain the cartilage homing function, but to add or increase ion channel inhibition, such as to Nav 1.7. Following expression or synthesis, the peptide is used directly. Following administration of the peptide, the peptide targets to the cartilage affected by pain. One or more peptides are administered to a human or animal subcutaneously, intravenously, or orally, or is injected directly into a joint.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

### Example 65

### Treatment of Chondrosarcoma

This example illustrates treatment of chondrosarcoma using peptides of the present disclosure. A peptide of the present disclosure is recombinantly expressed or chemically synthesized and are used directly, after radiolabeling, or after conjugation to a fluorophore or therapeutic compound, such as dasatinib. The peptide or peptide conjugate is administered in a pharmaceutical composition to a subject as a therapeutic for chondrosarcoma. One or more peptides or peptide conjugates of the present disclosure are administered to a subject. A subject can be a human or an animal. The pharmaceutical composition is administered subcutaneously, intravenously, orally, or injected directly into a joint. The peptides or peptide conjugates target cartilage affected by chondrosarcoma.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 66

### Treatment of Chordoma

This example illustrates treatment of chordoma using peptides of the present disclosure. A peptide of the present disclosure is recombinantly expressed or chemically synthesized and are used directly, after radiolabeling, or after conjugation to a fluorophore or therapeutic compound, such as dasatinib. The peptide or peptide conjugate is administered in a pharmaceutical composition to a subject as a therapeutic for chordoma. One or more peptides or peptide conjugates of the present disclosure are administered to a subject. A subject can be a human or an animal. The pharmaceutical composition is administered subcutaneously, intravenously, orally, or injected directly into a joint. The peptides or peptide conjugates target cartilage affected by chordoma.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 67

### Treatment for Rapid Pain Relief

This example illustrates rapid pain relief in patients treated for rheumatoid arthritis or osteoarthritis with the peptides or peptide conjugates of this disclosure. A peptide of this disclosure is expressed recombinantly or chemically synthesized, and then the N-terminus of the peptide is complexed, conjugated, or fused to an active agent via an NHS ester to produce a peptide-active agent conjugate. In some aspects the active agent such as a kidney therapeutic from TABLE 4 or TABLE 5. In some cases, the peptide alone is administered to the subject.

The peptide or peptide-active agent conjugate is administered to a subject in need thereof. The subject is a human or non-human animal. The subject in need thereof has rheumatoid arthritis or osteoarthritis. The peptide or peptide conjugate is delivered via intravenous administration. Upon administration, the peptide or peptide conjugate rapidly homes to cartilage. Rapid pain relief within five minutes to an hour is experienced by the subject, and pain relieve can last as long as over 3 hours.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 68

### Treatment for Systemic Lupus Erythematosus

This example illustrates treatment of systemic lupus erythematosus, including forms of the disease known as lupus nephritis and/or lupus arthritis using peptides or peptide conjugates of this disclosure. A peptide of the present disclosure is recombinantly expressed or chemically synthesized and are used directly, after radiolabeling, or after conjugation or fusion to a fluorophore or therapeutic compound, such as abatacept or BIIB023 or desciclesonide.

The peptide or peptide conjugate is administered in a pharmaceutical composition to a subject as a therapeutic for lupus. The peptide is selected from any one of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. One or more peptides or peptide conjugates of the present disclosure are administered to a subject. A subject can be a human or an animal. The pharmaceutical composition is administered subcutaneously, intravenously, orally, or injected directly. The peptides or peptide conjugates target kidney affected by lupus nephritis and/or cartilage affected by lupus arthritis. The lupus condition of the subject is slowed, mitigated or improved.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 69

### Treatment for Ankylosing Spondylitis

This example illustrates treatment of ankylosing spondylitis using peptides or peptide conjugates of this disclosure. A peptide of the present disclosure is recombinantly expressed or chemically synthesized and are used directly, after radiolabeling, or after conjugation or fusion to a fluorophore or therapeutic compound, such as abatacept or BIIB023 or desciclesonide.

The peptide or peptide conjugate is administered in a pharmaceutical composition to a subject as a therapeutic for ankylosing spondylitis. The peptide is selected from any one of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. One or more peptides or peptide conjugates of the present disclosure are administered to a subject. A subject can be a human or an animal. The pharmaceutical composition is administered subcutaneously, intravenously, orally, or injected directly. The peptides or peptide conjugates target cartilage affected by ankylosing spondylitis. The ankylosing spondylitis condition of the subject is improved.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 70

### Treatment for Acute Kidney Injury (AKI)

This example illustrates treatment of acute kidney injury (AKI) using peptides or peptide conjugates of this disclosure. A peptide of the present disclosure is recombinantly expressed or chemically synthesized and are used directly, after radiolabeling, or after conjugation to a fluorophore or therapeutic compound, such as such as a kidney therapeutic from TABLE 4 or TABLE 5.

The peptide or peptide conjugate is administered in a pharmaceutical composition to a subject as a therapeutic for acute kidney injury (AKI). The peptide is selected from any one of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. One or more peptides or peptide conjugates of the present disclosure are administered to a subject. A subject can be a human or an animal. The pharmaceutical composition is administered subcutaneously, intravenously, orally, or injected directly into a joint. The peptides or peptide conjugates target cartilage affected by acute kidney injury (AKI).

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 71

### Treatment for Chronic Kidney Disease (CKD)

This example illustrates treatment of chronic kidney disease (CKD) using peptides or peptide conjugates of this disclosure. A peptide of the present disclosure is recombinantly expressed or chemically synthesized and are used directly, after radiolabeling, or after conjugation to a fluorophore or therapeutic compound, such as a kidney therapeutic from TABLE 4 or TABLE 5.

The peptide or peptide conjugate is administered in a pharmaceutical composition to a subject as a therapeutic for chronic kidney disease (CKD). The peptide is selected from any one of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. One or more peptides or peptide conjugates of the present disclosure are administered to a subject. A subject can be a human or an animal. The pharmaceutical composition is administered subcutaneously, intravenously, orally, or injected directly into a joint. The peptides or peptide conjugates target cartilage affected by chronic kidney disease (CKD).

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 72

### Treatment for Hypertensive Kidney Damage

This example illustrates treatment of hypertensive kidney damage using peptides or peptide conjugates of this disclosure. A peptide of the present disclosure is recombinantly expressed or chemically synthesized and are used directly, after radiolabeling, or after conjugation to a fluorophore or therapeutic compound, such as such as a kidney therapeutic from TABLE 4 or TABLE 5.

The peptide or peptide conjugate is administered in a pharmaceutical composition to a subject as a therapeutic for hypertensive kidney damage. The peptide is selected from any one of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. One or more peptides or peptide conjugates of the present disclosure are administered to a subject. A subject can be a human or an animal. The pharmaceutical composition is administered subcutaneously, intravenously, orally, or injected directly into a joint. The peptides or peptide conjugates target cartilage affected by hypertensive kidney damage.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 73

### Treatment for Diabetic Nephropathy

This example illustrates treatment of diabetic nephropathy using peptides or peptide conjugates of this disclosure. A peptide of the present disclosure is recombinantly expressed or chemically synthesized and are used directly, after radiolabeling, or after conjugation to a fluorophore or therapeutic compound, such as such as a kidney therapeutic from TABLE 3 or TABLE 4.

The peptide or peptide conjugate is administered in a pharmaceutical composition to a subject as a therapeutic for diabetic nephropathy. The peptide is selected from any one of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. One or more peptides or peptide conjugates of the present disclosure are administered to a subject. A subject can be a human or an animal. The pharmaceutical composition is administered subcutaneously, intravenously, orally, or injected directly into a joint. The peptides or peptide conjugates target cartilage affected by diabetic nephropathy.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 74

### Treatment for Renal Fibrosis

This example illustrates treatment of renal fibrosis using peptides or peptide conjugates of this disclosure. A peptide of the present disclosure is recombinantly expressed or chemically synthesized and are used directly, after radiolabeling, or after conjugation to a fluorophore or therapeutic compound, such as such as a kidney therapeutic from TABLE 3 or TABLE 4.

The peptide or peptide conjugate is administered in a pharmaceutical composition to a subject as a therapeutic for renal fibrosis. The peptide is selected from any one of the peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. One or more peptides or peptide conjugates of the present disclosure are administered to a subject. A subject can be a human or an animal. The pharmaceutical composition is administered subcutaneously, intravenously, orally, or injected directly into a joint. The peptides or peptide conjugates target cartilage affected by renal fibrosis.

The peptide can be a peptide of SEQ ID NO: 115. The peptide can be a peptide of SEQ ID NO: 234. The peptide can also be a peptide of any one of SEQ ID NO: 109-SEQ ID NO: 126 or SEQ ID NO: 129-SEQ ID NO: 133. The peptide can be any peptide with the sequence selected SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263. Such peptide-drug conjugates can be made using either a cleavable or stable linker as described herein (e. g., EXAMPLES 19 and 20).

### Example 75

### Peptide Variants Based on Multiple Sequence Alignment

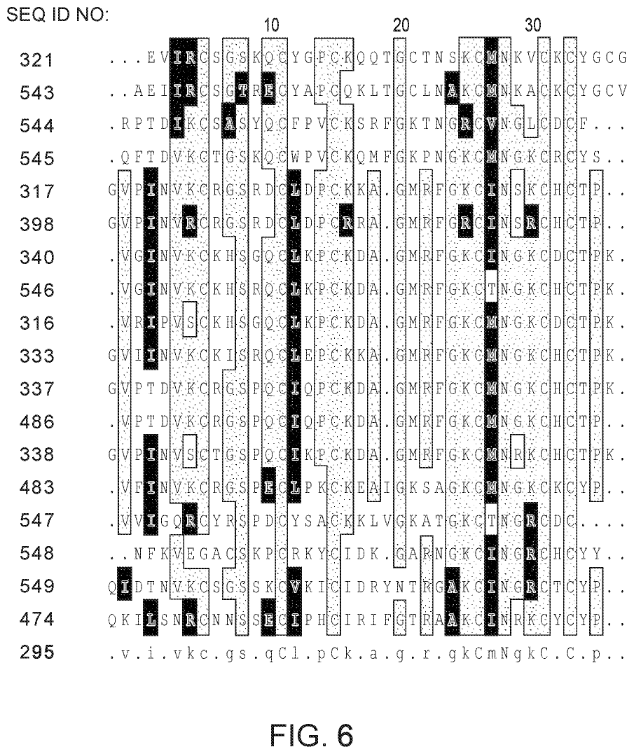

This example illustrates using multiple sequence alignment to design peptide variants with increased stability and decreased immunogenicity. An alignment was generated using R language and an “msa” software package, which codes for R language specific for multiple alignments (Bodenhofer, U et al. Bioinformatics, 31 (24): 3997-3999 (2015)). FIG. 6 illustrates a multiple sequence alignment of SEQ ID NO: 198-SEQ ID NO: 215. The alignment identified permissive or preferred amino acids at a given location, and provided a guide for discovery of novel peptide variants that could be generated and that could retain essential properties such as structure, function, peptide folding, biodistribution, or stability. SEQ ID NO: 21 and SEQ ID NO: 87 are consensus sequences based on the above multiple sequence alignment. SEQ ID NO: 21 is the same sequence as SEQ ID NO: 87 but with an N-terminal “GS.” SEQ ID NO: 219-SEQ ID NO: 222 show peptide sequences, wherein the SEQ ID NO: 87 and SEQ ID NO: 21 consensus sequences were fit to SEQ ID NO: 149 (also disclosed as SEQ ID NO: 46; non-GS version of SEQ ID NO: 149/SEQ ID NO: 46 are shown in SEQ ID NO: 128 and SEQ ID NO: 205), with or without an N-terminal GS. SEQ ID NO: 219-SEQ ID NO: 222 are variant consensus peptide sequences, included within the family of SEQ ID NO: 87 and SEQ ID NO: 21 consensus sequences, which variant consensus sequences include variants with improved properties of the peptides. Furthermore, based on the ability to substitute K residues with R residues the multiple sequence alignment identified peptides of the family of sequences of SEQ ID NO: 22 and SEQ ID NO: 88 as potential peptide variants that could be generated and that could retain essential properties such as structure, function, peptide folding, biodistribution, or stability. Additionally, the multiple sequence alignment identified SEQ ID NO: 104 as a conserved region within the sequences of the alignment, which may, at least in part, be important for maintaining the essential properties such as structure, function, peptide folding, biodistribution, binding, accumulation, retention, or stability. Other conserved regions within sequences of the present disclosure can be any one of SEQ ID NO: 227-SEQ ID NO: 232.

### Example 76

### Peptide Immunogenicity

This example illustrates the testing of the immunogenicity of a peptide. NetMHC II version 2.3 prediction software was used to identify immunogenic peptides based on a neural network alignment algorithm that predicts peptide binding to MHC Class II molecules.

The NetMHC II prediction software was utilized to determine the putative peptide binding capability to DR, DQ, and DP MHC II alleles and the strength of the interaction between peptide and MHC II molecules. TABLE 6 shows the resulting immunogenicity score of some select peptides. The numbers of strong versus weak peptides are tallied into each major MHC allele group (DR, DQ, and DP). Additionally, the numbers of ‘unique strong’ and ‘unique weak core’ peptides are also tallied. This data were used to predict which peptides are less likely to induce an immunogenic response in patients. For example, the stronger a peptide binds to an allele, the more likely itis to be presented in a MHC/peptide combination on an antigen presenting cell, thus triggering an immune response, and a peptide that is predicted to bind to fewer alleles is more likely to have weaker binding to given alleles and should be less immunogenic.

TABLE 6
Immunogenicity Scores of Peptides

| | Strong | Unique | Weak | Unique |
|---|---|---|---|---|
| | Binding | Strong | Binding | Weak |
| SEQ | Alleles | Core | Alleles | Core |
| ID NO: | (DR + DQ + DP) | Peptides | (DR + DQ + DP) | Peptides |
| 149 | 1 + 0 + 0 | 1 + 0 + 0 | 7 + 1 + 0 | 7 + 2 + 0 |
| 52 | 0 + 0 + 0 | 0 + 0 + 0 | 4 + 1 + 3 | 6 + 1 + 1 |
| 53 | 0 + 0 + 0 | 0 + 0 + 0 | 4 + 1 + 3 | 6 + 2 + 1 |
| 54 | 1 + 0 + 0 | 2 + 0 + 0 | 5 + 1 + 3 | 7 + 1 + 1 |
| 55 | 1 + 0 + 0 | 2 + 0 + 0 | 6 + 1 + 3 | 5 + 1 + 1 |
| 56 | 0 + 0 + 0 | 0 + 0 + 0 | 4 + 1 + 3 | 6 + 2 + 1 |
| 57 | 0 + 0 + 0 | 0 + 0 + 0 | 5 + 1 + 3 | 7 + 2 + 1 |
| 63 | 0 + 0 + 1 | 0 + 0 + 1 | 7 + 4 + 0 | 8 + 6 + 2 |
| 66 | 0 + 0 + 0 | 0 + 0 + 0 | 5 + 4 + 1 | 5 + 4 + 2 |
| 128 | 1 + 0 + 0 | 1 + 0 + 0 | 7 + 1 + 0 | 7 + 1 + 0 |
| 134 | 0 + 0 + 0 | 0 + 0 + 0 | 4 + 0 + 3 | 6 + 0 + 1 |
| 135 | 0 + 0 + 0 | 0 + 0 + 0 | 4 + 1 + 3 | 6 + 1 + 1 |
| 136 | 1 + 0 + 0 | 2 + 0 + 0 | 5 + 0 + 3 | 7 + 0 + 1 |
| 137 | 1 + 0 + 0 | 2 + 0 + 0 | 6 + 0 + 3 | 5 + 0 + 1 |
| 138 | 0 + 0 + 0 | 0 + 0 + 0 | 4 + 1 + 3 | 6 + 1 + 1 |
| 139 | 0 + 0 + 0 | 0 + 0 + 0 | 5 + 1 + 3 | 7 + 1 + 1 |
| 145 | 0 + 0 + 1 | 0 + 0 + 1 | 7 + 4 + 1 | 8 + 6 + 2 |
| 148 | 0 + 0 + 0 | 0 + 0 + 0 | 5 + 4 + 1 | 5 + 5 + 2 |
| 109 | 0 + 0 + 0 | 0 + 0 + 0 | 2 + 0 + 0 | 2 + 0 + 0 |
| 110 | 0 + 0 + 0 | 0 + 0 + 0 | 6 + 2 + 1 | 5 + 3 + 2 |
| 111 | 0 + 0 + 0 | 0 + 0 + 0 | 4 + 2 + 1 | 3 + 2 + 1 |
| 112 | 0 + 0 + 0 | 0 + 0 + 0 | 4 + 2 + 1 | 3 + 3 + 1 |
| 113 | 0 + 0 + 0 | 0 + 0 + 0 | 4 + 2 + 1 | 3 + 3 + 2 |
| 114 | 0 + 0 + 0 | 0 + 0 + 0 | 2 + 2 + 1 | 1 + 4 + 2 |
| 115 | 0 + 0 + 0 | 0 + 0 + 0 | 2 + 2 + 1 | 2 + 4 + 2 |
| 118 | 0 + 0 + 1 | 0 + 0 + 1 | 7 + 2 + 1 | 4 + 5 + 2 |
| 126 | 0 + 0 + 0 | 0 + 0 + 0 | 5 + 2 + 1 | 4 + 3 + 2 |

### Example 77

### Peptide Variants

This example illustrates the design of variant peptide sequences with increased stability, decreased regions of immunogenicity, and the substitution of a tyrosine for spectrophotometric reporting as compared to a parent peptide sequence. Potential mutations or corresponding substitutions to the parent peptide sequence, SEQ ID NO: 149 (also disclosed as SEQ ID NO: 46; non-GS version of SEQ ID NO: 149/SEQ ID NO: 46 are shown in SEQ ID NO: 128 and SEQ ID NO: 205), that may result in a peptide with increased stability, decreased immunogenicity, or increased absorbance at 270-280 nm (such as the substitution to a tyrosine or tryptophan residue for spectrophotometric reporting) were identified based on information from multiple sequence alignment from EXAMPLE 75 and immunogenicity testing from EXAMPLE 76.

In SEQ ID NO: 149, residue N7 is at risk for deamidation. Based on the multiple sequence alignment of SEQ ID NO: 198-SEQ ID NO: 215, the candidate residue mutations or corresponding substitutions to best reduce this risk were N7S and N7G. N7S was determined to be more likely to result in a peptide with desirable properties such as folding and stability as shown by matches in the alignment and conservationist presence in a peptide with high stability (SEQ ID NO: 206).

Residue D18 is at risk for cleavage. Based on the multiple sequence alignment, the candidate residue mutations or corresponding substitutions to best reduce cleavage at D18 are D18E and D18Q. D18E is the preferred choice based on retaining charge.

Residue M25 is at risk for oxidation. Based on the multiple sequence alignment, the candidate residue mutations or corresponding substitutions to best reduce oxidation were M25T and M25A. Based on the immunogenicity score of peptides with each mutation or corresponding substitutions, it was determined that M25T is the better mutation or corresponding substitutions, as it eliminates a significant source of immunogenicity as compared to SEQ ID NO: 108 as well as the variant with M25A, which did not eliminate the predicted immunogenicity of the parent peptide of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205).

Residue N32 is at risk for deamidation, at least in part due to the neighboring residue S33. However, N32 is conserved across Kv1.3 binding cystine-dense peptides in the alignment of EXAMPLE 75, and implicated in receptor binding (Peigneur, S., Biochemistry, 55(32): 2927-35 (2016)). For certain applications, peptides are designed to maintain this binding interaction, and for other applications, peptides are designed to remove this binding interaction. To maintain functionality, one candidate residue mutation or corresponding substitution based on the multiple sequence alignment is S33R, which would impact deamidation. However, it resulted in a predicted increased immunogenicity score. Another candidate residue mutation or corresponding substitution is S33G, but this may result in higher deamidation rates. If N32 is mutated, the best candidate residue mutation or corresponding substitution based the multiple sequence alignment in combination with the immunogenicity score was N32Q despite it having a slight increase in immunogenicity. Other options are N32A, N32S, or N32T. Alternatively, to remove functionality, candidate mutations or corresponding substitutions based on the multiple sequence alignment are N32A and N32L, which are the preferred choices.

For the substitution to a tyrosine for spectrophotometric reporting, the best candidate locations were T38Y (which had the strongest precedence in the multiple sequence alignment and is found in several of the stable peptides (e.g., SEQ ID NO: 206, SEQ ID NO: 210, and SEQ ID NO: 211)), L17Y, and H36Y. However, T38Y may slightly increase immunogenicity with respect to the DR allele. Another option for spectrophometric absorbance is to substitute Trp for the Leu at position 17.

Based on the above analysis, the following short list of potential mutations or corresponding substitutions for SEQ ID NO: 149 were compiled: N7S; D18E; M25T; N32Q, N32A, N32S, N32T, N32L, S33G, and S33R (variants both to retain function and to remove function of binding ion channel); and L17Y, H36Y, and T38Y.

TABLE 7A provides some exemplary sequences using various combinations of these mutations or corresponding substitutions with reference to SEQ ID NO: 149. Corresponding substitutions located at the corresponding location in another peptide relative to those amino acid residues (or with reference to those amino acid residues) as located within a sequence of the disclosure can be obtained, for example, using a sequence alignment or other methodology.

TABLE 7A
Exemplary Sequence Variants of SEQ ID NO: 149
(also disclosed as SEQ ID NO: 46; non-GS version of
SEQ ID NO: 149/SEQ ID NO: 46 are shown in
SEQ ID NO: 128 and SEQ ID NO: 205)

| SEQ | | |
|---|---|---|
| ID | | |
| NO: | Mutations | Sequence |
| 149 | Parent | GSGVPINVRCRGSRDCLDPC |
| | | RRAGMRFGRCINSRCHCTP |
| 52 | N7S, D18E, | GSGVPISVRCRGSRDCLEPC |
| | M25T, S33G | RRAGTRFGRCINGRCHCTP |
| 53 | N7S, D18E, | GSGVPISVRCRGSRDCLEPC |
| | M25T, N32Q | RRAGTRFGRCIQSRCHCTP |
| 54 | N7S, D18E, | GSGVPISVRCRGSRDCLEPC |
| | M25T, S33R | RRAGTRFGRCINRRCHCTP |
| 55 | D18E, M25T | GSGVPINVRCRGSRDCLEPC |
| | | RRAGTRFGRCINSRCHCTP |
| 56 | D18E, M25T, | GSGVPINVRCRGSRDCLEPC |
| | N32Q | RRAGTRFGRCIQSRCHCTP |
| 57 | D18E, M25T, | GSGVPINVRCRGSRDCLEPC |
| | N32Q, T38Y | RRAGTRFGRCIQSRCHCYP |
| 58 | L17Y, D18E, | GSGVPINVRCRGSRDCYEPC |
| | M25T, N32Q | RRAGTRFGRCIQSRCHCTP |
| 59 | D18E, M25T, | GSGVPINVRCRGSRDCLEPC |
| | N32Q, H36Y | RRAGTRFGRCIQSRCYCTP |
| 60 | N7S, D18E, | GSGVPISVRCRGSRDCLEPC |
| | M25T, N32Q, | RRAGTRFGRCIQSRCHCYP |
| | T38Y | |
| 61 | N7S, L17Y, | GSGVPISVRCRGSRDCYEPC |
| | D18E, M25T, | RRAGTRFGRCIQSRCHCTP |
| | N32Q | |
| 62 | N7S, D18E, | GSGVPISVRCRGSRDCLEPC |
| | M25T, N32Q, | RRAGTRFGRCIQSRCYCTP |
| | H36Y | |
| 63 | D18E, M25T, | GSGVPINVRCRGSRDCLEPC |
| | N32A, T38Y | RRAGTRFGRCIASRCHCYP |
| 64 | D18E, M25T, | GSGVPINVRCRGSRDCLEPC |
| | N32S, T38Y | RRAGTRFGRCISSRCHCYP |
| 65 | D18E, M25T, | GSGVPINVRCRGSRDCLEPC |
| | N32T, T38Y | RRAGTRFGRCITSRCHCYP |
| 66 | D18E, M25T, | GSGVPINVRCRGSRDCLEPC |
| | T38Y | RRAGTRFGRCINSRCHCYP |
| 241 | D18E, M25T, | GSGVPINVRCRGSRDCLEPC |
| | N32L | RRAGTRFGRCILSRCHCTP |
| 242 | D18E, M25T, | GSGVPINVRCRGSRDCLEPC |
| | N32L, T38Y | RRAGTRFGRCILSRCHCYP |
| 243 | D18E, M25T, | GSGVPINVRCRGSRDCLEPC |
| | N32L, H36Y | RRAGTRFGRCILSRCYCTP |
| 244 | N32Q, | GSGVPINVRCRGSRDCLDPC |
| | | RRAGMRFGRCIQSRCHCTP |
| 245 | N32A, | GSGVPINVRCRGSRDCLDPC |
| | | RRAGMRFGRCIASRCHCTP |
| 246 | N32S, | GSGVPINVRCRGSRDCLDPC |
| | | RRAGMRFGRCISSRCHCTP |
| 247 | N32T, | GSGVPINVRCRGSRDCLDPC |
| | | RRAGMRFGRCITSRCHCTP |
| 248 | N32L | GSGVPINVRCRGSRDCLDPC |
| | | RRAGMRFGRCILSRCHCTP |

TABLE 7B similarly provides some exemplary sequences using various combinations of these mutations or corresponding substitutions at corresponding locations but as applied to SEQ ID NO:128 (GVPVRCRGSRDCLDPCRRAGGRFGRCIRNSRCHCTP; also disclosed herein as SEQ ID NO: 205; GS versions of SEQ ID NO: 128 and SEQ ID NO: 205 are shown in SEQ ID NO:546 and SEQ TD NOS 149).

TABLE 7B
Exemplary Sequence Variants of SEQ ID NO: 128
(also disclosed herein as SEQ ID NO: 205)

| SEQ | | |
|---|---|---|
| ID | | |
| NO: | Mutations | Sequence |
| 128 | Parent | GVPINVRCRGSRDCLDPCR |
| | | RAGMRFGRCINSRCHCTP |
| 134 | N5S, D16E, | GVPISVRCRGSRDCLEPCR |
| | M23T, S31G | RAGTRFGRCINGRCHCTP |
| 135 | N5S, D16E, | GVPISVRCRGSRDCLEPCR |
| | M23T, N30Q | RAGTRFGRCIQSRCHCTP |
| 136 | N5S, D16E, | GVPISVRCRGSRDCLEPCR |
| | M23T, S31R | RAGTRFGRCINRRCHCTP |
| 137 | D16E, M23T | GVPINVRCRGSRDCLEPCR |
| | | RAGTRFGRCINSRCHCTP |
| 138 | D16E, M23T, | GVPINVRCRGSRDCLEPCR |
| | N30Q | RAGTRFGRCIQSRCHCTP |
| 139 | D16E, M23T, | GVPINVRCRGSRDCLEPCR |
| | N30Q, T36Y | RAGTRFGRCIQSRCHCYP |
| 140 | L15Y, D16E, | GVPINVRCRGSRDCYEPCR |
| | M23T, N30Q | RAGTRFGRCIQSRCHCTP |
| 141 | D16E, M23T, | GVPINVRCRGSRDCLEPCR |
| | N30Q, H34Y | RAGTRFGRCIQSRCYCTP |
| 142 | N5S, D16E, | GVPISVRCRGSRDCLEPCR |
| | M23T, N30Q, | RAGTRFGRCIQSRCHCYP |
| | T36Y | |
| 143 | N5S, L15Y, | GVPISVRCRGSRDCYEPCR |
| | D16E, M23T, | RAGTRFGRCIQSRCHCTP |
| | N30Q | |
| 144 | N5S, D16E, | GVPISVRCRGSRDCLEPCR |
| | M23T, N30Q, | RAGTRFGRCIQSRCYCTP |
| | H34Y | |
| 145 | D16E, M23T, | GVPINVRCRGSRDCLEPCR |
| | N30A, T36Y | RAGTRFGRCIASRCHCYP |
| 146 | D16E, M23T, | GVPINVRCRGSRDCLEPCR |
| | N30S, T36Y | RAGTRFGRCISSRCHCYP |
| 147 | D16E, M23T, | GVPINVRCRGSRDCLEPCR |
| | N30T, T36Y | RAGTRFGRCITSRCHCYP |
| 148 | D16E, M23T, | GVPINVRCRGSRDCLEPCR |
| | T36Y | RAGTRFGRCINSRCHCYP |
| 249 | D16E, M23T, | GVPINVRCRGSRDCLEPCR |
| | N30L | RAGTRFGRCILSRCHCTP |
| 250 | D16E, M23T, | GVPINVRCRGSRDCLEPCR |
| | N30L, T36Y | RAGTRFGRCILSRCHCYP |
| 251 | D16E, M23T, | GVPINVRCRGSRDCLEPCR |
| | N30L, H34Y | RAGTRFGRCILSRCYCTP |
| 252 | N30Q, | GVPINVRCRGSRDCLDPCR |
| | | RAGMRFGRCIQSRCHCTP |
| 253 | N30A, | GVPINVRCRGSRDCLDPCR |
| | | RAGMRFGRCIASRCHCTP |
| 254 | N30S, | GVPINVRCRGSRDCLDPCR |
| | | RAGMRFGRCISSRCHCTP |
| 255 | N30T, | GVPINVRCRGSRDCLDPCR |
| | | RAGMRFGRCITSRCHCTP |
| 256 | N30L | GVPINVRCRGSRDCLDPCR |
| | | RAGMRFGRCILSRCHCTP |

### Example 78

### Peptide-Budesonide Complex

This example describes conjugation of a peptide of any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 to budesonide. Budesonide is readily complexed, conjugated, or fused to any peptide disclosed herein via standard chemistries such as those described in, but not limited to, Bioconjugate Techniques by Greg Hermanson (Elsevier Inc., 3rd edition, 2013) or by any of the methods described in EXAMPLES 23-26.

The peptide-budesonide conjugates are administered to a subject in need thereof and home, target, are directed to, are retained by, accumulate in, migrate to, and/or bind to cartilage and/or kidneys. The subject is a human or animal and has inflammation in the cartilage or kidney tissues. Upon administration and homing of peptide-budesonide conjugates, the inflammation in the cartilage and/or kidney tissues is alleviated.

### Example 79

### Peptide-Dexamethasone Complex

This example describes conjugation of a peptide of any one SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 to dexamethasone. Dexamethasone is readily complexed, conjugated, or fused to any peptide disclosed herein via standard chemistries such as those described in, but not limited to, Bioconjugate Techniques by Greg Hermanson (Elsevier Inc., 3rd edition, 2013) or by any of the methods described in EXAMPLES 23-26.

The peptide-dexamethasone conjugates are administered to a subject in need thereof and home, target, are directed to, are retained by, accumulate in, migrate to, and/or bind to cartilage and/or kidneys. The subject is a human or animal and has inflammation in the cartilage or kidney tissues. Upon administration and homing of peptide-dexamethasone conjugates, the inflammation in the cartilage and/or kidney tissues is alleviated.

### Example 80

### Peptide-Triamcinalone Acetonide Complex

This example describes conjugation of a peptide of any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 to triamicinalone acetonide. Triamicinalone acetonide is readily complexed, conjugated, or fused to any peptide disclosed herein via standard chemistries such as those described in, but not limited to, Bioconjugate Techniques by Greg Hermanson (Elsevier Inc., 3rd edition, 2013) or by any of the methods described in EXAMPLES 23-26.

The peptide-triamicinalone acetonide conjugates are administered to a subject in need thereof and home, target, are directed to, are retained by, accumulate in, migrate to, and/or bind to cartilage and/or kidneys. The subject is a human or animal and has inflammation in the cartilage or kidney tissues. Upon administration and homing of peptide-triamicinalone acetonide conjugates, the inflammation in the cartilage and/or kidney tissues is alleviated.

### Example 81

### Peptide-Desciclesonide Complex

This example describes conjugation of a peptide of any one of SEQ ID NO: 115, SEQ ID NO: 234, SEQ ID NO: 139, SEQ ID NO: 242, or SEQ ID NO: 110 to desciclesonide. Desciclesonide is readily complexed, conjugated, or fused to any peptide disclosed herein via standard chemistries such as those described in, but not limited to, Bioconjugate Techniques by Greg Hermanson (Elsevier Inc., 3rd edition, 2013) or by any of the methods described in EXAMPLES 23-26.

The peptide-desciclesonide conjugates are administered to a subject in need thereof and home, target, are directed to, are retained by, accumulate in, migrate to, and/or bind to cartilage and/or kidneys. The subject is a human or animal and has inflammation in the cartilage or kidney tissues. Upon administration and homing of peptide-desciclesonide conjugates, the inflammation in the cartilage and/or kidney tissues is alleviated.

The peptide can be any one of SEQ ID NO: 52-SEQ ID NO: 66, SEQ ID NO: 241-SEQ ID NO: 248, SEQ ID NO: 134-SEQ ID NO: 148, SEQ ID NO: 249-SEQ ID NO: 256, SEQ ID NO: 111-SEQ ID NO: 126, or SEQ ID NO: 233-SEQ ID NO: 240. The peptide can be any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 233-SEQ ID NO: 256, or SEQ ID NO: 260-SEQ ID NO: 263.

### Example 82

### Method of Peptide Synthesis

This example describes the synthesis of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205).

A peptide of SEQ ID NO: 149 was made using Solid Phase Peptide Synthesis (SPPS). After release of the peptide from the solid phase, the peptide was purified prior to folding by oxidation in solution. The folded peptide was further purified by reversed-phase chromatography and lyophilized as a TFA salt. The final SEQ ID NO: 149 peptide product had a purity of 96.1% and a mass of 4,301.7 Da, which confirmed its identity as a peptide of SEQ ID NO: 149.

### Example 83

### Whole Body Autoradiography of Cartilage Homing Peptides

This example illustrates peptide homing to cartilage mice 5 minutes to 48 hours after administration of a radiolabeled peptide. Signal from the radiolabeled peptides was found in all types of cartilage at each time point examined. Each peptide was radiolabeled by methylating lysines at the N-terminus as described in EXAMPLE 2. As such, the peptide may contain methyl or dimethyl lysines and a methylated or dimethlyated amino terminus. A dose of 100 nmol radiolabeled peptide was administered via tail vein injection in Female Harlan athymic nude mice, weighing 20-25 g. The experiment was done in duplicate (n=2 animals per group). Each radiolabeled peptide was allowed to freely circulate within the animal for the described time period before the animals were euthanized and sectioned.

Whole body autoradiography (WBA) sagittal sectioning was performed as follows. At the end of the dosing period, mice were frozen in a hexane/dry ice bath and then embedded in a frozen block of carboxymethylcellulose. Whole animal sagittal slices were prepared that resulted in thin frozen sections for imaging. Sections were allowed to dessicate in a freezer prior to imaging. For the autoradiography imaging, tape mounted thin sections were freeze dried and radioactive samples were exposed to phosphoimager plates. These plates were developed and the signal (densitometry) from each organ was normalized to the signal found in the cardiac blood of each animal. A signal in tissue darker than the signal expected from blood in that tissue indicates accumulation in a region, tissue, structure, or cell.

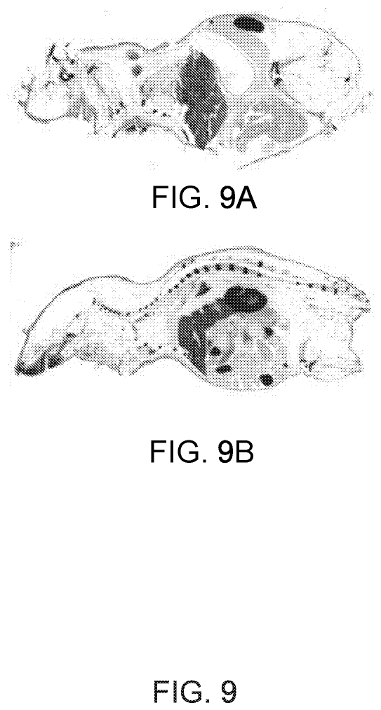

FIG. 9 illustrates autoradiography image of frozen sections from a mouse, 3 hours after administration of 100 nmol of a radiolabeled peptide of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205). FIG. 9A illustrates the 14C signal in a frozen section of a mouse, 3 hours after administration of 100 nmol of a radiolabeled peptide of SEQ ID NO: 149. The ¹⁴C signal identifies the radiolabeled peptide distribution in the cartilage of the mouse. FIG. 9B illustrates the ¹⁴C signal in a different frozen section of a mouse, 3 hours after administration of 100 nmol of a radiolabeled peptide of SEQ ID NO: 149. The ¹⁴C signal identifies the radiolabeled peptide distribution in the cartilage of the mouse.

TABLE 8 shows the signal of radiolabeled peptides of SEQ ID NO: 150 and SEQ ID NO: 149 in intervertebral discs (IVD) and knee joints as a percentage of the blood. Because the peptides may arrive at the joint within five minutes, a therapeutic effect from the peptide or a complexed, conjugated, or fused active agent may begin quickly. A therapeutic effect could be long lasting, due to continued presence of detected agents at 48 hours and/or due to long lasting pharmacodynamics effects.

TABLE 8
Signal of Radiolabled Peptides of SEQ ID
NO: 150 and SEQ ID NO: 149 in IVD and
Knee Joints as a Percentage of Blood

| | SEQ ID | SEQ ID | SEQ ID |
|---|---|---|---|
| | NO: 150 | NO: 149 | NO: 149 |
| Hours | IVD | IVD | Knee |
| 0.08 | | 164 | 404 |
| 0.5 | | 369 | 510 |
| 1 | | 961 | 1114 |
| 3 | 1779 | 3213 | 4059 |
| 8 | | 3777 | 4990 |
| 24 | 833 | 5391 | 2137 |
| 48 | | 3320 | 843 |

This data illustrates peptides of SEQ ID NO: 150 and SEQ ID NO: 149 homed to and accumulated in the cartilage of the animals. The peptide of SEQ ID NO: 149 is a K to R variant of a peptide of SEQ ID NO: 150. These data show that K to R variants of cartilage homing peptides retained their cartilage homing properties.

SEQ ID NO: 218 (GSGVPINVRSRGSRDSLDPSRRAGMRFGRSINSRSHSTP) is a linearized version of SEQ ID NO: 149, where the knotted scaffold of the peptide was removed by mutating out the cysteine residues that form the disulfide bonds of the peptide to serine residues, but retaining the rest of the sequence. TABLE 9 shows quantification of signal as a percentage of signal in blood from a linearized radiolabeled SEQ ID NO: 218 peptide in intervertebral discs (IVD).

TABLE 9
Signal of Radiolabled Peptides
of SEQ ID NO: 218 in IVD
as a Percentage of Blood

| | 3 hr | 3 hr | 24 hr |
|---|---|---|---|
| | Ligated | Intact | Intact |
| | Kidneys | Kidneys | Kidneys |
| IVD | 117 | 177 | 104 |

The peptide of SEQ ID NO: 218, a linearized version of the peptide of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205), homed to cartilage to a much lesser extent than the folded knotted peptide (SEQ ID NO: 149). The signal of the folded knotted peptide of SEQ ID NO: 149 was ˜20-fold greater at 3 hours and ˜50-fold greater at 24 hours (TABLE 8) as compared to the linearized peptide of SEQ ID NO: 218 (TABLE 9). These results indicate that in addition to changes in primary sequence or peptide charge, homing to cartilage can also be related to changes in conformation, or tertiary structure. Namely, in some cases, folded cystine-dense peptides can be exemplary cartilage homers in comparison to unfolded, linearized peptides of the same primary sequence (except for the mutated cysteine residues).

### Example 84

### Fluorescence of Cartilage Homing Peptides

This example illustrates peptide homing to cartilage mice after administration of a peptide-fluorophore complex. A peptide of SEQ ID NO: 149 was chemically conjugated to one molecule of Cyanine 5.5, and then imaged using the methods of EXAMPLE 11.

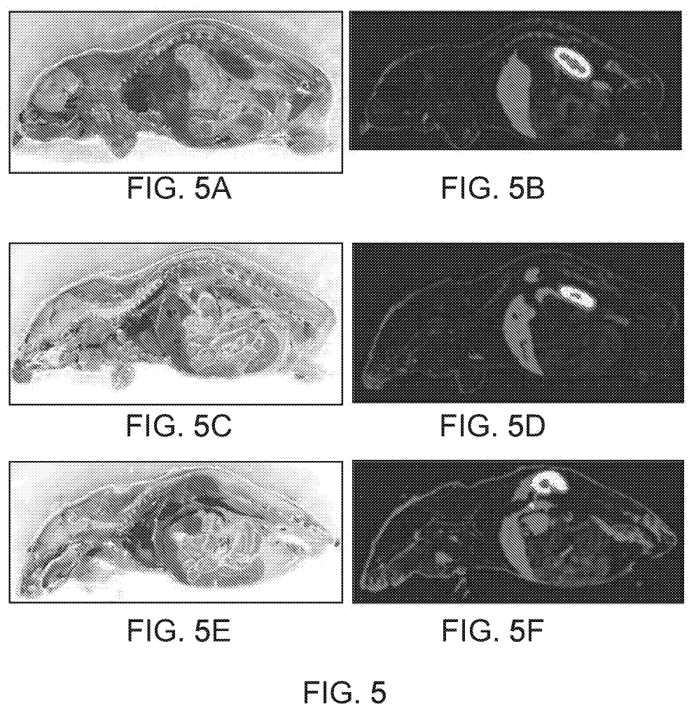

FIG. 5 shows white light images and corresponding whole body fluorescence images of a mouse administered 10 nmol of a peptide of SEQ ID NO: 149 conjugated to a Cy5.5 fluorophore (SEQ ID NO: 149A) at 24 hours post-administration. FIG. 5A illustrates an image of a frozen section of a mouse, 24 hours after administration of 10 nmol of a peptide of SEQ ID NO: 149 conjugated to a Cy5.5 fluorophore (SEQ ID NO: 149A). FIG. 5B illustrates the fluorescence signal in the mouse, corresponding to the section shown in FIG. 5A, 24 hours after administration of 10 nmol of a peptide of SEQ ID NO: 149 conjugated to a Cy5.5 fluorophore (SEQ ID NO: 149A). FIG. 5C illustrates an image of a different frozen section of the mouse, 24 hours after administration of 10 nmol of a peptide of SEQ ID NO: 46 (also disclosed herein as SEQ ID NO: 149) conjugated to a Cy5.5 fluorophore (SEQ ID NO: 149A). FIG. 5D illustrates the fluorescence signal in the mouse, corresponding to the section shown in FIG. 5C, 24 hours after administration of 10 nmol of a peptide of SEQ ID NO: 149 conjugated to a Cy5.5 fluorophore (SEQ ID NO: 149A). FIG. 5E illustrates an image of a different frozen section of the mouse, 24 hours after administration of 10 nmol of a peptide of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205) conjugated to a Cy5.5 fluorophore (SEQ ID NO: 149A). FIG. 5F illustrates a fluorescence signal in the mouse, corresponding to the section shown in FIG. 5E, 24 hours after administration of 10 nmol of a peptide of SEQ ID NO: 149 conjugated to a Cy5.5 fluorophore (SEQ ID NO: 149A).

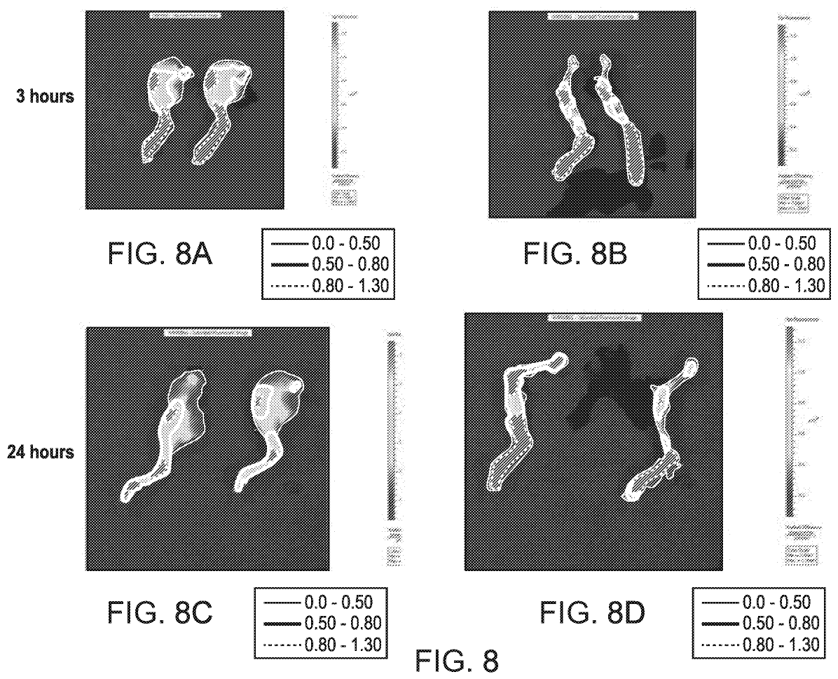

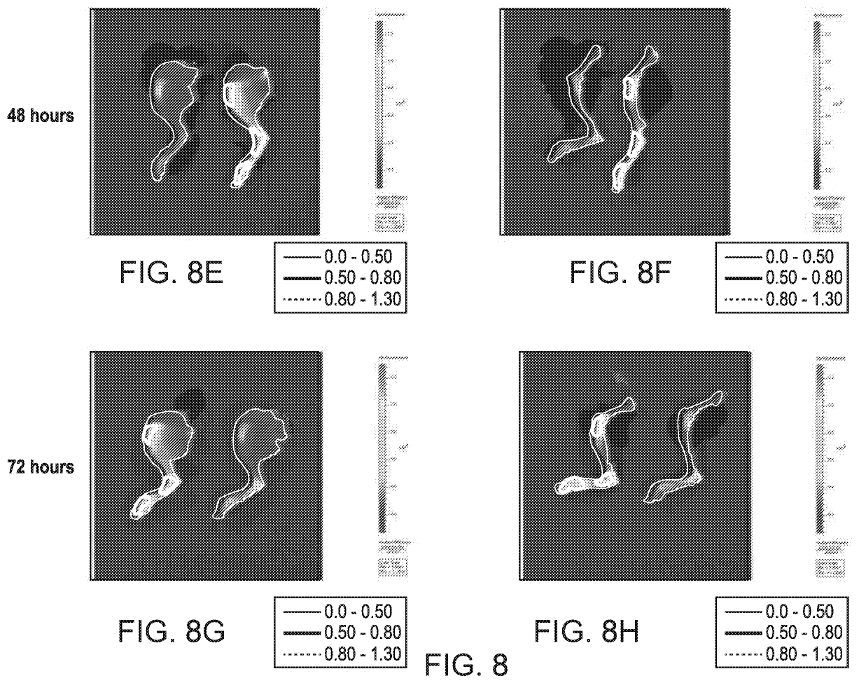

FIG. 8 shows IVIS fluorescence imaging of an isolated hind limb from a first mouse and an isolated hind limb from a second mouse after administration of 10 nmol SEQ ID NO: 108 peptide conjugated to a Cy5.5 fluorophore (SEQ ID NO: 149A). FIG. 8A shows the right hind limb with skin removed from a first mouse and from a second mouse 3 hours after peptide administration. FIG. 8B shows the right hind limb with muscle removed from a first mouse and from a second mouse 3 hours after peptide administration. FIG. 8C shows the right hind limb with skin removed from a first mouse and from a second mouse 24 hours after peptide administration. FIG. 8D shows the right hind limb with muscle removed from a first mouse and from a second mouse 24 hours after peptide administration. FIG. 8E shows the right hind limb with skin removed from a first mouse and from a second mouse 48 hours after peptide administration. FIG. 8F shows the right hind limb with muscle removed from a first mouse and from a second mouse 48 hours after peptide administration. FIG. 8G shows the right hind limb with skin removed from a first mouse and from a second mouse 72 hours after peptide administration. FIG. 811 shows the right hind limb with muscle removed from a first mouse and from a second mouse 72 hours after peptide administration. Peptide fluorescence was observed in the knee joints of isolated right hind limbs at all time points tested. The peptides of this disclosure can be complexed, conjugated, or fused to one or more Cy5.5 fluorophores.

### Example 85

### Peptide Resistance Under Various Conditions

This example illustrates peptide stability under various stress conditions such as high temperature, low pH, reducing agents, and proteases. To determine resistance to high temperatures, cystine-dense peptides (CDPs) were incubated at 0.5 mM in PBS at 75° C. or 100° C. for 1 h and pelleted, and the supernatant was analyzed with reversed-phase chromatography (RPC). To determine resistance to proteolytic digestion, CDPs were mixed with 50 U of porcine pepsin, in simulated gastric fluid at pH 1.0, or 50 U of porcine trypsin in PBS, incubated for 30 minutes at 37° C. and analyzed with RPC. Oxidized and reduced forms (prepared through addition 10 mM DTT) were compared. Circular Dichroism spectroscopy was used in order to measure the secondary structure of peptides with a Jasco J-720W spectropolarimeter in a cell with a 1.0-mm path length, and CDPs were diluted into 20 mM phosphate buffer, pH 7.4, at a concentration of 15-25 μM. These conditions were expected to denature or degrade conventional globular proteins and many peptides. In TABLE 10, “high” resistance indicated a high amount of the peptide remained or was retained as unmodified under the given experimental conditions and “low” resistance indicated a low amount of the peptide remained or was retained unmodified under the given experimental conditions. Notably, the experimental conditions described in this example were more extreme stress conditions than to many standard in vivo or physiologic conditions, in vitro conditions, conditions during manufacturing, and handling conditions. As such, even “low” resistance can indicate meaningful resistance to these stress conditions that may have applicability for a number of uses described herein. The data from these studies are shown in TABLE 10. The peptide tested, SEQ ID NO: 150, showed high resistance to one or more of the conditions tested.

TABLE 10
Resistance of SEQ ID NO: 150 to Various Conditions

| SEQ | Resistance to | Resistance | Resistance | Resistance | Resistance |
|---|---|---|---|---|---|
| ID NO: | Reduction | to 75° C. | to 100° C. | to Pepsin | to Trypsin |
| 150 | High | High | High | High | High |

### Example 86

### Truncated Peptide Variant Synthesis

This example illustrates the synthesis of truncated peptide variants of a parent peptide. SEQ ID NO: 149 (39 amino acids) (SEQ ID NO: 149 is also disclosed as SEQ ID NO: 46; non-GS version of SEQ ID NO: 149/SEQ ID NO: 46 are shown in SEQ ID NO: 128 and SEQ ID NO: 205) was synthesized using Solid Phase Peptide Synthesis (SPPS). After release of the peptide from the solid phase, the peptide was purified prior to folding by oxidation in solution. The folded peptide was further purified by reversed-phase chromatography and lyophilized as a TFA salt. The final product had a purity of 96.1% and a mass of 4,301.7 Da, which confirmed its identity as a peptide of SEQ ID NO: 149.

A truncated form of SEQ ID NO: 149, which is only 31 amino acids and is SEQ ID NO: 110, was synthesized using SPPS. After release of the peptide from the solid phase, the peptide was folded by oxidation in solution. The folded peptide was further purified by reversed-phase chromatography and lyophilized as a TFA salt. The truncation consisted of omitting the last eight amino acids from the N-terminus of SEQ ID NO: 149 to produce SEQ ID NO: 110.

The truncation was designed to remove a section of the peptide as to not disturb the tertiary structure and retain all disulfide bridges and charges. The synthesis was successful and the final product had a purity of 96.8% and a mass of 3,578.0 Da, which confirmed its identity as a peptide of SEQ ID NO: 110

A major cost of manufacturing peptides occurs during the solid phase synthesis. The cost of raw materials used in SPPS (i.e., Fmoc amino acids and organic solvents) along with labor costs and manufacturing time (e.g., 1-2 days per amino acid addition step) are major contributors to the overall cost of the peptide. Therefore, reducing the length of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205) by eight amino acids (20%) to create the SEQ ID NO: 110 lowers both the time (labor, facility time) and the amount of raw materials required and thus lowers the cost and time needed to produce the final product.

The same manufacturing advantages can also apply to any one of the peptides comprising SEQ ID NO: 24-SEQ ID NO: 44, SEQ ID NO: 47-SEQ ID NO: 51, SEQ ID NO: 111-SEQ ID NO: 126, SEQ ID NO: 106-SEQ ID NO: 108, SEQ ID NO: 129-SEQ ID NO: 133, SEQ ID NO: 221-224, SEQ ID NO: 233-SEQ ID NO: 240, SEQ ID NO: 260-263.

### Example 87

### Truncated Peptide Conjugation

This example tests whether a truncated peptide of this disclosure has accessible N-terminal primary amines for conjugation. The full length peptide of SEQ ID NO: 149 and truncated variant peptide of SEQ ID NO: 110 each contain a single primary amine in their sequence located at the N-terminus of the peptide chain. To determine whether the N-terminal primary amines are accessible for conjugation, a small-scale reaction was performed using the amine specific reactive dye Cy5.5-NHS ester on both the full length peptide of SEQ ID NO: 149 and the truncated variant peptide of SEQ ID NO: 110. The reactions were monitored by reversed-phase HPLC. The area of the peaks corresponding to the retention times of the unmodified full length peptide of SEQ ID NO: 149 was observed before initiation and after completion of the reaction. The peak corresponding to the full length peptide of SEQ ID NO: 149 showed a decrease in peak area after initiation of the reaction, and a new peak appeared with a different retention time, indicating the N-terminal primary amine of the full length peptide of SEQ ID NO: 149 was available and accessible for conjugation. Likewise, the area of the peak corresponding to the unmodified truncated variant peptide of SEQ ID NO: 110 showed a decrease in peak area after initiation of the reaction and a new peak appeared with a different retention time, indicating that the N-terminal primary amine of the truncated variant peptide of SEQ ID NO: 110 was available and accessible to conjugation.

### Example 88

### Truncated Peptide Variants

This example illustrates the design of variant peptide sequences with increased stability, decreased regions of immunogenicity, and the substitution of a tyrosine for spectrophotometric reporting as compared to a parent peptide sequence. Potential mutations to the parent peptide sequence, SEQ ID NO: 110, may result in a peptide with increased stability, decreased immunogenicity, or increased absorbance at 270-280 nm (such as the substitution to a tyrosine or tryptophan residue for spectrophotometric reporting) were identified based on information from multiple sequence alignment from EXAMPLE 75 and immunogenicity testing from EXAMPLE 76.

In SEQ ID NO: 110, residue D10 is at risk for cleavage. Based on the multiple sequence alignment, the candidate residue mutations to best reduce cleavage at D10 are D10E and D10Q. D10E is the preferred choice based on retaining charge.

Residue M17 is at risk for oxidation. Based on the multiple sequence alignment, the candidate residue mutations to best reduce oxidation were M17T and M17A. Based on the immunogenicity score of peptides with each mutation, it was determined that M17T is the better mutation, as it eliminates a significant source of immunogenicity as compared to SEQ ID NO: 110 as well as the variant with M17A, which did not eliminate the predicted immunogenicity of the parent peptide of SEQ ID NO: 110.

Residue N24 is at risk for deamidation, at least in part due to the neighboring residue S25. However, N24 is conserved across Kv1.3 binding cystine-dense peptides in the alignment of EXAMPLE 77, and implicated in receptor binding (Peigneur, S., Biochemistry, 55(32): 2927-35 (2016)). For certain applications, peptides are designed to maintain this binding interaction, and for other applications, peptides are designed to remove this binding interaction. To maintain functionality, one candidate residue mutation based on the multiple sequence alignment is S25R, which would impact deamidation. However, it resulted in a predicted increased immunogenicity score. Another candidate residue mutation is S25G, but this may result in higher deamidation rates. If N24 is mutated, the best candidate residue mutation based the multiple sequence alignment in combination with the immunogenicity score was N24Q despite it having a slight increase in immunogenicity. Other options are N24A, N24S, or N24T. Alternatively, to remove functionality, candidate mutations based on the multiple sequence alignment are N24A and N24L, which are the preferred choices.

For the substitution to a tyrosine for spectrophotometric reporting, the best candidate locations were T30Y (which had the strongest precedence in the multiple sequence alignment and is found in several of the stable peptides (e.g., SEQ ID NO: 206, SEQ ID NO: 210, and SEQ ID NO: 211)), and L9Y, and H28Y. However, T30Y may slightly increase immunogenicity with respect to the DR allele. Another option for spectrophometric absorbance is to substitute Trp for the Leu at position 9.

Additionally, variant peptide sequences with residues R1, R13, R21, and R26 mutated to K maintain similar stability and immunogenicity as the SEQ ID NO: 110.

Based on the above analysis, the following short list of potential mutations for SEQ ID NO: 110 were compiled: D10E; M17T; N24Q, N24A, N24S, N24T, N24L, S25G, and S25R (variants both to retain function and to remove function of binding ion channel); and L9Y, H28Y, T30Y, R1K, R13K, R14K, R21K and R26K.

Furthermore, mutations denoted above and in TABLE 11 below at the corresponding positions in SEQ ID NO: 27, SEQ ID NO: 28, and SEQ ID NO: 47-SEQ ID NO: 51, SEQ ID NO: 109, SEQ ID NO: 110, SEQ ID NO: 127-SEQ ID NO: 133, SEQ ID NO: 149, SEQ ID NO: 150, SEQ ID NO: 199, and SEQ ID NO: 260-SEQ ID NO: 263 can create variant peptide sequences which similarly can reduce immunogenicity, increase stability, increase manufacturability, or otherwise improve the properties of the peptides.

TABLE 11
Exemplary Sequence Variants of SEQ ID NO: 110

| SEQ | | |
|---|---|---|
| ID | | |
| NO: | Mutations | Sequence |
| 110 | Parent | RCRGSRDCLDPCRRAGMRFGRCINSRCHCTP |
| 111 | D10E, M17T, | RCRGSRDCLEPCRRAGTRFGRCINGRCHCTP |
| | S25G | |
| 112 | D10E, M17T, | RCRGSRDCLEPCRRAGTRFGRCINRRCHCTP |
| | S25R | |
| 113 | D10E, M17T | RCRGSRDCLEPCRRAGTRFGRCINSRCHCTP |
| 114 | D10E, M17T, | RCRGSRDCLEPCRRAGTRFGRCIQSRCHCTP |
| | N24Q | |
| 115 | D10E, M17T, | RCRGSRDCLEPCRRAGTRFGRCIQSRCHCYP |
| | N24Q, T30Y | |
| 116 | L9Y, D10E, | RCRGSRDCYEPCRRAGTRFGRCIQSRCHCTP |
| | M17T, N24Q | |
| 117 | D10E, M17T, | RCRGSRDCLEPCRRAGTRFGRCIQSRCYCTP |
| | N24Q, H28Y | |
| 118 | D10E, M17T, | RCRGSRDCLEPCRRAGTRFGRCIASRCHCYP |
| | N24A, T30Y | |
| 119 | D10E, M17T, | RCRGSRDCLEPCRRAGTRFGRCISSRCHCYP |
| | N24S, T30Y | |
| 120 | D10E, M17T, | RCRGSRDCLEPCRRAGTRFGRCITSRCHCYP |
| | N24T, T30Y | |
| 121 | R1K | KCRGSRDCLDPCRRAGMRFGRCINSRCHCTP |
| 122 | R13K | RCRGSRDCLDPCKRAGMRFGRCINSRCHCTP |
| 123 | R14K | RCRGSRDCLDPCRKAGMRFGRCINSRCHCTP |
| 124 | R21K | RCRGSRDCLDPCRRAGMRFGKCINSRCHCTP |
| 125 | R26K | RCRGSRDCLDPCRRAGMRFGRCINSKCHCTP |
| 126 | D10E, M17T, | RCRGSRDCLEPCRRAGTRFGRCINSRCHCYP |
| | T30Y | |
| 233 | D10E, M17T, | RCRGSRDCLEPCRRAGTRFGRCILSRCHCTP |
| | N24L | |
| 234 | D10E, M17T, | RCRGSRDCLEPCRRAGTRFGRCILSRCHCYP |
| | N24L, T30Y | |
| 235 | D10E, M17T, | RCRGSRDCLEPCRRAGTRFGRCILSRCYCTP |
| | N24L, H28Y | |
| 236 | N24Q | RCRGSRDCLDPCRRAGMRFGRCIQSRCHCTP |
| 237 | N24A | RCRGSRDCLDPCRRAGMRFGRCIASRCHCTP |
| 238 | N24S | RCRGSRDCLDPCRRAGMRFGRCISSRCHCTP |
| 239 | N24T | RCRGSRDCLDPCRRAGMRFGRCITSRCHCTP |
| 240 | N24L | RCRGSRDCLDPCRRAGMRFGRCILSRCHCTP |

### Example 89

### Peptide-Active Agent Use in Rodent Arthritis Models

This example illustrates peptide-active agent use in rodent arthritis models. A peptide of SEQ ID NO: 115, SEQ ID NO: 234, SEQ ID NO: 114, SEQ ID NO: 118, SEQ ID NO: 126, or SEQ ID NO: 109 or any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 is complexed, conjugated, or fused to an active agent. Optionally the active agent is a glucocorticoid such as desciclesonide and optionally the linker is a cleavable linker. The peptide-active agent conjugate is administered to animals in one or more rodent arthritis models.

Exemplary models include the streptococcal cell wall-induced arthritis (SCW) model. Streptococcal cell wall peptidoglycan polysaccharide (PGPS) is administered intra-articularly into the knee or ankle of a rodent such as a Lewis rat and inflammation and joint swelling is induced. After the initial administration subsides, such as after 14 days or 1 month post-administration, the arthritis is reactivated by administering PGPS intravenously, which causes another reaction in the same joint that was originally injected. The peptide-active agent conjugate is administered to the animal intravenously, subcutaneously, or intra-articularly into the joint that has been activated with PGPS, 1 hour, 3 hour, 8 hours, 24 hours, 1 day, 2 days, 3 days, 1 week, 2 weeks, or more prior to the joint reactivation, concurrent with, or after joint reactivation.

Exemplary models also include collagen-induced arthritis (CIA) model, a rodent such as a Lewis rat is immunized with intradermal injection of collagen in incomplete Fruend's adjuvant (day 0). A challenge dose is optionally performed 7 days later. Arthritis develops in one or more paws. The peptide-active agent is administered to the animal intravenously or subcutaneously once every 1, 2, 3, 4, 7, or 14 days, starting 0, 1, 2, 3, 4, 5, 6, 7, 8, 9, 10, 11, 12, 13, or 14 days after day 0.

In another model, inflammation is induced in a joint by injecting IL-1beta intra-articularly into the joint. The peptide-active agent is administered to the animal intravenously or subcutaneously 1 hour, 2 hour, 3 hours, 4 hours, 6 hours, 12 hours, 1 day, 2 days, 3 days, 4 days, 7 days, or 14 days before IL-1beta administration, concurrent with, or after IL-1beta administration.

Other optional models include adjuvant-induced or antigen-induced arthritis, adjuvant arthritis, or administration of anti-collagen or other antibodies.

By administering the peptide-active agent conjugate comprising a peptide of any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 in any of the above arthritis models, the arthritic reaction in the animal is blocked, slowed, mitigated, or reduced, as measured by readouts such as joint swelling (such as by diameter or volume), cytokine protein or mRNA levels (such as IL-1beta, IL-6, TNFalpha) in the joint or in the blood, histopathology, or pain measures such as gait.

### Example 90

### Peptide-Active Agent Use in Rodent Lupus Models

This example illustrates peptide-active agent use in rodent lupus models. Lupus is modeled in rodents using the spontaneous NZB/W or MRL/lpr or BXSB mice, or is induced in rodents using pristine or accelerated in rodents by administering IFNalpha in NZB/W mice, such as described by Celhar 2017 (Rheumatology (Oxford). 2017 Apr. 1; 56(suppl_1):i88-i99. Modelling clinical systemic lupus erythematosus: similarities, differences and success stories. Celhar T, Fairhurst A M). Symptoms of systemic lupus erythematosus are, optionally, present, such as autoantibodies, anti-dsNA, immune complex glomerolunephritis, ANA, synovitis arthritis, skin rash. Optionally, the animals develop lupus nephritis.

A peptide of SEQ ID NO: 115, SEQ ID NO: 234, SEQ ID NO: 114, SEQ ID NO: 118, SEQ ID NO: 126, or SEQ ID NO: 109 or any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 is complexed, conjugated, or fused to an active agent. Optionally the active agent is a glucocorticoid such as desciclesonide and optionally the linker is a cleavable linker. The peptide-active agent is administered to the animals intravenously or subcutaneously once every 1, 2, 3, 4, 7, or 14 or more days. By administering the test agent, the symptoms of lupus or nephritis are improved.

By administering the peptide-active agent conjugate comprising a peptide of any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 in any of the above lupus models, the lupus effects in the animal are blocked, slowed, mitigated or reduced.

### Example 91

### Immunogenicity Testing of Peptides

This example illustrates immunogenicity testing of peptides, including a peptide of SEQ ID NO: SEQ ID NO: 115, SEQ ID NO: 234, SEQ ID NO: 114, SEQ ID NO: 118, SEQ ID NO: 126, or SEQ ID NO: 109 or any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

Dendritic cells and/or CD4+ T cells and/or peripheral blood mononuclear cells are isolated from healthy or diseased, such as affected by rheumatoid arthritis, human donors. Multiple donors are tested. Any peptide of SEQ ID NO: 115, SEQ ID NO: 234, SEQ ID NO: 114, SEQ ID NO: 118, SEQ ID NO: 126, or SEQ ID NO: 109 or any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 is applied in separate wells to the cells. The cells are assessed for proliferation and for secretion of cytokines such as IL-2 or IFN-gamma. Control peptides, such as those known to be immunogenic, non-immunogenic, or of known immunogenic potential are also incubated with the cells. Peptides that are less immunogenic by inducing less cell activation are identified.

Separately, one or more peptides of SEQ ID NO: 115, SEQ ID NO: 234, SEQ ID NO: 114, SEQ ID NO: 118, SEQ ID NO: 126, or SEQ ID NO: 109 or any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 are injected into a mouse or rat or non-human primate one to four times, intravenously or subcutaneously. The serum from the animal is collected at various time points and tested for the presence of antibodies that bind or neutralize the peptide that was dosed. Control proteins or peptides, such as those known to be immunogenic, non-immunogenic, or of known immunogenic potential are also separately tested in the animals. Peptides that are less immunogenic by inducing less antibody formation are identified.

By administering the peptide-active agent conjugate comprising a peptide of any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 in any of the above models, the immunogenic effects in the animal are blocked, slowed, mitigated, or reduced.

### Example 92

### Accelerated Stability Testing of Peptides

This example illustrates accelerated stability testing of peptides. A peptide of any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263, or an active-agent conjugate thereof, is subjected to accelerated stability testing. The peptide or peptide-conjugate is formulated in a buffer and, optionally, lyophilized. This formulated material is incubated at 30° C.±2° C./65% relative humidity (RH)±5% RH or 40° C.±2° C./75% RH±5% RH or 25° C.±2° C./60%±RH 5% RH and analyzed. The peptide or peptide-conjugate stays within acceptance criteria, optionally ≥90% purity or ≥95% purity, for at least 1 week, 2 weeks, 1 month, 3 months, 6 months, 1 year, 2 years, or more.

### Example 93

### Cleavage Rate of Peptide-Conjugates

This example illustrates the cleavage rate of peptide-conjugates. A peptide of SEQ ID NO: 115, SEQ ID NO: 234, SEQ ID NO: 114, SEQ ID NO: 118, SEQ ID NO: 126, or SEQ ID NO: 109 or any of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 is complexed, conjugated, or fused to a drug such as desciclesonide by a cleavable linker. Optionally, the linker is an ester. The peptide-conjugate is incubated in buffer, rat plasma, human plasma, or synovial fluid of an animal species at 37° C. and analyzed at various time points for cleavage. The sample is processed and analyzed by liquid chromatography-mass spectrometry (LC-MS). The peptide-conjugate cleavage half-life is found to be greater than 1 h, 2 h, 4 h, 8 h, 12 h, 24 h, or more.

### Example 94

### Cartilage Binding of Peptides

This example illustrates cartilage binding of peptides of this disclosure. One or more peptides of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263 are either radiolabeled, such as with C¹⁴ using the methods of EXAMPLE 2, or labeled with a fluorophore, such as using the methods of EXAMPLE 3. The peptides are administered to a normal or diseased mouse or rat or other animal intravenously such as by the methods of EXAMPLES 6, 7 or 11. The peptides are allowed to circulate for 5 min, 1 h, 3 h, 8 h, 24 h, 48 h, or 96 h. The tissues are harvested and analyzed for signal. The labeled peptides are seen to accumulate in the cartilage of the treated animals, including the knee and intervertebral discs.

### Example 95

### Stability Testing of Peptide

This example describes stability testing of peptides of the present disclosure. A 5 mg/mL solution of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205) was prepared in 50 mM Tris, 5% Mannitol, pH 7.6. The stability of SEQ ID NO: 149 in solution was assessed by stressing the sample at 40° C. for 10 days and analyzed by RP-HPLC side-by-side with an unstressed sample prepared under the same conditions and stored at −20° C. The purity of SEQ ID NO: 149 when stressed decreased slightly compared to the non-stressed sample from an initial purity of 96.4% to 93.5% when analyzed by RP-HPLC. This change was predominantly the result of an increase in area of one predominant impurity peak eluting after the main peak in the sample that was exposed to the higher temperature. No other impurities present in the initial sample increased substantially and no substantial new impurities were seen. Visual inspection of the samples showed no change in solubility and no visible appearance of a pellet due to precipitation following centrifugation.

The samples were further analyzed by LC-MS to characterize two major impurities eluting before and after the main peak as well as the main peak. The predominant ions (m/z) observed in the main peak were 1076.2 and 1104.7; representing a +4 charge state resulting in an average mass of 4300.8 Da and 4415.0 Da respectively. The theoretical average mass of SEQ ID NO: 149 is 4300.1; thus this close match to 4300.8 Da confirming its identity. The 4415 Da mass differs by +114 amu and is consistent with the presence of bound trifluoroacetic acid TFA (TFA adduct).

A peak that was observed immediately preceding to the main peak, in both the non-stressed and the stressed sample, was also analyzed. The main ion observed in this preceding peak was 1080.5 (m/z); a +4 charge state resulting in an average mass of 4316.9. The difference of +16 amu is consistent with an oxidized species of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205), which was present in the non-stressed and the stressed sample, indicating it may have been formed during manufacturing of the peptide.

The predominant increasing peak on stress (eluting after the main peak) was also analyzed. One ion observed in this peak eluting after but not found in the main peak was 1076.4 m/z; representing a +4 charge state corresponding to an average mass of 4301.6 Da. The difference of +1 amu is consistent with the possible deamidation of asparagine. This deamidation may have occurred during manufacture and may have increased during stress. In addition, it is possible that isomerization events occurred (such as Asp isomerization), as those would not have a mass shift from the original species.

These results indicate that oxidation of the Met residue at position M25 could have occurred during the manufacture of the peptide of SEQ ID NO: 149 (also disclosed as SEQ ID NO: 46; non-GS version of SEQ ID NO: 149/SEQ ID NO: 46 are shown in SEQ ID NO: 128 and SEQ ID NO: 205). They also indicate that deamidation of the Asn residue(s) at position N7 or N32 could have occurred during the manufacture of and increased during the stressing of the peptide of SEQ ID NO: 149.

These results indicate that one or more of the M25, N7 and N32 residues in SEQ ID NO:149 can be less stable and hence by making modifications or substitutions at one or more of these residues in the peptide (or in corresponding positions in variant or shorter peptides), the peptide can be stabilized and its properties improved. Consequently, modifying or substituting one or more of the M25, N7, and/or N32 residues in corresponding peptides of the present disclosure, for example, can result in a more stable structure such as shown in or can be applied to the exemplary peptides of SEQ ID NO: 23, SEQ ID NO: 89, SEQ ID NO: 24, SEQ ID NO: 106, SEQ ID NO: 223, SEQ ID NO: 224, SEQ ID NO: 225, SEQ ID NO: 226 which are peptides with such improved properties. For example, many of the peptides as shown in in TABLE 7A and TABLE 7B, and TABLE 11 one or more of these of these changes. Further exemplary peptides comprising these modifications includes the peptides of SEQ ID NO: 52-SEQ ID NO: 66, SEQ ID NO: 241-SEQ ID NO: 248, SEQ ID NO: 134-SEQ ID NO: 148, SEQ ID NO: 249-SEQ ID NO: 256, SEQ ID NO: 111-SEQ ID NO: 126, and SEQ ID NO: 233-SEQ ID NO: 240. Such improved properties are also shown in can be applied to any one of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, SEQ ID NO: 198, SEQ ID NO: 200-SEQ ID NO: 215, SEQ ID NO: 233-SEQ ID NO: 256, or SEQ ID NO: 260-SEQ ID NO: 263.

While certain embodiments of the present disclosure have been exemplified or shown and described herein, it will be apparent to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all embodiments of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed:

1. A peptide comprising:
a) any one of SEQ ID NO: 87, SEQ ID NO: 88, SEQ ID NO: 107, SEQ ID NO: 108, SEQ ID NO: 219, SEQ ID NO: 223, SEQ ID NO: 225, or a functional fragment thereof, or
b) any one of SEQ ID NO: 89, SEQ ID NO: 106, and SEQ ID NO: 221, or a functional fragment thereof and further comprising at least one amino acid in each of SEQ ID NO: 89, SEQ ID NO: 106, and SEQ ID NO: 221 is selected from the group consisting of:
i) for SEQ ID NO: 89, wherein X¹ is selected from N, S, or G, wherein X² is selected from L or Y, wherein X³ is selected from D or E, wherein X⁴ is selected from M or T, wherein X⁵ is selected from N, Q, A, S, T, or L, wherein X⁶ is selected from S, G, or R, wherein X⁷ is selected from H or Y, and wherein X⁸ is selected from T or Y;
ii) for SEQ ID NO: 106, wherein X¹ is selected from L or Y, wherein X² is selected from D or E, wherein X³ is selected from M or T, wherein X⁴ is selected from N, Q, A, S, T, or L, wherein X⁵ is selected from S, G, or R, wherein X⁶ is selected from H or Y, and wherein X⁷ is selected from T or Y;
iii) for SEQ ID NO: 221, wherein each X and X₁₋₁₃ are individually any amino acid or no amino acid and at least one of the following residues at the denoted position, more than one of the following residues at the denoted position, or all of the following residues at the denoted position is included in SEQ ID NO: 221: X₁ is K, X₂ is Q, X₃ is Y, X₄ is E, X₅ is K, X₆ is T, X₇ is K, X₈ is M, X₉ is Q, A, S, T, or L, X₁₀ is G, X₁₁ is K, X₁₂ is Y, or X₁₃ is Y;
iv) a corresponding substitution selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

2. The peptide of claim 1, wherein the peptide comprises a sequence of any one of SEQ ID NO: 89, SEQ ID NO: 106, and SEQ ID NO: 221 and further comprises at least one of KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); or GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null.

3. The peptide of any one of claims 1-2, wherein the peptide comprises a sequence of any one of SEQ ID NO: 89, SEQ ID NO: 106, and SEQ ID NO: 221 and the peptide further comprises at least one of RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); or RFGRCI (SEQ ID NO: 259).

4. The peptide of any one of claims 1-3, wherein the peptide has a sequence selected from the group consisting of any one of SEQ ID NO: 106, and SEQ ID NO: 221.

5. The peptide of any one of claims 1-4, wherein the peptide comprises SEQ ID NO: 89 and wherein the peptide comprises one or more of the following characteristics:
a) X¹ is selected from S, or G;
b) X² is selected from Y;
c) X³ is selected from E;
d) X⁴ is selected from T;
e) X⁵ is selected from Q, A, S, T, or L;
f) X⁶ is selected from G, or R;
g) X⁷ is selected from Y; or
h) X⁸ is selected from Y.

6. The peptide of claim 5, wherein the peptide comprises two, three, four, five, six, seven or more of the characteristics, or wherein the peptide comprises all of the characteristics.

7. The peptide of any one of claims 1-6, wherein
a) the peptide comprises at least 70%, at least at least 75%, at least 80%, at least 85%, at least 90%, at least 95%, at least 97%, at least 98%, at least 99%, or 100% sequence identity with a sequence selected from the group SEQ ID NO: 111-SEQ ID NO: 126 or SEQ ID NO: 233-SEQ ID NO: 240; or
b) the peptide comprises at least 70%, at least at least 75%, at least 80%, at least 85%, at least 90%, at least 95%, at least 97%, at least 98%, at least 99%, or 100% sequence identity with a sequence selected from the group SEQ ID NO: 134-SEQ ID NO: 148 and SEQ ID NO: 249-SEQ ID NO: 256.

8. The peptide of any one of claims 1-7, wherein
a) the peptide comprises a sequence that has at least 70%, at least 75%, at least 80%, at least 85%, at least 90%, at least 92%, at least 95%, at least 97%, or at least 100% sequence identity with any one of SEQ ID NO: 109, SEQ ID NO: 110, SEQ ID NO: 260, or SEQ ID NO: 262 and wherein the peptide further comprises a joining sequence of one or more amino acid residues located immediately adjacent to an N-terminus or a C-terminus of the peptide and wherein
i) N−1 is selected from the group consisting of G, A, L, I, M, F, W, P, S, T, C, Y, N, Q, D, E, K, R, and H;
ii) N−2 is selected from the group consisting of G, A, V, L, I, M, F, W, P, S, T, C, Y, Q, D, E, K, R, and H;
iii) N−3 is selected from the group consisting of G, A, V, L, M, F, W, P, S, T, C, Y, N, Q, D, E, K, R, and H;
iv) N−4 is selected from the group consisting of G, A, V, L, I, M, F, W, S, T, C, Y, N, Q, D, E, K, R, and H;
v) N−5 is selected from the group consisting of G, A, L, I, M, F, W, P, S, T, C, Y, N, Q, D, E, K, R, and H; or
vi) N−6 is selected from the group consisting of A, V, L, I, M, F, W, P, S, T, C, Y, N, Q, D, E, K, R, and H
b) the peptide comprises a sequence that has at least 70%, at least 75%, at least 80%, at least 85%, at least 90%, at least 92%, at least 95%, at least 97%, or at least 100% sequence identity with SEQ ID NO: 129 and wherein the peptide further comprises a joining sequence of one or more amino acid residues located immediately adjacent to an N-terminus or a C-terminus of the peptide and wherein N−1 is selected from the group consisting of A, V, L, I, M, F, W, P, S, T, C, Y, N, Q, D, E, K, R, and H;
c) the peptide comprises a sequence that has at least 70%, at least 75%, at least 80%, at least 85%, at least 90%, at least 92%, at least 95%, at least 97%, or at least 100% sequence identity with SEQ ID NO: 130 and wherein the peptide further comprises a joining sequence of one or more amino acid residues located immediately adjacent to an N-terminus or a C-terminus of the peptide and wherein
i) N−1 is selected from the group consisting of G, A, L, I, M, F, W, P, S, T, C, Y, N, Q, D, E, K, R, and H; or
ii) N−2 is selected from the group consisting of A, V, L, I, M, F, W, P, S, T, C, Y, N, Q, D, E, K, R, and H;
d) the peptide comprises a sequence that has at least 70%, at least 75%, at least 80%, at least 85%, at least 90%, at least 92%, at least 95%, at least 97%, or at least 100% sequence identity with SEQ ID NO: 131 and wherein the peptide further comprises a joining sequence of one or more amino acid residues located immediately adjacent to an N-terminus or a C-terminus of the peptide and wherein
i) N−1 is selected from the group consisting of G, A, V, L, I, M, F, W, S, T, C, Y, N, Q, D, E, K, R, and H;
ii) N−2 is selected from the group consisting of G, A, L, I, M, F, W, P, S, T, C, Y, N, Q, D, E, K, R, and H; or
iii) N−3 is selected from the group consisting of A, V, L, I, M, F, W, P, S, T, C, Y, N, Q, D, E, K, R, and H;
e) the peptide comprises a sequence that has at least 70%, at least 75%, at least 80%, at least 85%, at least 90%, at least 92%, at least 95%, at least 97%, or at least 100% sequence identity with SEQ ID NO: 132 and wherein the peptide further comprises a joining sequence of one or more amino acid residues located immediately adjacent to an N-terminus or a C-terminus of the peptide and wherein
i) N−1 is selected from the group consisting of G, A, V, L, M, F, W, P, S, T, C, Y, N, Q, D, E, K, R, and H;
ii) N−2 is selected from the group consisting of G, A, V, L, I, M, F, W, S, T, C, Y, N, Q, D, E, K, R, and H;
iii) N−3 is selected from the group consisting of G, A, L, I, M, F, W, P, S, T, C, Y, N, Q, D, E, K, R, and H; or
iv) N−4 is selected from the group consisting of A, V, L, I, M, F, W, P, S, T, C, Y, N, Q, D, E, K, R, and H;
f) the peptide comprises a sequence that has at least 70%, at least 75%, at least 80%, at least 85%, at least 90%, at least 92%, at least 95%, at least 97%, or at least 100% sequence identity with SEQ ID NO: 133 and wherein the peptide further comprises a joining sequence of one or more amino acid residues located immediately adjacent to an N-terminus or a C-terminus of the peptide and wherein
i) N−1 is selected from the group consisting of G, A, V, L, I, M, F, W, P, S, T, C, Y, Q, D, E, K, R, and H;
ii) N−2 is selected from the group consisting of G, A, V, L, M, F, W, P, S, T, C, Y, N, Q, D, E, K, R, and H;
iii) N−3 is selected from the group consisting of G, A, V, L, I, M, F, W, S, T, C, Y, N, Q, D, E, K, R, and H;
iv) N−4 is selected from the group consisting of G, A, L, I, M, F, W, P, S, T, C, Y, N, Q, D, E, K, R, and H; or
v) N−5 is selected from the group consisting of A, V, L, I, M, F, W, P, S, T, C, Y, N, Q, D, E, K, R, and H;
g) the peptide comprises a sequence that has at least 70%, at least 75%, at least 80%, at least 85%, at least 90%, at least 92%, at least 95%, at least 97%, or at least 100% sequence identity with SEQ ID NO: 260 and wherein the peptide further comprises a joining sequence of one or more amino acid residues located immediately adjacent to an N-terminus or a C-terminus of the peptide and wherein
i) C+1 is selected from the group consisting of G, A, V, L, I, M, F, W, P, S, C, Y, N, Q, D, E, K, R, and H; or
ii) C+2 is selected from the group consisting of G, A, V, L, I, M, F, W, S, T, C, Y, N, Q, D, E, K, R, and H; or
h) the peptide comprises a sequence that has at least 70%, at least 75%, at least 80%, at least 85%, at least 90%, at least 92%, at least 95%, at least 97%, or at least 100% sequence identity with SEQ ID NO: 262 and wherein the peptide further comprises a joining sequence of one or more amino acid residues located immediately adjacent to an N-terminus or a C-terminus of the peptide and wherein C+1 is selected from the group consisting of G, A, V, L, I, M, F, W, S, T, C, Y, N, Q, D, E, K, R, and H.

9. The peptide of claim 8, wherein the joining sequence is at the N-terminus or a C-terminus of the peptide, or both the N-terminus and the C-terminus of the peptide.

10. The peptide of any one of claims 8-9, wherein the joining sequence comprises from 1 to 100 amino acid residues.

11. The peptide of any one of claims 1-10, wherein the peptide comprises a sequence that has at least 70%, at least 75%, at least 80%, at least 85%, at least 90%, at least 95%, at least 97%, at least 99% or 100% sequence identity with any one of SEQ ID NO: 109-SEQ ID NO: 110, SEQ ID NO: 260, or SEQ ID NO: 262 and further comprising no more than 5 additional amino acids at the N-terminus.

12. The peptide of any one of claims 1-11, wherein the peptide consists of a sequence that has at least 70%, at least 75%, at least 80%, at least 85%, at least 90%, at least 95%, at least 97%, at least 99% or 100% sequence identity with any one of the peptides selected from the group consisting of SEQ ID NO: 109-SEQ ID NO: 110, SEQ ID NO: 129-SEQ ID NO: 133, SEQ ID NO: 260, or SEQ ID NO: 262.

13. The peptide of claim 12, wherein the peptide further comprises a joining sequence of one or more amino acid residues located immediately adjacent to an N-terminus or a C-terminus of the peptide and wherein:
a) for any one of SEQ ID NO: 109, SEQ ID NO: 110, SEQ ID NO: 260, or SEQ ID NO: 262:
i) N−1 is selected from the group consisting of G, A, L, I, M, F, W, P, S, T, C, Y, N, Q, D, E, K, R, and H;
ii) N−2 is selected from the group consisting of G, A, V, L, I, M, F, W, P, S, T, C, Y, Q, D, E, K, R, and H;
iii) N−3 is selected from the group consisting of G, A, V, L, M, F, W, P, S, T, C, Y, N, Q, D, E, K, R, and H;
iv) N−4 is selected from the group consisting of G, A, V, L, I, M, F, W, S, T, C, Y, N, Q, D, E, K, R, and H;
v) N−5 is selected from the group consisting of G, A, L, I, M, F, W, P, S, T, C, Y, N, Q, D, E, K, R, and H; or
vi) N−6 is selected from the group consisting of A, V, L, I, M, F, W, P, S, T, C, Y, N, Q, D, E, K, R, and H; or
b) for SEQ ID NO: 129, N−1 is selected from the group consisting of A, V, L, I, M, F, W, P, S, T, C, Y, N, Q, D, E, K, R, and H;
c) for SEQ ID NO: 130:
i) N−1 is selected from the group consisting of G, A, L, I, M, F, W, P, S, T, C, Y, N, Q, D, E, K, R, and H; or
ii) N−2 is selected from the group consisting of A, V, L, I, M, F, W, P, S, T, C, Y, N, Q, D, E, K, R, and H;
d) for SEQ ID NO: 131:
i) N−1 is selected from the group consisting of G, A, V, L, I, M, F, W, S, T, C, Y, N, Q, D, E, K, R, and H;
ii) N−2 is selected from the group consisting of G, A, L, I, M, F, W, P, S, T, C, Y, N, Q, D, E, K, R, and H; or
iii) N−3 is selected from the group consisting of A, V, L, I, M, F, W, P, S, T, C, Y, N, Q, D, E, K, R, and H;
e) for SEQ ID NO: 132:
i) N−1 is selected from the group consisting of G, A, V, L, M, F, W, P, S, T, C, Y, N, Q, D, E, K, R, and H;
ii) N−2 is selected from the group consisting of G, A, V, L, I, M, F, W, S, T, C, Y, N, Q, D, E, K, R, and H;
iii) N−3 is selected from the group consisting of G, A, L, I, M, F, W, P, S, T, C, Y, N, Q, D, E, K, R, and H; or
iv) N−4 is selected from the group consisting of A, V, L, I, M, F, W, P, S, T, C, Y, N, Q, D, E, K, R, and H;
f) for SEQ ID NO: 133:
i) N−1 is selected from the group consisting of G, A, V, L, I, M, F, W, P, S, T, C, Y, Q, D, E, K, R, and H;
ii) N−2 is selected from the group consisting of G, A, V, L, M, F, W, P, S, T, C, Y, N, Q, D, E, K, R, and H;
iii) N−3 is selected from the group consisting of G, A, V, L, I, M, F, W, S, T, C, Y, N, Q, D, E, K, R, and H;
iv) N−4 is selected from the group consisting of G, A, L, I, M, F, W, P, S, T, C, Y, N, Q, D, E, K, R, and H; or
v) N−5 is selected from the group consisting of A, V, L, I, M, F, W, P, S, T, C, Y, N, Q, D, E, K, R, and H;
g) for SEQ ID NO: 260:
i) C+1 is selected from the group consisting of G, A, V, L, I, M, F, W, P, S, C, Y, N, Q, D, E, K, R, and H; or
ii) C+2 is selected from the group consisting of G, A, V, L, I, M, F, W, S, T, C, Y, N, Q, D, E, K, R, and H; or
h) for SEQ ID NO: 262, C+1 is selected from the group consisting of G, A, V, L, I, M, F, W, S, T, C, Y, N, Q, D, E, K, R, and H.

14. The peptide of any one of claims 1-13, wherein the peptide is selected from the group consisting of SEQ ID NO: 109-SEQ ID NO: 110, SEQ ID NO: 129-SEQ ID NO: 133, SEQ ID NO: 260, and SEQ ID NO: 262.

15. The peptide of any one of claims 1-14, wherein the peptide consists of a sequence that has at least 70%, at least 75%, at least 80%, at least 85%, at least 90%, at least 95%, at least 97%, at least 99% or 100% sequence identity with any one of the peptides selected from the group consisting of SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256.

16. The peptide of any one of claims 1-15, wherein the peptide is selected from the group consisting of SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256.

17. A peptide, wherein
a) the peptide comprises at least 70%, at least at least 75%, at least 80%, at least 85%, at least 90%, at least 95%, at least 97%, at least 98%, at least 99%, or 100% sequence identity with a sequence selected from the group SEQ ID NO: 111-SEQ ID NO: 126 or SEQ ID NO: 233-SEQ ID NO: 240; or
b) the peptide comprises at least 70%, at least at least 75%, at least 80%, at least 85%, at least 90%, at least 95%, at least 97%, at least 98%, at least 99%, or 100% sequence identity with a sequence selected from the group SEQ ID NO: 134-SEQ ID NO: 148 and SEQ ID NO: 249-SEQ ID NO: 256.

18. A peptide, wherein
a) the peptide comprises a sequence that has at least 70%, at least 75%, at least 80%, at least 85%, at least 90%, at least 92%, at least 95%, at least 97%, or at least 100% sequence identity with any one of SEQ ID NO: 109, SEQ ID NO: 110, SEQ ID NO: 260, or SEQ ID NO: 262 and wherein the peptide further comprises a joining sequence of one or more amino acid residues located immediately adjacent to an N-terminus or a C-terminus of the peptide and wherein
i) N−1 is selected from the group consisting of G, A, L, I, M, F, W, P, S, T, C, Y, N, Q, D, E, K, R, and H;
ii) N−2 is selected from the group consisting of G, A, V, L, I, M, F, W, P, S, T, C, Y, Q, D, E, K, R, and H;
iii) N−3 is selected from the group consisting of G, A, V, L, M, F, W, P, S, T, C, Y, N, Q, D, E, K, R, and H;
iv) N−4 is selected from the group consisting of G, A, V, L, I, M, F, W, S, T, C, Y, N, Q, D, E, K, R, and H;
v) N−5 is selected from the group consisting of G, A, L, I, M, F, W, P, S, T, C, Y, N, Q, D, E, K, R, and H; or
vi) N−6 is selected from the group consisting of A, V, L, I, M, F, W, P, S, T, C, Y, N, Q, D, E, K, R, and H
b) the peptide comprises a sequence that has at least 70%, at least 75%, at least 80%, at least 85%, at least 90%, at least 92%, at least 95%, at least 97%, or at least 100% sequence identity with SEQ ID NO: 129 and wherein the peptide further comprises a joining sequence of one or more amino acid residues located immediately adjacent to an N-terminus or a C-terminus of the peptide and wherein N−1 is selected from the group consisting of A, V, L, I, M, F, W, P, S, T, C, Y, N, Q, D, E, K, R, and H;
c) the peptide comprises a sequence that has at least 70%, at least 75%, at least 80%, at least 85%, at least 90%, at least 92%, at least 95%, at least 97%, or at least 100% sequence identity with SEQ ID NO: 130 and wherein the peptide further comprises a joining sequence of one or more amino acid residues located immediately adjacent to an N-terminus or a C-terminus of the peptide and wherein
i) N−1 is selected from the group consisting of G, A, L, I, M, F, W, P, S, T, C, Y, N, Q, D, E, K, R, and H; or
ii) N−2 is selected from the group consisting of A, V, L, I, M, F, W, P, S, T, C, Y, N, Q, D, E, K, R, and H;
d) the peptide comprises a sequence that has at least 70%, at least 75%, at least 80%, at least 85%, at least 90%, at least 92%, at least 95%, at least 97%, or at least 100% sequence identity with SEQ ID NO: 131 and wherein the peptide further comprises a joining sequence of one or more amino acid residues located immediately adjacent to an N-terminus or a C-terminus of the peptide and wherein
i) N−1 is selected from the group consisting of G, A, V, L, I, M, F, W, S, T, C, Y, N, Q, D, E, K, R, and H;
ii) N−2 is selected from the group consisting of G, A, L, I, M, F, W, P, S, T, C, Y, N, Q, D, E, K, R, and H; or
iii) N−3 is selected from the group consisting of A, V, L, I, M, F, W, P, S, T, C, Y, N, Q, D, E, K, R, and H;
e) the peptide comprises a sequence that has at least 70%, at least 75%, at least 80%, at least 85%, at least 90%, at least 92%, at least 95%, at least 97%, or at least 100% sequence identity with SEQ ID NO: 132 and wherein the peptide further comprises a joining sequence of one or more amino acid residues located immediately adjacent to an N-terminus or a C-terminus of the peptide and wherein
i) N−1 is selected from the group consisting of G, A, V, L, M, F, W, P, S, T, C, Y, N, Q, D, E, K, R, and H;
ii) N−2 is selected from the group consisting of G, A, V, L, I, M, F, W, S, T, C, Y, N, Q, D, E, K, R, and H;
iii) N−3 is selected from the group consisting of G, A, L, I, M, F, W, P, S, T, C, Y, N, Q, D, E, K, R, and H; or
iv) N−4 is selected from the group consisting of A, V, L, I, M, F, W, P, S, T, C, Y, N, Q, D, E, K, R, and H;
f) the peptide comprises a sequence that has at least 70%, at least 75%, at least 80%, at least 85%, at least 90%, at least 92%, at least 95%, at least 97%, or at least 100% sequence identity with SEQ ID NO: 133 and wherein the peptide further comprises a joining sequence of one or more amino acid residues located immediately adjacent to an N-terminus or a C-terminus of the peptide and wherein
i) N−1 is selected from the group consisting of G, A, V, L, I, M, F, W, P, S, T, C, Y, Q, D, E, K, R, and H;
ii) N−2 is selected from the group consisting of G, A, V, L, M, F, W, P, S, T, C, Y, N, Q, D, E, K, R, and H;
iii) N−3 is selected from the group consisting of G, A, V, L, I, M, F, W, S, T, C, Y, N, Q, D, E, K, R, and H;
iv) N−4 is selected from the group consisting of G, A, L, I, M, F, W, P, S, T, C, Y, N, Q, D, E, K, R, and H;
v) N−5 is selected from the group consisting of A, V, L, I, M, F, W, P, S, T, C, Y, N, Q, D, E, K, R, and H;
g) the peptide comprises a sequence that has at least 70%, at least 75%, at least 80%, at least 85%, at least 90%, at least 92%, at least 95%, at least 97%, or at least 100% sequence identity with SEQ ID NO: 260 and wherein the peptide further comprises a joining sequence of one or more amino acid residues located immediately adjacent to an N-terminus or a C-terminus of the peptide and wherein
i) C+1 is selected from the group consisting of G, A, V, L, I, M, F, W, P, S, C, Y, N, Q, D, E, K, R, and H; or
ii) C+2 is selected from the group consisting of G, A, V, L, I, M, F, W, S, T, C, Y, N, Q, D, E, K, R, and H; or
h) the peptide comprises a sequence that has at least 70%, at least 75%, at least 80%, at least 85%, at least 90%, at least 92%, at least 95%, at least 97%, or at least 100% sequence identity with SEQ ID NO: 262 and wherein the peptide further comprises a joining sequence of one or more amino acid residues located immediately adjacent to an N-terminus or a C-terminus of the peptide and wherein C+1 is selected from the group consisting of G, A, V, L, I, M, F, W, S, T, C, Y, N, Q, D, E, K, R, and H.

19. The peptide of claim 17, wherein the peptide further comprises a joining sequence of one or more amino acid residues located immediately adjacent to an N-terminus or a C-terminus of the peptide.

20. The peptide of any one of claims 18-19, wherein the joining sequence is at an N-terminus or a C-terminus of the peptide, or both the N-terminus and the C-terminus of the peptide.

21. The peptide of any one of claims 18-20, wherein the joining sequence comprises from 1 to 100 amino acid residues.

22. The peptide of any one of claims 18-21, wherein the peptide comprises a sequence that has at least 70%, at least 75%, at least 80%, at least 85%, at least 90%, at least 95%, at least 97%, at least 99% or 100% sequence identity with any one of SEQ ID NO: 109-SEQ ID NO: 110, SEQ ID NO: 260, or SEQ ID NO: 262 and further comprising no more than 5 additional amino acids at the N-terminus.

23. The peptide of any one of claims 18-22, wherein the peptide consists of a sequence that has at least 70%, at least 75%, at least 80%, at least 85%, at least 90%, at least 95%, at least 97%, at least 99% or 100% sequence identity with any one of the peptides selected from the group consisting of SEQ ID NO: 109-SEQ ID NO: 110, SEQ ID NO: 129-SEQ ID NO: 133, SEQ ID NO: 260, or SEQ ID NO: 262.

24. The peptide of claim 23, wherein the peptide further comprises a joining sequence of one or more amino acid residues located immediately adjacent to an N-terminus or a C-terminus of the peptide and wherein:
a) for any one of SEQ ID NO: 109, SEQ ID NO: 110, SEQ ID NO: 260, or SEQ ID NO: 262:
i) N−1 is selected from the group consisting of G, A, L, I, M, F, W, P, S, T, C, Y, N, Q, D, E, K, R, and H;
ii) N−2 is selected from the group consisting of G, A, V, L, I, M, F, W, P, S, T, C, Y, Q, D, E, K, R, and H;
iii) N−3 is selected from the group consisting of G, A, V, L, M, F, W, P, S, T, C, Y, N, Q, D, E, K, R, and H;
iv) N−4 is selected from the group consisting of G, A, V, L, I, M, F, W, S, T, C, Y, N, Q, D, E, K, R, and H;
v) N−5 is selected from the group consisting of G, A, L, I, M, F, W, P, S, T, C, Y, N, Q, D, E, K, R, and H; or
vi) N−6 is selected from the group consisting of A, V, L, I, M, F, W, P, S, T, C, Y, N, Q, D, E, K, R, and H; or
b) for SEQ ID NO: 129, N−1 is selected from the group consisting of A, V, L, I, M, F, W, P, S, T, C, Y, N, Q, D, E, K, R, and H; or
c) for SEQ ID NO: 130:
i) N−1 is selected from the group consisting of G, A, L, I, M, F, W, P, S, T, C, Y, N, Q, D, E, K, R, and H; or
ii) N−2 is selected from the group consisting of A, V, L, I, M, F, W, P, S, T, C, Y, N, Q, D, E, K, R, and H; or
d) for SEQ ID NO: 131:
i) N−1 is selected from the group consisting of G, A, V, L, I, M, F, W, S, T, C, Y, N, Q, D, E, K, R, and H;
ii) N−2 is selected from the group consisting of G, A, L, I, M, F, W, P, S, T, C, Y, N, Q, D, E, K, R, and H; or
iii) N−3 is selected from the group consisting of A, V, L, I, M, F, W, P, S, T, C, Y, N, Q, D, E, K, R, and H; or
e) for SEQ ID NO: 132:
i) N−1 is selected from the group consisting of G, A, V, L, M, F, W, P, S, T, C, Y, N, Q, D, E, K, R, and H;
ii) N−2 is selected from the group consisting of G, A, V, L, I, M, F, W, S, T, C, Y, N, Q, D, E, K, R, and H;
iii) N−3 is selected from the group consisting of G, A, L, I, M, F, W, P, S, T, C, Y, N, Q, D, E, K, R, and H; or
iv) N−4 is selected from the group consisting of A, V, L, I, M, F, W, P, S, T, C, Y, N, Q, D, E, K, R, and H;
f) for SEQ ID NO: 133:
i) N−1 is selected from the group consisting of G, A, V, L, I, M, F, W, P, S, T, C, Y, Q, D, E, K, R, and H;
ii) N−2 is selected from the group consisting of G, A, V, L, M, F, W, P, S, T, C, Y, N, Q, D, E, K, R, and H;
iii) N−3 is selected from the group consisting of G, A, V, L, I, M, F, W, S, T, C, Y, N, Q, D, E, K, R, and H;
iv) N−4 is selected from the group consisting of G, A, L, I, M, F, W, P, S, T, C, Y, N, Q, D, E, K, R, and H;
v) N−5 is selected from the group consisting of A, V, L, I, M, F, W, P, S, T, C, Y, N, Q, D, E, K, R, and H;
g) for SEQ ID NO: 260:
i) C+1 is selected from the group consisting of G, A, V, L, I, M, F, W, P, S, C, Y, N, Q, D, E, K, R, and H; or
ii) C+2 is selected from the group consisting of G, A, V, L, I, M, F, W, S, T, C, Y, N, Q, D, E, K, R, and H; or
h) for SEQ ID NO: 262, C+1 is selected from the group consisting of G, A, V, L, I, M, F, W, S, T, C, Y, N, Q, D, E, K, R, and H.

25. The peptide of any one of claims 18-24, wherein the peptide is selected from the group consisting of SEQ ID NO: 109-SEQ ID NO: 110, SEQ ID NO: 129-SEQ ID NO: 133, SEQ ID NO: 260, and SEQ ID NO: 262.

26. The peptide of any one of claims 17 or 19-25, wherein the peptide consists of a sequence that has at least 70%, at least 75%, at least 80%, at least 85%, at least 90%, at least 95%, at least 97%, at least 99% or 100% sequence identity with any one of the peptides selected from the group consisting of SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256.

27. The peptide of any one of claims 17 or 19-26, wherein the peptide is selected from the group consisting of SEQ ID NO: 111-SEQ ID NO: 126, SEQ ID NO: 134-SEQ ID NO: 148, SEQ ID NO: 233-SEQ ID NO: 240, SEQ ID NO: 249-SEQ ID NO: 256.

28. The peptide of any one of claims 1-27, wherein the peptide comprises SEQ ID NO: 28, SEQ ID NO: 45-SEQ ID NO: 51, SEQ ID NO: 109, SEQ ID NO: 150, SEQ ID NO: 199, SEQ ID NO: 110, SEQ ID NO: 127-SEQ ID NO: 133, SEQ ID NO: 149, or SEQ ID NO: 260-SEQ ID NO: 263 and further comprises one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null.

29. The peptide of any one of claims 1-28, wherein the peptide comprises SEQ ID NO: 27, SEQ ID NO: 29-SEQ ID NO: 44, SEQ ID NO: 52-SEQ ID NO: 66, SEQ ID NO: 109, SEQ ID NO: 150, SEQ ID NO: 199, SEQ ID NO: 111-SEQ ID NO: 126, SEQ ID NO: 134-SEQ ID NO: 148, SEQ ID NO: 233-SEQ ID NO: 256, and SEQ ID NO: 21-SEQ ID NO: 26, SEQ ID NO: 87-SEQ ID NO: 89, SEQ ID NO: 106-SEQ ID NO: 108, SEQ ID NO: 219-SEQ ID NO: 226 and further comprises one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259).

30. The peptide of any one of claims 1-29, wherein the peptide comprises one or more of the following peptide fragments within its sequence: GKCINKKCKC (SEQ ID NO: 90); KCIN (SEQ ID NO: 91); KKCK (SEQ ID NO: 92); PCKR (SEQ ID NO: 93); KRCSRR (SEQ ID NO: 94); KQC (SEQ ID NO: 95); GRCINRRCRC (SEQ ID NO: 96); RCIN (SEQ ID NO: 97); RRCR (SEQ ID NO: 98); PCRR (SEQ ID NO: 99); RRCSRR (SEQ ID NO: 100); RQC (SEQ ID NO: 101); PCKK (SEQ ID NO: 102), KKCSKK (SEQ ID NO: 103), GKCMNGKC (SEQ ID NO: 104); GRCMNGRC (SEQ ID NO: 105); GRCINSRC (SEQ ID NO: 227), GRCIXXRC (SEQ ID NO: 228) wherein each X can independently be any amino acid or amino acid analogue or null, GRCIX¹X²RC (SEQ ID NO: 229), wherein X¹=N, Q, A, S, T or L and X²=S, G, or R (SEQ ID NO: 229), PCR (SEQ ID NO: 230), CLDPCRRA (SEQ ID NO: 231), CLDPCRR (SEQ ID NO: 232), RCRGSRDC (SEQ ID NO: 257), PCRRAG (SEQ ID NO: 258), and RFGRCI (SEQ ID NO: 259).

31. The peptide of any one of claims 1-30, wherein the peptide comprises an N-terminal sequence comprising GG, SS, GS, SG, S, or G.

32. The peptide of any one of claims 1-31, wherein the peptide is SEQ ID NO: 219.

33. The peptide of any one of claims 1-31, wherein the peptide is SEQ ID NO: 220.

34. The peptide of any one of claims 1-31, wherein the peptide is SEQ ID NO: 221 and wherein:
a) the peptide further comprises one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null; or
b) the peptide further comprises one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259)
c) and the peptide further comprises at least one corresponding substitution selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

35. The peptide of any one of claims 1-31, wherein the peptide is SEQ ID NO: 222.

36. The peptide of any one of claims 1-31, wherein the peptide is SEQ ID NO: 223.

37. The peptide of any one of claims 1-31, wherein the peptide is SEQ ID NO: 224.

38. The peptide of any one of claims 1-31, wherein the peptide is SEQ ID NO: 225.

39. The peptide of any one of claims 1-31, wherein the peptide is SEQ ID NO: 226.

40. The peptide of any one of claims 1-31, wherein the peptide is SEQ ID NO: 87.

41. The peptide of any one of claims 1-31, wherein the peptide is SEQ ID NO: 89 and wherein:
a) the peptide further comprises one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null; or
b) the peptide further comprises one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259)
c) and the peptide further comprises at least one corresponding substitution selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

42. The peptide of any one of claims 1-31, wherein the peptide is SEQ ID NO: 106 and wherein:
a) the peptide further comprises one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null; or
b) the peptide further comprises one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259)
c) and the peptide further comprises at least one corresponding substitution selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

43. The peptide of any one of claims 1-31, wherein the peptide is SEQ ID NO: 110 and further comprising no more than 5 additional amino acids at the N-terminus.

44. The peptide of any one of claims 1-31, wherein the peptide is SEQ ID NO: 219.

45. The peptide of any one of claims 1-31, wherein the peptide is SEQ ID NO: 221
a) the peptide further comprises one or more of the following peptide fragments within its sequence: KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null; or
b) the peptide further comprises one or more of the following peptide fragments within its sequence: RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); and RFGRCI (SEQ ID NO: 259)
c) and the peptide further comprises at least one corresponding substitution selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.

46. A peptide comprising at least 70%, at least 75%, at least 80%, at least 85%, at least 90%, at least 92%, at least 95%, at least 97%, at least 99%, or 100% sequence identity with any one of SEQ ID NO: 209-SEQ ID NO: 215, wherein the peptide further comprises an active agent and wherein the active agent is complexed with the peptide to form a peptide active agent complex.

47. The peptide of any one of claims 1-46, further comprising an active agent, wherein the active agent is complexed with the peptide to form a peptide active agent complex.

48. The peptide active agent complex of claim 47, wherein the active agent is selected from TABLE 3, TABLE 4, or TABLE 5.

49. The peptide of any one of claims 1-46 or the peptide active agent complex of any one of claims 47-48, wherein the peptide comprises at least 70%, at least 80%, at least 90%, at least 95%, at least 97%, or 100% sequence identity to:
SEQ ID NO: 27;
SEQ ID NO: 28;
SEQ ID NO: 29;
SEQ ID NO: 30;
SEQ ID NO: 31;
SEQ ID NO: 32;
SEQ ID NO: 33;
SEQ ID NO: 34;
SEQ ID NO: 35;
SEQ ID NO: 36;
SEQ ID NO: 37;
SEQ ID NO: 38;
SEQ ID NO: 39;
SEQ ID NO: 40;
SEQ ID NO: 41;
SEQ ID NO: 42;
SEQ ID NO: 43;
SEQ ID NO: 44;
SEQ ID NO: 45;
SEQ ID NO: 234;
SEQ ID NO: 47;
SEQ ID NO: 48;
SEQ ID NO: 49;
SEQ ID NO: 50;
SEQ ID NO: 51;
SEQ ID NO: 109 and wherein the peptide further comprises a joining sequence of one or more amino acid residues located immediately adjacent to an N-terminus or a C-terminus of the peptide and wherein
i) N−1 is selected from the group consisting of G, A, L, I, M, F, W, P, S, T, C, Y, N, Q, D, E, K, R, and H;
ii) N−2 is selected from the group consisting of G, A, V, L, I, M, F, W, P, S, T, C, Y, Q, D, E, K, R, and H;
iii) N−3 is selected from the group consisting of G, A, V, L, M, F, W, P, S, T, C, Y, N, Q, D, E, K, R, and H;
iv) N−4 is selected from the group consisting of G, A, V, L, I, M, F, W, S, T, C, Y, N, Q, D, E, K, R, and H;
v) N−5 is selected from the group consisting of G, A, L, I, M, F, W, P, S, T, C, Y, N, Q, D, E, K, R, and H; or
vi) N−6 is selected from the group consisting of A, V, L, I, M, F, W, P, S, T, C, Y, N, Q, D, E, K, R, and H;
SEQ ID NO: 110 and wherein the peptide further comprises a joining sequence of one or more amino acid residues located immediately adjacent to an N-terminus or a C-terminus of the peptide and wherein
i) N−1 is selected from the group consisting of G, A, L, I, M, F, W, P, S, T, C, Y, N, Q, D, E, K, R, and H;
ii) N−2 is selected from the group consisting of G, A, V, L, I, M, F, W, P, S, T, C, Y, Q, D, E, K, R, and H;
iii) N−3 is selected from the group consisting of G, A, V, L, M, F, W, P, S, T, C, Y, N, Q, D, E, K, R, and H;
iv) N−4 is selected from the group consisting of G, A, V, L, I, M, F, W, S, T, C, Y, N, Q, D, E, K, R, and H;
v) N−5 is selected from the group consisting of G, A, L, I, M, F, W, P, S, T, C, Y, N, Q, D, E, K, R, and H; or
vi) N−6 is selected from the group consisting of A, V, L, I, M, F, W, P, S, T, C, Y, N, Q, D, E, K, R, and H;
SEQ ID NO: 111;
SEQ ID NO: 112;
SEQ ID NO: 113;
SEQ ID NO: 114;
SEQ ID NO: 115;
SEQ ID NO: 116;
SEQ ID NO: 117;
SEQ ID NO: 118;
SEQ ID NO: 119;
SEQ ID NO: 120;
SEQ ID NO: 121;
SEQ ID NO: 122;
SEQ ID NO: 123;
SEQ ID NO: 124;
SEQ ID NO: 125;
SEQ ID NO: 126;
SEQ ID NO: 87;
SEQ ID NO: 129 and wherein the peptide further comprises a joining sequence of one or more amino acid residues located immediately adjacent to an N-terminus or a C-terminus of the peptide and wherein N−1 is selected from the group consisting of A, V, L, I, M, F, W, P, S, T, C, Y, N, Q, D, E, K, R, and H;
SEQ ID NO: 130 and wherein the peptide further comprises a joining sequence of one or more amino acid residues located immediately adjacent to an N-terminus or a C-terminus of the peptide and wherein
i) N−1 is selected from the group consisting of G, A, L, I, M, F, W, P, S, T, C, Y, N, Q, D, E, K, R, and H; or
ii) N−2 is selected from the group consisting of A, V, L, I, M, F, W, P, S, T, C, Y, N, Q, D, E, K, R, and H;
SEQ ID NO: 131 and wherein the peptide further comprises a joining sequence of one or more amino acid residues located immediately adjacent to an N-terminus or a C-terminus of the peptide and wherein
i) N−1 is selected from the group consisting of G, A, V, L, I, M, F, W, S, T, C, Y, N, Q, D, E, K, R, and H;
ii) N−2 is selected from the group consisting of G, A, L, I, M, F, W, P, S, T, C, Y, N, Q, D, E, K, R, and H; or
iii) N−3 is selected from the group consisting of A, V, L, I, M, F, W, P, S, T, C, Y, N, Q, D, E, K, R, and H;
SEQ ID NO: 132 and wherein the peptide further comprises a joining sequence of one or more amino acid residues located immediately adjacent to an N-terminus or a C-terminus of the peptide and wherein
i) N−1 is selected from the group consisting of G, A, V, L, M, F, W, P, S, T, C, Y, N, Q, D, E, K, R, and H;
ii) N−2 is selected from the group consisting of G, A, V, L, I, M, F, W, S, T, C, Y, N, Q, D, E, K, R, and H;
iii) N−3 is selected from the group consisting of G, A, L, I, M, F, W, P, S, T, C, Y, N, Q, D, E, K, R, and H; or
iv) N−4 is selected from the group consisting of A, V, L, I, M, F, W, P, S, T, C, Y, N, Q, D, E, K, R, and H;
SEQ ID NO: 133 and wherein the peptide further comprises a joining sequence of one or more amino acid residues located immediately adjacent to an N-terminus or a C-terminus of the peptide and wherein
i) N−1 is selected from the group consisting of G, A, V, L, I, M, F, W, P, S, T, C, Y, Q, D, E, K, R, and H;
ii) N−2 is selected from the group consisting of G, A, V, L, M, F, W, P, S, T, C, Y, N, Q, D, E, K, R, and H;
iii) N−3 is selected from the group consisting of G, A, V, L, I, M, F, W, S, T, C, Y, N, Q, D, E, K, R, and H;
iv) N−4 is selected from the group consisting of G, A, L, I, M, F, W, P, S, T, C, Y, N, Q, D, E, K, R, and H; or
v) N−5 is selected from the group consisting of A, V, L, I, M, F, W, P, S, T, C, Y, N, Q, D, E, K, R, and H;
SEQ ID NO: 89;
SEQ ID NO: 106;
SEQ ID NO: 219;
SEQ ID NO: 221;
SEQ ID NO: 260 and wherein the peptide further comprises a joining sequence of one or more amino acid residues located immediately adjacent to an N-terminus or a C-terminus of the peptide and wherein
i) N−1 is selected from the group consisting of G, A, L, I, M, F, W, P, S, T, C, Y, N, Q, D, E, K, R, and H;
ii) N−2 is selected from the group consisting of G, A, V, L, I, M, F, W, P, S, T, C, Y, Q, D, E, K, R, and H;
iii) N−3 is selected from the group consisting of G, A, V, L, M, F, W, P, S, T, C, Y, N, Q, D, E, K, R, and H;
iv) N−4 is selected from the group consisting of G, A, V, L, I, M, F, W, S, T, C, Y, N, Q, D, E, K, R, and H;
v) N−5 is selected from the group consisting of G, A, L, I, M, F, W, P, S, T, C, Y, N, Q, D, E, K, R, and H;
vi) N−6 is selected from the group consisting of A, V, L, I, M, F, W, P, S, T, C, Y, N, Q, D, E, K, R, and H;
vii) C+1 is selected from the group consisting of G, A, V, L, I, M, F, W, P, S, C, Y, N, Q, D, E, K, R, and H; or
viii) C+2 is selected from the group consisting of G, A, V, L, I, M, F, W, S, T, C, Y, N, Q, D, E, K, R, and H; or
SEQ ID NO: 262 and wherein the peptide further comprises a joining sequence of one or more amino acid residues located immediately adjacent to an N-terminus or a C-terminus of the peptide and wherein
i) N−1 is selected from the group consisting of G, A, L, I, M, F, W, P, S, T, C, Y, N, Q, D, E, K, R, and H;
ii) N−2 is selected from the group consisting of G, A, V, L, I, M, F, W, P, S, T, C, Y, Q, D, E, K, R, and H;
iii) N−3 is selected from the group consisting of G, A, V, L, M, F, W, P, S, T, C, Y, N, Q, D, E, K, R, and H;
iv) N−4 is selected from the group consisting of G, A, V, L, I, M, F, W, S, T, C, Y, N, Q, D, E, K, R, and H;
v) N−5 is selected from the group consisting of G, A, L, I, M, F, W, P, S, T, C, Y, N, Q, D, E, K, R, and H;
vi) N−6 is selected from the group consisting of A, V, L, I, M, F, W, P, S, T, C, Y, N, Q, D, E, K, R, and H; or
vii) C+1 is selected from the group consisting of G, A, V, L, I, M, F, W, S, T, C, Y, N, Q, D, E, K, R, and H.

50. The peptide of any one of claims 1-46 or the peptide active agent complex of any one of claims 47-49, wherein the peptide comprises at least 70%, at least 80%, at least 90%, at least 95%, at least 97%, at least 99%, or 100% sequence identity to:
SEQ ID NO: 52;
SEQ ID NO: 53;
SEQ ID NO: 54;
SEQ ID NO: 55;
SEQ ID NO: 56;
SEQ ID NO: 57;
SEQ ID NO: 58;
SEQ ID NO: 59;
SEQ ID NO: 60;
SEQ ID NO: 61;
SEQ ID NO: 62;
SEQ ID NO: 63;
SEQ ID NO: 64;
SEQ ID NO: 65;
SEQ ID NO: 66;
SEQ ID NO: 134;
SEQ ID NO: 135;
SEQ ID NO: 136;
SEQ ID NO: 137;
SEQ ID NO: 138;
SEQ ID NO: 139;
SEQ ID NO: 140;
SEQ ID NO: 141;
SEQ ID NO: 142;
SEQ ID NO: 143;
SEQ ID NO: 144;
SEQ ID NO: 145;
SEQ ID NO: 146;
SEQ ID NO: 147; or
SEQ ID NO: 148.

51. The peptide of any one of claims 1-46 or the peptide active agent complex of any one of claims 47-50, wherein the peptide homes, targets, migrates to, accumulates in, binds to, is retained by, or is directed to cartilage, to kidney, or to cartilage and kidney.

52. The peptide of any one of claims 1-46 or the peptide active agent complex of any one of claims 47-51, wherein the peptide homes, targets, migrates to, accumulates in, binds to, is retained by, or is directed to proximal tubules of the kidney.

53. The peptide of any one of claims 1-46 or the peptide active agent complex of any one of claims 47-52, wherein the peptide is covalently complexed to the active agent.

54. The peptide active agent complex of any one of claims 47-53, wherein the peptide active agent complex homes, targets, migrates to, accumulates in, binds to, is retained by, or is directed to a cartilage or a kidney of the subject.

55. The peptide of any one of claims 1-46 or the peptide active agent complex of any one of claims 47-54, wherein the peptide comprises 4 or more cysteine residues.

56. The peptide of any one of claims 1-46 or the peptide active agent complex of any one of claims 47-55, wherein the peptide comprises three or more disulfide bridges formed between cysteine residues, wherein one of the disulfide bridges passes through a loop formed by two other disulfide bridges.

57. The peptide of any one of claims 1-46 or the peptide active agent complex of any one of claims 47-56, wherein the peptide comprises a plurality of disulfide bridges formed between cysteine residues.

58. The peptide of any one of claims 1-46 or the peptide active agent complex of any one of claims 47-57, wherein the peptide comprises a disulfide through a disulfide knot.

59. The peptide of any one of claims 1-46 or the peptide active agent complex of any one of claims 47-58, wherein at least one amino acid residue of the peptide is in an L configuration or, wherein at least one amino acid residue of the peptide is in a D configuration.

60. The peptide of any one of claims 1-46 or the peptide active agent complex of any one of claims 47-59, wherein the sequence comprises at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 residues.

61. The peptide of any one of claims 1-46 or the peptide active agent complex of any one of claims 47-60, wherein any one or more K residues are replaced by an R residue or wherein any one or more R residues are replaced by for a K residue.

62. The peptide of any one of claims 1-46 or the peptide active agent complex of any one of claims 47-61, wherein any one or more M residues are replaced by any one of the I, L, or V residues.

63. The peptide of any one of claims 1-46 or the peptide active agent complex of any one of claims 47-62, wherein any one or more L residues are replaced by any one of the V, I, or M residues.

64. The peptide of any one of claims 1-46 or the peptide active agent complex of any one of claims 47-63, wherein any one or more I residues are replaced by any of the M, L, or V residues.

65. The peptide of any one of claims 1-46 or the peptide active agent complex of any one of claims 47-64, wherein any one or more V residues are replaced by any of the M, I, or L residues.

66. The peptide of any one of claims 1-46 or the peptide active agent complex of any one of claims 47-65, wherein any one or more G residues are replaced by an A residue or wherein any one or more A residues are replaced by a G residue.

67. The peptide of any one of claims 1-46 or the peptide active agent complex of any one of claims 47-66, wherein any one or more S residues are replaced by a T residue or wherein any one or more T residues are replaced by for an S residue.

68. The peptide of any one of claims 1-46 or the peptide active agent complex of any one of claims 47-67, wherein any one or more Q residues are replaced by an N residue or wherein any one or more N residues are replaced by a Q residue.

69. The peptide of any one of claims 1-46 or the peptide active agent complex of any one of claims 47-68, wherein any one or more D residues are replaced by an E residue or wherein any one or more E residues are replaced by a D residue.

70. The peptide of any one of claims 1-46 or the peptide active agent complex of any one of claims 47-69, wherein the peptide has a charge distribution comprising an acidic region and a basic region.

71. The peptide of claim 70, wherein the acidic region is a nub.

72. The peptide of claim 70, wherein the basic region is a patch.

73. The peptide of any one of claims 1-46 or the peptide active agent complex of any one of claims 47-72, wherein the peptide comprises 5-12 basic residues.

74. The peptide of any one of claims 1-46 or the peptide active agent complex of any one of claims 47-73, wherein the peptide comprises 0-5 acidic residues.

75. The peptide of any one of claims 1-46 or the peptide active agent complex of any one of claims 47-74, wherein the peptide comprises 6 or more basic residues and 2 or fewer acidic residues.

76. The peptide of any one of claims 1-46 or the peptide active agent complex of any one of claims 47-75, wherein the peptide comprises a 4-19 amino acid residue fragment containing at least 2 cysteine residues, and at least 2 positively charged amino acid residues.

77. The peptide of any one of claims 1-46 or the peptide active agent complex of any one of claims 47-76, wherein the peptide comprises a 20-70 amino acid residue fragment containing at least 2 cysteine residues, no more than 2 basic residues and at least 2 positively charged amino acid residues.

78. The peptide of any one of claims 1-46 or the peptide active agent complex of any one of claims 47-77, wherein the peptide comprises at least 3 positively charged amino acid residues.

79. The peptide of any one of claims 1-46 or the peptide active agent complex of any one of claims 47-78, wherein the positively charged amino acid residues are selected from K, R, or a combination thereof.

80. The peptide of any one of claims 1-46 or the peptide active agent complex of any one of claims 47-79, wherein the peptide has a charge greater than 2 at physiological pH.

81. The peptide of any one of claims 1-46 or the peptide active agent complex of any one of claims 47-80, wherein the peptide has a charge greater than 3.5 at physiological pH.

82. The peptide of any one of claims 1-46 or the peptide active agent complex of any one of claims 47-81, wherein the peptide has a charge greater than 4.5 at physiological pH.

83. The peptide of any one of claims 1-46 or the peptide active agent complex of any one of claims 47-82, wherein the peptide has a charge greater than 5.5 at physiological pH.

84. The peptide of any one of claims 1-46 or the peptide active agent complex of any one of claims 47-83, wherein the peptide has a charge greater than 6.5 at physiological pH.

85. The peptide of any one of claims 1-46 or the peptide active agent complex of any one of claims 47-84, wherein the peptide has a charge greater than 7.5 at physiological pH.

86. The peptide of any one of claims 1-46 or the peptide active agent complex of any one of claims 47-85, wherein the peptide has a charge greater than 8.5 at physiological pH.

87. The peptide of any one of claims 1-46 or the peptide active agent complex of any one of claims 47-86, wherein the peptide has a charge greater than 9.5 at physiological pH.

88. The peptide of any one of claims 1-46 or the peptide active agent complex of any one of claims 47-87, wherein the peptide is selected from a potassium channel agonist, a potassium channel antagonist, a portion of a potassium channel, a sodium channel agonist, a sodium channel antagonist, a calcium channel agonist, a calcium channel antagonist, a hadrucalcin, a theraphotoxin, a huwentoxin, a kaliotoxin, a cobatoxin or a lectin.

89. The peptide active agent complex of claim 88 or peptide of claim 88, wherein the lectin is SHL-Ib2.

90. The peptide of any one of claims 1-46 or the peptide active agent complex of any one of claims 47-89, wherein the peptide is arranged in a multimeric structure with at least one other peptide.

91. The peptide of any one of claims 1-46 or the peptide active agent complex of any one of claims 47-90, wherein at least one residue of the peptide comprises a chemical modification.

92. The peptide active agent complex of claim 91 or the peptide of claim 91, wherein the chemical modification is blocking the N-terminus of the peptide.

93. The peptide active agent complex of claim 91 or the peptide of claim 91, wherein the chemical modification is methylation, acetylation, or acylation.

94. The peptide active agent complex of claim 91 or the peptide of claim 91, wherein the chemical modification is:
methylation of one or more lysine residues or analogue thereof,
methylation of the N-terminus; or
methylation of one or more lysine residue or analogue thereof and methylation of the N-terminus.

95. The peptide of any one of claims 1-46 or the peptide active agent complex of any one of claims 47-94, wherein the peptide is linked to an acyl adduct.

96. The peptide active agent complex of any one of claims 47-95, wherein the active agent is fused with the peptide at an N-terminus or a C-terminus of the peptide.

97. The peptide active agent complex of any one of claims 47-96, wherein the active agent is another peptide.

98. The peptide active agent complex of any one of claims 47-96, wherein the active agent is an antibody.

99. The peptide active agent complex of any one of claims 47-96, wherein the active agent is an Fc domain, Fab domain, scFv, or Fv fragment.

100. The peptide active agent complex of any one of claims 47-96, wherein the active agent is a glucocorticoid.

101. The peptide active agent complex of any one of claims 47-96, wherein the active agent is desciclesonide.

102. The peptide active agent complex of claim 99, wherein the peptide fused with the Fc domain comprises a contiguous sequence.

103. The peptide of any one of claims 1-46 or the peptide active agent complex of any one of claims 47-102, wherein 1, 2, 3, 4, 5, 6, 7, 8, 9, or 10 active agents are linked to the peptide.

104. The peptide active agent complex of any one of claims 47-103, wherein the peptide is linked to the active agent at an N-terminus, at the epsilon amine of a lysine residue, at the carboxylic acid of an aspartic acid or glutamic acid residue, or a C-terminus of the peptide by a linker.

105. The peptide active agent complex of any one of claims 47-104, wherein the peptide is linked to the active agent via a cleavable linker.

106. The peptide of any one of claims 1-46 or the peptide active agent complex of any one of claims 47-105 further comprising a non-natural amino acid, wherein the non-natural amino acid is an insertion, appendage, or substitution for another amino acid.

107. The peptide active agent complex of claim 106 or peptide of claim 106, wherein the peptide is linked to the active agent at the non-natural amino acid by a linker.

108. The peptide active agent complex of any one of claims 47-107, wherein the linker comprises an amide bond, an ester bond, a carbamate bond, a carbonate bond, a hydrazone bond, an oxime bond, a disulfide bond, a thioester bond, a thioether bond, a triazole, a carbon-carbon bond, or a carbon-nitrogen bond.

109. The peptide active agent complex of any one of claims 47-108, wherein the linker comprises an ester bond.

110. The peptide active agent complex of claim 105, wherein the cleavable linker comprises a cleavage site for matrix metalloproteinases, thrombin, cathepsins, or beta-glucuronidase.

111. The peptide active agent complex of any one of claims 104-110, wherein the linker is a hydrolytically labile linker.

112. The peptide active agent complex of any one of claims 104-111, wherein the linker is pH sensitive, reducible, glutathione-sensitive, or protease cleavable.

113. The peptide active agent complex of any one of claims 47-112, wherein the peptide is linked to the active agent via a stable linker.

114. The peptide of any one of claims 1-46 or the peptide active agent complex of any one of claims 47-113, wherein the peptide has an isoelectric point of about 9.

115. The peptide of any one of claims 1-46 or the peptide active agent complex of any one of claims 47-114, wherein the peptide is linked to a detectable agent.

116. The peptide active agent complex or peptide of claim 115, wherein the detectable agent is fused with the peptide at an N-terminus or a C-terminus of the peptide.

117. The peptide active agent complex or peptide any one of claims 115-116, wherein 1, 2, 3, 4, 5, 6, 7, 8, 9, or 10 detectable agents are linked to the peptide.

118. The peptide active agent complex or peptide of any one of claims 115-117, wherein the peptide is linked to the detectable agent via a cleavable linker.

119. The peptide active agent complex or peptide of any one of claims 115-118, wherein the peptide is linked to the detectable agent at an N-terminus, at the epsilon amine of an internal lysine residue, or a C-terminus of the peptide by a linker.

120. The peptide active agent complex or peptide of any one of claims 115-119, further comprising a non-natural amino acid, wherein the non-natural amino acid is an insertion, appendage, or substitution for another amino acid.

121. The peptide active agent complex or peptide of claim 120, wherein the peptide is linked to the detectable agent at the non-natural amino acid by a linker.

122. The peptide active agent complex or peptide of any one of claims 115-121, wherein the linker comprises an amide bond, an ester bond, a carbamate bond, a hydrazone bond, an oxime bond, or a carbon-nitrogen bond.

123. The peptide active agent complex or peptide of claim 118, wherein the cleavable linker comprises a cleavage site for matrix metalloproteinases, thrombin, cathepsins, or beta-glucuronidase.

124. The peptide active agent complex or peptide of any one of claims 115-118, wherein the peptide is linked to the detectable agent via a stable linker.

125. The peptide active agent complex or peptide of any one of claims 115-124, wherein the detectable agent is a fluorophore, a near-infrared dye, a contrast agent, a nanoparticle, a metal-containing nanoparticle, a metal chelate, an X-ray contrast agent, a PET agent, a radioisotope, or a radionuclide chelator.

126. The peptide active agent complex or peptide of any one of claims 115-125, wherein the detectable agent is a fluorescent dye.

127. The peptide of any one of claims 1-46 or the peptide active agent complex of any one of claims 47-126, wherein the peptide further comprises a joining sequence.

128. The peptide of claim 127, wherein the joining sequence is at an N-terminus or a C-terminus of the peptide.

129. The peptide of any one of claims 127-128, wherein the joining sequence comprises from 1 to 100 amino acid residues.

130. A pharmaceutical composition comprising the peptide of any one of claims 1-46 or a salt thereof, or the peptide active agent complex of any of claims 47-129 or a salt thereof, and a pharmaceutically acceptable carrier.

131. The pharmaceutical composition of claim 130, wherein the pharmaceutical composition is formulated for administration to a subject.

132. The pharmaceutical composition of any of claims 130-131, wherein the pharmaceutical composition is formulated for inhalation, intranasal administration, oral administration, topical administration, parenteral administration, intravenous administration, subcutaneous administration, intra-articular administration, intramuscular administration, intraperitoneal administration, dermal administration, transdermal administration, or a combination thereof.

133. A method of treating a condition in a subject in need thereof, the method comprising administering to the subject the peptide of any one of claims 1-46, the peptide active agent complex of any of claims 47-129, or a pharmaceutical composition of any one of claims 130-132.

134. The method of claim 133, wherein the peptide active agent complex, peptide, or pharmaceutical composition is administered by inhalation, intranasally, orally, topically, parenterally, intravenously, subcutaneously, intra-articularly, intramuscularly administration, intraperitoneally, dermally, transdermally, or a combination thereof.

135. The method any one of claims 133-134, wherein the peptide active agent complex or the peptide homes, targets, or migrates to cartilage of the subject following administration.

136. The method of any of claims 133-135, wherein the condition is associated with cartilage.

137. The method of any one of claims 133-135, wherein the condition is associated with a joint.

138. The method of any of claims 133-135, wherein the condition is an inflammation, a cancer, a degradation, a growth disturbance, genetic, a tear, an infection, a disease, or an injury.

139. The method of any of claims 133-135, wherein the condition is a chondrodystrophy.

140. The method of any of claims 133-135, wherein the condition is a traumatic rupture or detachment.

141. The method of any of claims 133-135, wherein the condition is a costochondritis.

142. The method of any of claims 133-135, wherein the condition is a herniation.

143. The method of any of claims 133-135, wherein the condition is a polychondritis.

144. The method of any of claims 133-135, wherein the condition is a chordoma.

145. The method of any of claims 133-135, wherein the condition is a type of arthritis.

146. The method of claim 145, wherein the type of arthritis is rheumatoid arthritis.

147. The method of claim 145, wherein the type of arthritis is osteoarthritis.

148. The method of claim 145, wherein the type of arthritis is lupus arthritis.

149. The method of any one of claims 133-135, wherein the condition is Systemic lupus erythematosus.

150. The method of any of claims 133-135, wherein the condition is achondroplasia.

151. The method of any of claims 133-135, wherein the condition is benign chondroma or malignant chondrosarcoma.

152. The method of any of claims 133-135, wherein the condition is bursitis, tendinitis, gout, pseudogout, an arthropathy, psoriatic arthritis, ankylosing spondylitis, or an infection.

153. The method of claim 138, wherein the peptide active agent complex, peptide, or pharmaceutical composition is administered to treat the injury, to repair a tissue damaged by the injury, or to treat a pain caused by the injury.

154. The method of claim 138, wherein the peptide active agent complex, peptide, or pharmaceutical composition is administered to treat the tear or to repair a tissue damaged by the tear.

155. The method of any one of claims 133-134, wherein the peptide active agent complex, peptide, or pharmaceutical composition homes, targets, or migrates to a kidney of the subject following administration.

156. The method of any one of claims 133-134 or 155, wherein the condition is associated with a kidney.

157. The method of claim 156, wherein the condition is lupus nephritis, acute kidney injury (AKI), chronic kidney disease (CKD), hypertensive kidney damage, diabetic nephropathy, lupus nephritis, or renal fibrosis.

158. A method of imaging an organ or body region of a subject, the method comprising:
administering to the subject the peptide of any one of claims 1-46, the peptide active agent complex of any of claims 47-129, or a pharmaceutical composition of any one of claims 130-132; and
imaging the subject.

159. The method of claim 158, wherein further comprising detecting a cancer or diseased region, tissue, structure or cell.

160. The method of any one of claims 158-159, further comprising performing surgery on the subject.

161. The method of any one of claims 158-160, further comprising treating the cancer.

162. The method of any one of claims 158-161, wherein the surgery comprises removing the cancer or the diseased region, tissue, structure or cell of the subject.

163. The method of claim 160, further comprising imaging the cancer or diseased region, tissue, structure, or cell of the subject after surgical removal.

164. The peptide active agent complex of any one of claims 47-129, wherein the peptide active agent complex is expressed as a fusion protein.

165. A method of treating or delivering a peptide or peptide agent complex to a subject in need thereof according to any one of claims 133-164, the method further comprising administering a companion diagnostic, therapeutic, or imaging agent, wherein the companion diagnostic or imaging agent comprises
a) the peptide active agent complex of any of claims 42-124,
b) the peptide of any of claims 1-41; or
c) a peptide of SEQ ID NO: 27-SEQ ID NO: 45, SEQ ID NO: 47-SEQ ID NO: 66, SEQ ID NO: 109-SEQ ID NO: 126, SEQ ID NO: 129-SEQ ID NO: 148, or SEQ ID NO: 233-SEQ ID NO: 256 further comprising a diagnostic, therapeutic, or imaging agent, wherein the diagnostic or imaging agent comprises a chemical agent, a radiolabel agent, radiosensitizing agent, fluorophore, an imaging agent, a diagnostic agent, a protein, a peptide, or a small molecule.

166. The method of claim 165, wherein the companion diagnostic, therapeutic, or imaging agent is detected by a device.

167. The method of claim 166, wherein the device is used to detect the companion diagnostic, therapeutic, or imaging agent or to assess an agent's safety and physiologic effect.

168. The method of claim 167, wherein the agent's safety and physiologic effect is bioavailability, uptake, distribution and clearance, metabolism, pharmacokinetics, localization, measurement of concentrations in blood and tissues, assessing therapeutic window, range and optimization.

169. The method of any one of claims 165-168, wherein the method is combined with or integrated into a surgical microscope, confocal microscope, fluorescence scope, exoscope, endoscope, or a surgical robot comprising a KINEVO 900, QEVO, CONVIVO, OMPI PENTERO 900, OMPI PENTERO 800, INFRARED 800, FLOW 800, OMPI LUMERIA, OMPI Vario, OMPI VARIO 700, OMPI Pico, TREMON 3DHD, a PROVido, ARvido, GLOW 800, Leica M530 OHX, Leica M530 OH6, Leica M720 OHX5, Leica M525 F50, Leica M525 F40, Leica M525 F20, Leica M525 OH4, Leica HD C100, Leica FL560, Leica FL400 Leica FL800, Leica DI C500, Leica ULT500, Leica Rotatable Beam Splitter, Leica M651 MSD, LIGHTENING, Leica TCS SP8, SP8 FALCON, SP8 DIVE, Leica TCS SP8 STED, Leica TCS SP8 DLS, Leica TCS SP8 X, Leica TCS SP8 CARS, Leica TCS SPE), Leica HyD, Leica HCS A, Leica DCM8, Haag-Streit 5-1000, Haag-Streit 3-1000, and Intuitive Surgical da Vinci surgical robot.

170. The method of any one of claims 166-169, wherein the device incorporates radiology or fluorescence, including the X-ray radiography, magnetic resonance imaging (MRI), ultrasound, endoscopy, elastography, tactile imaging, thermography, flow cytometry, medical photography, nuclear medicine functional imaging techniques, positron emission tomography (PET), single-photon emission computed tomography (SPECT), surgical instrument, operating microscope, confocal microscope, fluorescence scope, exoscope, or a surgical robot.

171. The peptide of any one of claims 2, 28, 34, 41, 42, or 45, wherein the peptide comprises at least two of, at least three of, at least four of, at least five of, at least six of, at least seven of, at least eight of, or all of KCIN (SEQ ID NO: 91); PCKR (SEQ ID NO: 93); KQC (SEQ ID NO: 95); RQC (SEQ ID NO: 101); PCKK (SEQ ID NO: 102); GKCMNGKC (SEQ ID NO: 104); GRCMNGRC (SEQ ID NO: 105); or GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R wherein each X can independently be any amino acid or amino acid analogue or null.

172. The peptide of any one of claims 3, 29, 34, 41, 42, or 45, wherein the peptide comprises at least two of, at least three of, at least four of, at least five of, at least six of, at least seven of, at least eight of, at least nine of, at least ten of, or all of RCIN (SEQ ID NO: 97); PCRR (SEQ ID NO: 99); GRCINSRC (SEQ ID NO: 227); GRCIXXRC (SEQ ID NO: 228) wherein each X can independently be any amino acid or amino acid analogue or null; GRCIX¹X²RC (SEQ ID NO: 229), wherein X¹=N, Q, A, S, T or L and X²=S, G, or R; PCR (SEQ ID NO: 230); CLDPCRRA (SEQ ID NO: 231); CLDPCRR (SEQ ID NO: 232); RCRGSRDC (SEQ ID NO: 257); PCRRAG (SEQ ID NO: 258); or RFGRCI (SEQ ID NO: 259).

173. The peptide of claim 30, wherein the peptide comprises at least two of, at least three of, at least four of, at least five of, at least six of, at least seven of, at least eight of, at least nine of, at least ten of, at least 11 of, at least 12 of, at least 13 of, at least 14 of, at least 15 of, at least 16 of, at least 17 of, at least 18 of, at least 19 of, at least 20 of, at least 21 of, at least 22 of, at least 23 of, at least 24 of, at least 25 of, or all of GKCINKKCKC (SEQ ID NO: 90); KCIN (SEQ ID NO: 91); KKCK (SEQ ID NO: 92); PCKR (SEQ ID NO: 93); KRCSRR (SEQ ID NO: 94); KQC (SEQ ID NO: 95); GRCINRRCRC (SEQ ID NO: 96); RCIN (SEQ ID NO: 97); RRCR (SEQ ID NO: 98); PCRR (SEQ ID NO: 99); RRCSRR (SEQ ID NO: 100); RQC (SEQ ID NO: 101); PCKK (SEQ ID NO: 102), KKCSKK (SEQ ID NO: 103), GKCMNGKC (SEQ ID NO: 104); GRCMNGRC (SEQ ID NO: 105); GRCINSRC (SEQ ID NO: 227), GRCIXXRC (SEQ ID NO: 228) wherein each X can independently be any amino acid or amino acid analogue or null, GRCIX¹X²RC (SEQ ID NO: 229), wherein X¹=N, Q, A, S, T or L and X²=S, G, or R (SEQ ID NO: 229), PCR (SEQ ID NO: 230), CLDPCRRA (SEQ ID NO: 231), CLDPCRR (SEQ ID NO: 232), RCRGSRDC (SEQ ID NO: 257), PCRRAG (SEQ ID NO: 258), and RFGRCI (SEQ ID NO: 259).

174. The peptide of any one of claims 1, 34, 41, 42, or 45, wherein the peptide further comprises at least two of, at least three of, at least four of, at least five of, at least six of, at least seven of, at least eight of, at least nine of, at least ten of, at least 11 of, at least 12 of, at least 13 of, at least 14 of, at least 15 of, at least 16 of, at least 17 of, at least 18 of, at least 19 of, at least 20 of, at least 21 of, at least 22 of, at least 23 of, at least 24 of, at least 25 of, at least 26 of, at least 27 of, at least 28 of, at least 29, of or all of the corresponding substitutions selected from the group consisting of N5S, D16E, M23T, N30Q, N30A, N30S, N30T, N30L, S31G, S31R, L15Y, H34Y, T36Y, D10E, M17T, N24Q, N24A, N24S, N24T, N24L, S25G, S25R, L9Y, H28Y, T30Y, R1K, R13K, R14K, R21K, and R26K.


SEQUENCE LISTING (266 sequences)

SEQ ID NO: 1-20 - skipped (20 entries)

SEQ ID NO: 21 - protein, 42 aa, Artificial Sequence
  Description of Artificial Sequence: Synthetic polypeptide
  MOD_RES (3)..(3): Any amino acid or amino acid analogue or null
  MOD_RES (5)..(5): Any amino acid or amino acid analogue or null
  MOD_RES (7)..(7): Any amino acid or amino acid analogue or null
  MOD_RES (11)..(11): Any amino acid or amino acid analogue or null
  MOD_RES (14)..(14): Any amino acid or amino acid analogue or null
  MOD_RES (18)..(18): Any amino acid or amino acid analogue or null
  MOD_RES (22)..(22): Any amino acid or amino acid analogue or null
  MOD_RES (24)..(24): Any amino acid or amino acid analogue or null
  MOD_RES (26)..(26): Any amino acid or amino acid analogue or null
  MOD_RES (28)..(28): Any amino acid or amino acid analogue or null
  MOD_RES (37)..(37): Any amino acid or amino acid analogue or null
  MOD_RES (39)..(39): Any amino acid or amino acid analogue or null
  MOD_RES (41)..(42): Any amino acid or amino acid analogue or null
GSXVXIXVKCXGSXQCLXPCKXAXGXRXGKCMNGKCXCXPXX

SEQ ID NO: 22 - protein, 42 aa, Artificial Sequence
  Description of Artificial Sequence: Synthetic polypeptide
  MOD_RES (3)..(3): Any amino acid or amino acid analogue or null
  MOD_RES (5)..(5): Any amino acid or amino acid analogue or null
  MOD_RES (7)..(7): Any amino acid or amino acid analogue or null
  MOD_RES (11)..(11): Any amino acid or amino acid analogue or null
  MOD_RES (14)..(14): Any amino acid or amino acid analogue or null
  MOD_RES (18)..(18): Any amino acid or amino acid analogue or null
  MOD_RES (22)..(22): Any amino acid or amino acid analogue or null
  MOD_RES (24)..(24): Any amino acid or amino acid analogue or null
  MOD_RES (26)..(26): Any amino acid or amino acid analogue or null
  MOD_RES (28)..(28): Any amino acid or amino acid analogue or null
  MOD_RES (37)..(37): Any amino acid or amino acid analogue or null
  MOD_RES (39)..(39): Any amino acid or amino acid analogue or null
  MOD_RES (41)..(42): Any amino acid or amino acid analogue or null
  See specification as filed for detailed description of substitutions and preferred embodiments
GSXVXIXVRCXGSXQCLXPCRXAXGXRXGRCMNGRCXCXPXX

SEQ ID NO: 23 - protein, 39 aa, Artificial Sequence
  Description of Artificial Sequence: Synthetic polypeptide
  MOD_RES (7)..(7): N, S, or G
  MOD_RES (17)..(17): L or Y
  MOD_RES (18)..(18): D or E
  MOD_RES (25)..(25): M or T
  MOD_RES (32)..(32): N, Q, A, S, T, or L
  MOD_RES (33)..(33): S, G, or R
  MOD_RES (36)..(36): H or Y
  MOD_RES (38)..(38): T or Y
  See specification as filed for detailed description of substitutions and preferred embodiments
GSGVPIXVRCRGSRDCXXPCRRAGXRFGRCIXXRCXCXP

SEQ ID NO: 24 - protein, 33 aa, Artificial Sequence
  Description of Artificial Sequence: Synthetic polypeptide
  MOD_RES (11)..(11): L or Y
  MOD_RES (12)..(12): D or E
  MOD_RES (19)..(19): M or T
  MOD_RES (26)..(26): N, Q, A, S, T, or L
  MOD_RES (27)..(27): S, G, or R
  MOD_RES (30)..(30): H or Y
  MOD_RES (32)..(32): T or Y
GSRCRGSRDCXXPCRRAGXRFGRCIXXRCXCXP

SEQ ID NO: 25 - protein, 36 aa, Artificial Sequence
  Description of Artificial Sequence: Synthetic polypeptide
  MOD_RES (5)..(5): Any amino acid or amino acid analogue or null
  MOD_RES (8)..(8): Any amino acid or amino acid analogue or null
  MOD_RES (12)..(12): Any amino acid or amino acid analogue or null
  MOD_RES (16)..(16): Any amino acid or amino acid analogue or null
  MOD_RES (18)..(18): Any amino acid or amino acid analogue or null
  MOD_RES (20)..(20): Any amino acid or amino acid analogue or null
  MOD_RES (22)..(22): Any amino acid or amino acid analogue or null
  MOD_RES (31)..(31): Any amino acid or amino acid analogue or null
  MOD_RES (33)..(33): Any amino acid or amino acid analogue or null
  MOD_RES (35)..(36): Any amino acid or amino acid analogue or null
GSKCXGSXQCLXPCKXAXGXRXGKCMNGKCXCXPXX

SEQ ID NO: 26 - protein, 36 aa, Artificial Sequence
  Description of Artificial Sequence: Synthetic polypeptide
  MOD_RES (5)..(5): Any amino acid or amino acid analogue or null
  MOD_RES (8)..(8): Any amino acid or amino acid analogue or null
  MOD_RES (12)..(12): Any amino acid or amino acid analogue or null
  MOD_RES (16)..(16): Any amino acid or amino acid analogue or null
  MOD_RES (18)..(18): Any amino acid or amino acid analogue or null
  MOD_RES (20)..(20): Any amino acid or amino acid analogue or null
  MOD_RES (22)..(22): Any amino acid or amino acid analogue or null
  MOD_RES (31)..(31): Any amino acid or amino acid analogue or null
  MOD_RES (33)..(33): Any amino acid or amino acid analogue or null
  MOD_RES (35)..(36): Any amino acid or amino acid analogue or null
GSRCXGSXQCLXPCRXAXGXRXGRCMNGRCXCXPXX

SEQ ID NO: 27 - protein, 33 aa, Artificial Sequence
  Description of Artificial Sequence: Synthetic polypeptide
GSKCRGSRDCLDPCKKAGMRFGKCINSKCHCTP

SEQ ID NO: 28 - protein, 33 aa, Artificial Sequence
  Description of Artificial Sequence: Synthetic polypeptide
GSRCRGSRDCLDPCRRAGMRFGRCINSRCHCTP

SEQ ID NO: 29 - protein, 33 aa, Artificial Sequence
  Description of Artificial Sequence: Synthetic polypeptide
GSRCRGSRDCLEPCRRAGTRFGRCINGRCHCTP

SEQ ID NO: 30 - protein, 33 aa, Artificial Sequence
  Description of Artificial Sequence: Synthetic polypeptide
GSRCRGSRDCLEPCRRAGTRFGRCINRRCHCTP

SEQ ID NO: 31 - protein, 33 aa, Artificial Sequence
  Description of Artificial Sequence: Synthetic polypeptide
GSRCRGSRDCLEPCRRAGTRFGRCINSRCHCTP

SEQ ID NO: 32 - protein, 33 aa, Artificial Sequence
  Description of Artificial Sequence: Synthetic polypeptide
GSRCRGSRDCLEPCRRAGTRFGRCIQSRCHCTP

SEQ ID NO: 33 - protein, 33 aa, Artificial Sequence
  Description of Artificial Sequence: Synthetic polypeptide
GSRCRGSRDCLEPCRRAGTRFGRCIQSRCHCYP

SEQ ID NO: 34 - protein, 33 aa, Artificial Sequence
  Description of Artificial Sequence: Synthetic polypeptide
GSRCRGSRDCYEPCRRAGTRFGRCIQSRCHCTP

SEQ ID NO: 35 - protein, 33 aa, Artificial Sequence
  Description of Artificial Sequence: Synthetic polypeptide
GSRCRGSRDCLEPCRRAGTRFGRCIQSRCYCTP

SEQ ID NO: 36 - protein, 33 aa, Artificial Sequence
  Description of Artificial Sequence: Synthetic polypeptide
GSRCRGSRDCLEPCRRAGTRFGRCIASRCHCYP

SEQ ID NO: 37 - protein, 33 aa, Artificial Sequence
  Description of Artificial Sequence: Synthetic polypeptide
GSRCRGSRDCLEPCRRAGTRFGRCISSRCHCYP

SEQ ID NO: 38 - protein, 33 aa, Artificial Sequence
  Description of Artificial Sequence: Synthetic polypeptide
GSRCRGSRDCLEPCRRAGTRFGRCITSRCHCYP

SEQ ID NO: 39 - protein, 33 aa, Artificial Sequence
  Description of Artificial Sequence: Synthetic polypeptide
GSKCRGSRDCLDPCRRAGMRFGRCINSRCHCTP

SEQ ID NO: 40 - protein, 33 aa, Artificial Sequence
  Description of Artificial Sequence: Synthetic polypeptide
GSRCRGSRDCLDPCKRAGMRFGRCINSRCHCTP

SEQ ID NO: 41 - protein, 33 aa, Artificial Sequence
  Description of Artificial Sequence: Synthetic polypeptide
GSRCRGSRDCLDPCRKAGMRFGRCINSRCHCTP

SEQ ID NO: 42 - protein, 33 aa, Artificial Sequence
  Description of Artificial Sequence: Synthetic polypeptide
GSRCRGSRDCLDPCRRAGMRFGKCINSRCHCTP

SEQ ID NO: 43 - protein, 33 aa, Artificial Sequence
  Description of Artificial Sequence: Synthetic polypeptide
GSRCRGSRDCLDPCRRAGMRFGRCINSKCHCTP

SEQ ID NO: 44 - protein, 33 aa, Artificial Sequence
  Description of Artificial Sequence: Synthetic polypeptide
GSRCRGSRDCLEPCRRAGTRFGRCINSRCHCYP

SEQ ID NO: 45 - protein, 40 aa, Artificial Sequence
  Description of Artificial Sequence: Synthetic polypeptide
GSSGVPINVRCRGSRDCLDPCRRAGMRFGRCINSRCHCTP

SEQ ID NO: 46 - protein, 39 aa, Artificial Sequence
  Description of Artificial Sequence: Synthetic polypeptide
GSGVPINVRCRGSRDCLDPCRRAGMRFGRCINSRCHCTP

SEQ ID NO: 47 - protein, 38 aa, Artificial Sequence
  Description of Artificial Sequence: Synthetic polypeptide
GSVPINVRCRGSRDCLDPCRRAGMRFGRCINSRCHCTP

SEQ ID NO: 48 - protein, 37 aa, Artificial Sequence
  Description of Artificial Sequence: Synthetic polypeptide
GSPINVRCRGSRDCLDPCRRAGMRFGRCINSRCHCTP

SEQ ID NO: 49 - protein, 36 aa, Artificial Sequence
  Description of Artificial Sequence: Synthetic polypeptide
GSINVRCRGSRDCLDPCRRAGMRFGRCINSRCHCTP

SEQ ID NO: 50 - protein, 35 aa, Artificial Sequence
  Description of Artificial Sequence: Synthetic polypeptide
GSNVRCRGSRDCLDPCRRAGMRFGRCINSRCHCTP

SEQ ID NO: 51 - protein, 34 aa, Artificial Sequence
  Description of Artificial Sequence: Synthetic polypeptide
GSVRCRGSRDCLDPCRRAGMRFGRCINSRCHCTP

SEQ ID NO: 52 - protein, 39 aa, Artificial Sequence
  Description of Artificial Sequence: Synthetic polypeptide
GSGVPISVRCRGSRDCLEPCRRAGTRFGRCINGRCHCTP

SEQ ID NO: 53 - protein, 39 aa, Artificial Sequence
  Description of Artificial Sequence: Synthetic polypeptide
GSGVPISVRCRGSRDCLEPCRRAGTRFGRCIQSRCHCTP

SEQ ID NO: 54 - protein, 39 aa, Artificial Sequence
  Description of Artificial Sequence: Synthetic polypeptide
GSGVPISVRCRGSRDCLEPCRRAGTRFGRCINRRCHCTP

SEQ ID NO: 55 - protein, 39 aa, Artificial Sequence
  Description of Artificial Sequence: Synthetic polypeptide
GSGVPINVRCRGSRDCLEPCRRAGTRFGRCINSRCHCTP

SEQ ID NO: 56 - protein, 39 aa, Artificial Sequence
  Description of Artificial Sequence: Synthetic polypeptide
GSGVPINVRCRGSRDCLEPCRRAGTRFGRCIQSRCHCTP

SEQ ID NO: 57 - protein, 39 aa, Artificial Sequence
  Description of Artificial Sequence: Synthetic polypeptide
GSGVPINVRCRGSRDCLEPCRRAGTRFGRCIQSRCHCYP

SEQ ID NO: 58 - protein, 39 aa, Artificial Sequence
  Description of Artificial Sequence: Synthetic polypeptide
GSGVPINVRCRGSRDCYEPCRRAGTRFGRCIQSRCHCTP

SEQ ID NO: 59 - protein, 39 aa, Artificial Sequence
  Description of Artificial Sequence: Synthetic polypeptide
GSGVPINVRCRGSRDCLEPCRRAGTRFGRCIQSRCYCTP

SEQ ID NO: 60 - protein, 39 aa, Artificial Sequence
  Description of Artificial Sequence: Synthetic polypeptide
GSGVPISVRCRGSRDCLEPCRRAGTRFGRCIQSRCHCYP

SEQ ID NO: 61 - protein, 39 aa, Artificial Sequence
  Description of Artificial Sequence: Synthetic polypeptide
GSGVPISVRCRGSRDCYEPCRRAGTRFGRCIQSRCHCTP

SEQ ID NO: 62 - protein, 39 aa, Artificial Sequence
  Description of Artificial Sequence: Synthetic polypeptide
GSGVPISVRCRGSRDCLEPCRRAGTRFGRCIQSRCYCTP

SEQ ID NO: 63 - protein, 39 aa, Artificial Sequence
  Description of Artificial Sequence: Synthetic polypeptide
GSGVPINVRCRGSRDCLEPCRRAGTRFGRCIASRCHCYP

SEQ ID NO: 64 - protein, 39 aa, Artificial Sequence
  Description of Artificial Sequence: Synthetic polypeptide
GSGVPINVRCRGSRDCLEPCRRAGTRFGRCISSRCHCYP

SEQ ID NO: 65 - protein, 39 aa, Artificial Sequence
  Description of Artificial Sequence: Synthetic polypeptide
GSGVPINVRCRGSRDCLEPCRRAGTRFGRCITSRCHCYP

SEQ ID NO: 66 - protein, 39 aa, Artificial Sequence
  Description of Artificial Sequence: Synthetic polypeptide
GSGVPINVRCRGSRDCLEPCRRAGTRFGRCINSRCHCYP

SEQ ID NO: 67-86 - skipped (20 entries)

SEQ ID NO: 87 - protein, 40 aa, Artificial Sequence
  Description of Artificial Sequence: Synthetic polypeptide
  MOD_RES (1)..(1): Any amino acid or amino acid analogue or null
  MOD_RES (3)..(3): Any amino acid or amino acid analogue or null
  MOD_RES (5)..(5): Any amino acid or amino acid analogue or null
  MOD_RES (9)..(9): Any amino acid or amino acid analogue or null
  MOD_RES (12)..(12): Any amino acid or amino acid analogue or null
  MOD_RES (16)..(16): Any amino acid or amino acid analogue or null
  MOD_RES (20)..(20): Any amino acid or amino acid analogue or null
  MOD_RES (22)..(22): Any amino acid or amino acid analogue or null
  MOD_RES (24)..(24): Any amino acid or amino acid analogue or null
  MOD_RES (26)..(26): Any amino acid or amino acid analogue or null
  MOD_RES (35)..(35): Any amino acid or amino acid analogue or null
  MOD_RES (37)..(37): Any amino acid or amino acid analogue or null
  MOD_RES (39)..(40): Any amino acid or amino acid analogue or null
XVXIXVKCXGSXQCLXPCKXAXGXRXGKCMNGKCXCXPXX

SEQ ID NO: 88 - protein, 40 aa, Artificial Sequence
  Description of Artificial Sequence: Synthetic polypeptide
  MOD_RES (1)..(1): Any amino acid or amino acid analogue or null
  MOD_RES (3)..(3): Any amino acid or amino acid analogue or null
  MOD_RES (5)..(5): Any amino acid or amino acid analogue or null
  MOD_RES (9)..(9): Any amino acid or amino acid analogue or null
  MOD_RES (12)..(12): Any amino acid or amino acid analogue or null
  MOD_RES (16)..(16): Any amino acid or amino acid analogue or null
  MOD_RES (20)..(20): Any amino acid or amino acid analogue or null
  MOD_RES (22)..(22): Any amino acid or amino acid analogue or null
  MOD_RES (24)..(24): Any amino acid or amino acid analogue or null
  MOD_RES (26)..(26): Any amino acid or amino acid analogue or null
  MOD_RES (35)..(35): Any amino acid or amino acid analogue or null
  MOD_RES (37)..(37): Any amino acid or amino acid analogue or null
  MOD_RES (39)..(40): Any amino acid or amino acid analogue or null
XVXIXVRCXGSXQCLXPCRXAXGXRXGRCMNGRCXCXPXX

SEQ ID NO: 89 - protein, 37 aa, Artificial Sequence
  Description of Artificial Sequence: Synthetic polypeptide
  MOD_RES (5)..(5): N, S, or G
  MOD_RES (15)..(15): L or Y
  MOD_RES (16)..(16): D or E
  MOD_RES (23)..(23): M or T
  MOD_RES (30)..(30): N, Q, A, S, T, or L
  MOD_RES (31)..(31): S, G, or R
  MOD_RES (34)..(34): H or Y
  MOD_RES (36)..(36): T or Y
  See specification as filed for detailed description of substitutions and preferred embodiments
GVPIXVRCRGSRDCXXPCRRAGXRFGRCIXXRCXCXP

SEQ ID NO: 90 - protein, 10 aa, Artificial Sequence
  Description of Artificial Sequence: Synthetic peptide
GKCINKKCKC

SEQ ID NO: 91 - protein, 4 aa, Artificial Sequence
  Description of Artificial Sequence: Synthetic peptide
KCIN

SEQ ID NO: 92 - protein, 4 aa, Artificial Sequence
  Description of Artificial Sequence: Synthetic peptide
KKCK

SEQ ID NO: 93 - protein, 4 aa, Artificial Sequence
  Description of Artificial Sequence: Synthetic peptide
PCKR

SEQ ID NO: 94 - protein, 6 aa, Artificial Sequence
  Description of Artificial Sequence: Synthetic peptide
KRCSRR

SEQ ID NO: 95 - protein, 3 aa, Artificial Sequence
  Description of Artificial Sequence: Synthetic peptide
KQC

SEQ ID NO: 96 - protein, 10 aa, Artificial Sequence
  Description of Artificial Sequence: Synthetic peptide
GRCINRRCRC

SEQ ID NO: 97 - protein, 4 aa, Artificial Sequence
  Description of Artificial Sequence: Synthetic peptide
RCIN

SEQ ID NO: 98 - protein, 4 aa, Artificial Sequence
  Description of Artificial Sequence: Synthetic peptide
RRCR

SEQ ID NO: 99 - protein, 4 aa, Artificial Sequence
  Description of Artificial Sequence: Synthetic peptide
PCRR

SEQ ID NO: 100 - protein, 6 aa, Artificial Sequence
  Description of Artificial Sequence: Synthetic peptide
RRCSRR

SEQ ID NO: 101 - protein, 3 aa, Artificial Sequence
  Description of Artificial Sequence: Synthetic peptide
RQC

SEQ ID NO: 102 - protein, 4 aa, Artificial Sequence
  Description of Artificial Sequence: Synthetic peptide
PCKK

SEQ ID NO: 103 - protein, 6 aa, Artificial Sequence
  Description of Artificial Sequence: Synthetic peptide
KKCSKK

SEQ ID NO: 104 - protein, 8 aa, Artificial Sequence
  Description of Artificial Sequence: Synthetic peptide
GKCMNGKC

SEQ ID NO: 105 - protein, 8 aa, Artificial Sequence
  Description of Artificial Sequence: Synthetic peptide
GRCMNGRC

SEQ ID NO: 106 - protein, 31 aa, Artificial Sequence
  Description of Artificial Sequence: Synthetic polypeptide
  MOD_RES (9)..(9): L or Y
  MOD_RES (10)..(10): D or E
  MOD_RES (17)..(17): M or T
  MOD_RES (24)..(24): N, Q, A, S, T, or L
  MOD_RES (25)..(25): S, G, or R
  MOD_RES (28)..(28): H or Y
  MOD_RES (30)..(30): T or Y
RCRGSRDCXXPCRRAGXRFGRCIXXRCXCXP

SEQ ID NO: 107 - protein, 34 aa, Artificial Sequence
  Description of Artificial Sequence: Synthetic polypeptide
  MOD_RES (3)..(3): Any amino acid or amino acid analogue or null
  MOD_RES (6)..(6): Any amino acid or amino acid analogue or null
  MOD_RES (10)..(10): Any amino acid or amino acid analogue or null
  MOD_RES (14)..(14): Any amino acid or amino acid analogue or null
  MOD_RES (16)..(16): Any amino acid or amino acid analogue or null
  MOD_RES (18)..(18): Any amino acid or amino acid analogue or null
  MOD_RES (20)..(20): Any amino acid or amino acid analogue or null
  MOD_RES (29)..(29): Any amino acid or amino acid analogue or null
  MOD_RES (31)..(31): Any amino acid or amino acid analogue or null
  MOD_RES (33)..(34): Any amino acid or amino acid analogue or null
KCXGSXQCLXPCKXAXGXRXGKCMNGKCXCXPXX

SEQ ID NO: 108 - protein, 34 aa, Artificial Sequence
  Description of Artificial Sequence: Synthetic polypeptide
  MOD_RES (3)..(3): Any amino acid or amino acid analogue or null
  MOD_RES (6)..(6): Any amino acid or amino acid analogue or null
  MOD_RES (10)..(10): Any amino acid or amino acid analogue or null
  MOD_RES (14)..(14): Any amino acid or amino acid analogue or null
  MOD_RES (16)..(16): Any amino acid or amino acid analogue or null
  MOD_RES (18)..(18): Any amino acid or amino acid analogue or null
  MOD_RES (20)..(20): Any amino acid or amino acid analogue or null
  MOD_RES (29)..(29): Any amino acid or amino acid analogue or null
  MOD_RES (31)..(31): Any amino acid or amino acid analogue or null
  MOD_RES (33)..(34): Any amino acid or amino acid analogue or null
RCXGSXQCLXPCRXAXGXRXGRCMNGRCXCXPXX

SEQ ID NO: 109 - protein, 31 aa, Artificial Sequence
  Description of Artificial Sequence: Synthetic polypeptide
KCRGSRDCLDPCKKAGMRFGKCINSKCHCTP

SEQ ID NO: 110 - protein, 31 aa, Artificial Sequence
  Description of Artificial Sequence: Synthetic polypeptide
RCRGSRDCLDPCRRAGMRFGRCINSRCHCTP

SEQ ID NO: 111 - protein, 31 aa, Artificial Sequence
  Description of Artificial Sequence: Synthetic polypeptide
RCRGSRDCLEPCRRAGTRFGRCINGRCHCTP

SEQ ID NO: 112 - protein, 31 aa, Artificial Sequence
  Description of Artificial Sequence: Synthetic polypeptide
RCRGSRDCLEPCRRAGTRFGRCINRRCHCTP

SEQ ID NO: 113 - protein, 31 aa, Artificial Sequence
  Description of Artificial Sequence: Synthetic polypeptide
RCRGSRDCLEPCRRAGTRFGRCINSRCHCTP

SEQ ID NO: 114 - protein, 31 aa, Artificial Sequence
  Description of Artificial Sequence: Synthetic polypeptide
RCRGSRDCLEPCRRAGTRFGRCIQSRCHCTP

SEQ ID NO: 115 - protein, 31 aa, Artificial Sequence
  Description of Artificial Sequence: Synthetic polypeptide
RCRGSRDCLEPCRRAGTRFGRCIQSRCHCYP

SEQ ID NO: 116 - protein, 31 aa, Artificial Sequence
  Description of Artificial Sequence: Synthetic polypeptide
RCRGSRDCYEPCRRAGTRFGRCIQSRCHCTP

SEQ ID NO: 117 - protein, 31 aa, Artificial Sequence
  Description of Artificial Sequence: Synthetic polypeptide
RCRGSRDCLEPCRRAGTRFGRCIQSRCYCTP

SEQ ID NO: 118 - protein, 31 aa, Artificial Sequence
  Description of Artificial Sequence: Synthetic polypeptide
RCRGSRDCLEPCRRAGTRFGRCIASRCHCYP

SEQ ID NO: 119 - protein, 31 aa, Artificial Sequence
  Description of Artificial Sequence: Synthetic polypeptide
RCRGSRDCLEPCRRAGTRFGRCISSRCHCYP

SEQ ID NO: 120 - protein, 31 aa, Artificial Sequence
  Description of Artificial Sequence: Synthetic polypeptide
RCRGSRDCLEPCRRAGTRFGRCITSRCHCYP

SEQ ID NO: 121 - protein, 31 aa, Artificial Sequence
  Description of Artificial Sequence: Synthetic polypeptide
KCRGSRDCLDPCRRAGMRFGRCINSRCHCTP

SEQ ID NO: 122 - protein, 31 aa, Artificial Sequence
  Description of Artificial Sequence: Synthetic polypeptide
RCRGSRDCLDPCKRAGMRFGRCINSRCHCTP

SEQ ID NO: 123 - protein, 31 aa, Artificial Sequence
  Description of Artificial Sequence: Synthetic polypeptide
RCRGSRDCLDPCRKAGMRFGRCINSRCHCTP

SEQ ID NO: 124 - protein, 31 aa, Artificial Sequence
  Description of Artificial Sequence: Synthetic polypeptide
RCRGSRDCLDPCRRAGMRFGKCINSRCHCTP

SEQ ID NO: 125 - protein, 31 aa, Artificial Sequence
  Description of Artificial Sequence: Synthetic polypeptide
RCRGSRDCLDPCRRAGMRFGRCINSKCHCTP

SEQ ID NO: 126 - protein, 31 aa, Artificial Sequence
  Description of Artificial Sequence: Synthetic polypeptide
RCRGSRDCLEPCRRAGTRFGRCINSRCHCYP

SEQ ID NO: 127 - protein, 38 aa, Artificial Sequence
  Description of Artificial Sequence: Synthetic polypeptide
SGVPINVRCRGSRDCLDPCRRAGMRFGRCINSRCHCTP

SEQ ID NO: 128 - protein, 37 aa, Artificial Sequence
  Description of Artificial Sequence: Synthetic polypeptide
GVPINVRCRGSRDCLDPCRRAGMRFGRCINSRCHCTP

SEQ ID NO: 129 - protein, 36 aa, Artificial Sequence
  Description of Artificial Sequence: Synthetic polypeptide
VPINVRCRGSRDCLDPCRRAGMRFGRCINSRCHCTP

SEQ ID NO: 130 - protein, 35 aa, Artificial Sequence
  Description of Artificial Sequence: Synthetic polypeptide
PINVRCRGSRDCLDPCRRAGMRFGRCINSRCHCTP

SEQ ID NO: 131 - protein, 34 aa, Artificial Sequence
  Description of Artificial Sequence: Synthetic polypeptide
INVRCRGSRDCLDPCRRAGMRFGRCINSRCHCTP

SEQ ID NO: 132 - protein, 33 aa, Artificial Sequence
  Description of Artificial Sequence: Synthetic polypeptide
NVRCRGSRDCLDPCRRAGMRFGRCINSRCHCTP

SEQ ID NO: 133 - protein, 32 aa, Artificial Sequence
  Description of Artificial Sequence: Synthetic polypeptide
VRCRGSRDCLDPCRRAGMRFGRCINSRCHCTP

SEQ ID NO: 134 - protein, 37 aa, Artificial Sequence
  Description of Artificial Sequence: Synthetic polypeptide
GVPISVRCRGSRDCLEPCRRAGTRFGRCINGRCHCTP

SEQ ID NO: 135 - protein, 37 aa, Artificial Sequence
  Description of Artificial Sequence: Synthetic polypeptide
GVPISVRCRGSRDCLEPCRRAGTRFGRCIQSRCHCTP

SEQ ID NO: 136 - protein, 37 aa, Artificial Sequence
  Description of Artificial Sequence: Synthetic polypeptide
GVPISVRCRGSRDCLEPCRRAGTRFGRCINRRCHCTP

SEQ ID NO: 137 - protein, 37 aa, Artificial Sequence
  Description of Artificial Sequence: Synthetic polypeptide
GVPINVRCRGSRDCLEPCRRAGTRFGRCINSRCHCTP

SEQ ID NO: 138 - protein, 37 aa, Artificial Sequence
  Description of Artificial Sequence: Synthetic polypeptide
GVPINVRCRGSRDCLEPCRRAGTRFGRCIQSRCHCTP

SEQ ID NO: 139 - protein, 37 aa, Artificial Sequence
  Description of Artificial Sequence: Synthetic polypeptide
GVPINVRCRGSRDCLEPCRRAGTRFGRCIQSRCHCYP

SEQ ID NO: 140 - protein, 37 aa, Artificial Sequence
  Description of Artificial Sequence: Synthetic polypeptide
GVPINVRCRGSRDCYEPCRRAGTRFGRCIQSRCHCTP

SEQ ID NO: 141 - protein, 37 aa, Artificial Sequence
  Description of Artificial Sequence: Synthetic polypeptide
GVPINVRCRGSRDCLEPCRRAGTRFGRCIQSRCYCTP

SEQ ID NO: 142 - protein, 37 aa, Artificial Sequence
  Description of Artificial Sequence: Synthetic polypeptide
GVPISVRCRGSRDCLEPCRRAGTRFGRCIQSRCHCYP

SEQ ID NO: 143 - protein, 37 aa, Artificial Sequence
  Description of Artificial Sequence: Synthetic polypeptide
GVPISVRCRGSRDCYEPCRRAGTRFGRCIQSRCHCTP

SEQ ID NO: 144 - protein, 37 aa, Artificial Sequence
  Description of Artificial Sequence: Synthetic polypeptide
GVPISVRCRGSRDCLEPCRRAGTRFGRCIQSRCYCTP

SEQ ID NO: 145 - protein, 37 aa, Artificial Sequence
  Description of Artificial Sequence: Synthetic polypeptide
GVPINVRCRGSRDCLEPCRRAGTRFGRCIASRCHCYP

SEQ ID NO: 146 - protein, 37 aa, Artificial Sequence
  Description of Artificial Sequence: Synthetic polypeptide
GVPINVRCRGSRDCLEPCRRAGTRFGRCISSRCHCYP

SEQ ID NO: 147 - protein, 37 aa, Artificial Sequence
  Description of Artificial Sequence: Synthetic polypeptide
GVPINVRCRGSRDCLEPCRRAGTRFGRCITSRCHCYP

SEQ ID NO: 148 - protein, 37 aa, Artificial Sequence
  Description of Artificial Sequence: Synthetic polypeptide
GVPINVRCRGSRDCLEPCRRAGTRFGRCINSRCHCYP

SEQ ID NO: 149 - protein, 39 aa, Artificial Sequence; identical to SEQ ID NO: 46
  Description of Artificial Sequence: Synthetic polypeptide

SEQ ID NO: 150 - protein, 39 aa, Artificial Sequence
  Description of Artificial Sequence: Synthetic polypeptide
GSGVPINVKCRGSRDCLDPCKKAGMRFGKCINSKCHCTP

SEQ ID NO: 151-197 - skipped (47 entries)

SEQ ID NO: 198 - protein, 37 aa, Artificial Sequence
  Description of Artificial Sequence: Synthetic polypeptide
VRIPVSCKHSGQCLKPCKDAGMRFGKCMNGKCDCTPK

SEQ ID NO: 199 - protein, 37 aa, Artificial Sequence
  Description of Artificial Sequence: Synthetic polypeptide
GVPINVKCRGSRDCLDPCKKAGMRFGKCINSKCHCTP

SEQ ID NO: 200 - protein, 37 aa, Artificial Sequence
  Description of Artificial Sequence: Synthetic polypeptide
EVIRCSGSKQCYGPCKQQTGCTNSKCMNKVCKCYGCG

SEQ ID NO: 201 - protein, 38 aa, Artificial Sequence
  Description of Artificial Sequence: Synthetic polypeptide
GVIINVKCKISRQCLEPCKKAGMRFGKCMNGKCHCTPK

SEQ ID NO: 202 - protein, 38 aa, Artificial Sequence
  Description of Artificial Sequence: Synthetic polypeptide
GVPTDVKCRGSPQCIQPCKDAGMRFGKCMNGKCHCTPK

SEQ ID NO: 203 - protein, 38 aa, Artificial Sequence
  Description of Artificial Sequence: Synthetic polypeptide
GVPINVSCTGSPQCIKPCKDAGMRFGKCMNRKCHCTPK

SEQ ID NO: 204 - protein, 37 aa, Artificial Sequence
  Description of Artificial Sequence: Synthetic polypeptide
VGINVKCKHSGQCLKPCKDAGMRFGKCINGKCDCTPK

SEQ ID NO: 205 - protein, 37 aa, Artificial Sequence; identical to SEQ ID NO: 128
  Description of Artificial Sequence: Synthetic polypeptide

SEQ ID NO: 206 - protein, 38 aa, Artificial Sequence
  Description of Artificial Sequence: Synthetic polypeptide
QKILSNRCNNSSECIPHCIRIFGTRAAKCINRKCYCYP

SEQ ID NO: 207 - protein, 37 aa, Artificial Sequence
  Description of Artificial Sequence: Synthetic polypeptide
VFINVKCRGSPECLPKCKEAIGKSAGKCMNGKCKCYP

SEQ ID NO: 208 - protein, 36 aa, Artificial Sequence
  Description of Artificial Sequence: Synthetic polypeptide
VPTDVKCRGSPQCIQPCKDAGMRFGKCMNGKCHCTP

SEQ ID NO: 209 - protein, 38 aa, Artificial Sequence
  Description of Artificial Sequence: Synthetic polypeptide
AEIIRCSGTRECYAPCQKLTGCLNAKCMNKACKCYGCV

SEQ ID NO: 210 - protein, 36 aa, Artificial Sequence
  Description of Artificial Sequence: Synthetic polypeptide
RPTDIKCSASYQCFPVCKSRFGKTNGRCVNGLCDCF

SEQ ID NO: 211 - protein, 37 aa, Artificial Sequence
  Description of Artificial Sequence: Synthetic polypeptide
QFTDVKCTGSKQCWPVCKQMFGKPNGKCMNGKCRCYS

SEQ ID NO: 212 - protein, 37 aa, Artificial Sequence
  Description of Artificial Sequence: Synthetic polypeptide
VGINVKCKHSRQCLKPCKDAGMRFGKCTNGKCHCTPK

SEQ ID NO: 213 - protein, 35 aa, Artificial Sequence
  Description of Artificial Sequence: Synthetic polypeptide
VVIGQRCYRSPDCYSACKKLVGKATGKCTNGRCDC

SEQ ID NO: 214 - protein, 35 aa, Artificial Sequence
  Description of Artificial Sequence: Synthetic polypeptide
NFKVEGACSKPCRKYCIDKGARNGKCINGRCHCYY

SEQ ID NO: 215 - protein, 38 aa, Artificial Sequence
  Description of Artificial Sequence: Synthetic polypeptide
QIDTNVKCSGSSKCVKICIDRYNTRGAKCINGRCTCYP

SEQ ID NO: 216 - protein, 292 aa, Artificial Sequence
  Description of Artificial Sequence: Synthetic polypeptide
METDTLLLWVLLLWVPGSTGGSGVPINVRCRGSRDCLDPCRRAGMRFGRCINSRCHCTPGGSGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 20 aa, Artificial Sequence
  Description of Artificial Sequence: Synthetic peptide
METDTLLLWVLLLWVPGSTG

SEQ ID NO: 218 - protein, 39 aa, Artificial Sequence
  Description of Artificial Sequence: Synthetic polypeptide
GSGVPINVRSRGSRDSLDPSRRAGMRFGRSINSRSHSTP

SEQ ID NO: 219 - protein, 38 aa, Artificial Sequence
  Description of Artificial Sequence: Synthetic polypeptide
  MOD_RES (1)..(1): Any amino acid or not present
  MOD_RES (3)..(3): Any amino acid or not present
  MOD_RES (5)..(5): Any amino acid or not present
  MOD_RES (7)..(7): Any amino acid or not present
  MOD_RES (9)..(9): Any amino acid or not present
  MOD_RES (12)..(13): Any amino acid or not present
  MOD_RES (15)..(16): Any amino acid or not present
  MOD_RES (19)..(20): Any amino acid or not present
  MOD_RES (22)..(22): Any amino acid or not present
  MOD_RES (24)..(24): Any amino acid or not present
  MOD_RES (26)..(26): Any amino acid or not present
  MOD_RES (28)..(28): Any amino acid or not present
  MOD_RES (30)..(33): Any amino acid or not present
  MOD_RES (35)..(35): Any amino acid or not present
  MOD_RES (37)..(37): Any amino acid or not present
  See specification as filed for detailed description of substitutions and preferred embodiments
XVXIXVXCXGSXXCXXPCXXAXGXRXGXCXXXXCXCXP

SEQ ID NO: 220 - protein, 40 aa, Artificial Sequence
  Description of Artificial Sequence: Synthetic polypeptide
  MOD_RES (3)..(3): Any amino acid or not present
  MOD_RES (5)..(5): Any amino acid or not present
  MOD_RES (7)..(7): Any amino acid or not present
  MOD_RES (9)..(9): Any amino acid or not present
  MOD_RES (11)..(11): Any amino acid or not present
  MOD_RES (14)..(15): Any amino acid or not present
  MOD_RES (17)..(18): Any amino acid or not present
  MOD_RES (21)..(22): Any amino acid or not present
  MOD_RES (24)..(24): Any amino acid or not present
  MOD_RES (26)..(26): Any amino acid or not present
  MOD_RES (28)..(28): Any amino acid or not present
  MOD_RES (30)..(30): Any amino acid or not present
  MOD_RES (32)..(35): Any amino acid or not present
  MOD_RES (37)..(37): Any amino acid or not present
  MOD_RES (39)..(39): Any amino acid or not present
  See specification as filed for detailed description of substitutions and preferred embodiments
GSXVXIXVXCXGSXXCXXPCXXAXGXRXGXCXXXXCXCXP

SEQ ID NO: 221 - protein, 32 aa, Artificial Sequence
  Description of Artificial Sequence: Synthetic polypeptide
  MOD_RES (1)..(1): Any amino acid or not present
  MOD_RES (3)..(3): Any amino acid or not present
  MOD_RES (6)..(7): Any amino acid or not present
  MOD_RES (9)..(10): Any amino acid or not present
  MOD_RES (13)..(14): Any amino acid or not present
  MOD_RES (16)..(16): Any amino acid or not present
  MOD_RES (18)..(18): Any amino acid or not present
  MOD_RES (20)..(20): Any amino acid or not present
  MOD_RES (22)..(22): Any amino acid or not present
  MOD_RES (24)..(27): Any amino acid or not present
  MOD_RES (29)..(29): Any amino acid or not present
  MOD_RES (31)..(31): Any amino acid or not present
  See specification as filed for detailed description of substitutions and preferred embodiments
XCXGSXXCXXPCXXAXGXRXGXCXXXXCXCXP

SEQ ID NO: 222 - protein, 34 aa, Artificial Sequence
  Description of Artificial Sequence: Synthetic polypeptide
  MOD_RES (3)..(3): Any amino acid or not present
  MOD_RES (5)..(5): Any amino acid or not present
  MOD_RES (8)..(9): Any amino acid or not present
  MOD_RES (11)..(12): Any amino acid or not present
  MOD_RES (15)..(16): Any amino acid or not present
  MOD_RES (18)..(18): Any amino acid or not present
  MOD_RES (20)..(20): Any amino acid or not present
  MOD_RES (22)..(22): Any amino acid or not present
  MOD_RES (24)..(24): Any amino acid or not present
  MOD_RES (26)..(29): Any amino acid or not present
  MOD_RES (31)..(31): Any amino acid or not present
  MOD_RES (33)..(33): Any amino acid or not present
  See specification as filed for detailed description of substitutions and preferred embodiments
GSXCXGSXXCXXPCXXAXGXRXGXCXXXXCXCXP

SEQ ID NO: 223 - protein, 32 aa, Artificial Sequence
  Description of Artificial Sequence: Synthetic polypeptide
  MOD_RES (9)..(9): L or Y
  MOD_RES (10)..(10): D or E
  MOD_RES (16)..(16): Any amino acid or not present
  MOD_RES (18)..(18): M or T
  MOD_RES (25)..(25): N, Q, A, S, T, or L
  MOD_RES (26)..(26): S, G, or R
  MOD_RES (29)..(29): H or Y
  MOD_RES (31)..(31): T or Y
KCRGSRQCXXPCKRAXGXRFGKCMXXKCXCXP

SEQ ID NO: 224 - protein, 34 aa, Artificial Sequence
  Description of Artificial Sequence: Synthetic polypeptide
  MOD_RES (11)..(11): L or Y
  MOD_RES (12)..(12): D or E
  MOD_RES (18)..(18): Any amino acid or not present
  MOD_RES (20)..(20): M or T
  MOD_RES (27)..(27): N, Q, A, S, T, or L
  MOD_RES (28)..(28): S, G, or R
  MOD_RES (31)..(31): H or Y
  MOD_RES (33)..(33): T or Y
GSKCRGSRQCXXPCKRAXGXRFGKCMXXKCXCXP

SEQ ID NO: 225 - protein, 38 aa, Artificial Sequence
  Description of Artificial Sequence: Synthetic polypeptide
  MOD_RES (1)..(1): Any amino acid or not present
  MOD_RES (3)..(3): Any amino acid or not present
  MOD_RES (5)..(5): Any amino acid or not present
  MOD_RES (15)..(15): L or Y
  MOD_RES (16)..(16): D or E
  MOD_RES (22)..(22): Any amino acid or not present
  MOD_RES (24)..(24): M or T
  MOD_RES (31)..(31): N, Q, A, S, T, or L
  MOD_RES (32)..(32): S, G, or R
  MOD_RES (35)..(35): H or Y
  MOD_RES (37)..(37): T or Y
XVXIXVKCRGSRQCXXPCKRAXGXRFGKCMXXKCXCXP

SEQ ID NO: 226 - protein, 40 aa, Artificial Sequence
  Description of Artificial Sequence: Synthetic polypeptide
  MOD_RES (3)..(3): Any amino acid or not present
  MOD_RES (5)..(5): Any amino acid or not present
  MOD_RES (7)..(7): Any amino acid or not present
  MOD_RES (17)..(17): L or Y
  MOD_RES (18)..(18): D or E
  MOD_RES (24)..(24): Any amino acid or not present
  MOD_RES (26)..(26): M or T
  MOD_RES (33)..(33): N, Q, A, S, T, or L
  MOD_RES (34)..(34): S, G, or R
  MOD_RES (37)..(37): H or Y
  MOD_RES (39)..(39): T or Y
GSXVXIXVKCRGSRQCXXPCKRAXGXRFGKCMXXKCXCXP

SEQ ID NO: 227 - protein, 8 aa, Artificial Sequence
  Description of Artificial Sequence: Synthetic peptide
GRCINSRC

SEQ ID NO: 228 - protein, 8 aa, Artificial Sequence
  Description of Artificial Sequence: Synthetic peptide
  MOD_RES (5)..(5): Any amino acid or amino acid analogue or null
  MOD_RES (6)..(6): Any amino acid or amino acid analogue or null
GRCIXXRC

SEQ ID NO: 229 - protein, 8 aa, Artificial Sequence
  Description of Artificial Sequence: Synthetic peptide
  MOD_RES (5)..(5): N, Q, A, S, T, or L
  MOD_RES (6)..(6): S, G, or R
GRCIXXRC

SEQ ID NO: 230 - protein, 3 aa, Artificial Sequence
  Description of Artificial Sequence: Synthetic peptide
PCR

SEQ ID NO: 231 - protein, 8 aa, Artificial Sequence
  Description of Artificial Sequence: Synthetic peptide
CLDPCRRA

SEQ ID NO: 232 - protein, 7 aa, Artificial Sequence
  Description of Artificial Sequence: Synthetic peptide
CLDPCRR

SEQ ID NO: 233 - protein, 31 aa, Artificial Sequence
  Description of Artificial Sequence: Synthetic polypeptide
RCRGSRDCLEPCRRAGTRFGRCILSRCHCTP

SEQ ID NO: 234 - protein, 31 aa, Artificial Sequence
  Description of Artificial Sequence: Synthetic polypeptide
RCRGSRDCLEPCRRAGTRFGRCILSRCHCYP

SEQ ID NO: 235 - protein, 31 aa, Artificial Sequence
  Description of Artificial Sequence: Synthetic polypeptide
RCRGSRDCLEPCRRAGTRFGRCILSRCYCTP

SEQ ID NO: 236 - protein, 31 aa, Artificial Sequence
  Description of Artificial Sequence: Synthetic polypeptide
RCRGSRDCLDPCRRAGMRFGRCIQSRCHCTP

SEQ ID NO: 237 - protein, 31 aa, Artificial Sequence
  Description of Artificial Sequence: Synthetic polypeptide
RCRGSRDCLDPCRRAGMRFGRCIASRCHCTP

SEQ ID NO: 238 - protein, 31 aa, Artificial Sequence
  Description of Artificial Sequence: Synthetic polypeptide
RCRGSRDCLDPCRRAGMRFGRCISSRCHCTP

SEQ ID NO: 239 - protein, 31 aa, Artificial Sequence
  Description of Artificial Sequence: Synthetic polypeptide
RCRGSRDCLDPCRRAGMRFGRCITSRCHCTP

SEQ ID NO: 240 - protein, 31 aa, Artificial Sequence
  Description of Artificial Sequence: Synthetic polypeptide
RCRGSRDCLDPCRRAGMRFGRCILSRCHCTP

SEQ ID NO: 241 - protein, 39 aa, Artificial Sequence
  Description of Artificial Sequence: Synthetic polypeptide
GSGVPINVRCRGSRDCLEPCRRAGTRFGRCILSRCHCTP

SEQ ID NO: 242 - protein, 39 aa, Artificial Sequence
  Description of Artificial Sequence: Synthetic polypeptide
GSGVPINVRCRGSRDCLEPCRRAGTRFGRCILSRCHCYP

SEQ ID NO: 243 - protein, 39 aa, Artificial Sequence
  Description of Artificial Sequence: Synthetic polypeptide
GSGVPINVRCRGSRDCLEPCRRAGTRFGRCILSRCYCTP

SEQ ID NO: 244 - protein, 39 aa, Artificial Sequence
  Description of Artificial Sequence: Synthetic polypeptide
GSGVPINVRCRGSRDCLDPCRRAGMRFGRCIQSRCHCTP

SEQ ID NO: 245 - protein, 39 aa, Artificial Sequence
  Description of Artificial Sequence: Synthetic polypeptide
GSGVPINVRCRGSRDCLDPCRRAGMRFGRCIASRCHCTP

SEQ ID NO: 246 - protein, 39 aa, Artificial Sequence
  Description of Artificial Sequence: Synthetic polypeptide
GSGVPINVRCRGSRDCLDPCRRAGMRFGRCISSRCHCTP

SEQ ID NO: 247 - protein, 39 aa, Artificial Sequence
  Description of Artificial Sequence: Synthetic polypeptide
GSGVPINVRCRGSRDCLDPCRRAGMRFGRCITSRCHCTP

SEQ ID NO: 248 - protein, 39 aa, Artificial Sequence
  Description of Artificial Sequence: Synthetic polypeptide
GSGVPINVRCRGSRDCLDPCRRAGMRFGRCILSRCHCTP

SEQ ID NO: 249 - protein, 37 aa, Artificial Sequence
  Description of Artificial Sequence: Synthetic polypeptide
GVPINVRCRGSRDCLEPCRRAGTRFGRCILSRCHCTP

SEQ ID NO: 250 - protein, 37 aa, Artificial Sequence
  Description of Artificial Sequence: Synthetic polypeptide
GVPINVRCRGSRDCLEPCRRAGTRFGRCILSRCHCYP

SEQ ID NO: 251 - protein, 37 aa, Artificial Sequence
  Description of Artificial Sequence: Synthetic polypeptide
GVPINVRCRGSRDCLEPCRRAGTRFGRCILSRCYCTP

SEQ ID NO: 252 - protein, 37 aa, Artificial Sequence
  Description of Artificial Sequence: Synthetic polypeptide
GVPINVRCRGSRDCLDPCRRAGMRFGRCIQSRCHCTP

SEQ ID NO: 253 - protein, 37 aa, Artificial Sequence
  Description of Artificial Sequence: Synthetic polypeptide
GVPINVRCRGSRDCLDPCRRAGMRFGRCIASRCHCTP

SEQ ID NO: 254 - protein, 37 aa, Artificial Sequence
  Description of Artificial Sequence: Synthetic polypeptide
GVPINVRCRGSRDCLDPCRRAGMRFGRCISSRCHCTP

SEQ ID NO: 255 - protein, 37 aa, Artificial Sequence
  Description of Artificial Sequence: Synthetic polypeptide
GVPINVRCRGSRDCLDPCRRAGMRFGRCITSRCHCTP

SEQ ID NO: 256 - protein, 37 aa, Artificial Sequence
  Description of Artificial Sequence: Synthetic polypeptide
GVPINVRCRGSRDCLDPCRRAGMRFGRCILSRCHCTP

SEQ ID NO: 257 - protein, 8 aa, Artificial Sequence
  Description of Artificial Sequence: Synthetic peptide
RCRGSRDC

SEQ ID NO: 258 - protein, 6 aa, Artificial Sequence
  Description of Artificial Sequence: Synthetic peptide
PCRRAG

SEQ ID NO: 259 - protein, 6 aa, Artificial Sequence
  Description of Artificial Sequence: Synthetic peptide
RFGRCI

SEQ ID NO: 260 - protein, 29 aa, Artificial Sequence
  Description of Artificial Sequence: Synthetic peptide
RCRGSRDCLDPCRRAGMRFGRCINSRCHC

SEQ ID NO: 261 - protein, 31 aa, Artificial Sequence
  Description of Artificial Sequence: Synthetic polypeptide
GSRCRGSRDCLDPCRRAGMRFGRCINSRCHC

SEQ ID NO: 262 - protein, 30 aa, Artificial Sequence
  Description of Artificial Sequence: Synthetic polypeptide
RCRGSRDCLDPCRRAGMRFGRCINSRCHCT

SEQ ID NO: 263 - protein, 32 aa, Artificial Sequence
  Description of Artificial Sequence: Synthetic polypeptide
GSRCRGSRDCLDPCRRAGMRFGRCINSRCHCT

SEQ ID NO: 264 - protein, 4 aa, Artificial Sequence
  Description of Artificial Sequence: Synthetic peptide
GAGA

SEQ ID NO: 265 - protein, 4 aa, Artificial Sequence
  Description of Artificial Sequence: Synthetic peptide
DEVD

SEQ ID NO: 266 - protein, 4 aa, Artificial Sequence
  Description of Artificial Sequence: Synthetic peptide
LEHD